SEQUENCE LISTING (141 sequences)

SEQ ID NO: 1 - protein, 45 aa, synthetic construct
DLAPQMLRELQETNAALQDVRELLRQQVKEITFLKNTVMECDACG

SEQ ID NO: 2 - protein, 45 aa, synthetic construct
DLGPQMLRELQETNAALQDVRELLRQQVKEITFLRNTVMECDACG

SEQ ID NO: 3 - protein, 498 aa, synthetic construct
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 4 - DNA, 1497 bp, synthetic construct
  source 1..1497: mol_type = other DNA
atgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 5 - protein, 243 aa, synthetic construct
DTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 6 - protein, 288 aa, synthetic construct
DLGPQMLRELQETNAALQDVRELLRQQVKEITFLRNTVMECDACGDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 7 - DNA, 867 bp, synthetic construct
  source 1..867: mol_type = other DNA
gatctgggcccgcagatgctgcgcgaactgcaggaaaccaacgcggcgctgcaggatgtgcgcgaactgctgcgccagcaggtgaaagaaattacctttctgcgcaacaccgtgatggaatgcgatgcgtgcggcgacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 8 - protein, 297 aa, synthetic construct
DYKDDDDKDLGPQMLRELQETNAALQDVRELLRQQVKEITFLRNTVMECDACGMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 9 - DNA, 891 bp, Synthetic construct
  source 1..891: mol_type = other DNA
gattataaagatgatgatgataaagatctgggcccgcagatgctgcgcgaactgcaggaaaccaacgcggcgctgcaggatgtgcgcgaactgctgcgccagcaggtgaaagaaattacctttctgcgcaacaccgtgatggaatgcgatgcgtgcggcgacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 10 - protein, 8 aa, synthetic construct
DYKDDDDK

SEQ ID NO: 11 - DNA, 24 bp, synthetic construct
  source 1..24: mol_type = other DNA
gattataaagatgatgatgataaa

SEQ ID NO: 12 - protein, 495 aa, synthetic construct
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 13 - DNA, 1803 bp, synthetic construct
  source 1..1803: mol_type = other DNA
ctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcagaagcagcaacatgatctcatggagacagttaataacttactgactatgatgtccacatcaaactctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggct

SEQ ID NO: 14 - skipped

SEQ ID NO: 15 - protein, 22 aa, synthetic construct
VKQTLNFDLLKLAGDVESNPGP

SEQ ID NO: 16 - protein, 25 aa, synthetic construct
RRKRKQTLNFDLLKLAGDVESNPGP

SEQ ID NO: 17-20 - skipped (4 entries)

SEQ ID NO: 21 - protein, 123 aa, synthetic construct [sequence omitted]

SEQ ID NO: 22 - protein, 112 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAA

SEQ ID NO: 23 - protein, 255 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAA

SEQ ID NO: 24 - protein, 467 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 204 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 26 - protein, 467 aa, synthetic construct; identical to SEQ ID NO: 24

SEQ ID NO: 27 - protein, 254 aa, synthetic construct
EVQLVESGGGLVQPGGSLRLSCAASGFTFSSYDIHWVRQATGKGLEWVSAIGPAGDTYYPGSVKGRFTISRENAKNSLYLQMNSLRAGDTAVYYCARGLITFGGLIAPFDYWGQGTLVTVSSGGGGSGGGGSGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQHYDNSQTFGQGTKVEIKRTVAA

SEQ ID NO: 28-30 - skipped (3 entries)

SEQ ID NO: 31 - protein, 431 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 231 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 33 - protein, 214 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 255 aa, Synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 35 - protein, 252 aa, synthetic construct
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 36-40 - skipped (5 entries)

SEQ ID NO: 41 - protein, 525 aa, Synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGGGGSGDLGPQMLRELQETNAALQDVRELLRQQVKEITFLRNTVMECDACGDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 42 - protein, 480 aa, Synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGGGGSGDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 43 - protein, 214 aa, synthetic construct; identical to SEQ ID NO: 33

SEQ ID NO: 44 - protein, 467 aa, synthetic construct; identical to SEQ ID NO: 24

SEQ ID NO: 45-50 - skipped (6 entries)

SEQ ID NO: 51 - DNA, 6934 bp, Synthetic construct
  source 1..6934: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgacctgggcccccagatgctgagggagctccaggagaccaatgctgctcttcaggatgttagggaactgctgaggcagcaggtgaaggagatcaccttcctcagaaacacagtgatggagtgtgatgcctgtggggacacagtccacaacctggtcaacctgtgcaccaaagagggtgtgctgctcaagggagggaagagggaggaggagaagcccttcagggactgtgctgatgtctaccaggctggcttcaacaagagtgggatctacaccatctacatcaacaacatgcctgagcccaagaaggtgttctgcaacatggatgtgaatggggggggctggactgtgatccagcacagagaagatggctccctggacttccagaggggctggaaggaatacaagatggggtttgggaacccctctggggagtactggctgggcaatgagttcatctttgccatcactagccagagacagtacatgctcagaattgagctgatggactgggagggcaacagagcctacagccaatatgacaggttccacattggaaatgaaaagcagaactacaggctgtacctgaagggccacactgggactgcaggcaagcagagctcactgatcctgcatggagctgacttctccaccaaggatgcagacaatgacaactgcatgtgcaagtgtgccctcatgctgactggtgggtggtggtttgatgcttgtgggcccagcaacctgaatggaatgttctacacagctgggcagaatcatggcaagctcaatggcatcaagtggcactacttcaagggccccagctacagcctgaggtccaccaccatgatgatcaggcctctggacttcgactacaaagacgatgacgacaagtgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 52 - DNA, 6934 bp, synthetic construct
  source 1..6934: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgacctgggcccccagatgctgagagagctgcaggagaccaacgccgccctgcaggacgtgagagagctgctgagacagcaggtgaaggagatcaccttcctgagaaacaccgtgatggagtgcgacgcctgcggcgacaccgtgcacaacctggtgaacctgtgcaccaaggagggcgtgctgctgaagggcggcaagagagaggaggagaagcccttcagagactgcgccgacgtgtaccaggccggcttcaacaagagcggcatctacaccatctacatcaacaacatgcccgagcccaagaaggtgttctgcaacatggacgtgaacggcggcggctggaccgtgatccagcacagagaggacggcagcctggacttccagagaggctggaaggagtacaagatgggcttcggcaaccccagcggcgagtactggctgggcaacgagttcatcttcgccatcaccagccagagacagtacatgctgagaatcgagctgatggactgggagggcaacagagcctacagccagtacgacagattccacatcggcaacgagaagcagaactacagactgtacctgaagggccacaccggcaccgccggcaagcagagcagcctgatcctgcacggcgccgacttcagcaccaaggacgccgacaacgacaactgcatgtgcaagtgcgccctgatgctgaccggcggctggtggttcgacgcctgcggccccagcaacctgaacggcatgttctacaccgccggccagaaccacggcaagctgaacggcatcaagtggcactacttcaagggccccagctacagcctgagaagcaccaccatgatgatcagacccctggacttcgactacaaggacgacgacgacaagtgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 53 - DNA, 8529 bp, synthetic construct
  source 1..8529: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggcatgctggggagagatcaaccgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgacctgggcccccagatgctgagggagctccaggagaccaatgctgctcttcaggatgttagggaactgctgaggcagcaggtgaaggagatcaccttcctcagaaacacagtgatggagtgtgatgcctgtggggacacagtccacaacctggtcaacctgtgcaccaaagagggtgtgctgctcaagggagggaagagggaggaggagaagcccttcagggactgtgctgatgtctaccaggctggcttcaacaagagtgggatctacaccatctacatcaacaacatgcctgagcccaagaaggtgttctgcaacatggatgtgaatggggggggctggactgtgatccagcacagagaagatggctccctggacttccagaggggctggaaggaatacaagatggggtttgggaacccctctggggagtactggctgggcaatgagttcatctttgccatcactagccagagacagtacatgctcagaattgagctgatggactgggagggcaacagagcctacagccaatatgacaggttccacattggaaatgaaaagcagaactacaggctgtacctgaagggccacactgggactgcaggcaagcagagctcactgatcctgcatggagctgacttctccaccaaggatgcagacaatgacaactgcatgtgcaagtgtgccctcatgctgactggtgggtggtggtttgatgcttgtgggcccagcaacctgaatggaatgttctacacagctgggcagaatcatggcaagctcaatggcatcaagtggcactacttcaagggccccagctacagcctgaggtccaccaccatgatgatcaggcctctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 54 - DNA, 7800 bp, synthetic construct
  source 1..7800: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagagaagaaagagagcccccgtgaagcagaccctgaacttcgacctgctgaagctggccggcgacgtggagagcaaccccggccccatggagttcggcctgagctggctgttcctggtggccatccttaagggcgtgcagtgcgacctgggcccccagatgctgagggagctccaggagaccaatgctgctcttcaggatgttagggaactgctgaggcagcaggtgaaggagatcaccttcctcagaaacacagtgatggagtgtgatgcctgtggggacacagtccacaacctggtcaacctgtgcaccaaagagggtgtgctgctcaagggagggaagagggaggaggagaagcccttcagggactgtgctgatgtctaccaggctggcttcaacaagagtgggatctacaccatctacatcaacaacatgcctgagcccaagaaggtgttctgcaacatggatgtgaatggggggggctggactgtgatccagcacagagaagatggctccctggacttccagaggggctggaaggaatacaagatggggtttgggaacccctctggggagtactggctgggcaatgagttcatctttgccatcactagccagagacagtacatgctcagaattgagctgatggactgggagggcaacagagcctacagccaatatgacaggttccacattggaaatgaaaagcagaactacaggctgtacctgaagggccacactgggactgcaggcaagcagagctcactgatcctgcatggagctgacttctccaccaaggatgcagacaatgacaactgcatgtgcaagtgtgccctcatgctgactggtgggtggtggtttgatgcttgtgggcccagcaacctgaatggaatgttctacacagctgggcagaatcatggcaagctcaatggcatcaagtggcactacttcaagggccccagctacagcctgaggtccaccaccatgatgatcaggcctctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 55 - DNA, 7891 bp, Synthetic construct
  source 1..7891: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgagatatctagagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggagttcggcctgagctggctgttcctggtggccatccttaagggcgtgcagtgcgacctgggcccccagatgctgagggagctccaggagaccaatgctgctcttcaggatgttagggaactgctgaggcagcaggtgaaggagatcaccttcctcagaaacacagtgatggagtgtgatgcctgtggggacacagtccacaacctggtcaacctgtgcaccaaagagggtgtgctgctcaagggagggaagagggaggaggagaagcccttcagggactgtgctgatgtctaccaggctggcttcaacaagagtgggatctacaccatctacatcaacaacatgcctgagcccaagaaggtgttctgcaacatggatgtgaatggggggggctggactgtgatccagcacagagaagatggctccctggacttccagaggggctggaaggaatacaagatggggtttgggaacccctctggggagtactggctgggcaatgagttcatctttgccatcactagccagagacagtacatgctcagaattgagctgatggactgggagggcaacagagcctacagccaatatgacaggttccacattggaaatgaaaagcagaactacaggctgtacctgaagggccacactgggactgcaggcaagcagagctcactgatcctgcatggagctgacttctccaccaaggatgcagacaatgacaactgcatgtgcaagtgtgccctcatgctgactggtgggtggtggtttgatgcttgtgggcccagcaacctgaatggaatgttctacacagctgggcagaatcatggcaagctcaatggcatcaagtggcactacttcaagggccccagctacagcctgaggtccaccaccatgatgatcaggcctctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 56 - DNA, 7719 bp, synthetic construct
  source 1..7719: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagggcggaggcggaagcggcggaggcggatctggcggaggcggcagcggcggcggcggctctgacctgggcccccagatgctgagggagctccaggagaccaatgctgctcttcaggatgttagggaactgctgaggcagcaggtgaaggagatcaccttcctcagaaacacagtgatggagtgtgatgcctgtggggacacagtccacaacctggtcaacctgtgcaccaaagagggtgtgctgctcaagggagggaagagggaggaggagaagcccttcagggactgtgctgatgtctaccaggctggcttcaacaagagtgggatctacaccatctacatcaacaacatgcctgagcccaagaaggtgttctgcaacatggatgtgaatggggggggctggactgtgatccagcacagagaagatggctccctggacttccagaggggctggaaggaatacaagatggggtttgggaacccctctggggagtactggctgggcaatgagttcatctttgccatcactagccagagacagtacatgctcagaattgagctgatggactgggagggcaacagagcctacagccaatatgacaggttccacattggaaatgaaaagcagaactacaggctgtacctgaagggccacactgggactgcaggcaagcagagctcactgatcctgcatggagctgacttctccaccaaggatgcagacaatgacaactgcatgtgcaagtgtgccctcatgctgactggtgggtggtggtttgatgcttgtgggcccagcaacctgaatggaatgttctacacagctgggcagaatcatggcaagctcaatggcatcaagtggcactacttcaagggccccagctacagcctgaggtccaccaccatgatgatcaggcctctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 57 - DNA, 7271 bp, synthetic construct
  source 1..7271: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttggttcaacggcattaaatatacctgacccatatatttaatgccgttgaacctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 58 - DNA, 7525 bp, synthetic construct
  source 1..7525: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcagaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttcgggggtggaggctctcaccatcaccaccatcatcaccatcaccactaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 59 - DNA, 7271 bp, synthetic construct
  source 1..7271: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttggaagcttgagaattataatacctgacccatattataattctcaagcttcctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 60 - DNA, 7271 bp, synthetic construct
  source 1..7271: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttgtgaagaactcaattataatacctgacccatattataattgagttcttcactttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 61 - DNA, 7271 bp, synthetic construct
  source 1..7271: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttgtaacattccctaattctatacctgacccatatagaattagggaatgttactttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 62 - DNA, 7271 bp, synthetic construct
  source 1..7271: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttgacttggaaagaatataaatacctgacccatatttatattctttccaagtctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 63 - DNA, 7271 bp, synthetic construct
  source 1..7271: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttggtgaagaactcaattatatacctgacccatatataattgagttcttcacctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 64 - DNA, 8529 bp, synthetic construct
  source 1..8529: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggcatgctggggagagatcaaccgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgacctgggcccccagatgctgagagagctgcaggagaccaacgccgccctgcaggacgtgagagagctgctgagacagcaggtgaaggagatcaccttcctgagaaacaccgtgatggagtgcgacgcctgcggcgacaccgtgcacaacctggtgaacctgtgcaccaaggagggcgtgctgctgaagggcggcaagagagaggaggagaagcccttcagagactgcgccgacgtgtaccaggccggcttcaacaagagcggcatctacaccatctacatcaacaacatgcccgagcccaagaaggtgttctgcaacatggacgtgaacggcggcggctggaccgtgatccagcacagagaggacggcagcctggacttccagagaggctggaaggagtacaagatgggcttcggcaaccccagcggcgagtactggctgggcaacgagttcatcttcgccatcaccagccagagacagtacatgctgagaatcgagctgatggactgggagggcaacagagcctacagccagtacgacagattccacatcggcaacgagaagcagaactacagactgtacctgaagggccacaccggcaccgccggcaagcagagcagcctgatcctgcacggcgccgacttcagcaccaaggacgccgacaacgacaactgcatgtgcaagtgcgccctgatgctgaccggcggctggtggttcgacgcctgcggccccagcaacctgaacggcatgttctacaccgccggccagaaccacggcaagctgaacggcatcaagtggcactacttcaagggccccagctacagcctgagaagcaccaccatgatgatcagacccctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 65 - DNA, 7800 bp, synthetic construct
  source 1..7800: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagagaagaaagagagcccccgtgaagcagaccctgaacttcgacctgctgaagctggccggcgacgtggagagcaaccccggccccatggagttcggcctgagctggctgttcctggtggccatccttaagggcgtgcagtgcgacctgggcccccagatgctgagagagctgcaggagaccaacgccgccctgcaggacgtgagagagctgctgagacagcaggtgaaggagatcaccttcctgagaaacaccgtgatggagtgcgacgcctgcggcgacaccgtgcacaacctggtgaacctgtgcaccaaggagggcgtgctgctgaagggcggcaagagagaggaggagaagcccttcagagactgcgccgacgtgtaccaggccggcttcaacaagagcggcatctacaccatctacatcaacaacatgcccgagcccaagaaggtgttctgcaacatggacgtgaacggcggcggctggaccgtgatccagcacagagaggacggcagcctggacttccagagaggctggaaggagtacaagatgggcttcggcaaccccagcggcgagtactggctgggcaacgagttcatcttcgccatcaccagccagagacagtacatgctgagaatcgagctgatggactgggagggcaacagagcctacagccagtacgacagattccacatcggcaacgagaagcagaactacagactgtacctgaagggccacaccggcaccgccggcaagcagagcagcctgatcctgcacggcgccgacttcagcaccaaggacgccgacaacgacaactgcatgtgcaagtgcgccctgatgctgaccggcggctggtggttcgacgcctgcggccccagcaacctgaacggcatgttctacaccgccggccagaaccacggcaagctgaacggcatcaagtggcactacttcaagggccccagctacagcctgagaagcaccaccatgatgatcagacccctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 66 - DNA, 7242 bp, synthetic construct
  source 1..7242: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgccagaagaaagagagcccccgtgaagcagaccctgaacttcgacctgctgaagctggccggcgacgtggagagcaaccccggccccatggagttcggcctgagctggctgttcctggtggccatccttaagggcgtgcagtgcgacctgggcccccagatgctgagagagctgcaggagaccaacgccgccctgcaggacgtgagagagctgctgagacagcaggtgaaggagatcaccttcctgagaaacaccgtgatggagtgcgacgcctgcggcgacaccgtgcacaacctggtgaacctgtgcaccaaggagggcgtgctgctgaagggcggcaagagagaggaggagaagcccttcagagactgcgccgacgtgtaccaggccggcttcaacaagagcggcatctacaccatctacatcaacaacatgcccgagcccaagaaggtgttctgcaacatggacgtgaacggcggcggctggaccgtgatccagcacagagaggacggcagcctggacttccagagaggctggaaggagtacaagatgggcttcggcaaccccagcggcgagtactggctgggcaacgagttcatcttcgccatcaccagccagagacagtacatgctgagaatcgagctgatggactgggagggcaacagagcctacagccagtacgacagattccacatcggcaacgagaagcagaactacagactgtacctgaagggccacaccggcaccgccggcaagcagagcagcctgatcctgcacggcgccgacttcagcaccaaggacgccgacaacgacaactgcatgtgcaagtgcgccctgatgctgaccggcggctggtggttcgacgcctgcggccccagcaacctgaacggcatgttctacaccgccggccagaaccacggcaagctgaacggcatcaagtggcactacttcaagggccccagctacagcctgagaagcaccaccatgatgatcagacccctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 67 - DNA, 7944 bp, synthetic construct
  source 1..7944: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaaataaaatatctttattttcattacatctgtgtgttggttttttgtgtgggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgacctgggcccccagatgctgagagagctgcaggagaccaacgccgccctgcaggacgtgagagagctgctgagacagcaggtgaaggagatcaccttcctgagaaacaccgtgatggagtgcgacgcctgcggcgacaccgtgcacaacctggtgaacctgtgcaccaaggagggcgtgctgctgaagggcggcaagagagaggaggagaagcccttcagagactgcgccgacgtgtaccaggccggcttcaacaagagcggcatctacaccatctacatcaacaacatgcccgagcccaagaaggtgttctgcaacatggacgtgaacggcggcggctggaccgtgatccagcacagagaggacggcagcctggacttccagagaggctggaaggagtacaagatgggcttcggcaaccccagcggcgagtactggctgggcaacgagttcatcttcgccatcaccagccagagacagtacatgctgagaatcgagctgatggactgggagggcaacagagcctacagccagtacgacagattccacatcggcaacgagaagcagaactacagactgtacctgaagggccacaccggcaccgccggcaagcagagcagcctgatcctgcacggcgccgacttcagcaccaaggacgccgacaacgacaactgcatgtgcaagtgcgccctgatgctgaccggcggctggtggttcgacgcctgcggccccagcaacctgaacggcatgttctacaccgccggccagaaccacggcaagctgaacggcatcaagtggcactacttcaagggccccagctacagcctgagaagcaccaccatgatgatcagacccctggacttctgagcgggactctggaattcgaaaataaaatatctttattttcattacatctgtgtgttggttttttgtgtgttctgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 68 - DNA, 6714 bp, synthetic construct
  source 1..6714: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttggttcaacggcattaaatatacctgacccatatatttaatgccgttgaacctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 69 - DNA, 6714 bp, synthetic construct
  source 1..6714: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttggaagcttgagaattataatacctgacccatattataattctcaagcttcctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 70 - DNA, 6713 bp, synthetic construct
  source 1..6713: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttgtgaagaactcaattataatacctgacccatattataattgagttcttcactttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 71 - DNA, 6713 bp, synthetic construct
  source 1..6713: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttgtaacattccctaattctatacctgacccatatagaattagggaatgttactttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 72 - DNA, 6708 bp, synthetic construct
  source 1..6708: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgataataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttgacttggaaagaatataaatacctgacccatatttatattctttccaagtctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 73 - DNA, 6708 bp, synthetic construct
  source 1..6708: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgataataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttggtgaagaactcaattatatacctgacccatatataattgagttcttcacctttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 74 - DNA, 6713 bp, synthetic construct
  source 1..6713: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaataaaatatctttattttcattacatctgtgtgttggttttttgtgtggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacaagcttgtgcatatgaacgtaactatacctgacccatatagttacgttcatatgcactttttgcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 75 - DNA, 8415 bp, synthetic construct
  source 1..8415: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagtgattcgaaaataaaatatctttattttcattacatctgtgtgttggttttttgtgtggcatgctggggagagatcaaccgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgaggtgcagctggtggagagcggaggcggtctggtgcagccaggcaggagcctgaggctgagctgcgccgccagcggcttcaccttcgacgactacgccatgcactgggtgaggcaggccccaggcaagggcctggagtgggtgagcgccatcacctggaacagcggtcacatcgactacgccgacagcgtggagggtaggttcaccatcagcagggacaacgccaagaacagcctgtacctgcagatgaacagcctgagggccgaggacaccgccgtgtactactgcgccaaggtgagctacctgagcaccgccagcagcctggactactggggtcagggcaccctggtgaccgtgagcagcggtggaggaggtagcggtggcggtggtagcggtggcggaggcagcggtggaggtggcagcgacatccagatgacccagagccctagcagcctgagcgctagcgtgggtgacagggtgaccatcacctgcagggccagccagggcatcaggaactacctggcctggtaccagcagaagcccggcaaggcccccaagctgctgatctacgccgccagcaccctgcagagcggcgtgcccagcaggttcagcggcagcggcagcggcaccgacttcaccctgaccatcagcagcctgcagcccgaggacgtggccacctactactgccagaggtacaacagggccccctacaccttcggccagggcaccaaggtggagatcaagaggtgattcgaaaataaaatatctttattttcattacatctgtgtgttggttttttgtgtggcatgctggggagagatcaacccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 76 - DNA, 7830 bp, synthetic construct
  source 1..7830: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagcttcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttgacattgattattgactaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatcctgaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaaataaaatatctttattttcattacatctgtgtgttggttttttgtgtgggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgaggtgcagctggtggagagcggaggcggtctggtgcagccaggcaggagcctgaggctgagctgcgccgccagcggcttcaccttcgacgactacgccatgcactgggtgaggcaggccccaggcaagggcctggagtgggtgagcgccatcacctggaacagcggtcacatcgactacgccgacagcgtggagggtaggttcaccatcagcagggacaacgccaagaacagcctgtacctgcagatgaacagcctgagggccgaggacaccgccgtgtactactgcgccaaggtgagctacctgagcaccgccagcagcctggactactggggtcagggcaccctggtgaccgtgagcagcggtggaggaggtagcggtggcggtggtagcggtggcggaggcagcggtggaggtggcagcgacatccagatgacccagagccctagcagcctgagcgctagcgtgggtgacagggtgaccatcacctgcagggccagccagggcatcaggaactacctggcctggtaccagcagaagcccggcaaggcccccaagctgctgatctacgccgccagcaccctgcagagcggcgtgcccagcaggttcagcggcagcggcagcggcaccgacttcaccctgaccatcagcagcctgcagcccgaggacgtggccacctactactgccagaggtacaacagggccccctacaccttcggccagggcaccaaggtggagatcaagaggtgattcgaaaataaaatatctttattttcattacatctgtgtgttggttttttgtgtggcatgctggggagagatcaacccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 77 - DNA, 6659 bp, synthetic construct
  source 1..6659: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctggcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagagttaactggtaagtttagtctttttgtcttttatttcaggtcccggatccggtggtggtgcaaatcaaagaactgctcctcagtggatgttgcctttacttctaggcctgccgccaccatggagttcggcctgagctggctgttcctggtggccatccttaagggcgtgcagtgcgagcaccccaacgcgcgcaaatacaaaggagccaacaagaagggcttgtccaagggctgcttcggcctcaagctggaccgaatcggctccatgagcggcctgggatgtagaagaaagagagcccccgtgaagcagaccctgaacttcgacctgctgaagctggccggcgacgtggagagcaaccccggccccatggagttcggcctgagctggctgttcctggtggccatccttaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgcctgattcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 78-80 - skipped (3 entries)

SEQ ID NO: 81 - RNA, 56 nt, synthetic construct
  source 1..56: mol_type = other RNA
ggttcaacggcattaaatatacctgacccatatatttaatgccgttgaaccttttt

SEQ ID NO: 82 - RNA, 56 nt, synthetic construct
  source 1..56: mol_type = other RNA
ggaagcttgagaattataatacctgacccatattataattctcaagcttccttttt

SEQ ID NO: 83 - RNA, 56 nt, synthetic construct
  source 1..56: mol_type = other RNA
gtgaagaactcaattataatacctgacccatattataattgagttcttcacttttt

SEQ ID NO: 84 - RNA, 56 nt, synthetic construct
  source 1..56: mol_type = other RNA
gtaacattccctaattctatacctgacccatatagaattagggaatgttacttttt

SEQ ID NO: 85 - RNA, 56 nt, synthetic construct
  source 1..56: mol_type = other RNA
gacttggaaagaatataaatacctgacccatatttatattctttccaagtcttttt

SEQ ID NO: 86 - RNA, 56 nt, synthetic construct
  source 1..56: mol_type = other RNA
ggtgaagaactcaattatatacctgacccatatataattgagttcttcaccttttt

SEQ ID NO: 87 - RNA, 56 nt, synthetic construct
  source 1..56: mol_type = other RNA
gtgcatatgaacgtaactatacctgacccatatagttacgttcatatgcacttttt

SEQ ID NO: 88-100 - skipped (13 entries)

SEQ ID NO: 101 - DNA, 49 bp, synthetic construct
  source 1..49: mol_type = other DNA
tcctggtggccatccttaagggcgtgcagtgcgacctgggcccccagat

SEQ ID NO: 102 - DNA, 40 bp, synthetic construct
  source 1..40: mol_type = other DNA
tcgtcatcgtctttgtagtcgaagtccagaggcctgatca

SEQ ID NO: 103 - DNA, 46 bp, synthetic construct
  source 1..46: mol_type = other DNA
atccagaggttgattctcgagtcacttgtcgtcatcgtctttgtag

SEQ ID NO: 104 - DNA, 49 bp, synthetic construct
  source 1..49: mol_type = other DNA
gtaatccagaggttgattctcgagtcacttgtcgtcgtcgtccttgtag

SEQ ID NO: 105 - DNA, 25 bp, synthetic construct
  source 1..25: mol_type = other DNA
attgactaggaagctgatctgaatt

SEQ ID NO: 106 - DNA, 49 bp, synthetic construct
  source 1..49: mol_type = other DNA
gtaagttatgtaacgggtaccgaattcggttgatctctccccagcatgc

SEQ ID NO: 107 - DNA, 79 bp, synthetic construct
  source 1..79: mol_type = other DNA
ctgtccctgtcccccggcaagagaagaaagagagcccccgtgaagcagaccctgaacttcgacctgctgaagctggccg

SEQ ID NO: 108 - DNA, 67 bp, synthetic construct
  source 1..67: mol_type = other DNA
cgacctgctgaagctggccggcgacgtggagagcaaccccggccccatggagttcggcctgagctgg

SEQ ID NO: 109 - DNA, 30 bp, synthetic construct
  source 1..30: mol_type = other DNA
cagatgtaatgaaaataaagatattttatt

SEQ ID NO: 110 - DNA, 47 bp, synthetic construct
  source 1..47: mol_type = other DNA
ctgtccctgtcccccggcaagtgagatatctagagcgcagaggcttg

SEQ ID NO: 111 - DNA, 40 bp, synthetic construct
  source 1..40: mol_type = other DNA
cagctcaggccgaactccatggtttcggaggccgtccggg

SEQ ID NO: 112 - DNA, 70 bp, synthetic construct
  source 1..70: mol_type = other DNA
ctgtccctgtcccccggcaagggcggaggcggaagcggcggaggcggatctggcggaggcggcagcggcg

SEQ ID NO: 113 - DNA, 59 bp, synthetic construct
  source 1..59: mol_type = other DNA
ggatctggcggaggcggcagcggcggcggcggctctgacctgggcccccagatgctgag

SEQ ID NO: 114 - DNA, 40 bp, synthetic construct
  source 1..40: mol_type = other DNA
gtgtgttggttttttgtgtggagggcctatttcccatgat

SEQ ID NO: 115 - DNA, 99 bp, synthetic construct
  source 1..99: mol_type = other DNA
tgatctctccccagcatgcaaaaaggttcaacggcattaaatatatgggtcaggtatatttaatgccgttgaaccaagcttgtcctttccacaagatat

SEQ ID NO: 116 - DNA, 66 bp, synthetic construct
  source 1..66: mol_type = other DNA
agcttggaagcttgagaattataatacctgacccatattataattctcaagcttcctttttgcatg

SEQ ID NO: 117 - DNA, 58 bp, synthetic construct
  source 1..58: mol_type = other DNA
caaaaaggaagcttgagaattataatatgggtcaggtattataattctcaagcttcca

SEQ ID NO: 118 - DNA, 66 bp, synthetic construct
  source 1..66: mol_type = other DNA
agcttgtgaagaactcaattataatacctgacccatattataattgagttcttcactttttgcatg

SEQ ID NO: 119 - DNA, 58 bp, synthetic construct
  source 1..58: mol_type = other DNA
caaaaagtgaagaactcaattataatatgggtcaggtattataattgagttcttcaca

SEQ ID NO: 120 - DNA, 66 bp, synthetic construct
  source 1..66: mol_type = other DNA
agcttgtgcatatgaacgtaactatacctgacccatatagttacgttcatatgcactttttgcatg

SEQ ID NO: 121 - DNA, 58 bp, synthetic construct
  source 1..58: mol_type = other DNA
caaaaagtgcatatgaacgtaactatatgggtcaggtatagttacgttcatatgcaca

SEQ ID NO: 122 - DNA, 66 bp, synthetic construct
  source 1..66: mol_type = other DNA
agcttgtaacattccctaattctatacctgacccatatagaattagggaatgttactttttgcatg

SEQ ID NO: 123 - DNA, 58 bp, synthetic construct
  source 1..58: mol_type = other DNA
caaaaagtaacattccctaattctatatgggtcaggtatagaattagggaatgttaca

SEQ ID NO: 124 - DNA, 66 bp, synthetic construct
  source 1..66: mol_type = other DNA
agcttgacttggaaagaatataaatacctgacccatatttatattctttccaagtctttttgcatg

SEQ ID NO: 125 - DNA, 58 bp, synthetic construct
  source 1..58: mol_type = other DNA
caaaaagacttggaaagaatataaatatgggtcaggtatttatattctttccaagtca

SEQ ID NO: 126 - DNA, 66 bp, synthetic construct
  source 1..66: mol_type = other DNA
agcttggtgaagaactcaattatatacctgacccatatataattgagttcttcacctttttgcatg

SEQ ID NO: 127 - DNA, 58 bp, synthetic construct
  source 1..58: mol_type = other DNA
caaaaaggtgaagaactcaattatatatgggtcaggtatataattgagttcttcacca

SEQ ID NO: 128 - DNA, 59 bp, synthetic construct
  source 1..59: mol_type = other DNA
gaaaataaagatattttattttcgaattccagagtcccgctcagaagtccaggggtctg

SEQ ID NO: 129 - DNA, 74 bp, synthetic construct
  source 1..74: mol_type = other DNA
gcggccgctcggtccgcacgtgcagaacacacaaaaaaccaacacacagatgtaatgaaaataaagatatttta

SEQ ID NO: 130 - DNA, 58 bp, synthetic construct
  source 1..58: mol_type = other DNA
aggcggatctggcggaggcggcagcggcggcggcggctctgacatccagctgacccag

SEQ ID NO: 131 - DNA, 49 bp, synthetic construct
  source 1..49: mol_type = other DNA
gaaaataaagatattttattttcgaatcaggcggccacggttctcttga

SEQ ID NO: 132 - DNA, 59 bp, synthetic construct
  source 1..59: mol_type = other DNA
agggcaccctggtgaccgtgggcggaggcggaagcggcggaggcggatctggcggaggc

SEQ ID NO: 133 - DNA, 20 bp, synthetic construct
  source 1..20: mol_type = other DNA
cacggtcaccagggtgccct

SEQ ID NO: 134 - DNA, 45 bp, synthetic construct
  source 1..45: mol_type = other DNA
aggaagctgatctgaattcggtacccgttacataacttacggtaa

SEQ ID NO: 135 - DNA, 45 bp, synthetic construct
  source 1..45: mol_type = other DNA
taccgtaagttatgtaacgggtacccacacaaaaaaccaacacac

SEQ ID NO: 136 - DNA, 45 bp, synthetic construct
  source 1..45: mol_type = other DNA
gctgttcctggtggccatccttaagggcgtgcagtgcgaggtgca

SEQ ID NO: 137 - DNA, 45 bp, synthetic construct
  source 1..45: mol_type = other DNA
gcggccgctcggtccgcacgtgggttgatctctccccagcatgcc

SEQ ID NO: 138 - DNA, 49 bp, synthetic construct
  source 1..49: mol_type = other DNA
gaatcggctccatgagcggcctgggatgtagaagaaagagagcccccgt

SEQ ID NO: 139 - protein, 129 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 140 - protein, 110 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSS

SEQ ID NO: 141 - protein, 253 aa, synthetic construct
EVQLVESGGGLVQPGGSLRLSCAASGFTFSSYDIHWVRQATGKGLEWVSAIGPAGDTYYPGSVKGRFTISRENAKNSLYLQMNSLRAGDTAVYYCARGLITFGGLIAPFYWGQGTLVTVSSGGGGSGGGGSGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQHYDNSQTFGQGTKVEIKRTVAA

(19) United States
(12) Patent Application Publication
(10) Pub. No.: US 2024/0269327 A1
(43) Pub. Date: Aug. 15, 2024

(54) COMPOSITIONS AND METHODS FOR TRANSGENE EXPRESSION

(71) Applicant: Avirmax Biopharma Inc., Hayward, CA (US)

(72) Inventor: Shengjiang LIU, Lafayette, CA (US)

(21) Appl. No.: 18/584,397

(22) Filed: Feb. 22, 2024

Related U.S. Application Data
(63) Continuation of application No. PCT/US2022/041084, filed on Aug. 22, 2022.
(60) Provisional application No. 63/236,168, filed on Aug. 23, 2021.

(51) Int. Cl. A61K 48/00; A61K 38/17; C07K 16/22; C07K 16/40; C12N 15/113; C12N 15/86

(52) U.S. Cl. CPC A61K 48/005; A61K 38/1709; C07K 16/22; C07K 16/40; C12N 15/1138; C12N 15/86; C12N 2310/14; C12N 2750/14143

(57) ABSTRACT

Described herein are compositions for modulating transgene expression. Also described herein are methods for using the compositions described herein for modulating transgene expression.

### CROSS-REFERENCE

This application is a continuation of International Patent Application No. PCT/US2022/041084, filed Aug. 22, 2022, which claims the benefit of U.S. Provisional Application No. 63/236,168, filed Aug. 23, 2021, each of which is herein incorporated by reference in its entirety.

### SEQUENCE LISTING

The instant application contains a Sequence Listing which has been submitted electronically in XML format and is herein incorporated by reference in its entirety. Said XML copy, created on Feb. 21, 2024, is named 59561_704_301_SL.xml, and is 315,392 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Neovascularization, including vasculogenesis, angiogenesis, and arteriogenesis, is regulated by a wide variety of cell signaling pathways. One of the signaling pathways is regulated by vascular endothelium growth factors (VEGFs). There are 4 major types of VEGF including VEGF-A, VEGF-B, VEGF-C, and VEGF-D. There are many isoforms of VEGF-A that result from alternative splicing of mRNA from the VEGF-A, including VEGF121, VEGF145, VEGF148, VEGF162, VEGF165, VEGF165b, VEGF183, VEGF189, and VEGF206. VEGFs are strong mitogens for endothelial cells, inducing proliferation, migration, blood vessel tubing formation, and permeability. As such, increase in VEGF signaling transduction pathway increases neovascularization signal, while decrease or inhibition of VEGF signaling transduction pathway decreases neovascularization signal.

VEGF inhibition is one of the most popular treatment options for disease or condition related to neovascularization. Current treatments employing VEGF inhibitors can be cumbersome due to the short half-life of the VEGF inhibitor, which leads to the need for repeated monthly injections for achieving and sustaining suppression of neovascularization. Therefore, it has become increasingly clear that the full potential of VEGF inhibition can only be realized with augmentation of the therapeutic effect of VEGF inhibition.

### SUMMARY

There remains a need for a biological product to modulate signaling transduction in the ligand and receptor interaction associated with neovascularization, thus complementing or leading to synergistic therapeutic effect when combined the VEGF inhibition. Accordingly, described herein is a non-naturally occurring polynucleotide comprising one or more expression cassettes encoding a VEGF inhibitor and a signaling transduction regulator (e.g., an activator of a receptor tyrosine kinase that is associated with VEGF signaling) that augments and complements the therapeutic effect of VEGF inhibition. Such combination can synergistically increase the therapeutic effects of VEGF inhibition and decrease neovascularization signaling.

Described herein, in some aspects, is a non-naturally occurring polynucleotide comprising one or more expression cassettes for expressing: a VEGF inhibitor; and a receptor tyrosine kinase (RTK)/Tie2 or an activator of RTK/Tie2. In some embodiments, the VEGF inhibitor and the RTK/Tie or the activator of RTK/Tie2 are expressed as separate polypeptides or as a contiguous polypeptide cleavable into separate polypeptides comprising the VEGF inhibitor, and the RTK/Tie2 or the activator of RTK/Tie2. In some embodiments, the contiguous polypeptide comprises a protease cleavable sequence. In some embodiments, the contiguous polypeptide comprises a Furin cleavable sequence. In some embodiments, the contiguous polypeptide comprises a self-cleaving polypeptide sequence. In some embodiments, the self-cleaving polypeptide sequence comprises a 2A self-cleaving peptide. In some embodiments, the self-cleaving polypeptide sequence comprises a F2A self-cleaving peptide. In some embodiments, the protease cleavable sequence comprises a Furin-F2A cleavage sequence. In some embodiments, the VEGF inhibitor binds to and inhibits VEGF-A, VEGF-B, VEGF-C, VEGF-D, or a combination thereof. In some embodiments, the VEGF inhibitor comprises an antibody. In some embodiments, the VEGF inhibitor comprises a monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof. In some embodiments, the VEGF inhibitor comprises a non-antibody VEGF inhibitor. In some embodiments, the non-antibody VEGF inhibitor is a VEGF receptor 1 (VEGFR1), a VEGF receptor 2 (VEGFR2), a VEGF receptor 3 (VEGFR3), a fragment thereof, or a combination thereof. In some embodiments, the non-antibody VEGF inhibitor comprises a soluble VEGFR1, a soluble VEGFR2, a soluble VEGFR3, a soluble fragment thereof, or a combination thereof. In some embodiments, the non-antibody VEGF inhibitor comprises a VEGF-Trap or a modified version thereof. In some embodiments, the activator of the RTK/Tie2 comprises a angiopoietin-1 (Ang-1), angiopoietin-2 (Ang-2), angiopoietin-3 (Ang-3), or angiopoietin-4 (Ang-4). In some embodiments, the activator of the RTK/Tie2 comprises Ang1. In some embodiments, the Ang1 comprises a full length Ang1. In some embodiments, the Ang1 comprises a polypeptide sequence that is at least 70%, at least 75%, at least 80%, at least 85%, at least 90%, at least 95%, or at least 99% identical to SEQ ID NO: 3. In some embodiments, the Ang1 comprises a functional fragment of Ang1. In some embodiments, the functional fragment of the Ang1 comprises a fibronectin-like domain (FLD). In some embodiments, the FLD comprises a polypeptide sequence that is at least 70%, at least 75%, at least 80%, at least 85%, at least 90%, at least 95%, or at least 99% identical to SEQ ID NO: 5. In some embodiments, the FLD is fused to a soluble polypeptide. In some embodiments, the soluble polypeptide comprises a polypeptide sequence that is at most 99%, at most 98%, at most 97%, at most 96%, at most 95%, at most 94%, or at most 93% identical to SEQ ID NO: 1. In some embodiments, the soluble polypeptide comprises a polypeptide sequence that is at least 70%, at least 75%, at least 80%, at least 85%, at least 90%, at least 95%, at least 99%, or more identical to SEQ ID NO: 2. In some embodiments, the soluble polypeptide comprises a polypeptide sequence that is SEQ ID NO: 2. In some embodiments, the activator of the RTK/Tie2 is at least 70%, at least 75%, at least 80%, at least 85%, at least 90%, at least 95%, or at least 99% identical to SEQ ID NO: 6. In some embodiments, the activator of the RTK/Tie2 comprises an antibody or a fragment thereof. In some embodiments, the activator of the RTK/Tie2 comprises a monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof. In some embodiments, the activator of the RTK/Tie2 binds to and inhibits Ang2. In some embodiments, the antibody or the fragment thereof binds to a polypeptide that is at least 70%, at least 75%, at least 80%, at least 85%, at least 90%, at least 95%, or at least 99% identical to SEQ ID NO: 12. In some embodiments, the antibody or the fragment thereof comprises a polypeptide sequence that is at least 70%, at least 75%, at least 80%, at least 85%, at least 90%, at least 95%, or at least 99% identical to any one of SEQ ID NOs: 139-141, a fragment thereof, or a combination thereof. In some embodiments, the activator of the RTK/Tie2 comprises an inhibitory RNA. In some embodiments, the inhibitory RNA comprises a shRNA, siRNA, miRNA, or a combination thereof. In some embodiments, the inhibitory RNA comprises shRNA. In some embodiments, the inhibitory RNA binds to an endogenous nucleic acid encoding an angiopoietin. In some embodiments, the angiopoietin comprises Ang2. In some embodiments, the Ang2 comprises a nucleic acid sequence that is at least 70%, at least 75%, at least 80%, at least 85%, at least 90%, at least 95%, or at least 99% identical to SEQ ID NO: 13. In some embodiments, the one or more expression cassettes comprise one or more promoters, one or more internal ribosome entry sites (IRES), or both. In some embodiments, the VEGF inhibitor and the activator of the RTK/Tie2, or the RTK/Tie2 decrease neovascularization signaling when expressed in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling in absence of the VEGF inhibitor and the activator of the RTK/Tie2 or the RTK/Tie2. In some embodiments, the VEGF inhibitor and the activator of the RTK/Tie2 or the RTK/TIE2 decrease neovascularization signaling when expressed in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling decreased by a comparable VEGF inhibitor and a comparable activator of a RTK/Tie2 or a comparable RTK/Tie2 encoded from two different non-naturally occurring polynucleotides.

Described herein, in some aspects, is a non-naturally occurring polynucleotide comprising one or more expression cassettes for expressing: a VEGF inhibitor; and an Ang1 polypeptide. Also descried herein, in certain aspects, is a non-naturally occurring polynucleotide comprising one or more expression cassettes for expressing: an VEGF inhibitor; and an Ang2 inhibitor. In some embodiments, the VEGF inhibitor binds to and inhibits VEGF-A, VEGF-B, VEGF-C, VEGF-D, or a combination thereof. In some embodiments, the VEGF inhibitor comprises an antibody. In some embodiments, the VEGF inhibitor comprises a monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof. In some embodiments, the VEGF inhibitor comprises an amino acid sequence that is at least 70%, at least 75%, at least 80%, at least 85%, at least 90%, at least 95%, or at least 99% identical to SEQ ID NO: 21, SEQ ID NO: 22, SEQ ID NO: 23, SEQ ID NO: 32, SEQ ID NO: 33, SEQ ID NO: 34, SEQ ID NO: 35, or a combination thereof, or a fragment thereof. In some embodiments, the VEGF inhibitor comprises a non-antibody VEGF inhibitor. In some embodiments, the non-antibody VEGF inhibitor comprises a VEGF receptor 1, a VEGF receptor 2, a VEGF receptor 3, a fragment thereof, or a combination thereof. In some embodiments, the non-antibody VEGF inhibitor comprises a soluble VEGFR1, a soluble VEGFR2, a soluble VEGFR3, a soluble fragment thereof, or a combination thereof. In some embodiments, the non-antibody VEGF inhibitor comprises a VEGF-Trap or a modified version thereof. In some embodiments, the non-antibody VEGF inhibitor comprises an amino acid sequence that is at least 70%, at least 75%, at least 80%, at least 85%, at least 90%, at least 95%, or at least 99% identical to SEQ ID NO: 24, SEQ ID NO: 25, SEQ ID NO: 26, or SEQ ID NO: 31, or a combination thereof, or a fragment thereof. In some embodiments, the Ang1 polypeptide is a full length Ang1. In some embodiments, the Ang1 polypeptide is at least 70%, at least 75%, at least 80%, at least 85%, at least 90%, at least 95%, at least 99%, or 100% identical to SEQ ID NO: 3. In some embodiments, the Ang1 polypeptide comprises an Ang1 functional fragment comprising a fibronectin-like domain (FLD) of Ang1. In some embodiments, the Ang1 polypeptide is at least 70%, at least 75%, at least 80%, at least 85%, at least 90%, at least 95%, at least 99%, or 100% identical to SEQ ID NO: 5. In some embodiments, the FLD is fused to a soluble polypeptide comprising a polypeptide sequence that is at most 99%, at most 98%, at most 96%, at most 95%, at most 94%, or at most 93% identical to SEQ ID NO: 6. In some embodiments, the Ang2 inhibitor comprises an antibody or a fragment thereof that binds to and inhibits Ang2. In some embodiments, the Ang2 inhibitor comprises a monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, camelid antibody, or a combination thereof, a binding fragment thereof, or a chemically modified derivative thereof. In some embodiments, the antibody or the fragment thereof binds to a polypeptide that is at least 70%, at least 75%, at least 80%, at least 85%, at least 90%, at least 95%, or at least 99% identical to SEQ ID NO: 12. In some embodiments, the antibody or the fragment comprises a polypeptide sequence that is at least 70%, at least 75%, at least 80%, at least 85%, at least 90%, at least 95%, or at least 99% identical to any one of SEQ ID NOs: 139-141, or a fragment thereof, or a combination thereof. In some embodiments, the Ang2 inhibitor comprises a RNA interference (RNAi).

In some embodiments, the RNAi comprises a shRNA, siRNA, miRNA, or a combination thereof. In some embodiments, the RNAi comprises shRNA that binds to endogenous nucleic acid encoding Ang2. In some embodiments, the RNAi binds to an Ang2 nucleic acid sequence that is at least 70%, at least 75%, at least 80%, at least 85%, at least 90%, at least 95%, or at least 99% identical to SEQ ID NO: 13. In some embodiments, the non-naturally occurring polynucleotide comprises a nucleic acid sequence that is at least 70%, at least 75%, at least 80%, at least 85%, at least 90%, at least 95%, at least 99%, or 100% identical to any one of SEQ ID NOs: 81-86. In some embodiments, the non-naturally occurring polynucleotide comprises a nucleic acid sequence that is at least 70%, at least 75%, at least 80%, at least 85%, at least 90%, at least 95%, at least 99%, or 100% identical to any one of SEQ ID NOs: 31-34 and 51-77. In some embodiments, the one or more expression cassettes comprise one or more promoters, one or more internal ribosome entry sites (IRES), or both. In some embodiments, the VEGF inhibitor and the Ang1 polypeptide decrease neovascularization signaling when expressed in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling in absence of the VEGF inhibitor and the Ang1 polypeptide. In some embodiments, the VEGF inhibitor and the Ang1 polypeptide decrease neovascularization signaling when expressed in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling decreased by a comparable VEGF inhibitor and a comparable Ang1 polypeptide encoded from two different non-naturally occurring polynucleotides.

In some embodiments, the VEGF inhibitor and the Ang2 inhibitor decrease neovascularization signaling when expressed in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling in absence of the VEGF inhibitor and the Ang2 inhibitor. In some embodiments, the VEGF inhibitor and the Ang2 inhibitor decrease neovascularization signaling when expressed in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling decreased by a comparable VEGF inhibitor and a comparable Ang2 inhibitor encoded from two different non-naturally occurring polynucleotides.

Described herein, in some aspects, is a viral vector comprising the non-naturally occurring polynucleotide described herein. In some embodiments, the viral vector is scAAV vector. In some embodiments, the viral vector comprises an AAV serotype comprising AAV1, AAV2, AAV3, AAV4, AAV5, AAV6, AAV7, AAV8, AAV9, AAV10, AAV11, AAV12, or any combination thereof.

Described herein, in some aspects, is a cell comprising the non-naturally occurring polynucleotide described herein. In some embodiments, the cell comprises an embryonic stem cell, an embryonic stem cell-derived differentiated cell, a retinal pigment epithelium (RPE) cell, a neural progenitor cell, a photoreceptor precursor cell, a bone marrow-derived hematopoietic stem cell, or a bone marrow-derived hematopoietic stem progenitor cell.

Described herein, in some aspects, is a pharmaceutical composition comprising the non-naturally occurring polynucleotide the cell described herein. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments, the pharmaceutical composition is for treating an ocular disease or condition. In some embodiments, the pharmaceutical composition decreases neovascularization, blood vessel leakage, inflammation, or a combination thereof in the subject.

Described herein, in some aspects, is a method for treating a disease or a condition in a subject in need thereof, the method comprising administering to the subject a therapeutically effective amount of the non-naturally occurring polynucleotide, the cell the pharmaceutical composition described herein. In some embodiments, the VEGF inhibitor and the activator of the RTK/Tie2 or the RTK/Tie2 decrease neovascularization signaling when expressed in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling in absence of the VEGF inhibitor and the activator of the RTK/Tie2 or the RTK/Tie2. In some embodiments, the VEGF inhibitor and the activator of the RTK/Tie2 or the RTK/Tie2 decrease neovascularization signaling in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling decreased by a comparable VEGF inhibitor and a comparable activator of a RTK/Tie2 or a comparable RTK/Tie2 encoded from two different non-naturally occurring polynucleotides. In some embodiments, the non-naturally occurring polynucleotide, the cell, or the pharmaceutical composition described herein decreases neovascularization, blood vessel leakage, inflammation, or a combination thereof in the subject. In some embodiments, the VEGF inhibitor and the activator of the RTK/Tie2 or the RTK/Tie2 decrease neovascularization in a cell by at least 10%, at least 20%, at least 30%, at least 40%, at least 50%, at least 60%, at least 70%, at least 80%, at least 90%, at least 100%, at least 200%, at least 500%, or more compared to neovascularization in absence of the VEGF inhibitor and the activator of the RTK/Tie2 or the RTK/Tie2. In some embodiments, the VEGF inhibitor and the activator of the RTK/Tie2 or the RTK/Tie2 decrease neovascularization in a cell by at least 10%, at least 20%, at least 30%, at least 40%, at least 50%, at least 60%, at least 70%, at least 80%, at least 90%, at least 100%, at least 200%, at least 500%, or more compared to neovascularization decreased by a comparable VEGF inhibitor and a comparable activator of a RTK/Tie2 or a comparable RTK/Tie2 encoded from two different non-naturally occurring polynucleotides. In some embodiments, the VEGF inhibitor and the activator of the RTK/Tie2 or the RTK/Tie2 decrease blood vessel leakage in a cell by at least 10%, at least 20%, at least 30%, at least 40%, at least 50%, at least 60%, at least 70%, at least 80%, at least 90%, at least 100%, at least 200%, at least 500%, or more compared to blood vessel leakage in absence of the VEGF inhibitor and the activator of the RTK/Tie2 or the RTK/Tie2. In some embodiments, the VEGF inhibitor and the activator of the RTK/Tie2 or the RTK/Tie2 decrease blood vessel leakage in a cell by at least 10%, at least 20%, at least 30%, at least 40%, at least 50%, at least 60%, at least 70%, at least 80%, at least 90%, at least 100%, at least 200%, at least 500%, or more compared to blood vessel leakage decreased by a comparable VEGF inhibitor and a comparable activator of a RTK/Tie2 or a comparable RTK/Tie2 encoded from two different non-naturally occurring polynucleotides. In some embodiments, the VEGF inhibitor and the activator of the RTK/Tie2 or the RTK/Tie2 decrease inflammation in a cell by at least 10%, at least 20%, at least 30%, at least 40%, at least 50%, at least 60%, at least 70%, at least 80%, at least 90%, at least 100%, at least 200%, at least 500%, or more compared to inflammation in absence of the VEGF inhibitor and the activator of the RTK/Tie2 or the RTK/Tie2. In some embodiments, the VEGF inhibitor and the activator of the RTK/Tie2 or the RTK/Tie2 decrease inflammation in a cell by at least 10%, at least 20%, at least 30%, at least 40%, at least 50%, at least 60%, at least 70%, at least 80%, at least 90%, at least 100%, at least 200%, at least 500%, or more compared to inflammation signaling decreased by a comparable VEGF inhibitor and a comparable activator of a RTK/Tie2 or a comparable RTK/Tie2 encoded from two different non-naturally occurring polynucleotides. In some embodiments, the disease or the condition comprises ocular ischemic syndrome, proliferative retinopathies, neovascular glaucoma (NG), uveitis, neovascular uveitis, achromatopsia, age-related macular degeneration (nAMD), diabetic macular edema (DME), diabetic macular retinopathy (DMR), retinal vein occlusion (RVO), glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), and blue-cone monochromacy. In some embodiments, the disease or the condition comprises diabetic macular edema (DME). In some embodiments, the disease or the condition comprises diabetic macular retinopathy (DMR).

Described herein, in some aspects, is kit comprising: the non-naturally occurring polynucleotide, the cell, or the pharmaceutical composition described herein; and a container.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 illustrates targets that can be inhibited or modulated by VEGF inhibitor, activator of RTK/Tie2, or RTK/Tie2 described herein for modulating or decreasing neovascularization.

FIG. 2 illustrates exemplary Adeno-associated virus (AAV) vector comprising the non-naturally occurring polynucleotide described herein, where the non-naturally occurring polynucleotide comprises two expression cassettes encoding a combination of VEGF inhibitor comprising VEGF antibody and either Ang1 fragment or Ang2 shRNA.

FIG. 3A illustrates the expression levels (μg/ml) of a non-antibody VEGF inhibitor (Aflibercept or VEGF-Trap) and Ang1 fragment (Ang1-FLD) encoded by exemplary AAV vectors comprising the non-naturally occurring polynucleotide described herein (AAV2.N54-120-136 or AAV2.N54-120-153) or endogenous Ang2 inhibited by Ang2 shRNA encoded by different exemplary AAV vectors (AAV2.N54-120-150 or AAV2.N54-120-148).

FIG. 3B illustrates additional AAV vectors, where the activator of RTK/Tie2 comprises either Ang1 fragment (Ang1-FOLD or Ang1-FTD) or Ang2 shRNA.

FIG. 4 illustrates expression levels (μg/ml) of a non-antibody VEGF inhibitor (Aflibercept or VEGF-Trap) and endogenous Ang2 inhibited by Ang2 shRNA, where the non-antibody VEGF inhibitor and the Ang2 shRNA were encoded by an exemplary AAV vector (AAV2.N54-120-150 or AAV2.N54-120-148) described herein.

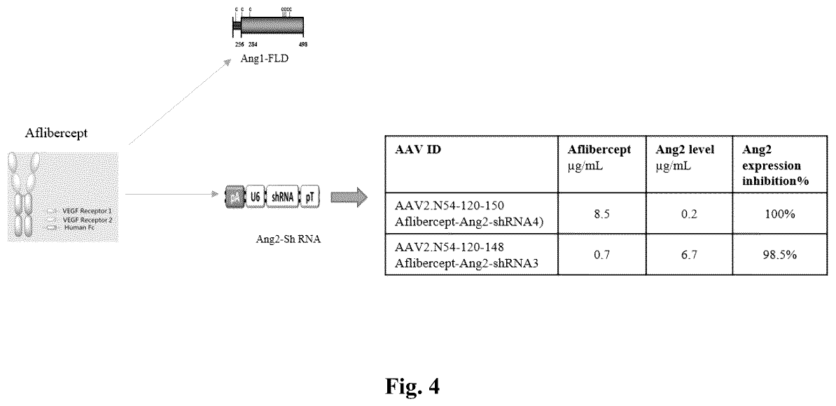

FIG. 5 illustrates expression levels (μg/ml) of a VEGF inhibitor (VEGF-scFv antibody) and endogenous Ang2 inhibited by Ang2 shRNA, where the VEGF antibody and the Ang2 shRNA were encoded by an exemplary AAV vector described herein.

FIG. 6A illustrates an exemplary pFB-AAV vector map comprising the at least two expression cassettes described herein, where the illustrated AAV vector is a baculovirus-based AAV vector.

FIG. 6B illustrates an exemplary pFB-AAV vector (AVMX103: Anti-VEGF-(Fab)2-hCOMP-Ang1) comprising VEGF antibody (top box, SEQ ID NO: 21 and SEQ ID NO: 22), hCOMP-Ang1 (middle box, SEQ ID NO: 6) or FLAG-hCOMP-Ang1 (lower box, SEQ ID NO: 8).

FIG. 7A illustrates an exemplary pFB-AAV vector (AVMX103: Anti-VEGF-(Fab)2-hCOMP-Ang1).

FIG. 7B illustrates an exemplary pFB-AAV vector (AVMX103: VEGF(Fab)2-linker-hCOMP-Ang-1) for expressing a VEGF antibody fused to an Ang1 fragment. The heavy chain of the VEGF antibody is fused with a soluble polypeptide (hCOMP, SEQ ID NO: 2) and Ang1 fragment (SEQ ID NO: 5) via a GGGGSG linker (top box, SEQ ID NO: 41), while the light chain of the anti-VEGF antibody is transcribed separately by a different expression cassette (lower box, SEQ ID NO: 43).

FIG. 7C illustrates an exemplary pFB-AAV vector (AVMX103b: Anti-VEGF-(Fab)2-Linker-hCOMP-An AVMX103: VEGF(Fab)2-linker-Ang-1) comprising VEGF antibody fused to Ang1 fragment (top box, SEQ ID NO: 42 and lower box, SEQ ID NO: 43). The heavy chain of the VEGF antibody is fused with the Ang1 fragment (SEQ ID NO: 5) via a GGGGSG linker (top box, SEQ ID NO: 42), while the light chain of the anti-VEGF antibody is transcribed separately (lower box, SEQ ID NO: 43).

FIG. 7D illustrates a non-limiting exemplary pFB-AAV vector (AVMX-110:VEGF-Trap-hCOMP-Ang1) encoding a non-antibody VEGF inhibitor (SEQ ID NO: 24) and an Ang1 fragment (SEQ ID NO: 6).

FIG. 8A illustrates a non-limiting exemplary pFB-AAV vector (AVMX110-hCOMP-Ang1.FLD) encoding a non-antibody VEGF inhibitor (SEQ ID NO: 24) fused with an Ang1 fragment (SEQ ID NO: 6) as denoted by “4×GGGGS”.

FIG. 8B illustrates a non-limiting exemplary pFB-AAV vector (AVMX110-Ang2-Antibody) encoding a non-antibody VEGF inhibitor (VEGF-Trap, SEQ ID NO: 24) and an ANG2 antibody (SEQ ID NO: 141).

FIG. 8C illustrates a non-limiting exemplary pFB-AAV vector encoding a scFv antibody VEGF inhibitor (SEQ ID NO: 23) linked (as denoted by “4×GGGGS”) to an ANG2 antibody (SEQ ID NO: 141).

FIG. 8D illustrates a non-limiting exemplary pFB-AAV vector (AVMX110-anti-Ang2 shRNA) encoding a non-antibody VEGF inhibitor (VEGF-Trap, SEQ ID NO: 24) and an ANG2 silence sequence (e.g., nucleic acid sequence encoding inhibitory RNA).

FIG. 9A illustrates a non-limiting exemplary pFB-AAV vector (AVMX104: Anti-VEGF-ScFV-hCOMP-Ang1) encoding an antibody VEGF inhibitor (SEQ ID NO: 34) and either hCOMP-Ang1 (for treatment purpose, SEQ ID NO: 6) or FLAG-hCOMP-Ang1 (for pharmacokinetic purpose. SEQ ID NO: 28).

FIG. 9B illustrates a non-limiting exemplary pFB-AAV vector (AVMX105: Flt1-D2/KDR-D2) encoding a soluble VEGF inhibitor (SEQ ID NO: 25). The AAV vector can comprise at least more expression cassette for expressing any one of the VEGF inhibitor, activator of RTK/Tie2, or RTK/Tie2 described herein.

FIG. 9C illustrates a non-limiting exemplary pFB-AAV vector (AVMX106: Flt1-D2/KDR-D2-COMP-Ang1) encoding soluble VEGF inhibitor (SEQ ID NO: 25) and a hCOMP-Ang1.

FIG. 9D illustrates a non-limiting exemplary pFB-AAV vector (Flt1-D2/KDR-D2-hCOMP-Ang1) encoding a soluble VEGF inhibitor (SEQ ID NO: 25, top box, or SEQ ID NO: 26, lower box) and a hCOMP-Ang1.

FIG. 9E illustrates a non-limiting exemplary pFB-AAV vector (AVMX108: VEGF-scFv) encoding an scFv antibody VEGF inhibitor (SEQ ID NO: 35). The AAV vector can comprise at least one more expression cassette for expressing any one of the VEGF inhibitor, activator of RTK/Tie2, or RTK/Tie2 described herein.

FIG. 9F illustrates a non-limiting exemplary pFB-AAV vector (VEGF-ScFV-hCOMP-Ang1) encoding an scFv antibody VEGF inhibitor (SEQ ID NO: 35) and a hCOMP-Ang1.

FIG. 10A illustrates exemplary information FOR Ang1, COMP-Ang1, and disadvantages of full length Ang1.

FIG. 10B illustrates an exemplary dual expression AAV construct.

FIG. 11 illustrates dual gene constructs as compared with single gene constructs.

FIG. 12 illustrates that VEGF promoted leakage but Aflibercept and Ang1 acted to reduce the leakage of FITC dextran.

FIG. 13A and FIG. 13B show the results of Ang1 and VEGF-Trap constructs comparison as bar graph±SEM and statistical analysis using one-way ANOVA and multiple comparison using Dunnett testing.

FIG. 14 illustrates representative FA images from different groups.

FIG. 15 illustrates VEGF-Trap concentration expressed in pg of Aflibercept per eye cup. Eye cup consisted of retina, sclera, choroid and retina.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Abnormal expression of VEGFs leads to the pathogenesis of retinal tissue such as neovascularization age-related macular degeneration (nAMD), diabetic retinopathy (DMR), polypoidal choroid vasculopathy (PCV), etc. Besides VEGFs, many other factors such as placental growth derived growth factor-B (PDGF-B), stromal-derived factor-1 (SDF-1), hypoxia-inducible factor-1 (HIF-1), receptor tyrosine kinase (RTK/Tie2), vascular cell adhesion molecule 1 (VCAM-1), neuropilin-1 (NP-1), neuropilin-2 (NP-2), ephrin, or the Eph (erythropoietin-producing hepatocellular carcinoma) are found to be associated with neovascularization.

Receptor tyrosine kinase TEK tyrosine kinase 2 (RTK/Tie2) and its related ligands, angiopoietin 1 (Ang1) and angiopoietin 2 (Ang2), are the most relevant factors responsible for assembling and disassembling the endothelial lining of blood vessels. Angiopoietins are involved with controlling microvascular permeability, vasodilation, and vasoconstriction by signaling smooth muscle cells, pericytes, and surrounding vessels. Ang1 is a physiological angiogenesis promoter during embryonic development and is produced by vascular smooth-muscle cells. The function of Ang1 is essential to endothelial cell survival, vascular branching, and pericyte recruitment. Ang1 is a glycoprotein of 498 amino acid residues and two isoforms, with a single amino acid mutation at glycine 269 position (G269) missing in the isoform 2. The functional regions of Ang1 aa 1-19 is the secretory signaling sequence (S); aa 20-158 is the super clustering domain (SCD); aa159-255 is the coiled-coil oligomeric domain (CCOD), aa256-83; and aa 284-498 is the fibrinogen-like domain (FLD), a RTK/Tie2 binding domain. Ang1 promotes formation and maturation of blood vessels of tissues and the retinal vascular network during postnatal development. Experimentally induced elevations in Ang1 can cause reductions in retinal vascular leukocyte adhesion, endothelial cell damage, and blood-retinal barrier breakdown in a diabetic retinopathy model, suppressed the development of CNV following laser wounding, and inhibited VEGF-mediated breakdown of the blood-retinal barrier in response to ischemia. Ang1 C-terminal FLD can be dimerized and binds to RTK/Tie2, when it is fused at its N-terminal, to a dimerization unit of a human unnamed protein sequence aligned to a coiled-coil domain of rat cartilage oligomeric matrix protein (COMP), a 45 amino acid peptide (termed hCOMP or Ang1-FLD dimerization unit). Ang2 is a growth factor belonging to the angiopoietin/Tie (tyrosine kinase with Ig and EGF homology domains) signaling pathway, one of the main pathways involved in angiogenesis. Ang2 was identified through a cDNA library screening, shortly after the identification of ANG1, a potent angiogenic factor. Ang2 is critical for in vivo angiogenesis. Ang2, a 496 amino acid-long protein, shares about 60% amino acid homology with Ang1 and lacks one of the nine cysteines found in mature ANG1. It has a secretion signaling peptide, an NH₂-terminal coiled-coil domain, and a COOH-terminal fibrinogen-like domain. Unlike Ang1, Ang2 acts in an autocrine manner, and its expression is highly regulated. Similar to Ang1, Ang2 binds to the Tie2 receptor with the same binding affinity, inducing antagonistic role opposing Ang1. Ang2 expression is triggered by inflammatory mediators such as thrombin accumulation, hypoxia, or cancer. RTK/Tie2 can be activated by expressing Ang1 or a fragment thereof or by expression of an inhibitor of Ang 2 (e.g., inhibitory RNA or antibody targeting Ang2), which in turn decreases neovascularization signal.

In some cases, additional RTK/Tie2 can be expressed in a cell. By expressing RTK/Tie2, the frequency of Ang1 (e.g., endogenously expressed Ang1) contacting and activating RTK/Tie2 is increased. In such scenario, expressing RTK/Tie2 asserts similar effects as expressing Ang1 or decreasing expression of Ang2.

Neovascularization plays an important role in tissue development and pathogenesis of many diseases, including ocular ischemic syndrome, proliferative retinopathies, neovascular glaucoma (NG), uveitis, or neovascular uveitis. Clinical efficacy of intravitreal (IVT) anti-VEGF drugs has been widely demonstrated as the benchmark treatment in several angiogenesis-driven eye diseases including diabetic macular edema (DMR), neovascular form of age-related macular degeneration (nAMD). Pegaptanib, ranibizumab (Lucentis), and aflibercept (Eylea) have been approved for use in the eye, whereas, bevacizumab (Avastin) is widely used by ophthalmologists to treat patients “off-label” to limit the treatment cost.

These drugs are active in the nanomolar to picomolar range, but effective period is short. Patients are required to be administrated once every 4-6 weeks. Most of them are associated with neovascularization, and patients rely on monthly administration of either one of these anti-VEGF therapies. The challenges residing in the anti-VEGF treatment of these eye diseases are short durability of bioavailability and frequent IVT administration of anti-VEGF drugs, thus, causing great inconvenience and financial burdens on patients. Once the IVT drugs are administered, anti-VEGF antagonists in vitreous humor (VH) fluctuate which leads to the instability of pathophysiology and vision changes. Accordingly, there remains needs for therapeutics that can augment the therapeutic effect of VEGF inhibition. There also remains needs for therapeutics that can decrease neovascularization or neovascularization signal.

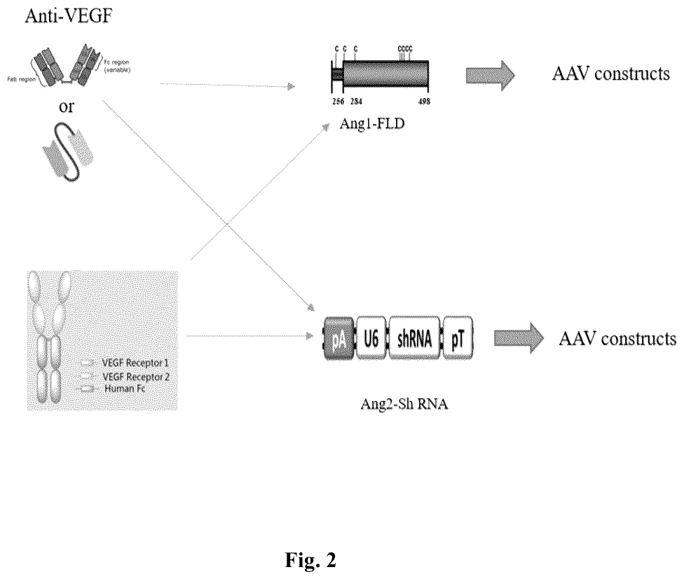

To address these needs, described herein is a non-naturally occurring polynucleotide functioning as a single delivery vehicle, where the non-naturally occurring polynucleotide comprises one or more expression cassettes encoding a VEGF inhibitor and an activator of the RTK/Tie2. FIG. 2 illustrates a non-limiting example of AAV vector comprising a non-naturally occurring polynucleotide for expressing a VEGF inhibitor (e.g., a VEGF antibody) and an activator of RTK/Tie2 comprising either: Ang1 fragment (Ang1-FLD, an agonist of RTK/Tie2); or Ang2 shRNA for decreasing endogenous Ang2 (an antagonist of RTK/Tie2 signaling transduction pathway) expression.

In some embodiments, the non-naturally occurring polynucleotide can be part of a viral vector such as an AAV vector. By utilizing such AAV vector with one or more expression cassettes, different combinations of VEGF inhibitor and activator of RTK/Tie2 comprising Ang1 can be constructed, such as, but not limited to, anti-VEGF antibody comprising IgG, Fab, F(ab)′2, or scFv or a fragment thereof and Ang1 full length protein; non-antibody VEGF inhibitor comprising soluble Flt1 VEGF binding domain and Ang1 full length or fragment protein (e.g., Ang1-FLD fused to hCOMP); or non-antibody VEGF inhibitor comprising soluble Flt1 and Flk1 VEGF binding domains and Ang1 full length or fragment protein. By using this approach, VEGF signaling transduction pathway is decreased by the VEGF inhibitor, while the level of Ang1 expression is increased, leading to activation of RTK/Tie2. The activation of RTK/Tie2 leads to proliferation of pericytes, which strengthens blood vessels and decreases leakage of blood vessels and inflammation associated with blood vessel leakage.

Alternatively, instead of increasing Ang1 expression, RTK/Tie2 can be activated by antagonizing Ang2 expression. Antagonists of Ang2 can include, without limitation, antibody or inhibitory RNA targeting Ang2. Using AAV vector comprising the one or more expression cassettes, VEGF inhibitor and antibody or inhibitory RNA (e.g., shRNA) targeting Ang2 can be delivered into a cell for inhibiting VEGF while simultaneously activating the RTK/Tie2.

Also described herein are methods for treating a disease or condition with the non-naturally occurring polynucleotide described herein. The disease or condition is associated with increased neovascularization, which leads to pathologies such as corneal neovascularization, retinopathy of prematurity, diabetic retinopathy, age-related macular degeneration, or choroidal neovascularization in a subject, In some cases, the non-naturally occurring polynucleotide described herein can lead to synergistic therapeutic effects for treating the disease or the condition. For example, the subject who is treated with a vector (e.g., as a single delivery vehicle) can exhibit a decrease of angiogenesis, neovascularization, blood vessel leakage, inflammation, or a combination thereof compared to if the subject has received: only VEGF inhibitor treatment; only treatment for activating RTK/Tie2; or VEGF inhibitor treatment and treatment for activating RTK/Tie2 via different modalities (e.g., at different times, by different routes, or by different delivery vehicles).

### Non-Naturally Occurring Polynucleotide

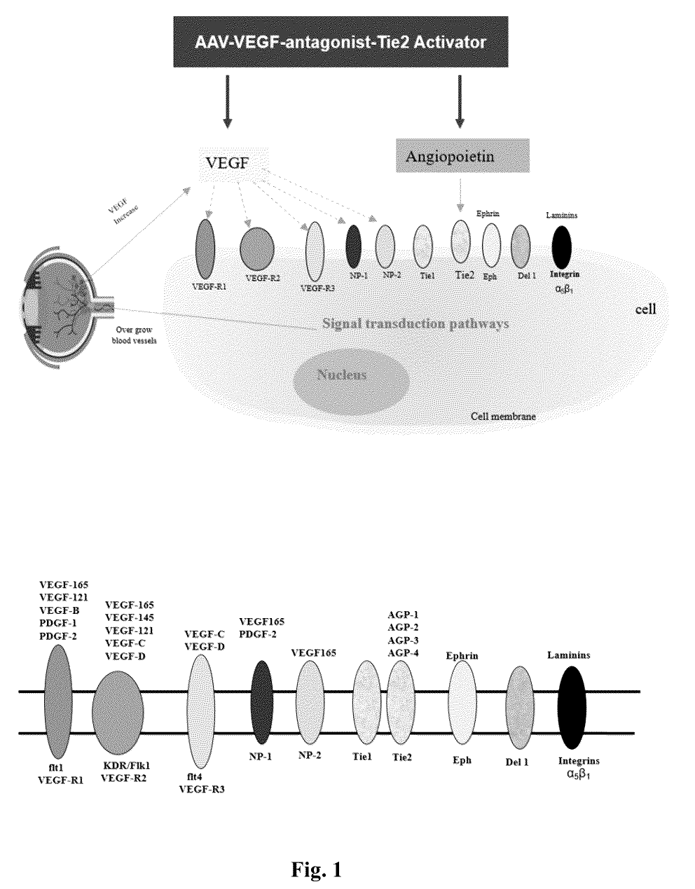

Described herein are non-naturally occurring polynucleotides comprising one or more expression cassettes for expressing a VEGF inhibitor; and a RTK/Tie2 or an activator of the RTK/Tie2. The VEGF inhibitor and the RTK/Tie2 or the activator of the RTK/Tie2 can modulate neovascularization signaling in a cell. In some embodiments, the VEGF inhibitor and the RTK/Tie2 or the activator of the RTK/Tie2 decreases neovascularization signaling in a cell. In some embodiments, the neovascularization signaling comprises signaling transduction pathways associated with vasculogenesis, angiogenesis, or arteriogenesis. In some embodiments, the neovascularization signaling comprises VEGF signaling transduction pathway or angiopoietin signaling transduction pathway. FIG. 1 illustrates a non-limiting example of the ligands and receptors involved in the VEGF signaling transduction pathway and the angiopoietin signaling transduction pathway. For example, VEGF signaling transduction pathway is modulated by multiple VEGFs or VEGF isoforms binding to multiple VEGF receptors, while the angiopoietin signaling transduction pathway is primarily modulated by angiopoietin (Ang1, Ang2, Ang3, and Ang4) binding to RTK/Tie2. FIG. 1 also illustrates potential VEGF signaling transduction pathway or angiopoietin signaling transduction pathway targets that can be modulated by the VEGF inhibitor and the RTK/Tie2 or the activator of the RTK/Tie2 described herein for decreasing neovascularization signaling.

In some embodiments, the non-naturally occurring polynucleotide comprises one expression cassette for expressing the VEGF inhibitor and the RTK/Tie2 or the activator of the RTK/Tie2 as one contiguous polypeptide, which is cleavable into separate polypeptides comprising the VEGF inhibitor, the RTK/Tie2, or activator of RTK/Tie2. In some embodiments, the contiguous polypeptide comprises a protease peptide sequence. In some embodiments, the protease peptide sequence is cleavable by a protease expressed endogenously in a cell. Non-limiting example of the protease can include a serine endoprotease, an aspartic endoprotease, a cysteine thiol endoprotease, a metalloendoprotease, or a glutamic acid and threonine endoprotease. In some embodiments, the protease peptide sequence is cleavable by a serine endoprotease. In some embodiments, the protease peptide sequence is cleavable by Furin. In some embodiments, the contiguous polypeptide comprises a protease cleavable sequence. In some embodiments, the protease cleavable sequence can be cleaved by any one of the proteases described herein. In some embodiments, the protease cleavable sequence can be cleaved by Furin.

In some embodiments, the contiguous polypeptide comprises a self-cleaving polypeptide sequence. In some embodiments, the self-cleaving polypeptide sequence comprises a 2A self-cleaving peptide sequence. Non-limiting examples of the 2A self-cleaving peptide sequence can include T2A, P2A, E2A, F2A, or a combination thereof. In some embodiments, the self-cleaving polypeptide sequence comprises a F2A peptide sequence. In some embodiments, the contiguous polypeptide comprises a protease cleavable sequence and a self-cleaving polypeptide sequence. For example, the contiguous polypeptide described herein can comprise a Furin-F2A fusion polypeptide sequence.

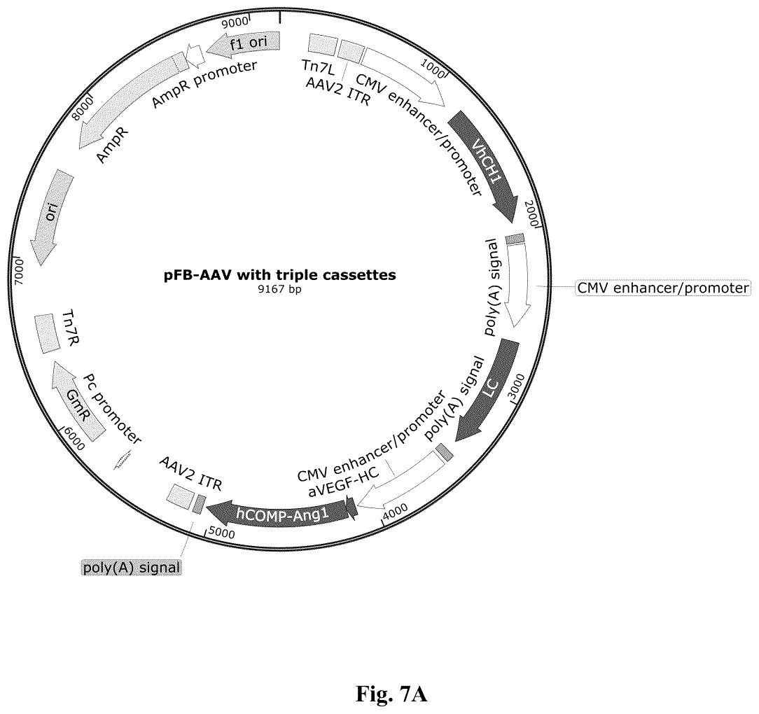

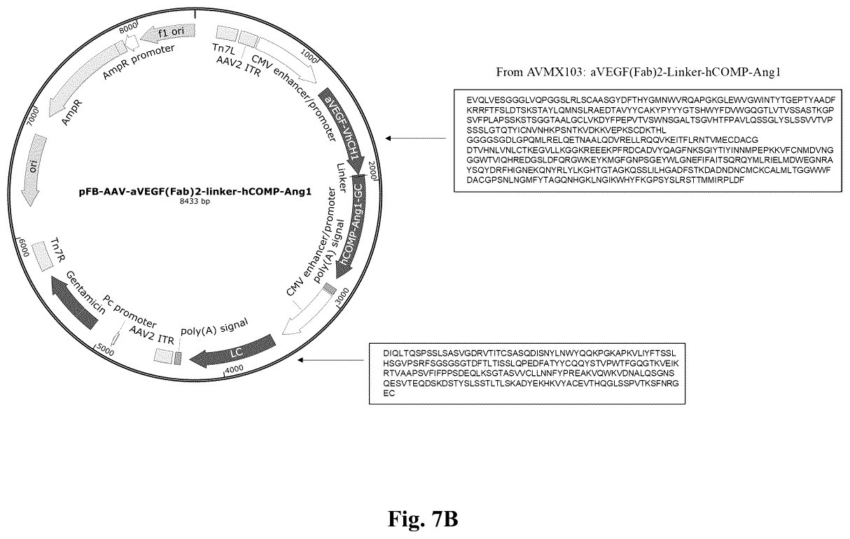

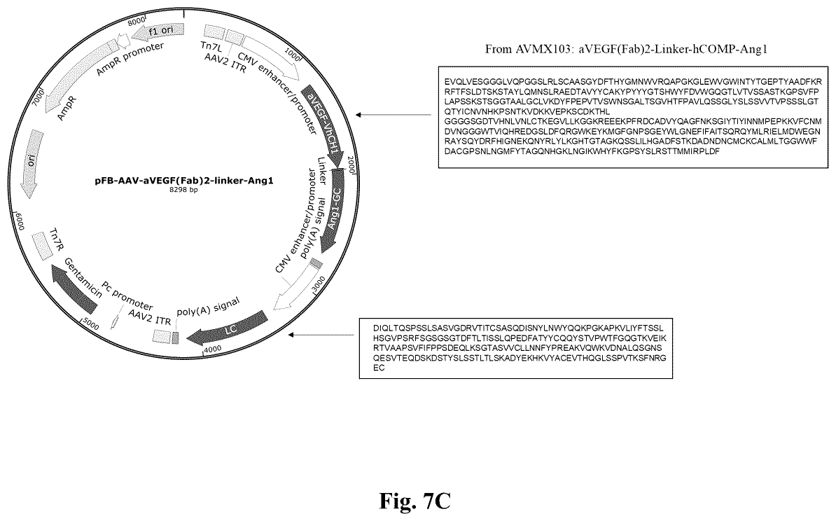

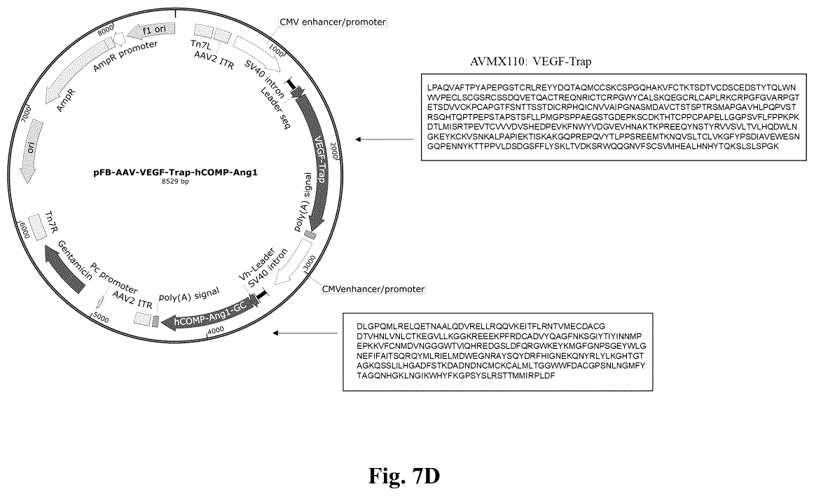

In some embodiments, the non-naturally occurring polynucleotide comprises at least two, at least three, at least four, at least five, or more expression cassettes for the VEGF inhibitor and the RTK/Tie2 or the activator of the RTK/Tie2. In some embodiments, the non-naturally occurring polynucleotide comprises two expression cassettes. In some embodiments, the VEGF inhibitor and the RTK/Tie2 or the activator of the RTK/Tie2 are each expressed from an expression cassette. In other cases, the VEGF inhibitor can be partially expressed as a fusion protein by one of the two expression cassettes, while the other expression cassette expresses the remaining portion of the VEGF inhibitor. For example, FIG. 7B illustrates VEGF antibody fused to an Ang1 fragment. In this example, the heavy chain of the VEGF antibody is fused with a soluble polypeptide (hCOMP, SEQ ID NO: 2) and Ang1 fragment (SEQ ID NO: 5) via a GGGGSG linker (top box, SEQ ID NO: 41), while the light chain of the anti-VEGF antibody is transcribed separately by a different expression cassette (lower box, SEQ ID NO: 43). Another example is FIG. 7C, which illustrates another exemplary AAV vector, where the heavy chain of the VEGF antibody is fused with an Ang1 fragment (SEQ ID NO: 5) via a GGGGSG linker (top box, SEQ ID NO: 41), while the light chain of the anti-VEGF antibody is transcribed separately by a different expression cassette (lower box, SEQ ID NO: 43).

In some embodiments, the expression cassette comprises one or more promoters or internal ribosome entry sites (IRES). In some embodiments, the expression cassette is under expression control of a promoter. In some embodiments, the expression cassette is under expression control of a promoter. In some embodiments, expression cassette can further exert expression control via at least one IRES. In such arrangements, expressions of the VEGF inhibitor and the RTK/Tie2 or the activator of the RTK/Tie2 can be accomplished with only one expression cassette.

In some embodiments, the VEGF inhibitor comprises an antibody or a fragment thereof. In some embodiments, the VEGF antibody binds to VEGF to decrease neovascularization signaling comprising the VEGF signaling transduction pathway. In some embodiments, the VEGF inhibitor is not an antibody. For example, the VEGF inhibitor described herein can comprise a VEGF receptor, a combination of VEGF receptors, or a fragment thereof for binding to VEGF for inhibiting or decreasing VEGF signaling transduction pathway. VEGF receptor can include a VEGF receptor 1 (FLT1), a VEGF receptor 2 (KDR/FLK1), a VEGF receptor 3 (FLT4), a fragment thereof, or a combination thereof. In some embodiments, the VEGF receptor can be a soluble VEGF receptor. For example, the soluble VEGF receptor can comprise a soluble VEGFR1, a soluble VEGFR2, a soluble VEGFR3, a soluble fragment thereof, or a combination thereof. In some embodiments, the non-antibody VEGF inhibitor comprises at least one of FLT1, KDR/FLK1, FLT4, a fragment thereof, or a combination thereof. In some embodiments, the non-antibody VEGF inhibitor comprises at least one of soluble FLT1, soluble KDR/FLK1, soluble FLT4, a fragment thereof, or a combination thereof. In some embodiments, the non-antibody inhibitor VEGF comprises a VEGF-Trap. In some embodiments, the non-antibody VEGF inhibitor comprises a polypeptide sequence that is at least 70%, at least 75%, at least 80%, is at least 85%, at least 90%, at least 95%, at least 99%, or more identical to SEQ ID NO: 24, SEQ ID NO: 25, SEQ ID NO: 26, or SEQ ID NO: 31 (Table 11). In some embodiments, the non-antibody VEGF inhibitor comprises a polypeptide sequence that is SEQ ID NO: 24. In some embodiments, the non-antibody VEGF inhibitor comprises a polypeptide sequence that is SEQ ID NO: 25. In some embodiments, the non-antibody VEGF inhibitor comprises a polypeptide sequence that is SEQ ID NO: 26. In some embodiments, the non-antibody VEGF inhibitor comprises a polypeptide sequence that is SEQ ID NO: 31.

TABLE 11
Non-limiting examples of
non-antibody VEGF inhibitors

| SEQ ID | |
|---|---|
| NO | Non-antibody VEGF inhibitor |
| SEQ ID | LPAQVAFTPYAPEPGSTCRLREYYDQTAQM |
| NO: 24 | CCSKCSPGQHAKVFCTKTSDTVCDSCEDST |
| | YTQLWNWVPECLSCGSRCSSDQVETQACTR |
| | EQNRICTCRPGWYCALSKQEGCRLCAPLRK |
| | CRPGFGVARPGTETSDVVCKPCAPGTFSNT |
| | TSSTDICRPHQICNVVAIPGNASMDAVCTS |
| | TSPTRSMAPGAVHLPQPVSTRSQHTQPTPE |
| | PSTAPSTSFLLPMGPSPPAEGSTGDEPKSC |
| | DKTHTCPPCPAPELLGGPSVFLFPPKPKDT |
| | LMISRTPEVTCVVVDVSHEDPEVKFNWYVD |
| | GVEVHNAKTKPREEQYNSTYRVVSVLTVLH |
| | QDWLNGKEYKCKVSNKALPAPIEKTISKAK |
| | GQPREPQVYTLPPSREEMTKNQVSLTCLVK |
| | GFYPSDIAVEWESNGQPENNYKTTPPVLDS |
| | DGSFFLYSKLTVDKSRWQQGNVFSCSVMHE |
| | ALHNHYTQKSLSLSPGK |
| SEQ ID | DTGRPFVEMYSEIPEIIHMTEGRELVIPCR |
| NO: 25 | VTSPNITVTLKKFPLDTLIPDGKRIIWDSR |
| | KGFIISNATYKEIGLLTCEATVNGHLYKTN |
| | YLTHRQTNTIIDVVLSPSHGIELSVGEKLV |
| | LNCTARTELNVGIDENWEYPSSKHQHKKLV |
| | NRDLKTQSGSEMKKFLSTLTIDGVTRSDQG |
| | LYTCAASSGLMTKKNSTFVRVHEK |
| SEQ ID | DTGRPFVEMYSEIPEIIHMTEGRELVIPCR |
| NO: 26 | VTSPNITVTLKKFPLDTLIPDGKRIIWDSR |
| | KGFIISNATYKEIGLLTCEATVNGHLYKTN |
| | YLTHRQTNTIIDVVLSPSHGIELSVGEKLV |
| | LNCTARTELNVGIDENWEYPSSKHQHKKLV |
| | NRDLKTQSGSEMKKFLSTLTIDGVTRSDQG |
| | LYTCAASSGLMTKKNSTFVRVHEKDKTHTC |
| | PPCPAPELLGGPSVFLFPPKPKDTLMISRT |
| | PEVTCVVVDVSHEDPEVKFNWYVDGVEVHN |
| | AKTKPREEQYNSTYRVVSVLTVLHQDWLNG |
| | KEYKCKVSNKALPAPIEKTISKAKGQPREP |
| | QVYTLPPSRDELTKNQVSLTCLVKGFYPSD |
| | IAVEWESNGQPENNYKTTPPVLDSDGSFFL |
| | YSKLTVDKSRWQQGNVFSCSVMHEALHNHY |
| | TQKSLSLSPGK |
| SEQ ID | SDTGRPFVEMYSEIPEIIHMTEGRELVIPC |
| NO: 31 | RVTSPNITVTLKKFPLDTLIPDGKRIIWDS |
| | RKGFIISNATYKEIGLLTCEATVNGHLYKT |
| | NYLTHRQTNTIIDVVLSPSHGIELSVGEKL |
| | VLNCTARTELNVGIDFNWEYPSSKHQHKKL |
| | VNRDLKTQSGSEMKKFLSTLTIDGVTRSDQ |
| | GLYTCAASSGLMTKKNSTFVRVHEKDKTHT |
| | CPPCPAPELLGGPSVFLFPPKPKDTLMISR |
| | TPEVTCVVVDVSHEDPEVKFNWYVDGVEVH |
| | NAKTKPREEQYNSTYRVVSVLTVLHQDWLN |
| | GKEYKCKVSNKALPAPIEKTISKAKGQPRE |
| | PQVYTLPPSRDELTKNQVSLTCLVKGFYPS |
| | DIAVEWESNGQPENNYKTTPPVLDSDGSFF |
| | LYSKLTVDKSRWQQGNVFSCSVMHEALHNH |
| | YTQKSLSLSPG |

In some embodiments, the activator of the RTK/Tie2 comprises a polypeptide or a polynucleotide for activating RTK/Tie2. In some embodiments, the activator of the RTK/Tie2 comprises a polypeptide comprising an inhibitor such as an antibody or a fragment thereof. In some embodiments, the activator of the RTK/Tie2 comprises a polypeptide comprising a non-antibody inhibitor for activating RTK/Tie2. In some embodiments, the activator of the RTK/Tie2 comprises a polypeptide corresponding to a full length protein. For example, the activator of the RTK/Tie2 can be a full length angiopoietin. In some cases, instead of a full length protein, a fragment of the protein can be utilized as the activator of the RTK/Tie2. For instance, instead of a full length angiopoietin, a fragment of angiopoietin can be utilized for activating RTK/Tie2.

In some embodiments, the activator of the RTK/Tie2 comprises a polypeptide corresponding to a full length protein. In some embodiments, the activator of the RTK/Tie2 comprises a polypeptide encoded from a full length Ang or Ang2 nucleic acid sequence (SEQ ID NO: 4 and SEQ ID NO: 13 respectively, Table 12). In some embodiments, the activator of the RTK/Tie2 comprises a polypeptide corresponding to a full length Ang1 or Ang2 (SEQ ID NO: 3 and SEQ ID NO: 12 respectively, Table 12). In some embodiments, the activator of the RTK/Tie2 comprises a polypeptide comprising a full length Ang1. In some embodiments, the activator of the RTK/Tie2 comprises a polypeptide sequence that is at least 7000, at least 7500 at least 800%, at least 850%, at least 900%, at least 95%, or at least 99% identical to SEQ ID NO: 3.

TABLE 12
Polypeptide and nucleic acid
sequences of Ang1 and Ang2

| | |
|---|---|
| | Ang1 polypeptide sequence |
| SEQ ID | MTVFLSFAFLAAILTHIGCSNQRRSPENSGRRYNR |
| NO: 3 | IQHGQCAYTFILPEHDGNCRESTTDQYNTNALQRD |
| | APHVEPDFSSQKLQHLEHVMENYTQWLQKLENYIV |
| | ENMKSEMAQIQQNAVQNHTATMLEIGTSLLSQTAE |
| | QTRKLTDVETQVLNQTSRLEIQLLENSLSTYKLEK |
| | QLLQQTNEILKIHEKNSLLEHKILEMEGKHKEELD |
| | TLKEEKENLQGLVTRQTYIIQELEKQLNRATTNNS |
| | VLQKQQLELMDTVHNLVNLCTKEGVLLKGGKREEE |
| | KPFRDCADVYQAGFNKSGIYTIYINNMPEPKKVFC |
| | NMDVNGGGWTVIQHREDGSLDFQRGWKEYKMGFGN |
| | PSGEYWLGNEFIFAITSQRQYMLRIELMDWEGNRA |
| | YSQYDRFHIGNEKQNYRLYLKGHTGTAGKQSSLIL |
| | HGADFSTKDADNDNCMCKCALMLTGGWWFDACGPS |
| | NLNGMFYTAGQNHGKLNGIKWHYFKGPSYSLRSTT |
| | MMIRPLDF |
| | Ang1 nucleic acid sequence |
| SEQ ID | ATGACAGTTTTCCTTTCCTTTGCTTTCCTCGCTGC |
| NO: 4 | CATTCTGACTCACATAGGGTGCAGCAATCAGCGCC |
| | GAAGTCCAGAAAACAGTGGGAGAAGATATAACCGG |
| | ATTCAACATGGGCAATGTGCCTACACTTTCATTCT |
| | TCCAGAACACGATGGCAACTGTCGTGAGAGTACGA |
| | CAGACCAGTACAACACAAACGCTCTGCAGAGAGAT |
| | GCTCCACACGTGGAACCGGATTTCTCTTCCCAGAA |
| | ACTTCAACATCTGGAACATGTGATGGAAAATTATA |
| | CTCAGTGGCTGCAAAAACTTGAGAATTACATTGTG |
| | GAAAACATGAAGTCGGAGATGGCCCAGATACAGCA |
| | GAATGCAGTTCAGAACCACACGGCTACCATGCTGG |
| | AGATAGGAACCAGCCTCCTCTCTCAGACTGCAGAG |
| | CAGACCAGAAAGCTGACAGATGTTGAGACCCAGGT |
| | ACTAAATCAAACTTCTCGACTTGAGATACAGCTGC |
| | TGGAGAATTCATTATCCACCTACAAGCTAGAGAAG |
| | CAACTTCTTCAACAGACAAATGAAATCTTGAAGAT |
| | CCATGAAAAAAACAGTTTATTAGAACATAAAATCT |
| | TAGAAATGGAAGGAAAACACAAGGAAGAGTTGGAC |
| | ACCTTAAAGGAAGAGAAAGAGAACCTTCAAGGCTT |
| | GGTTACTCGTCAAACATATATAATCCAGGAGCTGG |
| | AAAAGCAATTAAACAGAGCTACCACCAACAACAGT |
| | GTCCTTCAGAAGCAGCAACTGGAGCTGATGGACAC |
| | AGTCCACAACCTTGTCAATCTTTGCACTAAAGAAG |
| | GTGTTTTACTAAAGGGAGGAAAAAGAGAGGAAGAG |
| | AAACCATTTAGAGACTGTGCAGATGTATATCAAGC |
| | TGGTTTTAATAAAAGTGGAATCTACACTATTTATA |
| | TTAATAATATGCCAGAACCCAAAAAGGTGTTTTGC |
| | AATATGGATGTCAATGGGGGAGGTTGGACTGTAAT |
| | ACAACATCGTGAAGATGGAAGTCTAGATTTCCAAA |
| | GAGGCTGGAAGGAATATAAAATGGGTTTTGGAAAT |
| | CCCTCCGGTGAATATTGGCTGGGGAATGAGTTTAT |
| | TTTTGCCATTACCAGTCAGAGGCAGTACATGCTAA |
| | GAATTGAGTTAATGGACTGGGAAGGGAACCGAGCC |
| | TATTCACAGTATGACAGATTCCACATAGGAAATGA |
| | AAAGCAAAACTATAGGTTGTATTTAAAAGGTCACA |
| | CTGGGACAGCAGGAAAACAGAGCAGCCTGATCTTA |
| | CACGGTGCTGATTTCAGCACTAAAGATGCTGATAA |
| | TGACAACTGTATGTGCAAATGTGCCCTCATGTTAA |
| | CAGGAGGATGGTGGTTTGATGCTTGTGGCCCCTCC |
| | AATCTAAATGGAATGTTCTATACTGCGGGACAAAA |
| | CCATGGAAAACTGAATGGGATAAAGTGGCACTACT |
| | TCAAAGGGCCCAGTTACTCCTTACGTTCCACAACT |
| | ATGATGATTCGACCTTTAGATTTTTGA |
| | Ang2 polypeptide sequence |
| SEQ ID | MWQIVFFTLSCDLVLAAAYNNFRKSMDSIGKKQYQ |
| NO: 12 | VQHGSCSYTFLLPEMDNCRSSSSPYVSNAVQRDAP |
| | LEYDDSVQRLQVLENIMENNTQWLMKLENYIQDNM |
| | KKEMVEIQQNAVQNQTAVMIEIGTNLLNQTAEQTR |
| | KLTDVEAQVLNQTTRLELQLLEHSLSTNKLEKQIL |
| | DQTSEINKLQDKNSFLEKKVLAMEDKHIIQLQSIK |
| | EEKDQLQVLVSKQNSIIEELEKKIVTATVNNSVLQ |
| | KQQHDLMETVNNLLTMMSTSNSKDPTVAKEEQISF |
| | RDCAEVFKSGHTTNGIYTLTFPNSTEEIKAYCDME |
| | AGGGGWTIIQRREDGSVDFQRTWKEYKVGFGNPSG |
| | EYWLGNEFVSQLTNQQRYVLKIHLKDWEGNEAYSL |
| | YEHFYLSSEELNYRIHLKGLTGTAGKISSISQPGN |
| | DFSTKDGDNDKCICKCSQMLTGGWWFDACGPSNLN |
| | GMYYPQRQNTNKFNGIKWYYWKGSGYSLKATTMMI |
| | RPADF |
| | Ang2 nucleic acid sequence |
| SEQ ID | ctggacgtgtgtttgccctcaagtttgctaagctg |
| NO: 13 | ctggtttattactgaagaaagaatgtggcagattg |
| | ttttctttactctgagctgtgatcttgtcttggcc |
| | gcagcctataacaactttcggaagagcatggacag |
| | cataggaaagaagcaatatcaggtccagcatgggt |
| | cctgcagctacactttcctcctgccagagatggac |
| | aactgccgctcttcctccagcccctacgtgtccaa |
| | tgctgtgcagagggacgcgccgctcgaatacgatg |
| | actcggtgcagaggctgcaagtgctggagaacatc |
| | atggaaaacaacactcagtggctaatgaagcttga |
| | gaattatatccaggacaacatgaagaaagaaatgg |
| | tagagatacagcagaatgcagtacagaaccagacg |
| | gctgtgatgatagaaatagggacaaacctgttgaa |
| | ccaaacagcggagcaaacgcggaagttaactgatg |
| | tggaagcccaagtattaaatcagaccacgagactt |
| | gaacttcagctcttggaacactccctctcgacaaa |
| | caaattggaaaaacagattttggaccagaccagtg |
| | aaataaacaaattgcaagataagaacagtttccta |
| | gaaaagaaggtgctagctatggaagacaagcacat |
| | catccaactacagtcaataaaagaagagaaagatc |
| | agctacaggtgttagtatccaagcaaaattccatc |
| | attgaagaactagaaaaaaaaatagtgactgccac |
| | ggtgaataattcagttcttcagaagcagcaacatg |
| | atctcatggagacagttaataacttactgactatg |
| | atgtccacatcaaactctaaggaccccactgttgc |
| | taaagaagaacaaatcagcttcagagactgtgctg |
| | aagtattcaaatcaggacacaccacgaatggcatc |
| | tacacgttaacattccctaattctacagaagagat |
| | caaggcctactgtgacatggaagctggaggaggcg |
| | ggtggacaattattcagcgacgtgaggatggcagc |
| | gttgattttcagaggacttggaaagaatataaagt |
| | gggatttggtaacccttcaggagaatattggctgg |
| | gaaatgagtttgtttcgcaactgactaatcagcaa |
| | cgctatgtgcttaaaatacaccttaaagactggga |
| | agggaatgaggcttactcattgtatgaacatttct |
| | atctctcaagtgaagaactcaattataggattcac |
| | cttaaaggacttacagggacagccggcaaaataag |
| | cagcatcagccaaccaggaaatgattttagcacaa |
| | aggatggagacaacgacaaatgtatttgcaaatgt |
| | tcacaaatgctaacaggaggctggtggtttgatgc |
| | atgtggtccttccaacttgaacggaatgtactatc |
| | cacagaggcagaacacaaataagttcaacggcatt |
| | aaatggtactactggaaaggctcaggctattcgct |
| | caaggccacaaccatgatgatccgaccagcagatt |
| | tctaaacatcccagtccacctgaggaactgtctcg |
| | aactattttcaaagacttaagcccagtgcactgaa |
| | agtcacggctgcgcactgtgtcctcttccaccaca |
| | gagggcgtgtgctcggtgctgacgggacccacatg |
| | ctccagattagagcctgtaaactttatcacttaaa |
| | cttgcatcacttaacggaccaaagcaagaccctaa |
| | acatccataattgtgattagacagaacacctatgc |
| | aaagatgaacccgaggct |

In some embodiments, the activator of the RTK/Tie2 comprises a polypeptide corresponding to a fragment of a full length protein. In some embodiments, the activator of the RTK/Tie2 comprises a polypeptide corresponding to a fragment of a full length angiopoietin. In some embodiments, the activator of the RTK/Tie2 comprises a polypeptide corresponding to a fragment of the angiopoietin comprises a polypeptide sequence comprising at least 10 amino acids, at least 50 amino acids, at least 100 amino acids, at least 150 amino acids. at least 200 amino acids, at least 250 amino acids, at least 300 amino acids, at least 350 amino acids, or more amino acids in length. In some embodiments, the activator of the RTK/Tie2 comprising a fragment of angiopoietin comprises a polypeptide that is encoded from a nucleic acid sequence that is at least 70%, at least 75%, at least 80%, at least 85%, at least 90%, at least 95%, at least 99%, or more identical to SEQ ID NO: 4 or SEQ ID NO: 13. In some embodiments, the activator of the RTK/Tie2 comprising a fragment of angiopoietin comprises a polypeptide that is encoded from a nucleic acid sequence that is at least 70%, at least 75%, at least 80%, at least 85%, at least 90%, at least 95%, at least 99%, or more identical to Ang1 (SEQ ID NO: 4). In some embodiments, the activator of the RTK/Tie2 comprising a fragment of angiopoietin comprises a polypeptide sequence that is at least 70%, at least 75%, at least 80%, at least 85%, at least 90%, at least 95%, at least 99%, or more identical to SEQ ID NO: 3 or SEQ ID NO: 12.

In some embodiments, the activator of the RTK/Tie2 comprises a fragment of Ang1. The fragment of Ang1 can include: Ang1 amino acids 1-19, the secretory signaling sequence (S); Ang1 aa 20-158, the super clustering domain (SCD); Ang1 aa 159-255, the coiled-coil oligomeric domain (CCOD), aa 256-83; and Ang1 aa 284-498, the fibrinogen-like domain (FLD), which is a functional domain that binds to RTK/Tie2. In some embodiments, the activator of the RTK/Tie2 comprises a FLD fragment (functional fragment) of Ang1 (SEQ ID NO: 5). In some embodiments, the activator of the RTK/Tie2 comprises a polypeptide that is at least 70%, at least 75%, at least 80%, at least 85%, at least 90%, at least 95%, at least 99%, or more identical to SEQ ID NO: 5. In some embodiments, the activator of the RTK/Tie2 comprises a fragment of Ang1 that is fused to a soluble peptide for increasing the solubility of the VEGF inhibitor and the RTK/Tie2 or the activator of the RTK/Tie2. In some embodiments, the soluble peptide comprises a polypeptide sequence that is at least 70%, at least 75%, at least 80%, at least 85%, at least 90%, at least 95%, at least 99%, or more identical to SEQ ID NO: 1 or SEQ ID NO: 2. In some embodiments, the soluble peptide comprises a polypeptide sequence that is at most 99%, at most 98%, at most 97%, at most 96%, at most 95%, at most 94%, or at most 93% identical to SEQ ID NO: 1. In some embodiments, the soluble peptide comprises a polypeptide sequence that is at least 70%, at least 75%, at least 80%, at least 85%, at least 90%, at least 95%, at least 99%, or more identical to SEQ ID NO: 2. In some embodiments, the soluble peptide comprises a polypeptide sequence of SEQ ID NO: 2. In some embodiments, a peptide tag such as a FLAG tag (SEQ ID NO: 10, encoded from nucleic acid sequence of SEQ ID NO: 11) can be added to the FLD fusion. The additional of the FLAG tag can be used for pharmacokinetics purposes and measurements. In some embodiments, the activator of the RTK/Tie2 comprising a fragment of Ang1 is fused to a soluble peptide comprising a polypeptide sequence that is at least 70%, at least 75%, at least 80%, at least 85%, at least 90%, at least 95%, at least 99%, or more identical to SEQ ID NO: 6. In some embodiments, the activator of the RTK/Tie2 comprising a fragment of Ang1 is fused to a soluble peptide comprising a polypeptide sequence of SEQ ID NO: 6. In some embodiments, the activator of the RTK/Tie2 comprising a fragment of Ang1 is fused to a soluble peptide comprising a polypeptide encoded from a nucleic acid sequence that is at least 70%, at least 75%, at least 80%, at least 85%, at least 90%, at least 95%, at least 99%, or more identical to SEQ ID NO: 7. In some embodiments, the activator of the RTK/Tie2 comprising a fragment of Ang1 is fused to a soluble peptide comprising a polypeptide encoded from a nucleic acid sequence of SEQ ID NO: 7. In some embodiments, the activator of the RTK/Tie2 comprising a fragment of Ang1 is fused to a soluble peptide comprising a polypeptide sequence that is at least 70%, at least 75%, at least 80%, at least 85%, at least 90%, at least 95%, at least 99%, or more identical to SEQ ID NO: 8. In some embodiments, the activator of the RTK/Tie2 comprising a fragment of Ang1 is fused to a soluble peptide comprising a polypeptide sequence of SEQ ID NO: 8. In some embodiments, the activator of the RTK/Tie2 comprising a fragment of Ang1 is fused to a soluble peptide comprising a polypeptide encoded from a nucleic acid sequence that is at least 70%, at least 75%, at least 80%, at least 85%, at least 90%, at least 95%, at least 99%, or more identical to SEQ ID NO: 9. In some embodiments, the activator of the RTK/Tie2 comprising a fragment of Ang1 is fused to a soluble peptide comprising a polypeptide encoded from a nucleic acid sequence of SEQ ID NO: 9. Table 13 illustrates the nucleic acid and the polypeptide sequences of the variations of the activator of the RTK/Tie2 comprising FLD and soluble polypeptide fusion.

TABLE 13
Nucleic acid and polypeptide sequences
of exemplary activators of the RTK/Tie2
comprising FLD and soluble peptide

| | Soluble peptide |
|---|---|
| SEQ | DLAPQMLRELQETNAALQDVRELLRQQVKEITFLK |
| ID | NTVMECDACG |
| NO: | |
| 1 | |
| | Soluble peptide |
| SEQ | DLGPQMLRELQETNAALQDVRELLRQQVKEITFLR |
| ID | NTVMECDACG |
| NO: | |
| 2 | |
| | FLD AA 284-498 of Ang1 |
| SEQ | DTVHNLVNLCTKEGVLLKGGKREEEKPFRDCADVY |
| ID | QAGFNKSGIYTIYINNMPEPKKVFCNMDVNGGGWT |
| NO: | VIQHREDGSLDFQRGWKEYKMGFGNPSGEYWLGNE |
| 5 | FIFAITSQRQYMLRIELMDWEGNRAYSQYDRFHIG |
| | NEKQNYRLYLKGHTGTAGKQSSLILHGADFSTKDA |
| | DNDNCMCKCALMLTGGWWFDACGPSNLNGMFYTAG |
| | QNHGKLNGIKWHYFKGPSYSLRSTTMMIRPLDF |
| | Ang1 FLD fusion (for therapeutics) |
| SEQ | DLGPQMLRELQETNAALQDVRELLRQQVKEITFLR |
| ID | NTVMECDACGDTVHNLVNLCTKEGVLLKGGKREEE |
| NO: | KPFRDCADVYQAGFNKSGIYTIYINNMPEPKKVFC |
| 6 | NMDVNGGGWTVIQHREDGSLDFQRGWKEYKMGFGN |
| | PSGEYWLGNEFIFAITSQRQYMLRIELMDWEGNRA |
| | YSQYDRFHIGNEKQNYRLYLKGHTGTAGKQSSLIL |
| | HGADFSTKDADNDNCMCKCALMLTGGWWFDACGPS |
| | NLNGMFYTAGQNHGKLNGIKWHYFKGPSYSLRSTT |
| | MMIRPLDF |
| | Ang1 FLD fusion (for therapeutics) |
| SEQ | gatctgggcccgcagatgctgcgcgaactgcagga |
| ID | aaccaacgcggcgctgcaggatgtgcgcgaactgc |
| NO: | tgcgccagcaggtgaaagaaattacctttctgcgc |
| 7 | aacaccgtgatggaatgcgatgcgtgcggcgacac |
| | agtccacaaccttgtcaatctttgcactaaagaag |
| | gtgttttactaaagggaggaaaaagagaggaagag |
| | aaaccatttagagactgtgcagatgtatatcaagc |
| | tggttttaataaaagtggaatctacactatttata |
| | ttaataatatgccagaacccaaaaaggtgttttgc |
| | aatatggatgtcaatgggggaggttggactgtaat |
| | acaacatcgtgaagatggaagtctagatttccaaa |
| | gaggctggaaggaatataaaatgggttttggaaat |
| | ccctccggtgaatattggctggggaatgagtttat |
| | ttttgccattaccagtcagaggcagtacatgctaa |
| | gaattgagttaatggactgggaagggaaccgagcc |
| | tattcacagtatgacagattccacataggaaatga |
| | aaagcaaaactataggttgtatttaaaaggtcaca |
| | ctgggacagcaggaaaacagagcagcctgatctta |
| | cacggtgctgatttcagcactaaagatgctgataa |
| | tgacaactgtatgtgcaaatgtgccctcatgttaa |
| | caggaggatggtggtttgatgcttgtggcccctcc |
| | aatctaaatggaatgttctatactgcgggacaaaa |
| | ccatggaaaactgaatgggataaagtggcactact |
| | tcaaagggcccagttactccttacgttccacaact |
| | atgatgattcgacctttagatttttga |
| | FLD fusion with FLAG tag |
| | (for pharmacokinetics) |
| SEQ | DYKDDDDKDLGPQMLRELQETNAALQDVRELLRQQ |
| ID | VKEITFLRNTVMECDACGMDTVHNLVNLCTKEGVL |
| NO: | LKGGKREEEKPFRDCADVYQAGFNKSGIYTIYINN |
| 8 | MPEPKKVFCNMDVNGGGWTVIQHREDGSLDFQRGW |
| | KEYKMGFGNPSGEYWLGNEFIFAITSQRQYMLRIE |
| | LMDWEGNRAYSQYDRFHIGNEKQNYRLYLKGHTGT |
| | AGKQSSLILHGADFSTKDADNDNCMCKCALMLTGG |
| | WWFDACGPSNLNGMFYTAGQNHGKLNGIKWHYFKG |
| | PSYSLRSTTMMIRPLDF |
| | FLD fusion with FLAG tag (for |
| | pharmacokinetics) |
| SEQ | gattataaagatgatgatgataaagatctgggccc |
| ID | gcagatgctgcgcgaactgcaggaaaccaacgcgg |
| NO: | cgctgcaggatgtgcgcgaactgctgcgccagcag |
| 9 | gtgaaagaaattacctttctgcgcaacaccgtgat |
| | ggaatgcgatgcgtgcggcgacacagtccacaacc |
| | ttgtcaatctttgcactaaagaaggtgttttacta |
| | aagggaggaaaaagagaggaagagaaaccatttag |
| | agactgtgcagatgtatatcaagctggttttaata |
| | aaagtggaatctacactatttatattaataatatg |
| | ccagaacccaaaaaggtgttttgcaatatggatgt |
| | caatgggggaggttggactgtaatacaacatcgtg |
| | aagatggaagtctagatttccaaagaggctggaag |
| | gaatataaaatgggttttggaaatccctccggtga |
| | atattggctggggaatgagtttatttttgccatta |
| | ccagtcagaggcagtacatgctaagaattgagtta |
| | atggactgggaagggaaccgagcctattcacagta |
| | tgacagattccacataggaaatgaaaagcaaaact |
| | ataggttgtatttaaaaggtcacactgggacagca |
| | ggaaaacagagcagcctgatcttacacggtgctga |
| | tttcagcactaaagatgctgataatgacaactgta |
| | tgtgcaaatgtgccctcatgttaacaggaggatgg |
| | tggtttgatgcttgtggcccctccaatctaaatgg |
| | aatgttctatactgcgggacaaaaccatggaaaac |
| | tgaatgggataaagtggcactacttcaaagggccc |
| | agttactccttacgttccacaactatgatgattcg |
| | acctttagatttttga |
| | FLAG Tag |
| SEQ | DYKDDDDK |
| ID | |
| NO: | |
| 10 | |
| | FLAG Tag |
| SEQ | gattataaagatgatgatgataaa |
| ID | |
| NO: | |
| 11 | |

### Antibody

In some embodiments, the VEGF inhibitor is a VEGF antibody. In some embodiments, the VEGF antibody comprises a monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof.

In some embodiments, the VEGF antibody binds to VEGF and decreases VEGF signaling transduction pathway. In some embodiments, the VEGF inhibitor, when delivered in combination with the activator of the RTK/Tie2 by the non-naturally occurring polynucleotide described herein, synergistically decreases the VEGF signaling transduction pathway in the cell compared to a decrease of VEGF signaling transduction pathway induced by separately delivering the VEGF inhibitor and the activator of the RTK/Tie2 (e.g., the VEGF inhibitor and the activator of the RTK/Tie2 delivered into the cell by two separate vectors) and/or by delivering the VEGF inhibitor or the activator of the RTK/Tie2 alone. In some embodiments, the VEGF inhibitor, when delivered in combination with the activator of the RTK/Tie2 by the vector described herein, synergistically increases the RTK/Tie2 signaling transduction pathway in the cell compared to an increase of RTK/Tie2 signaling transduction pathway induced by separately delivering the VEGF inhibitor and the activator of the RTK/Tie2 (e.g., the VEGF inhibitor and the activator of the RTK/Tie2 delivered into the cell by two separate vectors) and/or by delivering the VEGF inhibitor or the activator of the RTK/Tie2 alone.

In some embodiments, the VEGF antibody binds to VEGF-A, VEGF-B, VEGF-C, VEGF-D, or a combination thereof. In some embodiments, the VEGF antibody binds to one or more isoforms of VEGF-A, including VEGF121, VEGF145, VEGF148, VEGF162, VEGF165, VEGF165b, VEGF183, VEGF189, or VEGF206. In some embodiments, the antibody comprises IgG, a Fab, a Fa(ab)′2, a single-chain fragment variable (scFv), a fragment thereof, or a combination thereof. Non-limiting examples of VEGF antibodies include ranibizumab or bevacizumab. In some embodiments, the VEGF antibody comprises a polypeptide sequence that is at least 70%, at least 75%, at least 80%, at least 85%, at least 90%, at least 95%, at least 99%, or more identical to SEQ ID NO: 21, SEQ ID NO: 22, SEQ ID NO: 32, SEQ ID NO: 33, SEQ ID NO: 34, SEQ ID NO: 35, or a combination thereof, or a fragment thereof (Table 13). In some embodiments, the VEGF antibody is a scFv antibody. In some embodiments, the VEGF scFv antibody comprises a polypeptide sequence that is at least 70%, at least 75%, at least 80%, at least 85%, at least 90%, at least 95%, at least 99%, or more identical to SEQ ID NO: 23, or a fragment thereof (Table 14).

TABLE 14
Non-limiting examples of polypeptide
sequences of VEGF antibodies

| SEQ ID | |
|---|---|
| NO | Polypeptide sequence of VEGF antibody |
| SEQ ID | EVQLVESGGGLVQPGGSLRLSCAASGYDFTHYGMN |
| NO: 21 | WVRQAPGKGLEWVGWINTYTGEPTYAADFKRRFTF |
| | SLDTSKSTAYLQMNSLRAEDTAVYYCAKYPYYYGT |
| | SHWYFDVWGQGTLVTVSS |
| SEQ ID | DIQLTQSPSSLSASVGDRVTITCSASQDISNYLNW |
| NO: 22 | YQQKPGKAPKVLIYFTSSLHSGVPSRFSGSGSGTD |
| | FTLTISSLQPEDFATYYCQQYSTVPWTFGQGTKVE |
| | IKRTVAA |
| SEQ ID | EVQLVESGGGLVQPGGSLRLSCAASGYDFTHYGMN |
| NO: 23 | WVRQAPGKGLEWVGWINTYTGEPTYAADFKRRFTF |
| | SLDTSKSTAYLQMNSLRAEDTAVYYCAKYPYYYGT |
| | SHWYFDVWGQGTLVTVSSGGGGSGGGGSGGGGSGG |
| | GGSDIQLTQSPSSLSASVGDRVTITCSASQDISNY |
| | LNWYQQKPGKAPKVLIYFTSSLHSGVPSRFSGSGS |
| | GTDFTLTISSLQPEDFATYYCQQYSTVPWTFGQGT |
| | KVEIKRTVAA |
| SEQ ID | EVQLVESGGGLVQPGGSLRLSCAASGYDFTHYGMN |
| NO: 32 | WVRQAPGKGLEWVGWINTYTGEPTYAADFKRRFTF |
| | SLDTSKSTAYLQMNSLRAEDTAVYYCAKYPYYYGT |
| | SHWYFDVWGQGTLVTVSSASTKGPSVFPLAPSSKS |
| | TSGGTAALGCLVKDYFPEPVTVSWNSGALTSGVHT |
| | FPAVLQSSGLYSLSSVVTVPSSSLGTQTYICNVNH |
| | KPSNTKVDKKVEPKSCDKTHL |
| SEQ ID | DIQLTQSPSSLSASVGDRVTITCSASQDISNYLNW |
| NO: 33 | YQQKPGKAPKVLIYFTSSLHSGVPSRFSGSGSGTD |
| | FTLTISSLQPEDFATYYCQQYSTVPWTFGQGTKVE |
| | IKRTVAAPSVFIFPPSDEQLKSGTASVVCLLNNFY |
| | PREAKVQWKVDNALQSGNSQESVTEQDSKDSTYSL |
| | SSTLTLSKADYEKHKVYACEVTHQGLSSPVTKSFN |
| | RGEC |
| SEQ ID | DIQLTQSPSSLSASVGDRVTITCSASQDISNYLNW |
| NO: 34 | YQQKPGKAPKVLIYFTSSLHSGVPSRFSGSGSGTD |
| | FTLTISSLQPEDFATYYCQQYSTVPWTFGQGTKVE |
| | IKRTVAAGGGGSGGGGSGGGGSGGGGSEVQLVESG |
| | GGLVQPGGSLRLSCAASGYDFTHYGMNWVRQAPGK |
| | GLEWVGWINTYTGEPTYAADFKRRFTFSLDTSKST |
| | AYLQMNSLRAEDTAVYYCAKYPYYYGTSHWYFDVW |
| | GQGTLVTVSS |
| SEQ ID | MEIVMTQSPSTLSASVGDRVIITCQASEIIHSWLA |
| NO: 35 | WYQQKPGKAPKLLIYLASTLASGVPSRFSGSGSGA |
| | EFTLTISSLQPDDFATYYCQNVYLASTNGANFGQG |
| | TKLTVLGGGGGSGGGGSGGGGSGGGGSEVQLVESG |
| | GGLVQPGGSLRLSCTASGFSLTDYYYMTWVRQAPG |
| | KGLEWVGFIDPDDDPYYATWAKGRFTISRDNSKNT |
| | LYLQMNSLRAEDTAVYYCAGGDHNSGWGLDIWGQG |
| | TLVTVSS |

In some embodiments, the VEGF antibody comprises at least one heavy chain and at least one light chain. In such scenario, the at least one heavy chain and the at least one light chain can be expressed separately via the at least two expression cassettes. Additionally, the heavy chain or the light chain can be further fused to any of the activators of the RTK/Tie2 described herein. For example, FIG. 7B illustrates a non-limiting example of a VEGF antibody fused to an Ang1 fragment. The heavy chain of the VEGF antibody is fused with a soluble polypeptide (hCOMP, SEQ ID NO: 2) and Ang1 fragment (SEQ ID NO: 5) via a GGGGSG linker (top box, SEQ ID NO: 41), while the light chain of the anti-VEGF antibody is transcribed separately by a different expression cassette (lower box, SEQ ID NO: 43). Another example is FIG. 7C, which illustrates another exemplary AAV vector, where the heavy chain of the VEGF antibody is fused with an Ang1 fragment (SEQ ID NO: 5) via a GGGGSG linker (top box, SEQ ID NO: 41), while the light chain of the anti-VEGF antibody is transcribed separately by a different expression cassette (lower box, SEQ ID NO: 43).

In some embodiments, the antibody encoded by the non-naturally occurring polynucleotide described herein is an activator of the RTK/Tie2. In some embodiments, the antibody is an Ang2 antibody. In some embodiments, the binding of the Ang2 antibody decreases the VEGF signaling transduction pathway described herein. In some embodiments, the binding of Ang2 antibody to Ang2, when in the presence of the VEGF inhibitor, synergistically decreases the VEGF signaling transduction pathway compared to the decrease of the VEGF signaling transduction pathway induced only by the VEGF inhibitor or only by Ang2 antibody.

In some embodiments, the Ang2 antibody binds to an Ang2 polypeptide or fragment thereof encoded from a nucleic acid sequence that is least 70%, at least 75%, at least 80%, at least 85%, at least 90%, at least 95%, at least 99%, or more identical to SEQ ID NO: 13, or a fragment thereof (Table 15). In some embodiments, the Ang2 antibody binds to an Ang2 polypeptide or fragment thereof comprising a peptide sequence that is least 70%, at least 75%, at least 80%, at least 85%, at least 90%, at least 95%, at least 99%, or more identical to SEQ ID NO: 12, or a fragment thereof (Table 15). In some embodiments, the Ang2 antibody comprises a polypeptide sequence that is least 70%, at least 75%, at least 80%, at least 85%, at least 90%, at least 95%, at least 99%, or more identical to SEQ ID NO: 139, SEQ ID NO: 140, SEQ ID NO: 141, or a fragment thereof, or a combination thereof. In some embodiments, the Ang2 antibody comprises a polypeptide sequence that is SEQ ID NO: 139 and SEQ ID NO: 140. In some embodiments, the Ang2 antibody comprises a polypeptide sequence that is SEQ ID NO: 141.

TABLE 15
Ang2 and Ang2 antibody nucleic
acid and polypeptide sequences

| SEQ | |
|---|---|
| ID | Ang2 and Ang2 antibody nucleic |
| NO | acid and polypeptide sequence |
| | Ang2 nucleic acid sequence |
| SEQ | ctggacgtgtgtttgccctcaagtttgctaagctgctggt |
| ID | ttattactgaagaaagaatgtggcagattgttttctttac |
| NO: | tctgagctgtgatcttgtcttggccgcagcctataacaac |
| 13 | tttcggaagagcatggacagcataggaaagaagcaatatc |
| | aggtccagcatgggtcctgcagctacactttcctcctgcc |
| | agagatggacaactgccgctcttcctccagcccctacgtg |
| | tccaatgctgtgcagagggacgcgccgctcgaatacgatg |
| | actcggtgcagaggctgcaagtgctggagaacatcatgga |
| | aaacaacactcagtggctaatgaagcttgagaattatatc |
| | caggacaacatgaagaaagaaatggtagagatacagcaga |
| | atgcagtacagaaccagacggctgtgatgatagaaatagg |
| | gacaaacctgttgaaccaaacagcggagcaaacgcggaag |
| | ttaactgatgtggaagcccaagtattaaatcagaccacga |
| | gacttgaacttcagctcttggaacactccctctcgacaaa |
| | caaattggaaaaacagattttggaccagaccagtgaaata |
| | aacaaattgcaagataagaacagtttcctagaaaagaagg |
| | tgctagctatggaagacaagcacatcatccaactacagtc |
| | aataaaagaagagaaagatcagctacaggtgttagtatcc |
| | aagcaaaattccatcattgaagaactagaaaaaaaaatag |
| | tgactgccacggtgaataattcagttcttcagaagcagca |
| | acatgatctcatggagacagttaataacttactgactatg |
| | atgtccacatcaaactctaaggaccccactgttgctaaag |
| | aagaacaaatcagcttcagagactgtgctgaagtattcaa |
| | atcaggacacaccacgaatggcatctacacgttaacattc |
| | cctaattctacagaagagatcaaggcctactgtgacatgg |
| | aagctggaggagggggtggacaattattcagcgacgtgag |
| | gatggcagcgttgattttcagaggacttggaaagaatata |
| | aagtgggatttggtaacccttcaggagaatattggctggg |
| | aaatgagtttgtttcgcaactgactaatcagcaacgctat |
| | gtgcttaaaatacaccttaaagactgggaagggaatgagg |
| | cttactcattgtatgaacatttctatctctcaagtgaaga |
| | actcaattataggattcaccttaaaggacttacagggaca |
| | gccggcaaaataagcagcatcagccaaccaggaaatgatt |
| | ttagcacaaaggatggagacaacgacaaatgtatttgcaa |
| | atgttcacaaatgctaacaggaggctggtggtttgatgca |
| | tgtggtccttccaacttgaacggaatgtactatccacaga |
| | ggcagaacacaaataagttcaacggcattaaatggtacta |
| | ctggaaaggctcaggctattcgctcaaggccacaaccatg |
| | atgatccgaccagcagatttctaaacatcccagtccacct |
| | gaggaactgtctcgaactattttcaaagacttaagcccag |
| | tgcactgaaagtcacggctgcgcactgtgtcctcttccac |
| | cacagagggcgtgtgctcggtgctgacgggacccacatgc |
| | tccagattagagcctgtaaactttatcacttaaacttgca |
| | tcacttaacggaccaaagcaagaccctaaacatccataat |
| | tgtgattagacagaacacctatgcaaagatgaacccgagg |
| | ct |
| | Ang2 polypeptide sequence |
| SEQ | MWQIVFFTLSCDLVLAAAYNNFRKSMDSIGKKQYQVQHGS |
| ID | CSYTFLLPEMDNCRSSSSPYVSNAVQRDAPLEYDDSVQRL |
| NO: | QVLENIMENNTQWLMKLENYIQDNMKKEMVEIQQNAVQNQ |
| 12 | TAVMIEIGTNLLNQTAEQTRKLTDVEAQVLNQTTRLELQL |
| | LEHSLSTNKLEKQILDQTSEINKLQDKNSFLEKKVLAMED |
| | KHIIQLQSIKEEKDQLQVLVSKQNSIIEELEKKIVTATVN |
| | NSVLQKQQHDLMETVNNLLTMMSTSNSKDPTVAKEEQISF |
| | RDCAEVFKSG HTTNGIYTLT FPNSTEEIKA YCDMEAG |
| | GGG WTIIQRREDGSVDFQRTWKEYKVGFGNPSGEYWLGN |
| | EFVSQLTNQQRYVLKIHLKDWEGNEAYSLYEHFYLSSEEL |
| | NYRIHLKGLTGTAGKISSISQPGNDFSTKDGDNDKCICKC |
| | SQMLTGGWWFDACGPSNLNGMYYPQRQNTNKFNGIKWYYW |
| | KGSGYSLKATTMMIRPADF |
| | Ang2 antibody VH polypeptide sequence |
| SEQ | QVQLVQSGAEVKKPGASVKVSCKASGYTFTGYYMHWVRQA |
| ID | PGQGLEWMGWINPNSGGTNYAQKFQGRVTMTRDTSISTAY |
| NO: | MELSRLRSDDTAVYYCARSPNPYYYDSSGYYYPGAFDIWG |
| 139 | QGTMVTVSS |
| | Ang2 antibody VL polypeptide sequence |
| SEQ | SYVLTQPPSVSVAPGQTARITCGGNNIGSKSVHWYQQKPG |
| ID | QAPVLVVYDDSDRPSGIPERFSGSNSGNTATLTISRVEAG |
| NO: | DEADYYCQVWDSSSDHWVFGGGTKLTVLSS |
| 140 | |
| | Ang2 scFv polypeptide sequence |
| SEQ | EVQLVESGGGLVQPGGSLRLSCAASGFTFSSYDIHWVRQA |
| ID | TGKGLEWVSAIGPAGDTYYPGSVKGRFTISRENAKNSLYL |
| NO: | QMNSLRAGDTAVYYCARGLITFGGLIAPFDYWGQGTLVTV |
| 141 | SSGGGGSGGGGSGGGGSGGGGSEIVLTQSPGTLSLSPGER |
| | ATLSCRASQSVSSTYLAWYQQKPGQAPRLLIYGASSRATG |
| | IPDRFSGSGSGT DFTLTISRLEPEDFAVYYCQHYDNSQT |
| | FGQ GTKVEIKRTVAA |

In some embodiments, the antibody or the antigen-binding fragment thereof of the present disclosure includes variants or derivatives thereof. For example, a non-human animal may be genetically modified to produce antibody variants or derivatives. In some embodiments, an antibody may be a single-domain antibody (sdAb), for example, a heavy chain only antibody (HCAb) VHH, or nanobody. Non-limiting examples of antigen-binding fragments include Fab, Fab′, F(ab′)2, dimers and trimers of Fab IL-6Rs, Fv, scFv, minibodies, dia-, tria-, and tetrabodies, and linear antibodies. Fab and Fab′ are antigen-binding fragments that comprise the VH and CH1 domains of the heavy chain linked to the VL and CL domains of the light chain via a disulfide bond. A F(ab′)2 comprises two Fab or Fab′ that are joined by disulfide bonds. A Fv comprises the VH and VL domains held together by non-covalent interactions. A scFv (single-chain variable fragment) is a fusion protein that comprises the VH and VL domains connected by a peptide linker. Manipulation of the orientation of the VH and VL domains and the linker length may be used to create different forms of molecules that may be monomeric, dimeric (diabody), trimeric (triabody), or tetrameric (tetrabody). Minibodies are scFv-CH3 fusion proteins that assemble into bivalent dimers.

In some embodiments, the antibody is a binding fragment thereof. In some cases, the antibody is a humanized antibody or binding fragment thereof, a chimeric antibody or binding fragment thereof, a monoclonal antibody or binding fragment thereof, a multi-specific antibody or binding fragment thereof, a bispecific antibody or binding fragment thereof, or a single-domain antibody (e.g. Nanobody®) thereof. In some embodiments, the antibody may be a multi-specific antibody. In some cases, the multi-specific antibody comprises two or more target binding moieties in which each of the two or more target binding moieties binds specifically to an antigen, and the two or more antigens are different. In some cases, the multi-specific antibody comprises target binding moieties that specifically bind to three or more different antigens, four or more different antigens, or five or more different antigens. In some embodiments, the antibody may be a bispecific antibody. In some cases, the bispecific antibody or binding fragment includes a Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), FcAAdp, XmAb, Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), or nanobody.

In some embodiments, the antibody described herein comprises an IgG framework, an IgA framework, an IgE framework, or an IgM framework. In some cases, the antibody comprises an IgG framework (e.g., IgG1, IgG2, IgG3, or IgG4). In some cases, the antibody comprises an IgG1 framework. In some cases, the antibody comprises an IgG2 (e.g., an IgG2a or IgG2b) framework. In some cases, the antibody comprises an IgG2a framework. In some cases, the antibody comprises an IgG2b framework. In some cases, the antibody comprises an IgG3 framework. In some cases, the antibody comprises an IgG4 framework.

In some cases, the antibody described herein comprises one or more mutations in a framework region, e.g., in the CH1 domain, CH2 domain, CH3 domain, hinge region, or a combination thereof. In some cases, the one or more mutations are to stabilize the antibody and/or to increase half-life. In some cases, the one or more mutations are to modulate Fc receptor interactions, to reduce or eliminate Fe effector functions such as FcγR, antibody-dependent cell-mediated cytotoxicity (ADCC), or complement-dependent cytotoxicity (CDC). In additional cases, the one or more mutations are to modulate glycosylation.

### Inhibitory RNA

In some embodiments, the activator of the RTK/Tie2 described herein comprises RNA or DNA. In some cases, the activator of the RTK/Tie2 comprises an inhibitory RNA for modulating a signaling transduction pathway by decreasing the expression of a protein. In some embodiments, the inhibitory RNA targets and decreases expression of VEGF. In some embodiments, the inhibitory RNA targets and decreases expression of an angiopoietin. In some embodiments, the inhibitory RNA targets and decreases expression of Ang2, leading to the increasing of RTK/Tie2 signaling transduction pathway. The inhibitory RNA can target and bind to a nucleic acid sequence of Ang2. In some embodiments, the inhibitory RNA targets and binds to a transcript of Ang2 comprising a nucleic acid sequence that is at least 70%, at least 75%, at least 80%, at least 85%, at least 90%, at least 95%, at least 99%, or more identical to SEQ ID NO: 13. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the RNA comprises siRNA. In some instances, the signaling transduction regulator comprises shRNA.

In some embodiments, the activator of the RTK/Tie2 comprising inhibitory RNA is from about 10 to about 50 nucleotides in length. In some instances, the signaling transduction regulator is from about 10 to about 30, from about 15 to about 30, from about 18 to about 25, from about 18 to about 24, from about 19 to about 23, or from about 20 to about 22 nucleotides in length. In some embodiments, the signaling transduction regulator hybridizes to at least 8, 9, 10, 11, 12, 13, 14, 15, 16, 17, 18, 19, 20, or more contiguous bases of a target sequence described herein.

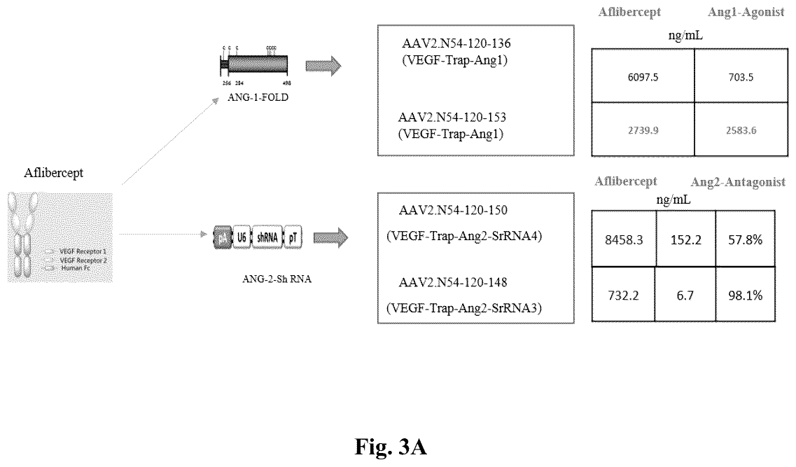

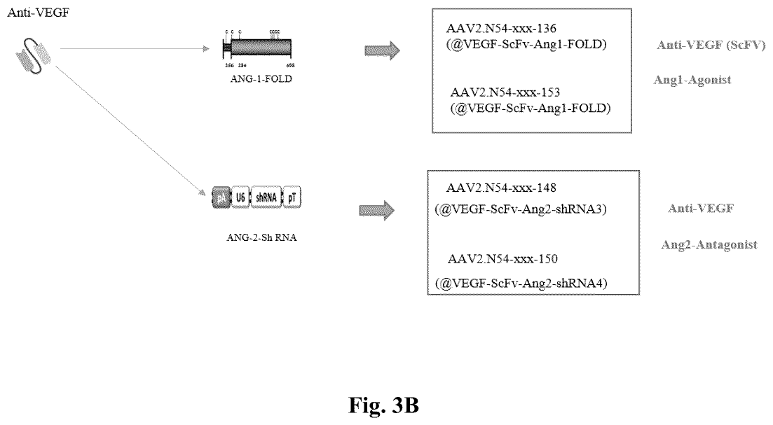

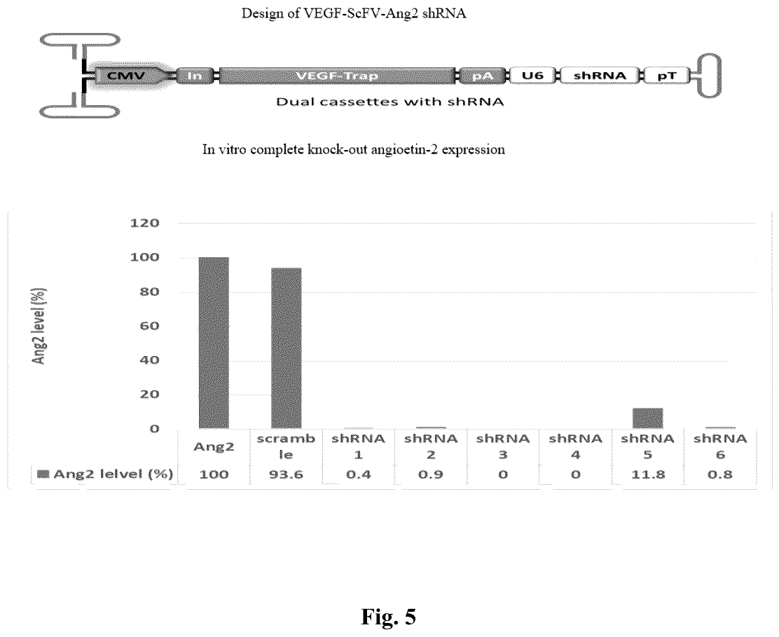

In some embodiments, the activator of the RTK/Tie2 comprises a shRNA for targeting and decreasing the endogenous expression of Ang2. FIG. 3B, FIG. 5, and Table 10 illustrate the inhibitory effect of Ang2 shRNA on endogenous Ang2. In some embodiments, the Ang2 shRNA comprises a nucleic acid sequence that is at least 70%, at least 75%, at least 80%, at least 85%, at least 90%, at least 95%, or at least 99% identical to any one of SEQ ID NOs: 81-86 (Table 16). In some embodiments, the Ang2 shRNA comprises a nucleic acid sequence that is any one of SEQ ID NOs: 81-86. In some embodiments, the Ang2 shRNA comprises a nucleic acid sequence that is SEQ ID NO: 81. In some embodiments, the Ang2 shRNA comprises a nucleic acid sequence that is SEQ ID NO: 82. In some embodiments, the Ang2 shRNA comprises a nucleic acid sequence that is SEQ ID NO: 83. In some embodiments, the Ang2 shRNA comprises a nucleic acid sequence that is SEQ ID NO: 84. In some embodiments, the Ang2 shRNA comprises a nucleic acid sequence that is SEQ ID NO: 85. In some embodiments, the Ang2 shRNA comprises a nucleic acid sequence that is SEQ ID NO: 86. In some embodiments, the Ang2 shRNA does not comprises a nucleic acid sequence that is SEQ ID NO: 87.

TABLE 16
Exemplary Ang2 shRNAs

| SEQ ID | | |
|---|---|---|
| NO | Exemplary Ang2 shRNA | Notes |
| SEQ ID | GGTTCAACGGCATTAAATAta | Ang2 shRNA 1 |
| NO: 81 | cctgacccataTATTTAATGC | |
| | CGTTGAACCTTTTT | |
| SEQ ID | GGAAGCTTGAGAATTATAAta | Ang2 shRNA 2 |
| NO: 82 | cctgacccataTTATAATTCT | |
| | CAAGCTTCCTTTTT | |
| SEQ ID | GTGAAGAACTCAATTATAAta | Ang2 shRNA 3 |
| NO: 83 | cctgacccataTTATAATTGA | |
| | GTTCTTCACTTTTT | |
| SEQ ID | GTAACATTCCCTAATTCTAta | Ang2 shRNA 4 |
| NO: 84 | cctgacccataTAGAATTAGG | |
| | GAATGTTACTTTTT | |
| SEQ ID | GACTTGGAAAGAATATAAAta | Ang2 shRNA 5 |
| NO: 85 | cctgacccataTTTATATTCT | |
| | TTCCAAGTCTTTTT | |
| SEQ ID | GGTGAAGAACTCAATTATAta | Ang2 shRNA 6 |
| NO: 86 | cctgacccataTATAATTGAG | |
| | TTCTTCACCTTTTT | |
| SEQ ID | GTGCATATGAACGTAACTAta | Ang2 shRNA |
| NO: 87 | cctgacccataTAGTTACGTT | scrambled |
| | CATATGCACTTTTT | |

### Methods

### Vector Construction and Delivery

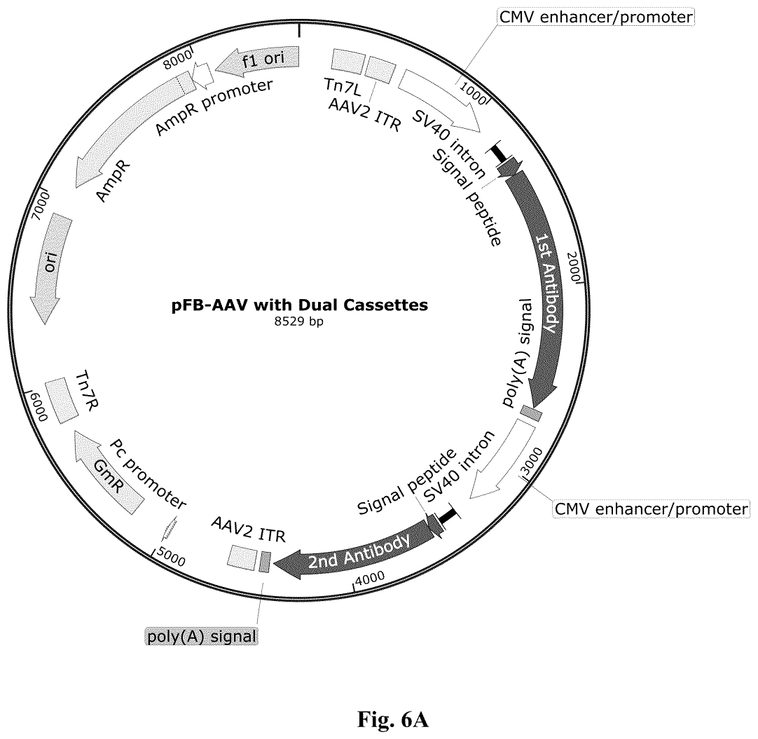

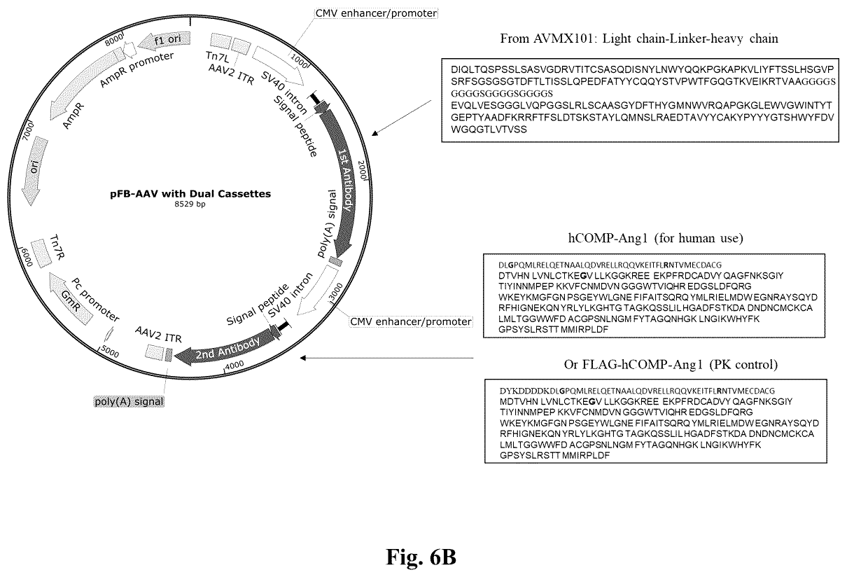

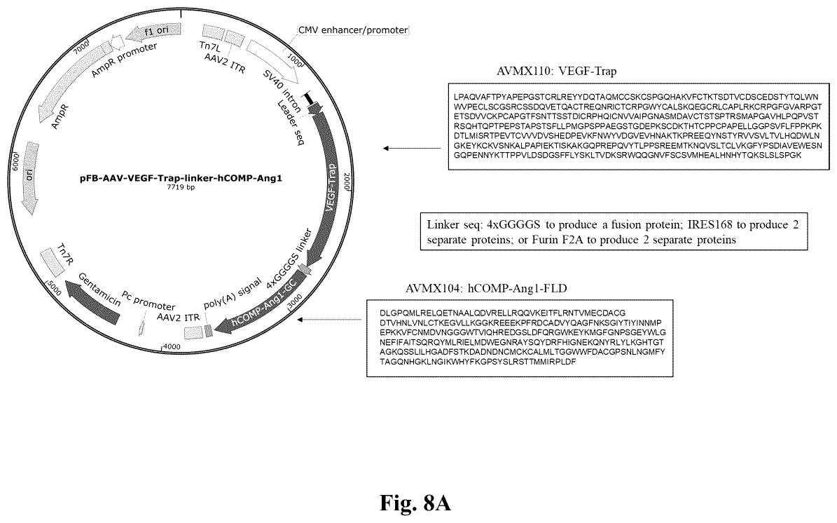

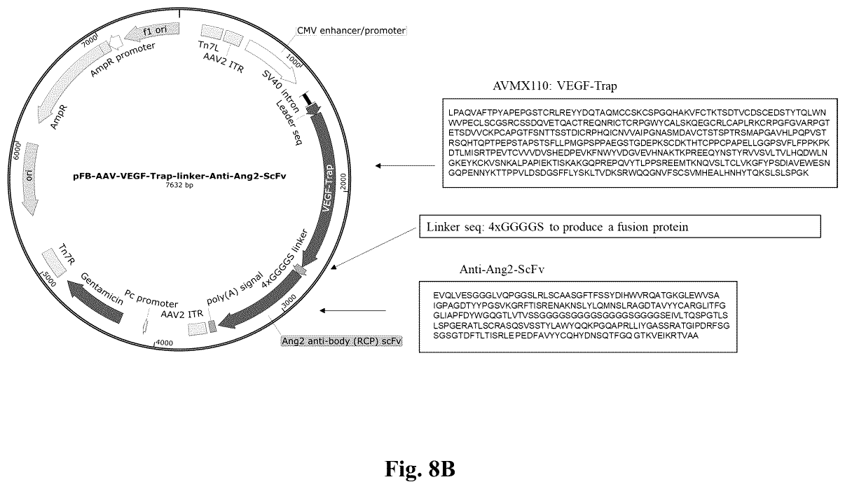

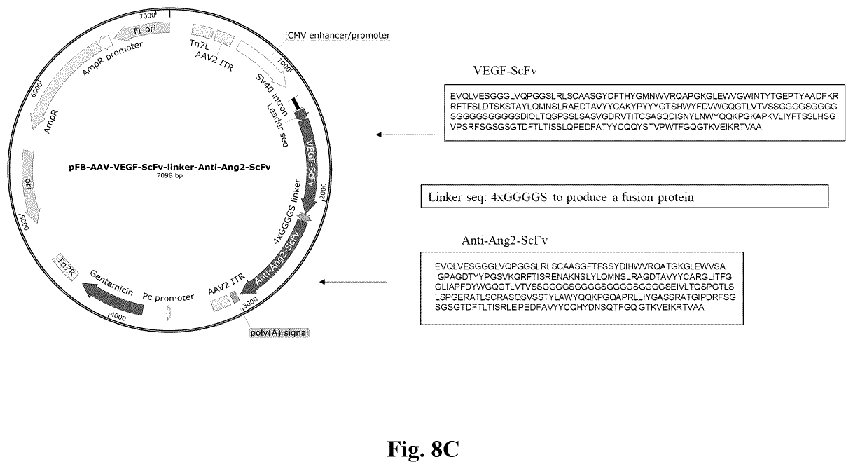

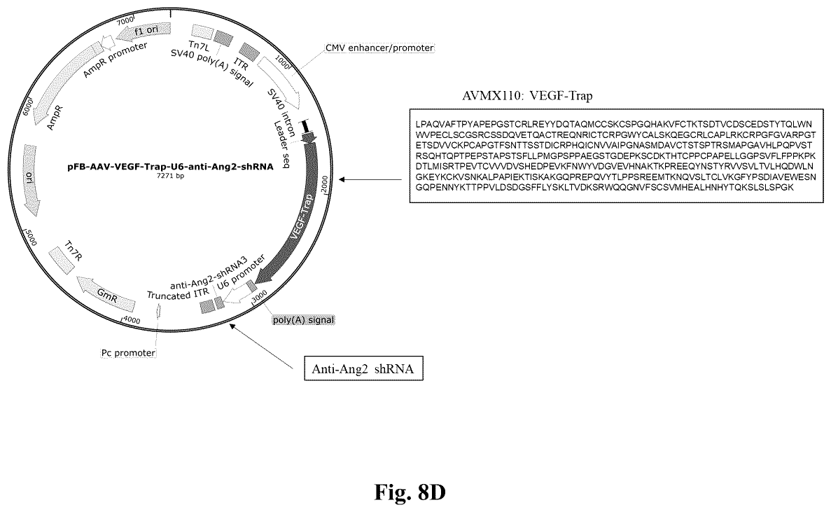

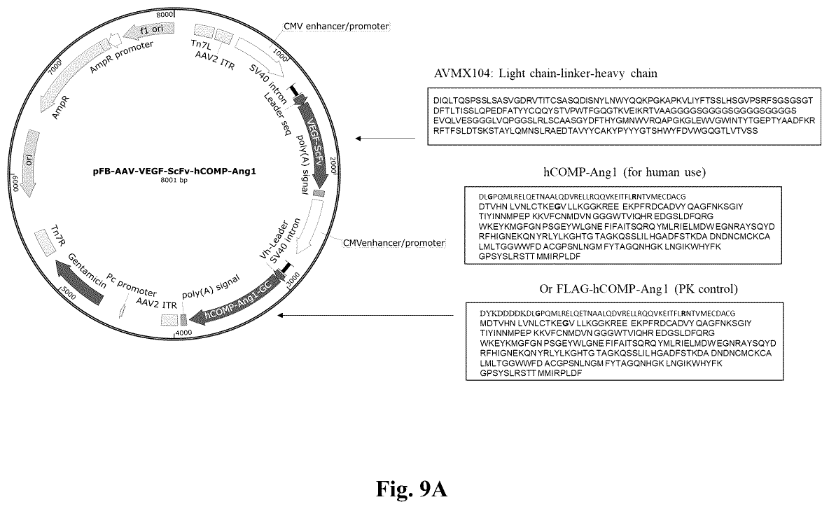

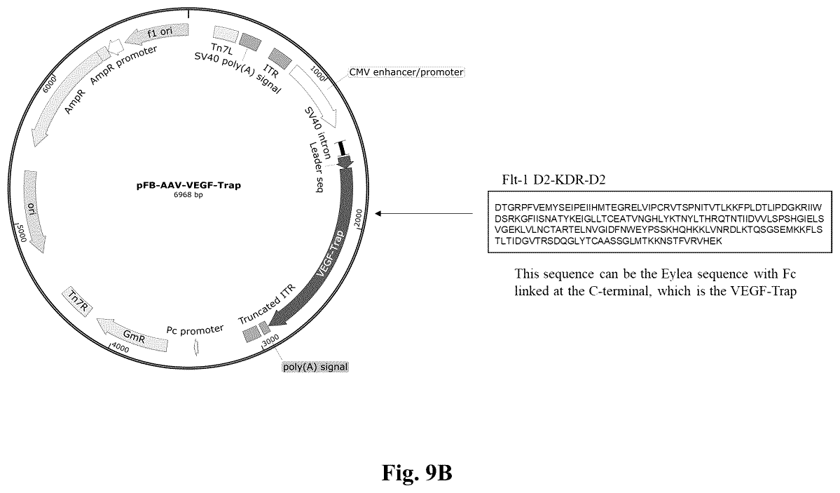

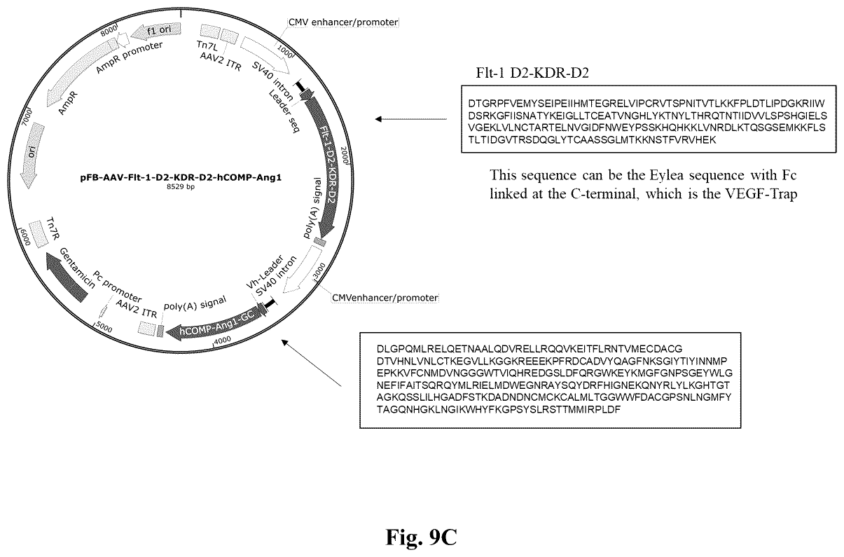

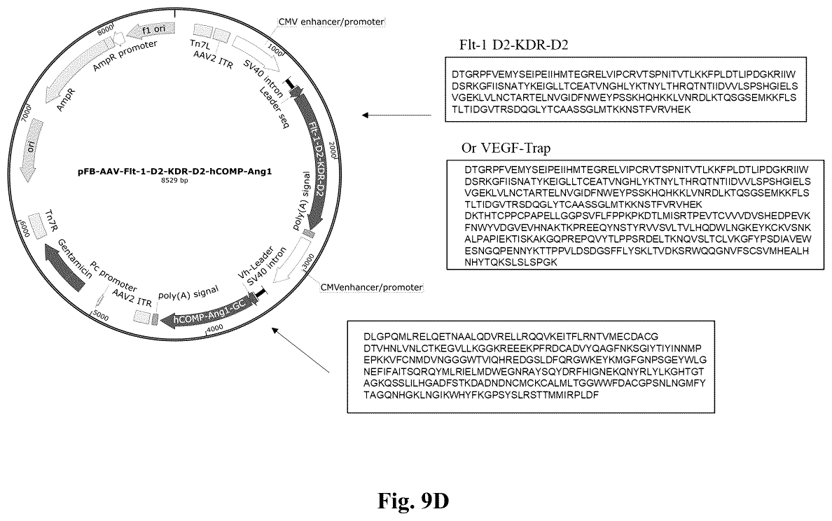

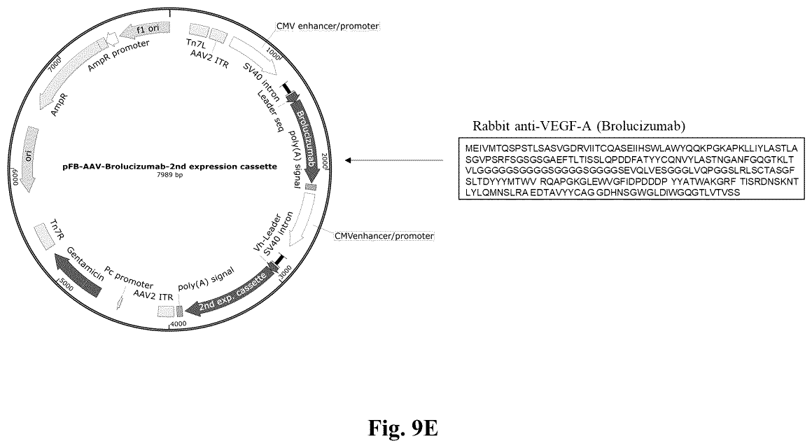

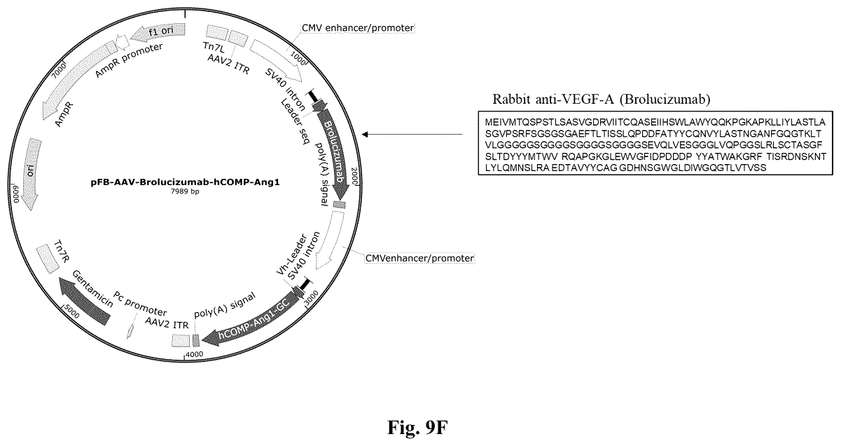

Described herein are methods for generating the non-naturally occurring polynucleotide comprising one or more expression cassettes. In some embodiments, the non-naturally occurring polynucleotide is part of an AAV vector. In some embodiments, the non-naturally occurring polynucleotide comprises one or more promoters or IRES. FIGS. 6-9 and Table 17 illustrate exemplary AAV vectors showing the non-naturally occurring polynucleotide comprising the arrangement of the one or more expression cassettes.

TABLE 17
Exemplary non-naturally occurring
polynucleotide for expressing polypeptide
sequence of VEGF inhibitor and Ang1 fusion

| SEQ | | |
|---|---|---|
| ID | Exemplary vector polypeptide | |
| NO | sequence | Notes |
| SEQ | EVQLVESGGGLVQPGGSLRLSCAASGYDFT | AVMX1 |
| ID | HYGMNWVRQAPGKGLEWVGWINTYTGEPTY | 03a: |
| NO: | AADFKRRFTFSLDTSKSTAYLQMNSLRAED | Anti- |
| 41 | TAVYYCAKYPYYYGTSHWYFDVWGQGTLVT | VEGF- |
| | VSSASTKGPSVFPLAPSSKSTSGGTAALGC | (Fab)2- |
| | LVKDYFPEPVTVSWNSGALTSGVHTFPAVL | Linker- |
| | QSSGLYSLSSVVTVPSSSLGTQTYICNVNH | hCOMP- |
| | KPSNTKVDKKVEPKSCDKTHLGGGGSGDLG | Ang1; |
| | PQMLRELQETNAALQDVRELLRQQVKEITF | CH |
| | LRNTVMECDACGDTVHNLVNLCTKEGVLLK | |
| | GGKREEEKPFRDCADVYQAGFNKSGIYTIY | |
| | INNMPEPKKVFCNMDVNGGGWTVIQHREDG | |
| | SLDFQRGWKEYKMGFGNPSGEYWLGNEFIF | |
| | AITSQRQYMLRIELMDWEGNRAYSQYDRFH | |
| | IGNEKQNYRLYLKGHTGTAGKQSSLILHGA | |
| | DFSTKDADNDNCMCKCALMLTGGWWFDACG | |
| | PSNLNGMFYTAGQNHGKLNGIKWHYFKGPS | |
| | YSLRSTTMMIRPLDF | |
| SEQ | EVQLVESGGGLVQPGGSLRLSCAASGYDFT | AVMX1 |
| ID | HYGMNWVRQAPGKGLEWVGWINTYTGEPTY | 03a: |
| NO: | AADFKRRFTFSLDTSKSTAYLQMNSLRAED | Anti- |
| 42 | TAVYYCAKYPYYYGTSHWYFDVWGQGTLVT | VEGF- |
| | VSSASTKGPSVFPLAPSSKSTSGGTAALGC | (Fab)2- |
| | LVKDYFPEPVTVSWNSGALTSGVHTFPAVL | Linker- |
| | QSSGLYSLSSVVTVPSSSLGTQTYICNVNH | Ang1; |
| | KPSNTKVDKKVEPKSCDKTHLGGGGSGDTV | no |
| | HNLVNLCTKEGVLLKGGKREEEKPFRDCAD | hCOMP; |
| | VYQAGFNKSGIYTIYINNMPEPKKVFCNMD | CH |
| | VNGGGWTVIQHREDGSLDFQRGWKEYKMGF | |
| | GNPSGEYWLGNEFIFAITSQRQYMLRIELM | |
| | DWEGNRAYSQYDRFHIGNEKQNYRLYLKGH | |
| | TGTAGKQSSLILHGADFSTKDADNDNCMCK | |
| | CALMLTGGWWFDACGPSNLNGMFYTAGQNH | |
| | GKLNGIKWHYFKGPSYSLRSTTMMIRPLDF | |
| SEQ | DIQLTQSPSSLSASVGDRVTITCSASQDIS | AVMX1 |
| ID | NYLNWYQQKPGKAPKVLIYFTSSLHSGVPS | 03a: |
| NO: | RFSGSGSGTDFTLTISSLQPEDFATYYCQQ | Anti- |
| 43 | YSTVPWTFGQGTKVEIKRTVAAPSVFIFPP | VEGF- |
| | SDEQLKSGTASVVCLLNNFYPREAKVQWKV | (Fab)2- |
| | DNALQSGNSQESVTEQDSKDSTYSLSSTLT | Linker- |
| | LSKADYEKHKVYACEVTHQGLSSPVTKSFN | hCOMP |
| | RGEC | -Ang1; |
| | | CL |
| SEQ | LPAQVAFTPYAPEPGSTCRLREYYDQTAQM | Dual |
| ID | CCSKCSPGQHAKVFCTKTSDTVCDSCEDST | Trans- |
| NO: | YTQLWNWVPECLSCGSRCSSDQVETQACTR | gene: |
| 44 | EQNRICTCRPGWYCALSKQEGCRLCAPLRK | AVMX- |
| | CRPGFGVARPGTETSDVVCKPCAPGTFSNT | 110: |
| | TSSTDICRPHQICNVVAIPGNASMDAVCTS | hCOMP- |
| | TSPTRSMAPGAVHLPQPVSTRSQHTQPTPE | Ang1.F |
| | PSTAPSTSFLLPMGPSPPAEGSTGDEPKSC | LD; |
| | DKTHTCPPCPAPELLGGPSVFLFPPKPKDT | VEGF |
| | LMISRTPEVTCVVVDVSHEDPEVKFNWYVD | Trap |
| | GVEVHNAKTKPREEQYNSTYRVVSVLTVLH | |
| | QDWLNGKEYKCKVSNKALPAPIEKTISKAK | |
| | GQPREPQVYTLPPSREEMTKNQVSLTCLVK | |
| | GFYPSDIAVEWESNGQPENNYKTTPPVLDS | |
| | DGSFFLYSKLTVDKSRWQQGNVFSCSVMHE | |
| | ALHNHYTQKSLSLSPGK | |

The non-naturally occurring polynucleotide can be readily introduced into a host cell, e.g., mammalian, bacterial, yeast, or insect cell by any method in the art. For example, the non-naturally occurring polynucleotide can be transferred into a host cell by physical, chemical, or biological means. In some embodiments, the non-naturally occurring polynucleotide can be delivered into the cell via physical methods such as calcium phosphate precipitation, lipofection, particle bombardment, microinjection, gene gun, electroporation, and the like.

Physical methods for introducing the non-naturally occurring polynucleotide encoding into the cell can include calcium phosphate precipitation, lipofection, particle bombardment, microinjection, gene gun, electroporation, and the like. One method for the introduction of the non-naturally occurring polynucleotide a host cell is calcium phosphate transfection.

Chemical means for introducing the non-naturally occurring polynucleotide encoding the non-naturally into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of non-naturally occurring polynucleotide or vector encoding the non-naturally occurring polynucleotide with targeted nanoparticles.

In the case where a non-viral delivery system is utilized, an example delivery vehicle is a liposome. The use of lipid formulations is contemplated for the introduction of the non-naturally occurring polynucleotide or vector encoding the non-naturally occurring polynucleotide into a cell (in vitro, ex vivo, or in vivo). In another aspect, the vector can be associated with a lipid. The vector associated with a lipid can be encapsulated in the aqueous interior of a liposome, interspersed within the lipid bilayer of a liposome, attached to a liposome via a linking molecule that is associated with both the liposome and the non-naturally occurring polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the non-naturally occurring polynucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the non-naturally occurring polynucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some embodiments, the non-naturally occurring polynucleotide can be delivered into the cell via biological methods such as the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAV vectors), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV comprises a serotype, including AAV1, AAV2, AAV4, AAV5, AAV6, AAV7, AAV8, AAV9, AAV10, AAV11, AAV12, or a combination thereof. Based on these initial serotypes, AAV capsid of each serotype can be engineered to make them better suited for biological functions, tissue or cell selection. In some embodiments, an AAV is AAV2 and variants AAV2.N53 and AAV2.N54 which are used in the examples of the invention. Chimeric AAVs are also contemplated that may contain at least 2 AAV serotypes. In some cases, at least 3, at least 4, at least 5, at least 6, at least 7, or up to 8 different serotypes are combined in a chimeric AAV. In some cases, only a portion of the AAV is chimeric. For example, suitable portions can include the capsid, VP1, VP2, or VP3 domains and/or Rep. In some cases, at least one of VP1, VP2, and VP3 has at least one amino acid substitution compared to an otherwise comparable wild-type AAV capsid protein. In some cases, a mutation can occur in VP1 and VP2, in VP1 and VP3, in VP2 and VP3, or in VP1, VP2, and VP3. In some embodiments, at least one of VP1, VP2, and VP3 has from one to about 25 amino acid substitutions compared to wild-type AAV VP1, VP2, and VP3, e.g., from about one to about 5, from about 5 to about 10, from about 10 to about 15, from about 15 to about 20, or from about 20 to about 25 amino acid substitutions compared to wild-type AAV VP1, VP2, and VP3. In some cases, a VP can be removed. For example, in some embodiments a mutant AAV does not comprise at least one of VP1, VP2, or VP3.

In some instances, the viral vector is a chimeric viral vector, comprising viral portions from two or more viruses. In additional instances, the viral vector is a recombinant viral vector. In some cases, the vector comprises additional features. Additional features can comprise sequences such as tags, signaling peptides, intronic sequences, promoters, stuffer sequences, and the like. In some cases, the vector comprises a signaling peptide. A signaling peptide is sometimes referred to as signaling sequence, targeting signal, localization signal, localization sequence, transit peptide, leader sequence or leader peptide, is a short peptide present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. In some cases, nucleic acids provided herein can comprise signaling peptides. A signaling peptide can be of any length but typically from 15-30 amino acids long. A signaling peptide can be from about: 10-15, 10-20, 10-30, 15-20, 15-25, 15-30, 20-30, or 25-30 amino acids long. Various signaling peptides can be utilized and include but are not limited to: human antibody heavy chain (Vh), human antibody light chain (Vl), and aflibercept.

In an embodiment, an additional feature of the vector includes promoter. Promoter is sequences of DNA to which proteins bind that initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. In some cases, the promoters can be inducible promoters. Various promoters are contemplated and can be employed in the vectors of the disclosure. In an embodiment, a promoter is: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof. In some cases, the promoter is the CMV promoter.

In some embodiments, the vector comprising the at least two expression cassettes under expression control of two different promoters. Such arrangement allows the two signaling transduction regulators to be expressed simultaneously or in a desired sequential order in a cell. For example, the vector comprising the VEGF inhibitor and Ang1 protein can be engineered to constitutively express the VEGF inhibitor (e.g., the VEGF inhibitor is under the control of a CMV promoter), while the Ang1 protein can be expressed at a later time (e.g., the Ang1 protein is under the control of an inducible promoter). In some cases, the use of two promoters also allow modulating the expressions of the two signaling transduction regulators. For example, VEGF inhibitor can be driven by a promoter with a strong expression activity in a specific cell type, while the Ang1 protein is driven by a different promoter with a weaker expression activity in the same cell type.

In an aspect, provided herein are also methods of modifying cells to thereby generate engineered cells. Cells can refer to primary cells, recombinant cells, or cell lines. In some cases, a cell is a packaging cell. A packaging cell can be any one of: HEK 293 cells, HeLa cells, and Vero cells to name a few. An engineered cell can be a primary cell. In some cases, an engineered cell can be an ocular cell. Suitable ocular cells include but are not limited to a: photoreceptor, ganglion cell, RPE cell, amacrine cell, horizontal cell, muller cell, and the like.

In some cases, a cell is a packaging cell utilized to generate viral particles. To generate AAV virions or viral particles, an AAV vector is introduced into a suitable host cell using known techniques, such as by transfection. In some cases, transfection techniques are used, e.g., CaPO₄ transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins). Suitable transfection methods include calcium phosphate co-precipitation, direct micro-injection, electroporation, liposome mediated gene transfer, and nucleic acid delivery using high-velocity microprojectiles, which are known in the art.

To engineer a cell, a plurality of cells may be contacted with an isolated non-naturally occurring nucleic acid. Contacting can comprise any length of time and may include from about 5 min to about 5 days. Contacting can last from about 5, about 10, about 15, about 20, about 25, about 30, about 35, about 40, about 45, about 50, about 55, or about 60 minutes. In some cases, the contacting can last from 1 hour, 3 hours, 5 hours, 10 hours, 15 hours, 20 hours, 1 day, 2 days, 3 days, 4 days or up to about 5 days.

In some cases, supernatant of the packaging cell line is treated by PEG precipitation for concentrating the virus. In other cases, a centrifugation step can be used to concentrate a virus. For example, a column can be used to concentration a virus during a centrifugation. In some embodiments, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some embodiments, the recombinant AAV is isolated from the PEG-precipitated supernatant by low-speed centrifugation followed by CsCl gradient. The low-speed centrifugation can be to can be about 4000 rpm, about 4500 rpm, about 5000 rpm, or about 6000 rpm for about 20 minutes, about 30 minutes, about 40 minutes, about 50 minutes or about 60 minutes. In some cases, recombinant AAV is isolated from the PEG-precipitated supernatant by centrifugation at about 5000 rpm for about 30 minutes followed by CsCl gradient. In some cases, CsCl purification can be replaced with IDX gradient ultracentrifugation. Supernatant can be collected at about 12 hours, about 24 hours, about 36 hours, about 48 hours, about 72 hours, about 96 hours, about 120 hours, or a time between any of these two time points after a transfection. Supernatant can also be purified, concentrated, or a combination thereof. For example, a concentration or viral titer can be determined by qPCR or silver stain.

In an aspect, provided is also a plurality of AAV particles (containing the non-naturally occurring polynucleotide described herein) isolated from an engineered cell. A viral titer can be from about 10² vp/mL, about 10³ vp/mL, about 10⁴ vp/mL, about 10⁵ vp/mL, about 10⁶ vp/mL, about 10⁷ vp/mL, about 10⁸ vp/mL, or up to about 10⁹ vp/mL. A viral titer can be from about 10² GC/mL, about 103 GC/mL, about 10⁴ GC/mL, about 10⁵ GC/mL, about 10⁶ GC/mL, about 10⁷ GC/mL, about 10⁸ GC/mL, or up to about 10⁹ GC/mL. In some cases, a viral titer can be from about 10² TU/mL, about 103 TU/mL, about 10⁴ TU/mL, about 10⁵ TU/mL, about 10⁶ TU/mL, about 10⁷ TU/mL, 108 TU/mL, or up to about 10⁹ TU/mL. An optimal viral titer can vary depending on cell type to be transduced. A range of virus can be from about 1000 MOI to about 2000 MOI, from about 1500 MOI to about 2500 MOI, from about 2000 MOI to about 3000 MOI, from about 3000 MOI to about 4000 MOI, from about 4000 MOI to about 5000 MOI, from about 5000 MOI to about 6000 MOI, from about 6000 MOI to about 7000 MOI, from about 7000 MOI to about 8000 MOI, from about 8000 MOI to about 9000 MOI, from about 9000 MOI to about 10,000 MOI. For example, to infect 1 million cells using a MOI of 10,000, one will need 10,000×1,000,000=10¹⁰ GC.

In some cases, a plurality of AAV particles can be formulated into unit dose form. Various formulations are contemplated for adult or pediatric delivery and include but are not limited to: 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, or up to 1.5×10¹⁵ vg. Compositions of viral particles can be cryopreserved or otherwise stored in suitable containers.

Provided compositions and methods herein can be sufficient to enhance delivery and/or expression of subject biologic by at least about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or up to 100% more than an otherwise comparable unmodified nucleic acid. In some cases, the otherwise comparable unmodified nucleic acid is one that encodes VEGF-Trap. In some cases, modifications can be sufficient to enhance delivery and/or expression of subject biologics by at least about 1-fold, about 6-fold, about 11-fold, about 16-fold, about 21-fold, about 26-fold, about 31-fold, about 36-fold, about 41-fold, about 46-fold, about 51-fold, about 56-fold, about 61-fold, about 66-fold, about 71-fold, about 76-fold, about 81-fold, about 86-fold, about 91-fold, about 96-fold, about 101-fold, about 106-fold, about 111-fold, about 116-fold, about 121-fold, about 126-fold, about 131-fold, about 136-fold, about 141-fold, about 146-fold, about 151-fold, about 156-fold, about 161-fold, about 166-fold, about 171-fold, about 176-fold, about 181-fold, about 186-fold, about 191-fold, about 196-fold, about 201-fold, about 206-fold, about 211-fold, about 216-fold, about 221-fold, about 226-fold, about 231-fold, about 236-fold, about 241-fold, about 246-fold, about 251-fold, about 256-fold, about 261-fold, about 266-fold, about 271-fold, about 276-fold, about 281-fold, about 286-fold, about 291-fold, about 296-fold, about 301-fold, about 306-fold, about 311-fold, about 316-fold, about 321-fold, about 326-fold, about 331-fold, about 336-fold, about 341-fold, about 346-fold, or about 350-fold more than an otherwise comparable unmodified nucleic acid. In an embodiment, increased expression comprises at least a 5-fold, at least a 10-fold, at least a 20-fold, at least a 50-fold, at least a 100-fold, at least a 200-fold, or at least a 500-fold increase as determined by in in vitro assay. Suitable in vitro assays include ELISA, western blot, Luminex, microscopy, imaging, and/or flow cytometry.

A subject AAV virion can exhibit at least 1-fold, at least 6-fold, at least 10-fold, at least 15-fold, at least 20-fold, at least 25-fold, at least 50-fold, or more than 50-fold, increased infectivity of a retinal cell, compared to the infectivity of the retinal cell (photoreceptor, ganglion cell, RPE cell, amacrine cell, horizontal cell, muller cell, and the like) by an AAV virion comprising an otherwise comparable WT AAV capsid protein.

### Treatment

Provided herein are methods of treating a disease or condition described here. A method of treatment can comprise introducing to a subject in need a non-naturally occurring polynucleotide, an AAV vector comprising the non-naturally occurring polynucleotide, or an AAV comprising the non-naturally occurring polynucleotide. Also provided is a method of treating disease or condition that comprises administering a pharmaceutical composition to a subject in need thereof. A pharmaceutical composition can comprise a sequence that encodes a biologic that comprises the non-naturally occurring polynucleotide, the AAV vector comprising the non-naturally occurring polynucleotide, or the AAV comprising the non-naturally occurring polynucleotide. In some embodiments, administration is by any suitable mode of administration, including systemic administration (e.g., intravenous, inhalation, vitreous, or etc.). In some embodiments, the subject is human.

In some embodiments, the non-naturally occurring polynucleotide, the AAV vector comprising the non-naturally occurring polynucleotide, the AAV comprising the non-naturally occurring polynucleotide, or the pharmaceutical composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time.

In some embodiments, the method comprises administering the non-naturally occurring polynucleotide, the AAV vector comprising the non-naturally occurring polynucleotide, the AAV comprising the non-naturally occurring polynucleotide, or the pharmaceutical composition in a therapeutically-effective amount by various forms and routes including, for example, oral, or topical administration. In some embodiments, a composition may be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the method comprises administering the non-naturally occurring polynucleotide, the AAV vector comprising the non-naturally occurring polynucleotide, the AAV comprising the non-naturally occurring polynucleotide, or the pharmaceutical composition by intravenous infusion. In some embodiments, the non-naturally occurring polynucleotide, the AAV vector comprising the non-naturally occurring polynucleotide, the AAV comprising the non-naturally occurring polynucleotide, or the pharmaceutical composition is administered by slow continuous infusion over a long period, such as more than 24 hours. In some embodiments, t the non-naturally occurring polynucleotide, the AAV vector comprising the non-naturally occurring polynucleotide, the AAV comprising the non-naturally occurring polynucleotide, or the pharmaceutical composition is administered as an intravenous injection or a short infusion. In some embodiments, t the non-naturally occurring polynucleotide, the AAV vector comprising the non-naturally occurring polynucleotide, the AAV comprising the non-naturally occurring polynucleotide, or the pharmaceutical composition is administered via vitreous route. In some embodiments, the non-naturally occurring polynucleotide, the AAV vector comprising the non-naturally occurring polynucleotide, the AAV comprising the non-naturally occurring polynucleotide, or the pharmaceutical composition may be administered in a local manner, for example, via injection of the agent directly into an organ, optionally in a depot or sustained release formulation or implant.

In some embodiments, the non-naturally occurring polynucleotide, the AAV vector comprising the non-naturally occurring polynucleotide, the AAV comprising the non-naturally occurring polynucleotide, or the pharmaceutical composition may be administered in conjunction with other therapies, for example, an antiviral therapy, a chemotherapy, an antibiotic, a cell therapy, a cytokine therapy, or an anti-inflammatory agent. In some embodiments, the non-naturally occurring polynucleotide or a pharmaceutical composition comprising the non-naturally occurring polynucleotide may be administered before, during, or after the occurrence of a disease or condition, and the timing of administering the composition containing a therapeutic agent may vary. In some cases, the composition may be used as a prophylactic and may be administered continuously to subjects (e.g., the subject for immunization or the subject for treatment) with a susceptibility to a coronavirus or a propensity to a condition or disease associated with a coronavirus. Prophylactic administration may lessen a likelihood of the occurrence of the infection, disease or condition, or may reduce the severity of the infection, disease or condition.

The non-naturally occurring polynucleotide, the AAV vector comprising the non-naturally occurring polynucleotide, the AAV comprising the non-naturally occurring polynucleotide, or the pharmaceutical composition e may be administered to a subject before the onset of the symptoms. The non-naturally occurring polynucleotide, the AAV vector comprising the non-naturally occurring polynucleotide, the AAV comprising the non-naturally occurring polynucleotide, or the pharmaceutical composition may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen levels. A therapeutic agent may be administered after (e.g., as soon as is practicable after) the onset of a disease or condition is detected or suspected. A therapeutic agent may be administered after (e.g., as soon as is practicable after) a potential exposure to a coronavirus, for example, after a subject (e.g., the subject for immunization or the subject for treatment) has contact with an infected subject, or learns they had contact with an infected subject that may be contagious.

Actual dosage levels of an agent of the disclosure (e.g., the non-naturally occurring polynucleotide or a pharmaceutical composition) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

The non-naturally occurring polynucleotide, the AAV vector comprising the non-naturally occurring polynucleotide, the AAV comprising the non-naturally occurring polynucleotide, or the pharmaceutical composition described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

In some cases, an increased level of a biologic in a subject is at least a 5-fold, a 10-fold, a 20-fold, a 50-fold, a 100-fold, a 200-fold, or a 500-fold increased, as determined by a diagnostic assay.

Suitable diagnostic assays can include ocular diagnostic assays. Ocular diagnostic assays can include ophthalmic testing such as refraction testing, ocular scans, Ocular coherence tomography, Farnworth-Munsell 100 Hue Test, Computerized Optic Disc Imaging and Nerve Fiber Layer Analysis (GDX, HRT, OCT), Corneal Topography, Electroretinography (ERG), electro-oculography (EOG), visual evoked potentials (VEP), visual evoked response (VER), Fluorescein Angiography, Ocular Coherence Tomography (OCT), retinal photography, fundus photography, Specular Microscopy, Goldmann, Humphrey, FDT, Octopus, Biometry/IOL calculation, A-Scan, B-Scan, and combinations thereof.

In some cases, a retinal test can be utilized. Nonlimiting methods for assessing retinal function and changes thereof include assessing visual acuity (e.g. best-corrected visual acuity [BCVA], ambulation, navigation, object detection and discrimination), assessing visual field (e.g. static and kinetic visual field perimetry), performing a clinical examination (e.g. slit lamp examination of the anterior and posterior segments of the eye), assessing electrophysiological responsiveness to all wavelengths of light and dark (e.g. all forms of electroretinography (ERG) [full-field, multifocal and pattern], all forms of visual evoked potential (VEP), electrooculography (EOG), color vision, dark adaptation and/or contrast sensitivity). Nonlimiting methods for assessing anatomy and retinal health and changes thereof include Optical Coherence Tomography (OCT), fundus photography, adaptive optics scanning laser ophthalmoscopy (AO-SLO), fluorescence and/or autofluorescence; measuring ocular motility and eye movements (e.g. nystagmus, fixation preference, and stability), measuring reported outcomes (patient-reported changes in visual and non-visually-guided behaviors and activities, patient-reported outcomes [PRO], questionnaire-based assessments of quality-of-life, daily activities and measures of neurological function (e.g. functional Magnetic Resonance Imaging (MRI)).

In some embodiments, the non-naturally occurring polynucleotide, the AAV vector comprising the non-naturally occurring polynucleotide, the AAV comprising the non-naturally occurring polynucleotide, or the pharmaceutical composition decreases neovascularization signaling in a cell by at least 10%, at least 20%, at least 30%, at least 40%, at least 50%, at least 60%, at least 70%, at least 80%, at least 90%, at least 100%, at least 200%, at least 500%, or more compared to a comparable cell that is not contacted with the non-naturally occurring polynucleotide, the AAV vector comprising the non-naturally occurring polynucleotide, the AAV comprising the non-naturally occurring polynucleotide, or the pharmaceutical composition. In some embodiments, the non-naturally occurring polynucleotide, the AAV vector comprising the non-naturally occurring polynucleotide, the AAV comprising the non-naturally occurring polynucleotide, or the pharmaceutical composition decreases neovascularization signaling in a cell by at least 10%, at least 20%, at least 30%, at least 40%, at least 50%, at least 60%, at least 70%, at least 80%, at least 90%, at least 100%, at least 200%, at least 500%, or more compared to a comparable cell that is treated with a comparable VEGF inhibitor and a RTK/Tie2 or an activator of a RTK/Tie2 encoded from two different non-naturally occurring polynucleotides. In some embodiments, the non-naturally occurring polynucleotide, the AAV vector comprising the non-naturally occurring polynucleotide, the AAV comprising the non-naturally occurring polynucleotide, or the pharmaceutical composition decreases blood vessel leakage in a cell by at least 10%, at least 20%, at least 30%, at least 40%, at least 50%, at least 60%, at least 70%, at least 80%, at least 90%, at least 100%, at least 200%, at least 500%, or more compared to a comparable cell that is not contacted with the non-naturally occurring polynucleotide, the AAV vector comprising the non-naturally occurring polynucleotide, the AAV comprising the non-naturally occurring polynucleotide, or the pharmaceutical composition. In some embodiments, the non-naturally occurring polynucleotide, the AAV vector comprising the non-naturally occurring polynucleotide, the AAV comprising the non-naturally occurring polynucleotide, or the pharmaceutical composition decreases blood vessel leakage in a cell by at least 10%, at least 20%, at least 30%, at least 40%, at least 50%, at least 60%, at least 70%, at least 80%, at least 90%, at least 100%, at least 200%, at least 500%, or more compared to a comparable cell that is treated with a comparable VEGF inhibitor and a comparable RTK/Tie2 or the activator of the RTK/Tie2 encoded from two different non-naturally occurring polynucleotides. In some embodiments, the non-naturally occurring polynucleotide, the AAV vector comprising the non-naturally occurring polynucleotide, the AAV comprising the non-naturally occurring polynucleotide, or the pharmaceutical composition decreases inflammation by at least 10%, at least 20%, at least 30%, at least 40%, at least 50%, at least 60%, at least 70%, at least 80%, at least 90%, at least 100%, at least 200%, at least 500%, or more compared to a comparable cell that is not contacted with the non-naturally occurring polynucleotide, the AAV vector comprising the non-naturally occurring polynucleotide, the AAV comprising the non-naturally occurring polynucleotide, or the pharmaceutical composition. In some embodiments, the non-naturally occurring polynucleotide, the AAV vector comprising the non-naturally occurring polynucleotide, the AAV comprising the non-naturally occurring polynucleotide, or the pharmaceutical composition decreases inflammation by at least 10%, at least 20%, at least 30%, at least 40%, at least 50%, at least 60%, at least 70%, at least 80%, at least 90%, at least 100%, at least 200%, at least 500%, or more compared to a comparable cell that is treated with a comparable VEGF inhibitor and a RTK/Tie2 or the activator of the RTK/Tie2encoded from two different non-naturally occurring polynucleotides.

In some embodiments, the method of treatment described herein can treat an ocular disease. Relevant ocular diseases and conditions can include but are not limited to: blindness, Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In an embodiment, the ocular disease or condition is AMD. AMD can be wet AMD or dry AMD.

In some cases, an administration of a pharmaceutical composition is sufficient to reduce at least a symptom of a disease or condition, treat the disease or condition, and/or eliminate the disease or condition. In some cases, improvements of diseases or conditions can be ascertained by any of the provided diagnostic assays. In other cases, an improvement can be obtained via an interview with the treated subject. For example, a subject may be able to communicate to an attending physician that their vision is improved as compared to their vision prior to administration of a subject pharmaceutical. In other cases, an in vivo animal model may be used to ascertain reduction of a disease or condition after treatment. Suitable animal models include mouse models, primate models, rat models, canine models, and the like.

### Pharmaceutical Compositions

Described herein are pharmaceutical compositions comprising the non-naturally occurring polynucleotide, the AAV vector comprising the non-naturally occurring polynucleotide, or the AAV comprising the non-naturally occurring polynucleotide described herein. In some embodiments, the pharmaceutical composition further comprise as pharmaceutically acceptable: carrier, excipient, or diluent. In some embodiments, the pharmaceutical composition comprises two or more active agents as disclosed herein. In some embodiments, the pharmaceutical composition comprising the non-naturally occurring polynucleotide, the AAV vector comprising the non-naturally occurring polynucleotide, or the AAV comprising the non-naturally occurring polynucleotide treats a disease or condition described herein. In some embodiments, the disease or condition comprises an ocular disease. In some embodiments, the disease or condition comprises ocular ischemic syndrome, proliferative retinopathies, neovascular glaucoma (NG), uveitis, neovascular uveitis, achromatopsia, age-related macular degeneration (nAMD), diabetic macular edema (DME), diabetic macular retinopathy (DMR), retinal vein occlusion (RVO), glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), and blue-cone monochromacy.

For in vivo delivery, the non-naturally occurring polynucleotide, AAV vector, or AAV virion comprising the non-naturally occurring polynucleotide can be formulated into pharmaceutical compositions and can generally be administered intravitreally or parenterally (e.g., administered via an intramuscular, subcutaneous, intratumoral, transdermal, intrathecal, etc., route of administration). In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

In some aspects, a pharmaceutical composition can be used to treat a subject such as a human or mammal, in need thereof. In some cases, a subject can be diagnosed with a disease, e.g., ocular disease. In some aspects, subject pharmaceutical compositions are co-administered with secondary therapies. A secondary therapy can comprise any therapy for ocular use. In some cases, a secondary therapy comprises nutritional therapy, vitamins, laser treatment, such as laser photocoagulation, photodynamic therapy, Visudyne, anti-VEGF therapy, eye-wear, eye drops, numbing agents, Orthoptic vision therapy, Behavioral/perceptual vision therapy, and the like. In some aspects, any of the previously described biologics can be considered a secondary therapy.

In some embodiments, an effective amount of the pharmaceutical composition results in a decrease in the rate of loss of retinal function, anatomical integrity, or retinal health, e.g. a 2-fold, 3-fold, 4-fold, or 5-fold or more decrease in the rate of loss and hence progression of disease, for example, a 10-fold decrease or more in the rate of loss and hence progression of disease.

In some embodiments, an effective amount of the pharmaceutical composition decreases neovascularization signaling in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling in a cell that is not treated with the pharmaceutical composition. In some embodiments, an effective amount of the pharmaceutical composition decreases neovascularization in a subject in need thereof at least 10%, at least 20%, at least 30%, at least 40%, at least 50%, at least 60%, at least 70%, at least 80%, at least 90%, at least 100%, at least 200%, at least 500%, or more compared to neovascularization in the subject if the subject is not treated with the pharmaceutical composition. In some embodiments, an effective amount of the pharmaceutical composition decreases blood vessel leakage in a subject in need thereof at least 10%, at least 20%, at least 30%, at least 40%, at least 50%, at least 60%, at least 70%, at least 80%, at least 90%, at least 100%, at least 200%, at least 500%, or more compared to blood vessel leakage in the subject if the subject is not treated with the pharmaceutical composition. In some embodiments, an effective amount of the pharmaceutical composition decreases inflammation in a subject in need thereof at least 10%, at least 20%, at least 30%, at least 40%, at least 50%, at least 60%, at least 70%, at least 80%, at least 90%, at least 100%, at least 200%, at least 500%, or more compared to inflammation in the subject if the subject is not treated with the pharmaceutical composition.

In some embodiments, the effective amount of the subject rAAV virion results in a gain in visual function, retinal function, an improvement in retinal anatomy or health, and/or an improvement in ocular motility and/or improvement in neurological function, e.g. a 2-fold, 3-fold, 4-fold or 5-fold improvement or more in retinal function, retinal anatomy or health, and/or improvement in ocular motility, e.g. a 10-fold improvement or more in retinal function, retinal anatomy or health, and/or improvement in ocular motility. As will be readily appreciated by the ordinarily skilled artisan, the dose required to achieve the desired treatment effect will typically be in the range of 1×10⁸ to about 1×10¹⁵ recombinant virions, typically referred to by the ordinarily skilled artisan as 1×10⁸ to about 1×10¹⁵ “vector genomes”.

In some aspects, compositions provided herein, such as pharmaceutical compositions are administered to a subject in need thereof. In some cases, an administration comprises delivering a dosage of an AAV of about vector 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, 1.5×10¹⁵ vg. For example, for in vivo injection, e.g., injection directly into the eye, a therapeutically effective dose can be on the order of from about 10⁶ to about 10¹⁵ of subject AAV virions, e.g., from about 10⁸ to 10¹² engineered AAV virions. For in vitro transduction, an effective amount of engineered AAV virions to be delivered to cells will be on the order of from about 10⁸ to about 10¹³ of the engineered AAV virions. Other effective dosages can be readily established by one of ordinary skill in the art through routine trials establishing dose response curves.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skill in the art can readily determine an appropriate number of doses. In some aspects, a pharmaceutical composition is administered via intravitreal injection, subretinal injection, microinjection, or supraocular injection.

In some aspects, a subject can be screened via genetic testing for a mutation before, during, and/or after administration of a pharmaceutical composition provided herein. Relevant genes that can be screened for mutations include RPE65, CRB1, AIPL1, CFH, or RPGRIP.

In practicing the methods of treatment or use provided herein, therapeutically effective amounts of the pharmaceutical composition described herein are administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner, such as, by way of example only, by means of conventional mixing, dissolving, granulating, levigating, emulsifying, encapsulating, entrapping or compression processes.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical composition described herein is formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

For intravenous injections or drips or infusions, the pharmaceutical composition described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Pharmaceutical composition for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations that include a pharmaceutical composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical composition described herein is self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a pharmaceutical composition is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the pharmaceutical composition is provided that include particles of a therapeutic agent and at least one dispersing agent or suspending agent for oral administration to a subject. The formulations may be a powder and/or granules for suspension, and upon admixture with water, a substantially uniform suspension is obtained.

Furthermore, the pharmaceutical composition optionally includes one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical composition optionally includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

### Kits

Disclosed herein, in some embodiments, are kits for using the non-naturally occurring polynucleotide, the AAV vector comprising the non-naturally occurring polynucleotide, AAV comprising the non-naturally occurring polynucleotide, or the pharmaceutical composition described herein. In some embodiments, the kit disclosed herein may be used to treat a disease or condition in a subject. In some embodiments, the kit comprises an assemblage of materials or components apart from the non-naturally occurring polynucleotide, the AAV vector comprising the non-naturally occurring polynucleotide, AAV comprising the non-naturally occurring polynucleotide or the pharmaceutical composition.

In some embodiments, the kits described herein comprise components for selecting for a homogenous population of AAV containing the non-naturally occurring polynucleotide described herein. In some embodiments, the kit comprises the components for assaying the number of units of a biomolecule (e.g., the AAV) synthesized, and/or released or expressed on the surface by a host cell.

In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the non-naturally occurring polynucleotide, the AAV vector comprising the non-naturally occurring polynucleotide, or the pharmaceutical composition to a subject in need thereof. In some embodiments, the kit comprises instructions for further engineering a cell to express a biomolecule (e.g., the non-naturally occurring polynucleotide, the AAV vector comprising the non-naturally occurring polynucleotide, or the AAV comprising the non-naturally occurring polynucleotide). In some embodiments, the kit comprises instructions for thawing or otherwise restoring biological activity of the non-naturally occurring polynucleotide, the AAV vector comprising the non-naturally occurring polynucleotide, or the AAV comprising the non-naturally occurring polynucleotide, which may have been cryopreserved, lyophilized, or cryo-hibernated during storage or transportation. In some embodiments, the kit comprises instructions for measuring the viability of the restored non-naturally occurring polynucleotide, the AAV vector comprising the non-naturally occurring polynucleotide, or the AAV comprising the non-naturally occurring polynucleotide to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s).

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1. AAV Vector Designs and Expression Studies

Example 1 illustrates experiments for measuring the expression levels of the V/EGF inhibitor in combination with either: Ang1 protein (full length or fragment); or Ang2 inhibitory RNA (e.g., Ang2 shRNA) with Ang2 inhibitory RNA inhibition determined by endogenous Ang2 expression level.

### Materials and Methods

The standard methods were used for the molecular cloning of DNA constructs encoding VEGF antagonists (Aflibercept, Lucentis, anti-VEGF F(ab)′, and single chain fragment of variable regions (scFv), hCOMP-Ang1-FLD, and hCOMP-Ang1-FLD-FLAG, Ang2 antibody scFv and Ang2 short hairpin RNA fragments. These proteins of interests (POIs) are listed in the Table 1. Non-limiting exemplary AAV vectors comprising different combinations of VEGF inhibitor and activator of the RTK/Tie2 for modulating Ang1 or Ang2 expression are listed in Table 2. Table 3 lists DNA primers used for PCR amplifications and DNA sequencing analyses of the AAV vectors and the expression cassettes described herein.

TABLE 1
Summary of building blocks of peptides for
the therapeutic protein and AAV construction

| | | | SEQ |
|---|---|---|---|
| | | | ID |
| Function | Protein | Polypeptide sequence | NO: |
| Anti- | Aflibercept | SDTGRPFVEMYSEIPEIIHMTEGRELVIPCRVTSPNITVTLK | SEQ |
| VEGF | | KFPLDTLIPDGKRIIWDSRKGFIISNATYKEIGLLTCEATVN | ID |
| | | GHLYKTNYLTHRQTNTIIDVVLSPSHGIELSVGEKLVLNCTA | NO: |
| | | RTELNVGIDFNWEYPSSKHQHKKLVNRDLKTQSGSEMKKFLS | 31 |
| | | TLTIDGVTRSDQGLYTCAASSGLMTKKNSTFVRVHEKDKTHT | |
| | | CPPCPAPELLGGPSVFLFPPKPKDTLMISRTPEVTCVVVDVS | |
| | | HEDPEVKFNWYVDGVEVHNAKTKPREEQYNSTYRVVSVLTVL | |
| | | HQDWLNGKEYKCKVSNKALPAPIEKTISKAKGQPREPQVYTL | |
| | | PPSRDELTKNQVSLTCLVKGFYPSDIAVEWESNGQPENNYKT | |
| | | TPPVLDSDGSFFLYSKLTVDKSRWQQGNVFSCSVMHEALHNH | |
| | | YTQKSLSLSPG | |
| Anti- | Ranibizumab, | EVQLVESGGGLVQPGGSLRLSCAASGYDFTHYGMNWVRQA | SEQ |
| VEGF | heavy | PGKGLEWVGWINTYTGEPTYAADFKRRFTFSLDTSKSTAY | ID |
| | chain | LQMNSLRAEDTAVYYCAKYPYYYGTSHWYFDVWGQGTLVT | NO: |
| | | VSSASTKGPSVFPLAPSSKSTSGGTAALGCLVKDYFPEPV | 32 |
| | | TVSWNSGALTSGVHTFPAVLQSSGLYSLSSVVTVPSSSLG | |
| | | TQTYICNVNHKPSNTKVDKKVEPKSCDKTHL | |
| Anti- | Ranibizumab, | DIQLTQSPSSLSASVGDRVTITCSASQDISNYLNWYQQKPG | SEQ |
| VEGF | light | KAPKVLIYFTSSLHSGVPSRFSGSGSGTDFTLTISSLQPED | ID |
| | chain | FATYYCQQYSTVPWTFGQGTKVEIKRTVAAPSVFIFPPSDE | NO: |
| | | QLKSGTASVVCLLNNFYPREAKVQWKVDNALQSGNSQESVT | 33 |
| | | EQDSKDSTYSLSSTLTLSKADYEKHKVYACEVTHQGLSSPV | |
| | | TKSFNRGEC | |
| Angio | Fibronectin | DTVHNLVNLCTKEGVLLKGGKREEEKPFRDCADVYQAGEN | SEQ |
| poietin | -like | KSGIYTIYINNMPEPKKVFCNMDVNGGGWTVIQHREDGSL | ID |
| 1 | domain | DFQRGWKEYKMGFGNPSGEYWLGNEFIFAITSQRQYMLRI | NO: |
| (Ang1) | (FLD) | ELMDWEGNRAYSQYDRFHIGNEKQNYRLYLKGHTGTAGKQ | 5 |
| | | SSLILHGADFSTKDADNDNCMCKCALMLTGGWWFDACGPS | |
| | | NLNGMFYTAGQNHGKLNGIKWHYFKGPSYSLRSTTMMIRP | |
| | | LDF | |
| cartilage | Soluble | DLAPQMLRELQETNAALQDVRELLRQQVKEITFLKNTVMEC | SEQ |
| oligomeric | peptide | DACG | ID |
| matrix | | | NO: |
| protein | | | 1 |
| (COMP) | | | |
| Unnamed | Soluble | DLGPQMLRELQETNAALQDVRELLRQQVKEITFLRNTVMEC | SEQ |
| protein | peptide | DACG | ID |
| [Homo | | | NO: |
| sapiens] | | | 2 |
| Angio | For shRNA | mwqivfftls cdlvlaaayn nfrksmdsig | SEQ |
| poietin 2 | design | kkqyqvqhgs csytfllpem dncrsssspy | ID |
| (Ang2) | | vsnavqrdap leyddsvqrl qvlenimenn | NO: |
| | | tqwlmkleny iqdnmkkemv eiqqnavqnq | 12 |
| | | tavmieigtn llnqtaeqtr kltdveaqvl | |
| | | nqttrlelql lehslstnkl ekqildqtse | |
| | | inklqdknsf lekkvlamed khiiqlqsik | |
| | | eekdqlqvlv skqnsiieel ekkivtatvn | |
| | | nsvlqkqqhd lmetvnnllt mmstsnskdp | |
| | | tvakeeqisf rdcaevfksg httngiytlt | |
| | | fpnsteeika ycdmeagggg wtiiqrredg | |
| | | svdfqrtwke ykvgfgnpsg eywlgnefvs | |
| | | qltnqqryvl kihlkdwegn eayslyehfy | |
| | | Isseelnyri hlkgltgtag kissisqpgn | |
| | | dfstkdgdnd kcickcsqml tggwwfdacg | |
| | | psnlngmyyp qrqntnkfng ikwyywkgsg | |
| | | yslkattmmi rpadf | |
| Anti- | Nesvacumab | EVQLVESGGGLVQPGGSLRLSCAASGFTFSSYDIHWVRQAT | SEQ |
| human | scFv | GKGLEWVSAIGPAGDTYYPGSVKGRFTISRENAKNSLYLQM | ID |
| Ang2 | | NSLRAGDTAVYYCARGLITFGGLIAPFYWGQGTLVTVSSGG | NO: |
| | | GGSGGGGSGGGGSGGGGSEIVLTQSPGTLSLSPGERATLSC | 141 |
| | | RASQSVSSTYLAWYQQKPGQAPRLLIYGASSRATGIPDRFS | |
| | | GSGSGTDFTLTISRLEPEDFAVYYCQHYDNSQTFGQGTKVE | |
| | | IKRTVAA | |

TABLE 2
Molecular cloning of plasmids encoding proteins and combinations via the use of exemplary
AAV vector for VEGF inhibition and modulation of Ang1 or Ang2 expression

AMI071-pFB-scCMV-SV40intron-Vh-hCOMP-Ang1-FLAG was created by assembling the
hCOMP-Ang1-FLAG fragment PCR-amplified from AMI063 with primers A095, A096, and
A097 into the AflII and XhoI sites of AMI060
AMI077-pFB-scCMV-SV40intron-Vh-hCOMP-Ang1-FLAG-GC was created by assembling the
codon-optimized hCOMP-Ang1-GC-FLAG fragment PCR-amplified from Twist synthesized
DNA with primers A095 and A135 into the AflII and Xhol sites of AMI071
AMI136-pFB-CMV-SV40in-Aflibercept-GCRS(TCC)-CMV-SV40in-hCOMP-Ang1 was created
by assembling the CMV-SV40in-Aflibercept-GCRS (TCC) fragment PCR-amplified from
AMI120 with primers A426 and A427 into the KpnI sites of AMI092
AMI142-pFB-CMV-SV40in-Aflibercept-GCRS(TCC)-Furin-F2A-hCOMP-Ang1 was created by
assembling the Furin-F2A-hCOMP-Ang1 fragment PCR-amplified with primers A522, A523, and
A524, into the BstBI sites of AMI136
AMI143-pFB-CMV-SV40in-Aflibercept-GCRS(TCC)-QBI_SP163-hCOMP-Ang1 was created by
assembling the joined PCR fragment (the QBI SP163 fragment PCR-amplified with primers A525
and A526, and AMI131 as template; the hCOMP-Ang1 fragment PCR-amplified with primers
A527 and A524, and AMI063 as template, and these two fragments were joined together by PCR
with primers A525 and A524) into the BstBI sites of AMI136
AMI144-pFB-CMV-SV40in-Aflibercept-GCRS(TCC)-4xGGGGS-hCOMP-Ang1 was created by
assembling the 4xGGGGS-hCOMP-Ang1 fragment PCR-amplified with primers A528 and A529,
and AMI063 as template into the BstBI sites of AMI136
AMI145-pFB-scCMV-SV40in-Aflibercept-GCRS(TCC)-hU6-shRNA1-Ang2 was created by
assembling the hU6-shRNA1-Ang2 fragment PCR-amplified with primers A530 and A531 and
V402 as template into SphI site of AMI120
AMI146-pFB-scCMV-SV40in-Vh-Ang2-10xHis was created by assembling the Vh-Ang2-10xHis
fragment PCR-amplified with primers A532 and A533 and Ang2 purchased from Sinobiological
HG10691-CH) as template into the AflII and XhoI sites of AMI071
AMI147-, AMI148-, AMI149-, AMI150-, AMI151-, and AMI152-pFB-scCMV-SV40in-
Aflibercept-GCRS(TCC)-hU6-shRNA2-, 3-, scramble-, 4-, 5-, and 6-Ang2 were created by
ligating the annealed oligo pairs A534-A535, A536-A537, A544-A545, A546-A547, A548-A549,
A550-A551 respectively into the SphI and HindIII sites of AMI145
AMI153-pFB-CMV-SV40in-Aflibercept-GCRS(TCC)-CMV-SV40in-hCOMP-Ang1-GC was
created by assembling the hCOMP-Ang1-GC-pA fragment PCR-amplified with primers A095,
A555, and A556, and Twist synthesized hCOMP-Ang1-GC DNA fragment as template into the
AflII and PmlI sites of AMI136
AMI154-pFB-CMV-SV40in-Aflibercept-GCRS(TCC)-Furin-F2A-hCOMP-Ang1-GC was created
by assembling the hCOMP-Ang1-pA fragment PCR-amplified with primers A095, A555 and
A556, and Twist synthesized hCOMP-Ang1-GC DNA fragment as template into the AflII and
PmlI sites of AMI142
AMI155-pFB-CMV-Lucentis-ScFv-GC-Furin-F2A-hCOMP-Ang1-GC
AMI156-pFB-CMV-Vh-Lucentis-ScFv-GC-CMV-Vh-hCOMP-Ang1-GC was created by
assembling the Lucentis-ScFv fragment PCR-amplified with primers A579 and A580, and
AMI157 as template into the KpnI sites of AMI153
AMI157-pFB-scCMV-SV40in-Lucentis-ScFv-GC-hU6-shRNA1-Ang2 was created by first PCR-
amplifying the 5′-Lucentis ScFv fragment with primers A270 and A565 and Twist-Luc-Vh-opt
DNA fragment as template, and the 3′-Lucentis fragment with primers A564, A558 and A561,
and Twist Luc-Vl-Furin-F2A-opt as template, then joining these two PCR fragments with primers
A270 and A561, and finally assembling the joined PCR fragment into the StuI and BstBI sites of
AMI145
AMI158-pFB-scCMV-SV40in-Lucentis-ScFv-GC-hU6-shRNA2-Ang2 was created by first PCR-
amplifying the 5′-Lucentis ScFv fragment with primers A270 and A565 and Twist-Luc-Vh-opt
DNA fragment as template, and the 3′-Lucentis fragment with primers A564, A558 and A561,
and Twist Luc-Vl-Furin-F2A-opt as template, then joining these two PCR fragments with primers
A270 and A561, and finally assembling the joined PCR fragment into the StuI and BstBI sites of
AMI147
AMI159-pFB-scCMV-SV40in-Lucentis-ScFv-GC-hU6-shRNA3-Ang2 was created by first PCR-
amplifying the 5′-Lucentis ScFv fragment with primers A270 and A565 and Twist-Luc-Vh-opt
DNA fragment as template, and the 3′-Lucentis fragment with primers A564, A558 and A561,
and Twist Luc-Vl-Furin-F2A-opt as template, then joining these two PCR fragments with primers
A270 and A561, and finally assembling the joined PCR fragment into the StuI and BstBI sites of
AMI148
AMI160-pFB-scCMV-SV40in-Lucentis-ScFv-GC-hU6-shRNA4-Ang2 was created by first PCR-
amplifying the 5′-Lucentis ScFv fragment with primers A270 and A565 and Twist-Luc-Vh-opt
DNA fragment as template, and the 3′-Lucentis fragment with primers A564, A558 and A561,
and Twist Luc-Vl-Furin-F2A-opt as template, then joining these two PCR fragments with primers
A270 and A561, and finally assembling the joined PCR fragment into the StuI and BstBI sites of
AMI150
AMI161-pFB-scCMV-SV40in-Lucentis-ScFv-GC-hU6-shRNA5-Ang2 was created by first PCR-
amplifying the 5′-Lucentis ScFv fragment with primers A270 and A565 and Twist-Luc-Vh-opt
DNA fragment as template, and the 3′-Lucentis fragment with primers A564, A558 and A561,
and Twist Luc-Vl-Furin-F2A-opt as template, then joining these two PCR fragments with primers
A270 and A561, and finally assembling the joined PCR fragment into the StuI and BstBI sites of
AMI151
AMI162-pFB-scCMV-SV40in-Lucentis-ScFv-GC-hU6-shRNA6-Ang2 was created by first PCR-
amplifying the 5′-Lucentis ScFv fragment with primers A270 and A565 and Twist-Luc-Vh-opt
DNA fragment as template, and the 3′-Lucentis fragment with primers A564, A558 and A561,
and Twist Luc-Vl-Furin-F2A-opt as template, then joining these two PCR fragments with primers
A270 and A561, and finally assembling the joined PCR fragment into the StuI and BstBI sites of
AMI152
AMI163-pFB-scCMV-SV40in-Lucentis-ScFv-GC-hU6-shRNA-scramble-Ang2 was created by
first PCR-amplifying the 5′-Lucentis ScFv fragment with primers A270 and A565 and Twist-Luc-
Vh-opt DNA fragment as template, and the 3′-Lucentis fragment with primers A564, A558 and
A561, and Twist Luc-Vl-Furin-F2A-opt as template, then joining these two PCR fragments with
primers A270 and A561, and finally assembling the joined PCR fragment into the StuI and BstBI
sites of AMI149
AMI166-pFB-CMV-SV40in-VEGF-Trap-CMV-SV40in-TNFa-ScFv was created by PCR
amplification of the TNFa-ScFv fragment with primers A581 and A582 and AMI095 as template
The PCR fragment was cloned into the AflII and PmlI sites of AMI136 through HiFi reaction
AMI167-pFB-CMV-SV40in-Lucentis-ScFv-CMV-SV40in-TNFa-ScFv was created by PCR
amplify the TNFa-ScFv fragment with primers A581 and A582 and AMI095 as template The PCR
fragment was cloned into the AflII and PmlI sites of AMI156 through HiFi reaction
AMI169-pFB-scCMV-SV40 intron-Vh-CNP-Furin-F2A-Vh-Lucentis-ScFv was created by PCR
amplification of a Furin-F2A fragment with primers A585 and A052 and A155 as template, a
Lucentis ScFv fragment with primers A581 and A561 and AMI156 as template, joining both PCR
fragments together with primers AA585 and A561, and ligating to the BstBI and XcmI sites of
AMI087 via HiFi assembly

TABLE 3
DNA primers used for PCR amplifications and DNA sequencing analyses

| | Primer | | SEQ ID |
|---|---|---|---|
| Item | ID | DNA Sequence | NO |
| 1 | A095 | 5′TCCTGGTGGCCATCCTTAAGGGCGTGCAGTGCGACCTGGG | SEQ ID |
| | | CCCCCAGAT-3′ | NO: |
| | | | 101 |
| 2 | A096 | 5′-TCGTCATCGTCTTTGTAGTCGAAGTCCAGAGGCCTGATCA- | SEQ ID |
| | | 3′ | NO: |
| | | | 102 |
| 3 | A097 | 5′ATCCAGAGGTTGATTCTCGAGTCACTTGTCGTCATCGTCTT | SEQ ID |
| | | TGTAG-3′ | NO: |
| | | | 103 |
| 4 | A135 | 5′GTAATCCAGAGGTTGATTCTCGAGTCACTTGTCGTCGTCGT | SEQ ID |
| | | CCTTGTAG-3′ | NO: |
| | | | 104 |
| 5 | A426 | 5′-ATTGACTAGGAAGCTGATCTGAATT-3′ | SEQ ID |
| | | | NO: |
| | | | 105 |
| 6 | A427 | 5′GTAAGTTATGTAACGGGTACCGAATTCGGTTGATCTCTCCC | SEQ ID |
| | | CAGCATGC-3′ | NO: |
| | | | 106 |
| 7 | A522 | 5′CTGTCCCTGTCCCCCGGCAAGAGAAGAAAGAGAGCCCCCG | SEQ ID |
| | | TGAAGCAGACCCTGAACTTCGACCTGC TGAAGCTGGCCG-3′ | NO: |
| | | | 107 |
| 8 | A523 | 5′CGACCTGCTGAAGCTGGCCGGCGACGTGGAGAGCAACCCC | SEQ ID |
| | | GGCCCCATGGAGTTCGGCCTGAGCTGG-3′ | NO: |
| | | | 108 |
| 9 | A524 | 5′-CAGATGTAATGAAAATAAAGATATTTTATT-3′ | SEQ ID |
| | | | NO: |
| | | | 109 |
| 10 | A525 | 5′CTGTCCCTGTCCCCCGGCAAGTGAGATATCTAGAGCGCAG | SEQ ID |
| | | AGGCTTG-3′ | NO: |
| | | | 110 |
| 11 | A526 | 5′- | SEQ ID |
| | | CAGCTCAGGCCGAACTCCATGGTTTCGGAGGCCGTCCGGG-3′ | NO: |
| | | | 111 |
| 12 | A528 | 5′CTGTCCCTGTCCCCCGGCAAGGGCGGAGGCGGAAGCGGCG | SEQ ID |
| | | GAGGCGGATCTGGCGGAGGCGGCA GCGGCG-3′ | NO: |
| | | | 112 |
| 13 | A529 | 5′GGATCTGGCGGAGGCGGCAGCGGCGGCGGCGGCTCTGACC | SEQ ID |
| | | TGGGCCCCCAGATGCTGAG-3′ | NO: |
| | | | 113 |
| 14 | A530 | GTGTGTTGGTTTTTTGTGTGGAGGGCCTATTTCCCATGAT | SEQ ID |
| | | | NO: |
| | | | 114 |
| 15 | A531 | TGATCTCTCCCCAGCATGCAAAAAGGTTCAACGGCATTAAAT | SEQ ID |
| | | ATATGGGTCAGGTATATTTAATGCCGTTGAACCAAGCTTGTC | NO: |
| | | CTTTCCACAAGATAT | 115 |
| 16 | A534 | 5′AGCTTGGAAGCTTGAGAATTATAATACCTGACCCATATTAT | SEQ ID |
| | | AATTCTCAAGCTTCCTTTTTGCATG-3′ | NO: |
| | | | 116 |
| 17 | A535 | 5′CAAAAAGGAAGCTTGAGAATTATAATATGGGTCAGGTATT | SEQ ID |
| | | ATAATTCTCAAGCTTCCA-3′ | NO: |
| | | | 117 |
| 18 | A536 | 5′AGCTTGTGAAGAACTCAATTATAATACCTGACCCATATTAT | SEQ ID |
| | | AATTGAGTTCTTCACTTTTTGCATG-3′ | NO: |
| | | | 118 |
| 19 | A537 | 5′CAAAAAGTGAAGAACTCAATTATAATATGGGTCAGGTATT | SEQ ID |
| | | ATAATTGAGTTCTTCACA-3′ | NO: |
| | | | 119 |
| 20 | A544 | 5′AGCTTGTGCATATGAACGTAACTATACCTGACCCATATAGT | SEQ ID |
| | | TACGTTCATATGCACTTTTTGCATG-3′ | NO: |
| | | | 120 |
| 21 | A545 | 5′CAAAAAGTGCATATGAACGTAACTATATGGGTCAGGTATA | SEQ ID |
| | | GTTACGTTCATATGCACA-3′ | NO: |
| | | | 121 |
| 22 | A546 | 5′AGCTTGTAACATTCCCTAATTCTATACCTGACCCATATAGA | SEQ ID |
| | | ATTAGGGAATGTTACTTTTTGCATG-3′ | NO: |
| | | | 122 |
| 23 | A547 | 5′CAAAAAGTAACATTCCCTAATTCTATATGGGTCAGGTATA | SEQ ID |
| | | GAATTAGGGAATGTTACA-3′ | NO: |
| | | | 123 |
| 24 | A548 | 5′AGCTTGACTTGGAAAGAATATAAATACCTGACCCATATTT | SEQ ID |
| | | ATATTCTTTCCAAGTCTTTTTGCATG-3′ | NO: |
| | | | 124 |
| 25 | A549 | 5′CAAAAAGACTTGGAAAGAATATAAATATGGGTCAGGTATT | SEQ ID |
| | | TATATTCTTTCCAAGTCA-3′ | NO: |
| | | | 125 |
| 26 | A550 | 5′AGCTTGGTGAAGAACTCAATTATATACCTGACCCATATATA | SEQ ID |
| | | ATTGAGTTCTTCACCTTTTTGCATG-3′ | NO: |
| | | | 126 |
| 27 | A551 | 5′CAAAAAGGTGAAGAACTCAATTATATATGGGTCAGGTATA | SEQ ID |
| | | TAATTGAGTTCTTCACCA-3′ | NO: |
| | | | 127 |
| 28 | A555 | 5′GAAAATAAAGATATTTTATTTTCGAATTCCAGAGTCCCGCT | SEQ ID |
| | | CAGAAGTCCAGGGGTCTG-3′ | NO: |
| | | | 128 |
| 29 | A556 | 5′GCGGCCGCTCGGTCCGCACGTGCAGAACACACAAAAAACC | SEQ ID |
| | | AACACACAGATGTAATGAAAATAAAGAT ATTTTA-3′ | NO: |
| | | | 129 |
| 30 | A558 | 5′AGGCGGATCTGGCGGAGGCGGCAGCGGCGGCGGCGGCTCT | SEQ ID |
| | | GACATCCAGCTGACCCAG-3′ | NO: |
| | | | 130 |
| 31 | A561 | 5′GAAAATAAAGATATTTTATTTTCGAATCAGGCGGCCACGG | SEQ ID |
| | | TTCTCTTGA-3′ | NO: |
| | | | 131 |
| 32 | A564 | 5′AGGGCACCCTGGTGACCGTGGGCGGAGGCGGAAGCGGCG | SEQ ID |
| | | GAGGCGGATCTGGCGGAGGC-3′ | NO: |
| | | | 132 |
| 33 | A565 | 5′-CACGGTCACCAGGGTGCCCT-3′ | SEQ ID |
| | | | NO: |
| | | | 133 |
| 34 | A579 | 5′- | SEQ ID |
| | | AGGAAGCTGATCTGAATTCGGTACCCGTTACATAACTTACGG | NO: |
| | | TAA-3′ | 134 |
| 35 | A580 | 5′- | SEQ ID |
| | | TACCGTAAGTTATGTAACGGGTACCCACACAAAAAACCAAC | NO: |
| | | ACAC-3′ | 135 |
| 36 | A581 | 5′- | SEQ ID |
| | | GCTGTTCCTGGTGGCCATCCTTAAGGGCGTGCAGTGCGAGGT | NO: |
| | | GCA-3′ | 136 |
| 37 | A582 | 5′- | SEQ ID |
| | | GCGGCCGCTCGGTCCGCACGTGGGTTGATCTCTCCCCAGCAT | NO: |
| | | GCC-3′ | 137 |
| 38 | A585 | 5′GAATCGGCTCCATGAGCGGCCTGGGATGTAGAAGAAAGAG | SEQ ID |
| | | AGCCCCCGT-3′ | NO: |
| | | | 138 |

TABLE 4
AAV vector sequences and respective
regulator elements are listed

| Clone | |
|---|---|
| no.; | |
| SEQ | |
| ID NO | Regulatory elements and DNA sequences |
| AMI071; | 611-755: Full ITR |
| SEQ ID | 801-104: CMV enhancer |
| NO: 51 | 1105-1308: CMV promoter |
| | 1412-1508: SV40 intron |
| | 1513-1521: Kozak sequence |
| | 1522-1578: Human IgG heavy chain secretion sequence |
| | 1579-2442: hCOMP-Ang1 coding sequence before optimization |
| | 2443-2446: FLAG epitope |
| | 2476-2858: WPRE minimum sequence |
| | 2871-2919: poly A signal |
| | 2942-3047: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTTAAGGGCGTGCAG |
| | TGCGACCTGGGCCCCCAGATGCTGAGGGAGCTCCAGGAGACCAAT |
| | GCTGCTCTTCAGGATGTTAGGGAACTGCTGAGGCAGCAGGTGAAG |
| | GAGATCACCTTCCTCAGAAACACAGTGATGGAGTGTGATGCCTGT |
| | GGGGACACAGTCCACAACCTGGTCAACCTGTGCACCAAAGAGGGT |
| | GTGCTGCTCAAGGGAGGGAAGAGGGAGGAGGAGAAGCCCTTCAGG |
| | GACTGTGCTGATGTCTACCAGGCTGGCTTCAACAAGAGTGGGATC |
| | TACACCATCTACATCAACAACATGCCTGAGCCCAAGAAGGTGTTC |
| | TGCAACATGGATGTGAATGGGGGGGGCTGGACTGTGATCCAGCAC |
| | AGAGAAGATGGCTCCCTGGACTTCCAGAGGGGCTGGAAGGAATAC |
| | AAGATGGGGTTTGGGAACCCCTCTGGGGAGTACTGGCTGGGCAAT |
| | GAGTTCATCTTTGCCATCACTAGCCAGAGACAGTACATGCTCAGA |
| | ATTGAGCTGATGGACTGGGAGGGCAACAGAGCCTACAGCCAATAT |
| | GACAGGTTCCACATTGGAAATGAAAAGCAGAACTACAGGCTGTAC |
| | CTGAAGGGCCACACTGGGACTGCAGGCAAGCAGAGCTCACTGATC |
| | CTGCATGGAGCTGACTTCTCCACCAAGGATGCAGACAATGACAAC |
| | TGCATGTGCAAGTGTGCCCTCATGCTGACTGGTGGGTGGTGGTTT |
| | GATGCTTGTGGGCCCAGCAACCTGAATGGAATGTTCTACACAGCT |
| | GGGCAGAATCATGGCAAGCTCAATGGCATCAAGTGGCACTACTTC |
| | AAGGGCCCCAGCTACAGCCTGAGGTCCACCACCATGATGATCAGG |
| | CCTCTGGACTTCGACTACAAAGACGATGACGACAAGTGACTCGAG |
| | AATCAACCTCTGGATTACAAAATTTGTGAAAGATTGACTGGTATT |
| | CTTAACTATGTTGCTCCTTTTACGCTATGTGGATACGCTGCTTTA |
| | ATGCCTTTGTATCATGCTATTGCTTCCCGTATGGCTTTCATTTTC |
| | TCCTCCTTGTATAAATCCTGGTTGCTGTCTCTTTATGAGGAGTTG |
| | TGGCCCGTTGTCAGGCAACGTGGCGTGGTGTGCACTGTGTTTGCT |
| | GACGCAACCCCCACTGGTTGGGGCATTGCCACCACCTGTCAGCTC |
| | CTTTCCGGGACTTTCGCTTTCCCCCTCCCTATTGCCACGGCGGAA |
| | CTCATCGCCGCCTGCCTTGCCCGCTGCTGGACAGGGGCTCGGCTG |
| | TTGGGCACTGACAATTCCGTGGTGGTACCTTCGAAAATAAAATAT |
| | CTTTATTTTCATTACATCTGTGTGTTGGTTTTTTGTGTGGCATGC |
| | TGGGGAGAGATCAACCCCACTCCCTCTCTGCGCGCTCGCTCGCTC |
| | ACTGAGGCCGGGCGACCAAAGGTCGCCCGACGCCCGGGCTTTGCC |
| | CGGGCGGCCTCAGTGAGCGAGCGAGCGCGCAGCAAGCTGTAGCCA |
| | ACCACTAGAACTATAGCTAGAGTCCTGGGCGAACAAACGATGCTC |
| | GCCTTCCAGAAAACCGAGGATGCGAACCACTTCATCCGGGGTCAG |
| | CACCACCGGCAAGCGCCGCGACGGCCGAGGTCTTCCGATCTCCTG |
| | AAGCCAGGGCAGATCCGTGCACAGCACCTTGCCGTAGAAGAACAG |
| | CAAGGCCGCCAATGCCTGACGATGCGTGGAGACCGAAACCTTGCG |
| | CTCGTTCGCCAGCCAGGACAGAAATGCCTCGACTTCGCTGCTGCC |
| | CAAGGTTGCCGGGTGACGCACACCGTGGAAACGGATGAAGGCACG |
| | AACCCAGTTGACATAAGCCTGTTCGGTTCGTAAACTGTAATGCAA |
| | GTAGCGTATGCGCTCACGCAACTGGTCCAGAACCTTGACCGAACG |
| | CAGCGGTGGTAACGGCGCAGTGGCGGTTTTCATGGCTTGTTATGA |
| | CTGTTTTTTTGTACAGTCTATGCCTCGGGCATCCAAGCAGCAAGC |
| | GCGTTACGCCGTGGGTCGATGTTTGATGTTATGGAGCAGCAACGA |
| | TGTTACGCAGCAGCAACGATGTTACGCAGCAGGGCAGTCGCCCTA |
| | AAACAAAGTTAGGTGGCTCAAGTATGGGCATCATTCGCACATGTA |
| | GGCTCGGCCCTGACCAAGTCAAATCCATGCGGGCTGCTCTTGATC |
| | TTTTCGGTCGTGAGTTCGGAGACGTAGCCACCTACTCCCAACATC |
| | AGCCGGACTCCGATTACCTCGGGAACTTGCTCCGTAGTAAGACAT |
| | TCATCGCGCTTGCTGCCTTCGACCAAGAAGCGGTTGTTGGCGCTC |
| | TCGCGGCTTACGTTCTGCCCAGGTTTGAGCAGCCGCGTAGTGAGA |
| | TCTATATCTATGATCTCGCAGTCTCCGGCGAGCACCGGAGGCAGG |
| | GCATTGCCACCGCGCTCATCAATCTCCTCAAGCATGAGGCCAACG |
| | CGCTTGGTGCTTATGTGATCTACGTGCAAGCAGATTACGGTGACG |
| | ATCCCGCAGTGGCTCTCTATACAAAGTTGGGCATACGGGAAGAAG |
| | TGATGCACTTTGATATCGACCCAAGTACCGCCACCTAACAATTCG |
| | TTCAAGCCGAGATCGGCTTCCCGGCCGCGGAGTTGTTCGGTAAAT |
| | TGTCACAACGCCGCGAATATAGTCTTTACCATGCCCTTGGCCACG |
| | CCCCTCTTTAATACGACGGGCAATTTGCACTTCAGAAAATGAAGA |
| | GTTTGCTTTAGCCATAACAAAAGTCCAGTATGCTTTTTCACAGCA |
| | TAACTGGACTGATTTCAGTTTACAACTATTCTGTCTAGTTTAAGA |
| | CTTTATTGTCATAGTTTAGATCTATTTTGTTCAGTTTAAGACTTT |
| | ATTGTCCGCCCACACCCGCTTACGCAGGGCATCCATTTATTACTC |
| | AACCGTAACCGATTTTGCCAGGTTACGCGGCTGGTCTGCGGTGTG |
| | AAATACCGCACAGATGCGTAAGGAGAAAATACCGCATCAGGCGCT |
| | CTTCCGCTTCCTCGCTCACTGACTCGCTGCGCTCGGTCGTTCGGC |
| | TGCGGCGAGCGGTATCAGCTCACTCAAAGGCGGTAATACGGTTAT |
| | CCACAGAATCAGGGGATAACGCAGGAAAGAACATGTGAGCAAAAG |
| | GCCAGCAAAAGGCCAGGAACCGTAAAAAGGCCGCGTTGCTGGCGT |
| | TTTTCCATAGGCTCCGCCCCCCTGACGAGCATCACAAAAATCGAC |
| | GCTCAAGTCAGAGGTGGCGAAACCCGACAGGACTATAAAGATACC |
| | AGGCGTTTCCCCCTGGAAGCTCCCTCGTGCGCTCTCCTGTTCCGA |
| | CCCTGCCGCTTACCGGATACCTGTCCGCCTTTCTCCCTTCGGGAA |
| | GCGTGGCGCTTTCTCAATGCTCACGCTGTAGGTATCTCAGTTCGG |
| | TGTAGGTCGTTCGCTCCAAGCTGGGCTGTGTGCACGAACCCCCCG |
| | TTCAGCCCGACCGCTGCGCCTTATCCGGTAACTATCGTCTTGAGT |
| | CCAACCCGGTAAGACACGACTTATCGCCACTGGCAGCAGCCACTG |
| | GTAACAGGATTAGCAGAGCGAGGTATGTAGGCGGTGCTACAGAGT |
| | TCTTGAAGTGGTGGCCTAACTACGGCTACACTAGAAGGACAGTAT |
| | TTGGTATCTGCGCTCTGCTGAAGCCAGTTACCTTCGGAAAAAGAG |
| | TTGGTAGCTCTTGATCCGGCAAACAAACCACCGCTGGTAGCGGTG |
| | GTTTTTTTGTTTGCAAGCAGCAGATTACGCGCAGAAAAAAAGGAT |
| | CTCAAGAAGATCCTTTGATCTTTTCTACGGGGTCTGACGCTCAGT |
| | GGAACGAAAACTCACGTTAAGGGATTTTGGTCATGAGATTATCAA |
| | AAAGGATCTTCACCTAGATCCTTTTAAATTAAAAATGAAGTTTTA |
| | AATCAATCTAAAGTATATATGAGTAAACTTGGTCTGACAGTTACC |
| | AATGCTTAATCAGTGAGGCACCTATCTCAGCGATCTGTCTATTTC |
| | GTTCATCCATAGTTGCCTGACTCCCCGTCGTGTAGATAACTACGA |
| | TACGGGAGGGCTTACCATCTGGCCCCAGTGCTGCAATGATACCGC |
| | GAGACCCACGCTCACCGGCTCCAGATTTATCAGCAATAAACCAGC |
| | CAGCCGGAAGGGCCGAGCGCAGAAGTGGTCCTGCAACTTTATCCG |
| | CCTCCATCCAGTCTATTAATTGTTGCCGGGAAGCTAGAGTAAGTA |
| | GTTCGCCAGTTAATAGTTTGCGCAACGTTGTTGCCATTGCTACAG |
| | GCATCGTGGTGTCACGCTCGTCGTTTGGTATGGCTTCATTCAGCT |
| | CCGGTTCCCAACGATCAAGGCGAGTTACATGATCCCCCATGTTGT |
| | GCAAAAAAGCGGTTAGCTCCTTCGGTCCTCCGATCGTTGTCAGAA |
| | GTAAGTTGGCCGCAGTGTTATCACTCATGGTTATGGCAGCACTGC |
| | ATAATTCTCTTACTGTCATGCCATCCGTAAGATGCTTTTCTGTGA |
| | CTGGTGAGTACTCAACCAAGTCATTCTGAGAATAGTGTATGCGGC |
| | GACCGAGTTGCTCTTGCCCGGCGTCAATACGGGATAATACCGCGC |
| | CACATAGCAGAACTTTAAAAGTGCTCATCATTGGAAAACGTTCTT |
| | CGGGGCGAAAACTCTCAAGGATCTTACCGCTGTTGAGATCCAGTT |
| | CGATGTAACCCACTCGTGCACCCAACTGATCTTCAGCATCTTTTA |
| | CTTTCACCAGCGTTTCTGGGTGAGCAAAAACAGGAAGGCAAAATG |
| | CCGCAAAAAAGGGAATAAGGGCGACACGGAAATGTTGAATACTCA |
| | TACTCTTCCTTTTTCAATATTATTGAAGCATTTATCAGGGTTATT |
| | GTCTCATGAGCGGATACATATTTGAATGTATTTAGAAAAATAAAC |
| | AAATAGGGGTTCCGCGCACATTTCCCCGAAAAGTGCCACCTGAAA |
| | TTGTAAACGTTAATATTTTGTTAAAATTCGCGTTAAATTTTTGTT |
| | AAATCAGCTCATTTTTTAACCAATAGGCCGAAATCGGCAAAATCC |
| | CTTATAAATCAAAAGAATAGACCGAGATAGGGTTGAGTGTTGTTC |
| | CAGTTTGGAACAAGAGTCCACTATTAAAGAACGTGGACTCCAACG |
| | TCAAAGGGCGAAAAACCGTCTATCAGGGCGATGGCCCACTACGTG |
| | AACCATCACCCTAATCAAGTTTTTTGGGGTCGAGGTGCCGTAAAG |
| | CACTAAATCGGAACCCTAAAGGGAGCCCCCGATTTAGAGCTTGAC |
| | GGGGAAAGCCGGCGAACGTGGCGAGAAAGGAAGGGAAGAAAGCGA |
| | AAGGAGCGGGCGCTAGGGCGCTGGCAAGTGTAGCGGTCACGCTGC |
| | GCGTAACCACCACACCCGCCGCGCTTAATGCGCCGCTACAGGGCG |
| | CGTC |
| AMI077; | 611-755: Full ITR |
| SEQ ID | 801-104: CMV enhancer |
| NO: 52 | 1105-1308: CMV promoter |
| | 1412-1508: SV40 intron |
| | 1513-1521: Kozak sequence |
| | 1522-1578: Human IgG heavy chain secretion sequence |
| | 1579-2442: hCOMP-Ang1 coding sequence after optimization |
| | 2443-2446: FLAG epitope |
| | 2476-2858: WPRE minimum sequence |
| | 2871-2919: poly A signal |
| | 2942-3047: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTTAAGGGCGTGCAG |
| | TGCGACCTGGGCCCCCAGATGCTGAGAGAGCTGCAGGAGACCAAC |
| | GCCGCCCTGCAGGACGTGAGAGAGCTGCTGAGACAGCAGGTGAAG |
| | GAGATCACCTTCCTGAGAAACACCGTGATGGAGTGCGACGCCTGC |
| | GGCGACACCGTGCACAACCTGGTGAACCTGTGCACCAAGGAGGGC |
| | GTGCTGCTGAAGGGCGGCAAGAGAGAGGAGGAGAAGCCCTTCAGA |
| | GACTGCGCCGACGTGTACCAGGCCGGCTTCAACAAGAGCGGCATC |
| | TACACCATCTACATCAACAACATGCCCGAGCCCAAGAAGGTGTTC |
| | TGCAACATGGACGTGAACGGCGGCGGCTGGACCGTGATCCAGCAC |
| | AGAGAGGACGGCAGCCTGGACTTCCAGAGAGGCTGGAAGGAGTAC |
| | AAGATGGGCTTCGGCAACCCCAGCGGCGAGTACTGGCTGGGCAAC |
| | GAGTTCATCTTCGCCATCACCAGCCAGAGACAGTACATGCTGAGA |
| | ATCGAGCTGATGGACTGGGAGGGCAACAGAGCCTACAGCCAGTAC |
| | GACAGATTCCACATCGGCAACGAGAAGCAGAACTACAGACTGTAC |
| | CTGAAGGGCCACACCGGCACCGCCGGCAAGCAGAGCAGCCTGATC |
| | CTGCACGGCGCCGACTTCAGCACCAAGGACGCCGACAACGACAAC |
| | TGCATGTGCAAGTGCGCCCTGATGCTGACCGGCGGCTGGTGGTTC |
| | GACGCCTGCGGCCCCAGCAACCTGAACGGCATGTTCTACACCGCC |
| | GGCCAGAACCACGGCAAGCTGAACGGCATCAAGTGGCACTACTTC |
| | AAGGGCCCCAGCTACAGCCTGAGAAGCACCACCATGATGATCAGA |
| | CCCCTGGACTTCGACTACAAGGACGACGACGACAAGTGACTCGAG |
| | AATCAACCTCTGGATTACAAAATTTGTGAAAGATTGACTGGTATT |
| | CTTAACTATGTTGCTCCTTTTACGCTATGTGGATACGCTGCTTTA |
| | ATGCCTTTGTATCATGCTATTGCTTCCCGTATGGCTTTCATTTTC |
| | TCCTCCTTGTATAAATCCTGGTTGCTGTCTCTTTATGAGGAGTTG |
| | TGGCCCGTTGTCAGGCAACGTGGCGTGGTGTGCACTGTGTTTGCT |
| | GACGCAACCCCCACTGGTTGGGGCATTGCCACCACCTGTCAGCTC |
| | CTTTCCGGGACTTTCGCTTTCCCCCTCCCTATTGCCACGGCGGAA |
| | CTCATCGCCGCCTGCCTTGCCCGCTGCTGGACAGGGGCTCGGCTG |
| | TTGGGCACTGACAATTCCGTGGTGGTACCTTCGAAAATAAAATAT |
| | CTTTATTTTCATTACATCTGTGTGTTGGTTTTTTGTGTGGCATGC |
| | TGGGGAGAGATCAACCCCACTCCCTCTCTGCGCGCTCGCTCGCTC |
| | ACTGAGGCCGGGCGACCAAAGGTCGCCCGACGCCCGGGCTTTGCC |
| | CGGGCGGCCTCAGTGAGCGAGCGAGCGCGCAGCAAGCTGTAGCCA |
| | ACCACTAGAACTATAGCTAGAGTCCTGGGCGAACAAACGATGCTC |
| | GCCTTCCAGAAAACCGAGGATGCGAACCACTTCATCCGGGGTCAG |
| | CACCACCGGCAAGCGCCGCGACGGCCGAGGTCTTCCGATCTCCTG |
| | AAGCCAGGGCAGATCCGTGCACAGCACCTTGCCGTAGAAGAACAG |
| | CAAGGCCGCCAATGCCTGACGATGCGTGGAGACCGAAACCTTGCG |
| | CTCGTTCGCCAGCCAGGACAGAAATGCCTCGACTTCGCTGCTGCC |
| | CAAGGTTGCCGGGTGACGCACACCGTGGAAACGGATGAAGGCACG |
| | AACCCAGTTGACATAAGCCTGTTCGGTTCGTAAACTGTAATGCAA |
| | GTAGCGTATGCGCTCACGCAACTGGTCCAGAACCTTGACCGAACG |
| | CAGCGGTGGTAACGGCGCAGTGGCGGTTTTCATGGCTTGTTATGA |
| | CTGTTTTTTTGTACAGTCTATGCCTCGGGCATCCAAGCAGCAAGC |
| | GCGTTACGCCGTGGGTCGATGTTTGATGTTATGGAGCAGCAACGA |
| | TGTTACGCAGCAGCAACGATGTTACGCAGCAGGGCAGTCGCCCTA |
| | AAACAAAGTTAGGTGGCTCAAGTATGGGCATCATTCGCACATGTA |
| | GGCTCGGCCCTGACCAAGTCAAATCCATGCGGGCTGCTCTTGATC |
| | TTTTCGGTCGTGAGTTCGGAGACGTAGCCACCTACTCCCAACATC |
| | AGCCGGACTCCGATTACCTCGGGAACTTGCTCCGTAGTAAGACAT |
| | TCATCGCGCTTGCTGCCTTCGACCAAGAAGCGGTTGTTGGCGCTC |
| | TCGCGGCTTACGTTCTGCCCAGGTTTGAGCAGCCGCGTAGTGAGA |
| | TCTATATCTATGATCTCGCAGTCTCCGGCGAGCACCGGAGGCAGG |
| | GCATTGCCACCGCGCTCATCAATCTCCTCAAGCATGAGGCCAACG |
| | CGCTTGGTGCTTATGTGATCTACGTGCAAGCAGATTACGGTGACG |
| | ATCCCGCAGTGGCTCTCTATACAAAGTTGGGCATACGGGAAGAAG |
| | TGATGCACTTTGATATCGACCCAAGTACCGCCACCTAACAATTCG |
| | TTCAAGCCGAGATCGGCTTCCCGGCCGCGGAGTTGTTCGGTAAAT |
| | TGTCACAACGCCGCGAATATAGTCTTTACCATGCCCTTGGCCACG |
| | CCCCTCTTTAATACGACGGGCAATTTGCACTTCAGAAAATGAAGA |
| | GTTTGCTTTAGCCATAACAAAAGTCCAGTATGCTTTTTCACAGCA |
| | TAACTGGACTGATTTCAGTTTACAACTATTCTGTCTAGTTTAAGA |
| | CTTTATTGTCATAGTTTAGATCTATTTTGTTCAGTTTAAGACTTT |
| | ATTGTCCGCCCACACCCGCTTACGCAGGGCATCCATTTATTACTC |
| | AACCGTAACCGATTTTGCCAGGTTACGCGGCTGGTCTGCGGTGTG |
| | AAATACCGCACAGATGCGTAAGGAGAAAATACCGCATCAGGCGCT |
| | CTTCCGCTTCCTCGCTCACTGACTCGCTGCGCTCGGTCGTTCGGC |
| | TGCGGCGAGCGGTATCAGCTCACTCAAAGGCGGTAATACGGTTAT |
| | CCACAGAATCAGGGGATAACGCAGGAAAGAACATGTGAGCAAAAG |
| | GCCAGCAAAAGGCCAGGAACCGTAAAAAGGCCGCGTTGCTGGCGT |
| | TTTTCCATAGGCTCCGCCCCCCTGACGAGCATCACAAAAATCGAC |
| | GCTCAAGTCAGAGGTGGCGAAACCCGACAGGACTATAAAGATACC |
| | AGGCGTTTCCCCCTGGAAGCTCCCTCGTGCGCTCTCCTGTTCCGA |
| | CCCTGCCGCTTACCGGATACCTGTCCGCCTTTCTCCCTTCGGGAA |
| | GCGTGGCGCTTTCTCAATGCTCACGCTGTAGGTATCTCAGTTCGG |
| | TGTAGGTCGTTCGCTCCAAGCTGGGCTGTGTGCACGAACCCCCCG |
| | TTCAGCCCGACCGCTGCGCCTTATCCGGTAACTATCGTCTTGAGT |
| | CCAACCCGGTAAGACACGACTTATCGCCACTGGCAGCAGCCACTG |
| | GTAACAGGATTAGCAGAGCGAGGTATGTAGGCGGTGCTACAGAGT |
| | TCTTGAAGTGGTGGCCTAACTACGGCTACACTAGAAGGACAGTAT |
| | TTGGTATCTGCGCTCTGCTGAAGCCAGTTACCTTCGGAAAAAGAG |
| | TTGGTAGCTCTTGATCCGGCAAACAAACCACCGCTGGTAGCGGTG |
| | GTTTTTTTGTTTGCAAGCAGCAGATTACGCGCAGAAAAAAAGGAT |
| | CTCAAGAAGATCCTTTGATCTTTTCTACGGGGTCTGACGCTCAGT |
| | GGAACGAAAACTCACGTTAAGGGATTTTGGTCATGAGATTATCAA |
| | AAAGGATCTTCACCTAGATCCTTTTAAATTAAAAATGAAGTTTTA |
| | AATCAATCTAAAGTATATATGAGTAAACTTGGTCTGACAGTTACC |
| | AATGCTTAATCAGTGAGGCACCTATCTCAGCGATCTGTCTATTTC |
| | GTTCATCCATAGTTGCCTGACTCCCCGTCGTGTAGATAACTACGA |
| | TACGGGAGGGCTTACCATCTGGCCCCAGTGCTGCAATGATACCGC |
| | GAGACCCACGCTCACCGGCTCCAGATTTATCAGCAATAAACCAGC |
| | CAGCCGGAAGGGCCGAGCGCAGAAGTGGTCCTGCAACTTTATCCG |
| | CCTCCATCCAGTCTATTAATTGTTGCCGGGAAGCTAGAGTAAGTA |
| | GTTCGCCAGTTAATAGTTTGCGCAACGTTGTTGCCATTGCTACAG |
| | GCATCGTGGTGTCACGCTCGTCGTTTGGTATGGCTTCATTCAGCT |
| | CCGGTTCCCAACGATCAAGGCGAGTTACATGATCCCCCATGTTGT |
| | GCAAAAAAGCGGTTAGCTCCTTCGGTCCTCCGATCGTTGTCAGAA |
| | GTAAGTTGGCCGCAGTGTTATCACTCATGGTTATGGCAGCACTGC |
| | ATAATTCTCTTACTGTCATGCCATCCGTAAGATGCTTTTCTGTGA |
| | CTGGTGAGTACTCAACCAAGTCATTCTGAGAATAGTGTATGCGGC |
| | GACCGAGTTGCTCTTGCCCGGCGTCAATACGGGATAATACCGCGC |
| | CACATAGCAGAACTTTAAAAGTGCTCATCATTGGAAAACGTTCTT |
| | CGGGGCGAAAACTCTCAAGGATCTTACCGCTGTTGAGATCCAGTT |
| | CGATGTAACCCACTCGTGCACCCAACTGATCTTCAGCATCTTTTA |
| | CTTTCACCAGCGTTTCTGGGTGAGCAAAAACAGGAAGGCAAAATG |
| | CCGCAAAAAAGGGAATAAGGGCGACACGGAAATGTTGAATACTCA |
| | TACTCTTCCTTTTTCAATATTATTGAAGCATTTATCAGGGTTATT |
| | GTCTCATGAGCGGATACATATTTGAATGTATTTAGAAAAATAAAC |
| | AAATAGGGGTTCCGCGCACATTTCCCCGAAAAGTGCCACCTGAAA |
| | TTGTAAACGTTAATATTTTGTTAAAATTCGCGTTAAATTTTTGTT |
| | AAATCAGCTCATTTTTTAACCAATAGGCCGAAATCGGCAAAATCC |
| | CTTATAAATCAAAAGAATAGACCGAGATAGGGTTGAGTGTTGTTC |
| | CAGTTTGGAACAAGAGTCCACTATTAAAGAACGTGGACTCCAACG |
| | TCAAAGGGCGAAAAACCGTCTATCAGGGCGATGGCCCACTACGTG |
| | AACCATCACCCTAATCAAGTTTTTTGGGGTCGAGGTGCCGTAAAG |
| | CACTAAATCGGAACCCTAAAGGGAGCCCCCGATTTAGAGCTTGAC |
| | GGGGAAAGCCGGCGAACGTGGCGAGAAAGGAAGGGAAGAAAGCGA |
| | AAGGAGCGGGCGCTAGGGCGCTGGCAAGTGTAGCGGTCACGCTGC |
| | GCGTAACCACCACACCCGCCGCGCTTAATGCGCCGCTACAGGGCG |
| | CGTC |
| AMI136; | 372-512: Full ITR |
| SEQ ID | 570-873: CMV enhancer |
| NO: 53 | 874-1077: CMV promoter |
| | 1181-1277: SV40 intron |
| | 1282-1290: Kozak sequence |
| | 1291-1371: Aflibercept secretion sequence |
| | 1372-2664: Aflibercept coding sequence after optimization |
| | 2674-2722: Poly A sequence |
| | 2757-3060: CMV enhancer |
| | 3061-3264: CMV promoter |
| | 3368-3464: SV40 intron |
| | 3469-3477: Kozak sequence |
| | 3478-3534: Human IgG heavy chain secretion sequence |
| | 3535-4398: hCOMP-Ang1 coding sequence before optimization |
| | 4422-4470: Poly A sequence |
| | 4498-4638: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGTGAGCTACTGG |
| | GACACCGGCGTGCTGCTGTGCGCCCTGCTGAGCTGCCTGCTGCTG |
| | ACCGGCAGCAGCAGCGGCAGCGACACCGGCAGGCCCTTCGTGGAG |
| | ATGTACTCCGAGATCCCCGAGATCATCCACATGACCGAGGGCAGG |
| | GAGCTGGTGATCCCCTGCAGGGTGACCTCCCCCAACATCACCGTG |
| | ACCCTGAAGAAGTTCCCCCTGGACACCCTGATCCCCGACGGCAAG |
| | AGGATCATCTGGGACTCCAGGAAGGGCTTCATCATCTCCAACGCC |
| | ACCTACAAGGAGATCGGCCTGCTGACCTGCGAGGCCACCGTGAAC |
| | GGCCACCTGTACAAGACCAACTACCTGACCCACAGGCAGACCAAC |
| | ACCATCATCGACGTGGTGCTGTCCCCCTCCCACGGCATCGAGCTG |
| | TCCGTGGGCGAGAAGCTGGTGCTGAACTGCACCGCCAGGACCGAG |
| | CTGAACGTGGGCATCGACTTCAACTGGGAGTACCCCTCCTCCAAG |
| | CACCAGCACAAGAAGCTGGTGAACAGGGACCTGAAGACCCAGTCC |
| | GGCTCCGAGATGAAGAAGTTCCTGTCCACCCTGACCATCGACGGC |
| | GTGACCAGGTCCGACCAGGGCCTGTACACCTGCGCCGCCTCCTCC |
| | GGCCTGATGACCAAGAAGAACTCCACCTTCGTGAGGGTGCACGAG |
| | AAGGACAAGACCCACACCTGCCCCCCCTGCCCCGCCCCCGAGCTG |
| | CTGGGCGGCCCCTCCGTGTTCCTGTTCCCCCCCAAGCCCAAGGAC |
| | ACCCTGATGATCTCCAGGACCCCCGAGGTGACCTGCGTGGTGGTG |
| | GACGTGTCCCACGAGGACCCCGAGGTGAAGTTCAACTGGTACGTG |
| | GACGGCGTGGAGGTGCACAACGCCAAGACCAAGCCCAGGGAGGAG |
| | CAGTACAACTCCACCTACAGGGTGGTGTCCGTGCTGACCGTGCTG |
| | CACCAGGACTGGCTGAACGGCAAGGAGTACAAGTGCAAGGTGTCC |
| | AACAAGGCCCTGCCCGCCCCCATCGAGAAGACCATCTCCAAGGCC |
| | AAGGGCCAGCCCAGGGAGCCCCAGGTGTACACCCTGCCCCCCTCC |
| | AGGGACGAGCTGACCAAGAACCAGGTGTCCCTGACCTGCCTGGTG |
| | AAGGGCTTCTACCCCTCCGACATCGCCGTGGAGTGGGAGTCCAAC |
| | GGCCAGCCCGAGAACAACTACAAGACCACCCCCCCCGTGCTGGAC |
| | TCCGACGGCTCCTTCTTCCTGTACTCCAAGCTGACCGTGGACAAG |
| | TCCAGGTGGCAGCAGGGCAACGTGTTCTCCTGCTCCGTGATGCAC |
| | GAGGCCCTGCACAACCACTACACCCAGAAGTCCCTGTCCCTGTCC |
| | CCCGGCAAGTGATTCGAAAATAAAATATCTTTATTTTCATTACAT |
| | CTGTGTGTTGGTTTTTTGTGTGGCATGCTGGGGAGAGATCAACCG |
| | AATTCGGTACCCGTTACATAACTTACGGTAAATGGCCCGCCTGGC |
| | TGACCGCCCAACGACCCCCGCCCATTGACGTCAATAATGACGTAT |
| | GTTCCCATAGTAACGCCAATAGGGACTTTCCATTGACGTCAATGG |
| | GTGGAGTATTTACGGTAAACTGCCCACTTGGCAGTACATCAAGTG |
| | TATCATATGCCAAGTACGCCCCCTATTGACGTCAATGACGGTAAA |
| | TGGCCCGCCTGGCATTATGCCCAGTACATGACCTTATGGGACTTT |
| | CCTACTTGGCAGTACATCTACGTATTAGTCATCGCTATTACCATG |
| | GTGATGCGGTTTTGGCAGTACATCAATGGGCGTGGATAGCGGTTT |
| | GACTCACGGGGATTTCCAAGTCTCCACCCCATTGACGTCAATGGG |
| | AGTTTGTTTTGGCACCAAAATCAACGGGACTTTCCAAAATGTCGT |
| | AACAACTCCGCCCCATTGACGCAAATGGGCGGTAGGCGTGTACGG |
| | TGGGAGGTCTATATAAGCAGAGCTCGTTTAGTGAACCGTCAGATC |
| | GCCTGGAGACGCCATCCACGCTGTTTTGACCTCCATAGAAGACAC |
| | CGGGACCGATCCAGCCTCCGGACTCTAGAGTTAACTGGTAAGTTT |
| | AGTCTTTTTGTCTTTTATTTCAGGTCCCGGATCCGGTGGTGGTGC |
| | AAATCAAAGAACTGCTCCTCAGTGGATGTTGCCTTTACTTCTAGG |
| | CCTGCCGCCACCATGGAGTTCGGCCTGAGCTGGCTGTTCCTGGTG |
| | GCCATCCTTAAGGGCGTGCAGTGCGACCTGGGCCCCCAGATGCTG |
| | AGGGAGCTCCAGGAGACCAATGCTGCTCTTCAGGATGTTAGGGAA |
| | CTGCTGAGGCAGCAGGTGAAGGAGATCACCTTCCTCAGAAACACA |
| | GTGATGGAGTGTGATGCCTGTGGGGACACAGTCCACAACCTGGTC |
| | AACCTGTGCACCAAAGAGGGTGTGCTGCTCAAGGGAGGGAAGAGG |
| | GAGGAGGAGAAGCCCTTCAGGGACTGTGCTGATGTCTACCAGGCT |
| | GGCTTCAACAAGAGTGGGATCTACACCATCTACATCAACAACATG |
| | CCTGAGCCCAAGAAGGTGTTCTGCAACATGGATGTGAATGGGGGG |
| | GGCTGGACTGTGATCCAGCACAGAGAAGATGGCTCCCTGGACTTC |
| | CAGAGGGGCTGGAAGGAATACAAGATGGGGTTTGGGAACCCCTCT |
| | GGGGAGTACTGGCTGGGCAATGAGTTCATCTTTGCCATCACTAGC |
| | CAGAGACAGTACATGCTCAGAATTGAGCTGATGGACTGGGAGGGC |
| | AACAGAGCCTACAGCCAATATGACAGGTTCCACATTGGAAATGAA |
| | AAGCAGAACTACAGGCTGTACCTGAAGGGCCACACTGGGACTGCA |
| | GGCAAGCAGAGCTCACTGATCCTGCATGGAGCTGACTTCTCCACC |
| | AAGGATGCAGACAATGACAACTGCATGTGCAAGTGTGCCCTCATG |
| | CTGACTGGTGGGTGGTGGTTTGATGCTTGTGGGCCCAGCAACCTG |
| | AATGGAATGTTCTACACAGCTGGGCAGAATCATGGCAAGCTCAAT |
| | GGCATCAAGTGGCACTACTTCAAGGGCCCCAGCTACAGCCTGAGG |
| | TCCACCACCATGATGATCAGGCCTCTGGACTTCTGAGCGGGACTC |
| | TGGAATTCGAAAATAAAATATCTTTATTTTCATTACATCTGTGTG |
| | TTGGTTTTTTGTGTGTTCTGCACGTGCGGACCGAGCGGCCGCAGG |
| | AACCCCTAGTGATGGAGTTGGCCACTCCCTCTCTGCGCGCTCGCT |
| | CGCTCACTGAGGCCGGGCGACCAAAGGTCGCCCGACGCCCGGGCT |
| | TTGCCCGGGCGGCCTCAGTGAGCGAGCGAGCGCGCAGCTGCCTGC |
| | AGGCATGCAAGCTGTAGCCAACCACTAGAACTATAGCTAGAGTCC |
| | TGGGCGAACAAACGATGCTCGCCTTCCAGAAAACCGAGGATGCGA |
| | ACCACTTCATCCGGGGTCAGCACCACCGGCAAGCGCCGCGACGGC |
| | CGAGGTCTTCCGATCTCCTGAAGCCAGGGCAGATCCGTGCACAGC |
| | ACCTTGCCGTAGAAGAACAGCAAGGCCGCCAATGCCTGACGATGC |
| | GTGGAGACCGAAACCTTGCGCTCGTTCGCCAGCCAGGACAGAAAT |
| | GCCTCGACTTCGCTGCTGCCCAAGGTTGCCGGGTGACGCACACCG |
| | TGGAAACGGATGAAGGCACGAACCCAGTTGACATAAGCCTGTTCG |
| | GTTCGTAAACTGTAATGCAAGTAGCGTATGCGCTCACGCAACTGG |
| | TCCAGAACCTTGACCGAACGCAGCGGTGGTAACGGCGCAGTGGCG |
| | GTTTTCATGGCTTGTTATGACTGTTTTTTTGTACAGTCTATGCCT |
| | CGGGCATCCAAGCAGCAAGCGCGTTACGCCGTGGGTCGATGTTTG |
| | ATGTTATGGAGCAGCAACGATGTTACGCAGCAGCAACGATGTTAC |
| | GCAGCAGGGCAGTCGCCCTAAAACAAAGTTAGGTGGCTCAAGTAT |
| | GGGCATCATTCGCACATGTAGGCTCGGCCCTGACCAAGTCAAATC |
| | CATGCGGGCTGCTCTTGATCTTTTCGGTCGTGAGTTCGGAGACGT |
| | AGCCACCTACTCCCAACATCAGCCGGACTCCGATTACCTCGGGAA |
| | CTTGCTCCGTAGTAAGACATTCATCGCGCTTGCTGCCTTCGACCA |
| | AGAAGCGGTTGTTGGCGCTCTCGCGGCTTACGTTCTGCCCAGGTT |
| | TGAGCAGCCGCGTAGTGAGATCTATATCTATGATCTCGCAGTCTC |
| | CGGCGAGCACCGGAGGCAGGGCATTGCCACCGCGCTCATCAATCT |
| | CCTCAAGCATGAGGCCAACGCGCTTGGTGCTTATGTGATCTACGT |
| | GCAAGCAGATTACGGTGACGATCCCGCAGTGGCTCTCTATACAAA |
| | GTTGGGCATACGGGAAGAAGTGATGCACTTTGATATCGACCCAAG |
| | TACCGCCACCTAACAATTCGTTCAAGCCGAGATCGGCTTCCCGGC |
| | CGCGGAGTTGTTCGGTAAATTGTCACAACGCCGCGAATATAGTCT |
| | TTACCATGCCCTTGGCCACGCCCCTCTTTAATACGACGGGCAATT |
| | TGCACTTCAGAAAATGAAGAGTTTGCTTTAGCCATAACAAAAGTC |
| | CAGTATGCTTTTTCACAGCATAACTGGACTGATTTCAGTTTACAA |
| | CTATTCTGTCTAGTTTAAGACTTTATTGTCATAGTTTAGATCTAT |
| | TTTGTTCAGTTTAAGACTTTATTGTCCGCCCACACCCGCTTACGC |
| | AGGGCATCCATTTATTACTCAACCGTAACCGATTTTGCCAGGTTA |
| | CGCGGCTGGTCTGCGGTGTGAAATACCGCACAGATGCGTAAGGAG |
| | AAAATACCGCATCAGGCGCTCTTCCGCTTCCTCGCTCACTGACTC |
| | GCTGCGCTCGGTCGTTCGGCTGCGGCGAGCGGTATCAGCTCACTC |
| | AAAGGCGGTAATACGGTTATCCACAGAATCAGGGGATAACGCAGG |
| | AAAGAACATGTGAGCAAAAGGCCAGCAAAAGGCCAGGAACCGTAA |
| | AAAGGCCGCGTTGCTGGCGTTTTTCCATAGGCTCCGCCCCCCTGA |
| | CGAGCATCACAAAAATCGACGCTCAAGTCAGAGGTGGCGAAACCC |
| | GACAGGACTATAAAGATACCAGGCGTTTCCCCCTGGAAGCTCCCT |
| | CGTGCGCTCTCCTGTTCCGACCCTGCCGCTTACCGGATACCTGTC |
| | CGCCTTTCTCCCTTCGGGAAGCGTGGCGCTTTCTCAATGCTCACG |
| | CTGTAGGTATCTCAGTTCGGTGTAGGTCGTTCGCTCCAAGCTGGG |
| | CTGTGTGCACGAACCCCCCGTTCAGCCCGACCGCTGCGCCTTATC |
| | CGGTAACTATCGTCTTGAGTCCAACCCGGTAAGACACGACTTATC |
| | GCCACTGGCAGCAGCCACTGGTAACAGGATTAGCAGAGCGAGGTA |
| | TGTAGGCGGTGCTACAGAGTTCTTGAAGTGGTGGCCTAACTACGG |
| | CTACACTAGAAGGACAGTATTTGGTATCTGCGCTCTGCTGAAGCC |
| | AGTTACCTTCGGAAAAAGAGTTGGTAGCTCTTGATCCGGCAAACA |
| | AACCACCGCTGGTAGCGGTGGTTTTTTTGTTTGCAAGCAGCAGAT |
| | TACGCGCAGAAAAAAAGGATCTCAAGAAGATCCTTTGATCTTTTC |
| | TACGGGGTCTGACGCTCAGTGGAACGAAAACTCACGTTAAGGGAT |
| | TTTGGTCATGAGATTATCAAAAAGGATCTTCACCTAGATCCTTTT |
| | AAATTAAAAATGAAGTTTTAAATCAATCTAAAGTATATATGAGTA |
| | AACTTGGTCTGACAGTTACCAATGCTTAATCAGTGAGGCACCTAT |
| | CTCAGCGATCTGTCTATTTCGTTCATCCATAGTTGCCTGACTCCC |
| | CGTCGTGTAGATAACTACGATACGGGAGGGCTTACCATCTGGCCC |
| | CAGTGCTGCAATGATACCGCGAGACCCACGCTCACCGGCTCCAGA |
| | TTTATCAGCAATAAACCAGCCAGCCGGAAGGGCCGAGCGCAGAAG |
| | TGGTCCTGCAACTTTATCCGCCTCCATCCAGTCTATTAATTGTTG |
| | CCGGGAAGCTAGAGTAAGTAGTTCGCCAGTTAATAGTTTGCGCAA |
| | CGTTGTTGCCATTGCTACAGGCATCGTGGTGTCACGCTCGTCGTT |
| | TGGTATGGCTTCATTCAGCTCCGGTTCCCAACGATCAAGGCGAGT |
| | TACATGATCCCCCATGTTGTGCAAAAAAGCGGTTAGCTCCTTCGG |
| | TCCTCCGATCGTTGTCAGAAGTAAGTTGGCCGCAGTGTTATCACT |
| | CATGGTTATGGCAGCACTGCATAATTCTCTTACTGTCATGCCATC |
| | CGTAAGATGCTTTTCTGTGACTGGTGAGTACTCAACCAAGTCATT |
| | CTGAGAATAGTGTATGCGGCGACCGAGTTGCTCTTGCCCGGCGTC |
| | AATACGGGATAATACCGCGCCACATAGCAGAACTTTAAAAGTGCT |
| | CATCATTGGAAAACGTTCTTCGGGGCGAAAACTCTCAAGGATCTT |
| | ACCGCTGTTGAGATCCAGTTCGATGTAACCCACTCGTGCACCCAA |
| | CTGATCTTCAGCATCTTTTACTTTCACCAGCGTTTCTGGGTGAGC |
| | AAAAACAGGAAGGCAAAATGCCGCAAAAAAGGGAATAAGGGCGAC |
| | ACGGAAATGTTGAATACTCATACTCTTCCTTTTTCAATATTATTG |
| | AAGCATTTATCAGGGTTATTGTCTCATGAGCGGATACATATTTGA |
| | ATGTATTTAGAAAAATAAACAAATAGGGGTTCCGCGCACATTTCC |
| | CCGAAAAGTGCCACCTGAAATTGTAAACGTTAATATTTTGTTAAA |
| | ATTCGCGTTAAATTTTTGTTAAATCAGCTCATTTTTTAACCAATA |
| | GGCCGAAATCGGCAAAATCCCTTATAAATCAAAAGAATAGACCGA |
| | GATAGGGTTGAGTGTTGTTCCAGTTTGGAACAAGAGTCCACTATT |
| | AAAGAACGTGGACTCCAACGTCAAAGGGCGAAAAACCGTCTATCA |
| | GGGCGATGGCCCACTACGTGAACCATCACCCTAATCAAGTTTTTT |
| | GGGGTCGAGGTGCCGTAAAGCACTAAATCGGAACCCTAAAGGGAG |
| | CCCCCGATTTAGAGCTTGACGGGGAAAGCCGGCGAACGTGGCGAG |
| | AAAGGAAGGGAAGAAAGCGAAAGGAGCGGGCGCTAGGGCGCTGGC |
| | AAGTGTAGCGGTCACGCTGCGCGTAACCACCACACCCGCCGCGCT |
| | TAATGCGCCGCTACAGGGCGCGTC |
| AMI142; | 372-512: Full ITR |
| SEQ ID | 570-873: CMV enhancer |
| NO: 54 | 874-1077: CMV promoter |
| | 1181-1277: SV40 intron |
| | 1282-1290: Kozak sequence |
| | 1291-1371: Aflibercept secretion sequence |
| | 1372-2664: Aflibercept coding sequence after optimization |
| | 2665-2682: Furin sequence |
| | 2683-2748: F2A sequence |
| | 2749-2805: Human IgG heavy chain secretion sequence |
| | 2806-3669: hCOMP-Ang1 coding sequence before optimization |
| | 3693-3741: Poly A sequence |
| | 3769-3909: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGTGAGCTACTGG |
| | GACACCGGCGTGCTGCTGTGCGCCCTGCTGAGCTGCCTGCTGCTG |
| | ACCGGCAGCAGCAGCGGCAGCGACACCGGCAGGCCCTTCGTGGAG |
| | ATGTACTCCGAGATCCCCGAGATCATCCACATGACCGAGGGCAGG |
| | GAGCTGGTGATCCCCTGCAGGGTGACCTCCCCCAACATCACCGTG |
| | ACCCTGAAGAAGTTCCCCCTGGACACCCTGATCCCCGACGGCAAG |
| | AGGATCATCTGGGACTCCAGGAAGGGCTTCATCATCTCCAACGCC |
| | ACCTACAAGGAGATCGGCCTGCTGACCTGCGAGGCCACCGTGAAC |
| | GGCCACCTGTACAAGACCAACTACCTGACCCACAGGCAGACCAAC |
| | ACCATCATCGACGTGGTGCTGTCCCCCTCCCACGGCATCGAGCTG |
| | TCCGTGGGCGAGAAGCTGGTGCTGAACTGCACCGCCAGGACCGAG |
| | CTGAACGTGGGCATCGACTTCAACTGGGAGTACCCCTCCTCCAAG |
| | CACCAGCACAAGAAGCTGGTGAACAGGGACCTGAAGACCCAGTCC |
| | GGCTCCGAGATGAAGAAGTTCCTGTCCACCCTGACCATCGACGGC |
| | GTGACCAGGTCCGACCAGGGCCTGTACACCTGCGCCGCCTCCTCC |
| | GGCCTGATGACCAAGAAGAACTCCACCTTCGTGAGGGTGCACGAG |
| | AAGGACAAGACCCACACCTGCCCCCCCTGCCCCGCCCCCGAGCTG |
| | CTGGGCGGCCCCTCCGTGTTCCTGTTCCCCCCCAAGCCCAAGGAC |
| | ACCCTGATGATCTCCAGGACCCCCGAGGTGACCTGCGTGGTGGTG |
| | GACGTGTCCCACGAGGACCCCGAGGTGAAGTTCAACTGGTACGTG |
| | GACGGCGTGGAGGTGCACAACGCCAAGACCAAGCCCAGGGAGGAG |
| | CAGTACAACTCCACCTACAGGGTGGTGTCCGTGCTGACCGTGCTG |
| | CACCAGGACTGGCTGAACGGCAAGGAGTACAAGTGCAAGGTGTCC |
| | AACAAGGCCCTGCCCGCCCCCATCGAGAAGACCATCTCCAAGGCC |
| | AAGGGCCAGCCCAGGGAGCCCCAGGTGTACACCCTGCCCCCCTCC |
| | AGGGACGAGCTGACCAAGAACCAGGTGTCCCTGACCTGCCTGGTG |
| | AAGGGCTTCTACCCCTCCGACATCGCCGTGGAGTGGGAGTCCAAC |
| | GGCCAGCCCGAGAACAACTACAAGACCACCCCCCCCGTGCTGGAC |
| | TCCGACGGCTCCTTCTTCCTGTACTCCAAGCTGACCGTGGACAAG |
| | TCCAGGTGGCAGCAGGGCAACGTGTTCTCCTGCTCCGTGATGCAC |
| | GAGGCCCTGCACAACCACTACACCCAGAAGTCCCTGTCCCTGTCC |
| | CCCGGCAAGAGAAGAAAGAGAGCCCCCGTGAAGCAGACCCTGAAC |
| | TTCGACCTGCTGAAGCTGGCCGGCGACGTGGAGAGCAACCCCGGC |
| | CCCATGGAGTTCGGCCTGAGCTGGCTGTTCCTGGTGGCCATCCTT |
| | AAGGGCGTGCAGTGCGACCTGGGCCCCCAGATGCTGAGGGAGCTC |
| | CAGGAGACCAATGCTGCTCTTCAGGATGTTAGGGAACTGCTGAGG |
| | CAGCAGGTGAAGGAGATCACCTTCCTCAGAAACACAGTGATGGAG |
| | TGTGATGCCTGTGGGGACACAGTCCACAACCTGGTCAACCTGTGC |
| | ACCAAAGAGGGTGTGCTGCTCAAGGGAGGGAAGAGGGAGGAGGAG |
| | AAGCCCTTCAGGGACTGTGCTGATGTCTACCAGGCTGGCTTCAAC |
| | AAGAGTGGGATCTACACCATCTACATCAACAACATGCCTGAGCCC |
| | AAGAAGGTGTTCTGCAACATGGATGTGAATGGGGGGGGCTGGACT |
| | GTGATCCAGCACAGAGAAGATGGCTCCCTGGACTTCCAGAGGGGC |
| | TGGAAGGAATACAAGATGGGGTTTGGGAACCCCTCTGGGGAGTAC |
| | TGGCTGGGCAATGAGTTCATCTTTGCCATCACTAGCCAGAGACAG |
| | TACATGCTCAGAATTGAGCTGATGGACTGGGAGGGCAACAGAGCC |
| | TACAGCCAATATGACAGGTTCCACATTGGAAATGAAAAGCAGAAC |
| | TACAGGCTGTACCTGAAGGGCCACACTGGGACTGCAGGCAAGCAG |
| | AGCTCACTGATCCTGCATGGAGCTGACTTCTCCACCAAGGATGCA |
| | GACAATGACAACTGCATGTGCAAGTGTGCCCTCATGCTGACTGGT |
| | GGGTGGTGGTTTGATGCTTGTGGGCCCAGCAACCTGAATGGAATG |
| | TTCTACACAGCTGGGCAGAATCATGGCAAGCTCAATGGCATCAAG |
| | TGGCACTACTTCAAGGGCCCCAGCTACAGCCTGAGGTCCACCACC |
| | ATGATGATCAGGCCTCTGGACTTCTGAGCGGGACTCTGGAATTCG |
| | AAAATAAAATATCTTTATTTTCATTACATCTGTGTGTTGGTTTTT |
| | TGTGTGTTCTGCACGTGCGGACCGAGCGGCCGCAGGAACCCCTAG |
| | TGATGGAGTTGGCCACTCCCTCTCTGCGCGCTCGCTCGCTCACTG |
| | AGGCCGGGCGACCAAAGGTCGCCCGACGCCCGGGCTTTGCCCGGG |
| | CGGCCTCAGTGAGCGAGCGAGCGCGCAGCTGCCTGCAGGCATGCA |
| | AGCTGTAGCCAACCACTAGAACTATAGCTAGAGTCCTGGGCGAAC |
| | AAACGATGCTCGCCTTCCAGAAAACCGAGGATGCGAACCACTTCA |
| | TCCGGGGTCAGCACCACCGGCAAGCGCCGCGACGGCCGAGGTCTT |
| | CCGATCTCCTGAAGCCAGGGCAGATCCGTGCACAGCACCTTGCCG |
| | TAGAAGAACAGCAAGGCCGCCAATGCCTGACGATGCGTGGAGACC |
| | GAAACCTTGCGCTCGTTCGCCAGCCAGGACAGAAATGCCTCGACT |
| | TCGCTGCTGCCCAAGGTTGCCGGGTGACGCACACCGTGGAAACGG |
| | ATGAAGGCACGAACCCAGTTGACATAAGCCTGTTCGGTTCGTAAA |
| | CTGTAATGCAAGTAGCGTATGCGCTCACGCAACTGGTCCAGAACC |
| | TTGACCGAACGCAGCGGTGGTAACGGCGCAGTGGCGGTTTTCATG |
| | GCTTGTTATGACTGTTTTTTTGTACAGTCTATGCCTCGGGCATCC |
| | AAGCAGCAAGCGCGTTACGCCGTGGGTCGATGTTTGATGTTATGG |
| | AGCAGCAACGATGTTACGCAGCAGCAACGATGTTACGCAGCAGGG |
| | CAGTCGCCCTAAAACAAAGTTAGGTGGCTCAAGTATGGGCATCAT |
| | TCGCACATGTAGGCTCGGCCCTGACCAAGTCAAATCCATGCGGGC |
| | TGCTCTTGATCTTTTCGGTCGTGAGTTCGGAGACGTAGCCACCTA |
| | CTCCCAACATCAGCCGGACTCCGATTACCTCGGGAACTTGCTCCG |
| | TAGTAAGACATTCATCGCGCTTGCTGCCTTCGACCAAGAAGCGGT |
| | TGTTGGCGCTCTCGCGGCTTACGTTCTGCCCAGGTTTGAGCAGCC |
| | GCGTAGTGAGATCTATATCTATGATCTCGCAGTCTCCGGCGAGCA |
| | CCGGAGGCAGGGCATTGCCACCGCGCTCATCAATCTCCTCAAGCA |
| | TGAGGCCAACGCGCTTGGTGCTTATGTGATCTACGTGCAAGCAGA |
| | TTACGGTGACGATCCCGCAGTGGCTCTCTATACAAAGTTGGGCAT |
| | ACGGGAAGAAGTGATGCACTTTGATATCGACCCAAGTACCGCCAC |
| | CTAACAATTCGTTCAAGCCGAGATCGGCTTCCCGGCCGCGGAGTT |
| | GTTCGGTAAATTGTCACAACGCCGCGAATATAGTCTTTACCATGC |
| | CCTTGGCCACGCCCCTCTTTAATACGACGGGCAATTTGCACTTCA |
| | GAAAATGAAGAGTTTGCTTTAGCCATAACAAAAGTCCAGTATGCT |
| | TTTTCACAGCATAACTGGACTGATTTCAGTTTACAACTATTCTGT |
| | CTAGTTTAAGACTTTATTGTCATAGTTTAGATCTATTTTGTTCAG |
| | TTTAAGACTTTATTGTCCGCCCACACCCGCTTACGCAGGGCATCC |
| | ATTTATTACTCAACCGTAACCGATTTTGCCAGGTTACGCGGCTGG |
| | TCTGCGGTGTGAAATACCGCACAGATGCGTAAGGAGAAAATACCG |
| | CATCAGGCGCTCTTCCGCTTCCTCGCTCACTGACTCGCTGCGCTC |
| | GGTCGTTCGGCTGCGGCGAGCGGTATCAGCTCACTCAAAGGCGGT |
| | AATACGGTTATCCACAGAATCAGGGGATAACGCAGGAAAGAACAT |
| | GTGAGCAAAAGGCCAGCAAAAGGCCAGGAACCGTAAAAAGGCCGC |
| | GTTGCTGGCGTTTTTCCATAGGCTCCGCCCCCCTGACGAGCATCA |
| | CAAAAATCGACGCTCAAGTCAGAGGTGGCGAAACCCGACAGGACT |
| | ATAAAGATACCAGGCGTTTCCCCCTGGAAGCTCCCTCGTGCGCTC |
| | TCCTGTTCCGACCCTGCCGCTTACCGGATACCTGTCCGCCTTTCT |
| | CCCTTCGGGAAGCGTGGCGCTTTCTCAATGCTCACGCTGTAGGTA |
| | TCTCAGTTCGGTGTAGGTCGTTCGCTCCAAGCTGGGCTGTGTGCA |
| | CGAACCCCCCGTTCAGCCCGACCGCTGCGCCTTATCCGGTAACTA |
| | TCGTCTTGAGTCCAACCCGGTAAGACACGACTTATCGCCACTGGC |
| | AGCAGCCACTGGTAACAGGATTAGCAGAGCGAGGTATGTAGGCGG |
| | TGCTACAGAGTTCTTGAAGTGGTGGCCTAACTACGGCTACACTAG |
| | AAGGACAGTATTTGGTATCTGCGCTCTGCTGAAGCCAGTTACCTT |
| | CGGAAAAAGAGTTGGTAGCTCTTGATCCGGCAAACAAACCACCGC |
| | TGGTAGCGGTGGTTTTTTTGTTTGCAAGCAGCAGATTACGCGCAG |
| | AAAAAAAGGATCTCAAGAAGATCCTTTGATCTTTTCTACGGGGTC |
| | TGACGCTCAGTGGAACGAAAACTCACGTTAAGGGATTTTGGTCAT |
| | GAGATTATCAAAAAGGATCTTCACCTAGATCCTTTTAAATTAAAA |
| | ATGAAGTTTTAAATCAATCTAAAGTATATATGAGTAAACTTGGTC |
| | TGACAGTTACCAATGCTTAATCAGTGAGGCACCTATCTCAGCGAT |
| | CTGTCTATTTCGTTCATCCATAGTTGCCTGACTCCCCGTCGTGTA |
| | GATAACTACGATACGGGAGGGCTTACCATCTGGCCCCAGTGCTGC |
| | AATGATACCGCGAGACCCACGCTCACCGGCTCCAGATTTATCAGC |
| | AATAAACCAGCCAGCCGGAAGGGCCGAGCGCAGAAGTGGTCCTGC |
| | AACTTTATCCGCCTCCATCCAGTCTATTAATTGTTGCCGGGAAGC |
| | TAGAGTAAGTAGTTCGCCAGTTAATAGTTTGCGCAACGTTGTTGC |
| | CATTGCTACAGGCATCGTGGTGTCACGCTCGTCGTTTGGTATGGC |
| | TTCATTCAGCTCCGGTTCCCAACGATCAAGGCGAGTTACATGATC |
| | CCCCATGTTGTGCAAAAAAGCGGTTAGCTCCTTCGGTCCTCCGAT |
| | CGTTGTCAGAAGTAAGTTGGCCGCAGTGTTATCACTCATGGTTAT |
| | GGCAGCACTGCATAATTCTCTTACTGTCATGCCATCCGTAAGATG |
| | CTTTTCTGTGACTGGTGAGTACTCAACCAAGTCATTCTGAGAATA |
| | GTGTATGCGGCGACCGAGTTGCTCTTGCCCGGCGTCAATACGGGA |
| | TAATACCGCGCCACATAGCAGAACTTTAAAAGTGCTCATCATTGG |
| | AAAACGTTCTTCGGGGCGAAAACTCTCAAGGATCTTACCGCTGTT |
| | GAGATCCAGTTCGATGTAACCCACTCGTGCACCCAACTGATCTTC |
| | AGCATCTTTTACTTTCACCAGCGTTTCTGGGTGAGCAAAAACAGG |
| | AAGGCAAAATGCCGCAAAAAAGGGAATAAGGGCGACACGGAAATG |
| | TTGAATACTCATACTCTTCCTTTTTCAATATTATTGAAGCATTTA |
| | TCAGGGTTATTGTCTCATGAGCGGATACATATTTGAATGTATTTA |
| | GAAAAATAAACAAATAGGGGTTCCGCGCACATTTCCCCGAAAAGT |
| | GCCACCTGAAATTGTAAACGTTAATATTTTGTTAAAATTCGCGTT |
| | AAATTTTTGTTAAATCAGCTCATTTTTTAACCAATAGGCCGAAAT |
| | CGGCAAAATCCCTTATAAATCAAAAGAATAGACCGAGATAGGGTT |
| | GAGTGTTGTTCCAGTTTGGAACAAGAGTCCACTATTAAAGAACGT |
| | GGACTCCAACGTCAAAGGGCGAAAAACCGTCTATCAGGGCGATGG |
| | CCCACTACGTGAACCATCACCCTAATCAAGTTTTTTGGGGTCGAG |
| | GTGCCGTAAAGCACTAAATCGGAACCCTAAAGGGAGCCCCCGATT |
| | TAGAGCTTGACGGGGAAAGCCGGCGAACGTGGCGAGAAAGGAAGG |
| | GAAGAAAGCGAAAGGAGCGGGCGCTAGGGCGCTGGCAAGTGTAGC |
| | GGTCACGCTGCGCGTAACCACCACACCCGCCGCGCTTAATGCGCC |
| | GCTACAGGGCGCGTC |
| AMI143; | 372-512: Full ITR |
| SEQ ID | 570-873: CMV enhancer |
| NO: 55 | 874-1077: CMV promoter |
| | 1181-1277: SV40 intron |
| | 1282-1290: Kozak sequence |
| | 1291-1371: Aflibercept secretion sequence |
| | 1372-2664: Aflibercept coding sequence after optimization |
| | 2677-2839: QBI SP163 sequence |
| | 2840-2896: Human IgG heavy chain secretion sequence |
| | 2897-3760: hCOMP-Ang1 coding sequence before optimization |
| | 3784-3832: Poly A sequence |
| | 3860-4000: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGTGAGCTACTGG |
| | GACACCGGCGTGCTGCTGTGCGCCCTGCTGAGCTGCCTGCTGCTG |
| | ACCGGCAGCAGCAGCGGCAGCGACACCGGCAGGCCCTTCGTGGAG |
| | ATGTACTCCGAGATCCCCGAGATCATCCACATGACCGAGGGCAGG |
| | GAGCTGGTGATCCCCTGCAGGGTGACCTCCCCCAACATCACCGTG |
| | ACCCTGAAGAAGTTCCCCCTGGACACCCTGATCCCCGACGGCAAG |
| | AGGATCATCTGGGACTCCAGGAAGGGCTTCATCATCTCCAACGCC |
| | ACCTACAAGGAGATCGGCCTGCTGACCTGCGAGGCCACCGTGAAC |
| | GGCCACCTGTACAAGACCAACTACCTGACCCACAGGCAGACCAAC |
| | ACCATCATCGACGTGGTGCTGTCCCCCTCCCACGGCATCGAGCTG |
| | TCCGTGGGCGAGAAGCTGGTGCTGAACTGCACCGCCAGGACCGAG |
| | CTGAACGTGGGCATCGACTTCAACTGGGAGTACCCCTCCTCCAAG |
| | CACCAGCACAAGAAGCTGGTGAACAGGGACCTGAAGACCCAGTCC |
| | GGCTCCGAGATGAAGAAGTTCCTGTCCACCCTGACCATCGACGGC |
| | GTGACCAGGTCCGACCAGGGCCTGTACACCTGCGCCGCCTCCTCC |
| | GGCCTGATGACCAAGAAGAACTCCACCTTCGTGAGGGTGCACGAG |
| | AAGGACAAGACCCACACCTGCCCCCCCTGCCCCGCCCCCGAGCTG |
| | CTGGGCGGCCCCTCCGTGTTCCTGTTCCCCCCCAAGCCCAAGGAC |
| | ACCCTGATGATCTCCAGGACCCCCGAGGTGACCTGCGTGGTGGTG |
| | GACGTGTCCCACGAGGACCCCGAGGTGAAGTTCAACTGGTACGTG |
| | GACGGCGTGGAGGTGCACAACGCCAAGACCAAGCCCAGGGAGGAG |
| | CAGTACAACTCCACCTACAGGGTGGTGTCCGTGCTGACCGTGCTG |
| | CACCAGGACTGGCTGAACGGCAAGGAGTACAAGTGCAAGGTGTCC |
| | AACAAGGCCCTGCCCGCCCCCATCGAGAAGACCATCTCCAAGGCC |
| | AAGGGCCAGCCCAGGGAGCCCCAGGTGTACACCCTGCCCCCCTCC |
| | AGGGACGAGCTGACCAAGAACCAGGTGTCCCTGACCTGCCTGGTG |
| | AAGGGCTTCTACCCCTCCGACATCGCCGTGGAGTGGGAGTCCAAC |
| | GGCCAGCCCGAGAACAACTACAAGACCACCCCCCCCGTGCTGGAC |
| | TCCGACGGCTCCTTCTTCCTGTACTCCAAGCTGACCGTGGACAAG |
| | TCCAGGTGGCAGCAGGGCAACGTGTTCTCCTGCTCCGTGATGCAC |
| | GAGGCCCTGCACAACCACTACACCCAGAAGTCCCTGTCCCTGTCC |
| | CCCGGCAAGTGAGATATCTAGAGCGCAGAGGCTTGGGGCAGCCGA |
| | GCGGCAGCCAGGCCCCGGCCCGGGCCTCGGTTCCAGAAGGGAGAG |
| | GAGCCCGCCAAGGCGCGCAAGAGAGCGGGCTGCCTCGCAGTCCGA |
| | GCCGGAGAGGGAGCGCGAGCCGCGCCGGCCCCGGACGGCCTCCGA |
| | AACCATGGAGTTCGGCCTGAGCTGGCTGTTCCTGGTGGCCATCCT |
| | TAAGGGCGTGCAGTGCGACCTGGGCCCCCAGATGCTGAGGGAGCT |
| | CCAGGAGACCAATGCTGCTCTTCAGGATGTTAGGGAACTGCTGAG |
| | GCAGCAGGTGAAGGAGATCACCTTCCTCAGAAACACAGTGATGGA |
| | GTGTGATGCCTGTGGGGACACAGTCCACAACCTGGTCAACCTGTG |
| | CACCAAAGAGGGTGTGCTGCTCAAGGGAGGGAAGAGGGAGGAGGA |
| | GAAGCCCTTCAGGGACTGTGCTGATGTCTACCAGGCTGGCTTCAA |
| | CAAGAGTGGGATCTACACCATCTACATCAACAACATGCCTGAGCC |
| | CAAGAAGGTGTTCTGCAACATGGATGTGAATGGGGGGGGCTGGAC |
| | TGTGATCCAGCACAGAGAAGATGGCTCCCTGGACTTCCAGAGGGG |
| | CTGGAAGGAATACAAGATGGGGTTTGGGAACCCCTCTGGGGAGTA |
| | CTGGCTGGGCAATGAGTTCATCTTTGCCATCACTAGCCAGAGACA |
| | GTACATGCTCAGAATTGAGCTGATGGACTGGGAGGGCAACAGAGC |
| | CTACAGCCAATATGACAGGTTCCACATTGGAAATGAAAAGCAGAA |
| | CTACAGGCTGTACCTGAAGGGCCACACTGGGACTGCAGGCAAGCA |
| | GAGCTCACTGATCCTGCATGGAGCTGACTTCTCCACCAAGGATGC |
| | AGACAATGACAACTGCATGTGCAAGTGTGCCCTCATGCTGACTGG |
| | TGGGTGGTGGTTTGATGCTTGTGGGCCCAGCAACCTGAATGGAAT |
| | GTTCTACACAGCTGGGCAGAATCATGGCAAGCTCAATGGCATCAA |
| | GTGGCACTACTTCAAGGGCCCCAGCTACAGCCTGAGGTCCACCAC |
| | CATGATGATCAGGCCTCTGGACTTCTGAGCGGGACTCTGGAATTC |
| | GAAAATAAAATATCTTTATTTTCATTACATCTGTGTGTTGGTTTT |
| | TTGTGTGTTCTGCACGTGCGGACCGAGCGGCCGCAGGAACCCCTA |
| | GTGATGGAGTTGGCCACTCCCTCTCTGCGCGCTCGCTCGCTCACT |
| | GAGGCCGGGCGACCAAAGGTCGCCCGACGCCCGGGCTTTGCCCGG |
| | GCGGCCTCAGTGAGCGAGCGAGCGCGCAGCTGCCTGCAGGCATGC |
| | AAGCTGTAGCCAACCACTAGAACTATAGCTAGAGTCCTGGGCGAA |
| | CAAACGATGCTCGCCTTCCAGAAAACCGAGGATGCGAACCACTTC |
| | ATCCGGGGTCAGCACCACCGGCAAGCGCCGCGACGGCCGAGGTCT |
| | TCCGATCTCCTGAAGCCAGGGCAGATCCGTGCACAGCACCTTGCC |
| | GTAGAAGAACAGCAAGGCCGCCAATGCCTGACGATGCGTGGAGAC |
| | CGAAACCTTGCGCTCGTTCGCCAGCCAGGACAGAAATGCCTCGAC |
| | TTCGCTGCTGCCCAAGGTTGCCGGGTGACGCACACCGTGGAAACG |
| | GATGAAGGCACGAACCCAGTTGACATAAGCCTGTTCGGTTCGTAA |
| | ACTGTAATGCAAGTAGCGTATGCGCTCACGCAACTGGTCCAGAAC |
| | CTTGACCGAACGCAGCGGTGGTAACGGCGCAGTGGCGGTTTTCAT |
| | GGCTTGTTATGACTGTTTTTTTGTACAGTCTATGCCTCGGGCATC |
| | CAAGCAGCAAGCGCGTTACGCCGTGGGTCGATGTTTGATGTTATG |
| | GAGCAGCAACGATGTTACGCAGCAGCAACGATGTTACGCAGCAGG |
| | GCAGTCGCCCTAAAACAAAGTTAGGTGGCTCAAGTATGGGCATCA |
| | TTCGCACATGTAGGCTCGGCCCTGACCAAGTCAAATCCATGCGGG |
| | CTGCTCTTGATCTTTTCGGTCGTGAGTTCGGAGACGTAGCCACCT |
| | ACTCCCAACATCAGCCGGACTCCGATTACCTCGGGAACTTGCTCC |
| | GTAGTAAGACATTCATCGCGCTTGCTGCCTTCGACCAAGAAGCGG |
| | TTGTTGGCGCTCTCGCGGCTTACGTTCTGCCCAGGTTTGAGCAGC |
| | CGCGTAGTGAGATCTATATCTATGATCTCGCAGTCTCCGGCGAGC |
| | ACCGGAGGCAGGGCATTGCCACCGCGCTCATCAATCTCCTCAAGC |
| | ATGAGGCCAACGCGCTTGGTGCTTATGTGATCTACGTGCAAGCAG |
| | ATTACGGTGACGATCCCGCAGTGGCTCTCTATACAAAGTTGGGCA |
| | TACGGGAAGAAGTGATGCACTTTGATATCGACCCAAGTACCGCCA |
| | CCTAACAATTCGTTCAAGCCGAGATCGGCTTCCCGGCCGCGGAGT |
| | TGTTCGGTAAATTGTCACAACGCCGCGAATATAGTCTTTACCATG |
| | CCCTTGGCCACGCCCCTCTTTAATACGACGGGCAATTTGCACTTC |
| | AGAAAATGAAGAGTTTGCTTTAGCCATAACAAAAGTCCAGTATGC |
| | TTTTTCACAGCATAACTGGACTGATTTCAGTTTACAACTATTCTG |
| | TCTAGTTTAAGACTTTATTGTCATAGTTTAGATCTATTTTGTTCA |
| | GTTTAAGACTTTATTGTCCGCCCACACCCGCTTACGCAGGGCATC |
| | CATTTATTACTCAACCGTAACCGATTTTGCCAGGTTACGCGGCTG |
| | GTCTGCGGTGTGAAATACCGCACAGATGCGTAAGGAGAAAATACC |
| | GCATCAGGCGCTCTTCCGCTTCCTCGCTCACTGACTCGCTGCGCT |
| | CGGTCGTTCGGCTGCGGCGAGCGGTATCAGCTCACTCAAAGGCGG |
| | TAATACGGTTATCCACAGAATCAGGGGATAACGCAGGAAAGAACA |
| | TGTGAGCAAAAGGCCAGCAAAAGGCCAGGAACCGTAAAAAGGCCG |
| | CGTTGCTGGCGTTTTTCCATAGGCTCCGCCCCCCTGACGAGCATC |
| | ACAAAAATCGACGCTCAAGTCAGAGGTGGCGAAACCCGACAGGAC |
| | TATAAAGATACCAGGCGTTTCCCCCTGGAAGCTCCCTCGTGCGCT |
| | CTCCTGTTCCGACCCTGCCGCTTACCGGATACCTGTCCGCCTTTC |
| | TCCCTTCGGGAAGCGTGGCGCTTTCTCAATGCTCACGCTGTAGGT |
| | ATCTCAGTTCGGTGTAGGTCGTTCGCTCCAAGCTGGGCTGTGTGC |
| | ACGAACCCCCCGTTCAGCCCGACCGCTGCGCCTTATCCGGTAACT |
| | ATCGTCTTGAGTCCAACCCGGTAAGACACGACTTATCGCCACTGG |
| | CAGCAGCCACTGGTAACAGGATTAGCAGAGCGAGGTATGTAGGCG |
| | GTGCTACAGAGTTCTTGAAGTGGTGGCCTAACTACGGCTACACTA |
| | GAAGGACAGTATTTGGTATCTGCGCTCTGCTGAAGCCAGTTACCT |
| | TCGGAAAAAGAGTTGGTAGCTCTTGATCCGGCAAACAAACCACCG |
| | CTGGTAGCGGTGGTTTTTTTGTTTGCAAGCAGCAGATTACGCGCA |
| | GAAAAAAAGGATCTCAAGAAGATCCTTTGATCTTTTCTACGGGGT |
| | CTGACGCTCAGTGGAACGAAAACTCACGTTAAGGGATTTTGGTCA |
| | TGAGATTATCAAAAAGGATCTTCACCTAGATCCTTTTAAATTAAA |
| | AATGAAGTTTTAAATCAATCTAAAGTATATATGAGTAAACTTGGT |
| | CTGACAGTTACCAATGCTTAATCAGTGAGGCACCTATCTCAGCGA |
| | TCTGTCTATTTCGTTCATCCATAGTTGCCTGACTCCCCGTCGTGT |
| | AGATAACTACGATACGGGAGGGCTTACCATCTGGCCCCAGTGCTG |
| | CAATGATACCGCGAGACCCACGCTCACCGGCTCCAGATTTATCAG |
| | CAATAAACCAGCCAGCCGGAAGGGCCGAGCGCAGAAGTGGTCCTG |
| | CAACTTTATCCGCCTCCATCCAGTCTATTAATTGTTGCCGGGAAG |
| | CTAGAGTAAGTAGTTCGCCAGTTAATAGTTTGCGCAACGTTGTTG |
| | CCATTGCTACAGGCATCGTGGTGTCACGCTCGTCGTTTGGTATGG |
| | CTTCATTCAGCTCCGGTTCCCAACGATCAAGGCGAGTTACATGAT |
| | CCCCCATGTTGTGCAAAAAAGCGGTTAGCTCCTTCGGTCCTCCGA |
| | TCGTTGTCAGAAGTAAGTTGGCCGCAGTGTTATCACTCATGGTTA |
| | TGGCAGCACTGCATAATTCTCTTACTGTCATGCCATCCGTAAGAT |
| | GCTTTTCTGTGACTGGTGAGTACTCAACCAAGTCATTCTGAGAAT |
| | AGTGTATGCGGCGACCGAGTTGCTCTTGCCCGGCGTCAATACGGG |
| | ATAATACCGCGCCACATAGCAGAACTTTAAAAGTGCTCATCATTG |
| | GAAAACGTTCTTCGGGGCGAAAACTCTCAAGGATCTTACCGCTGT |
| | TGAGATCCAGTTCGATGTAACCCACTCGTGCACCCAACTGATCTT |
| | CAGCATCTTTTACTTTCACCAGCGTTTCTGGGTGAGCAAAAACAG |
| | GAAGGCAAAATGCCGCAAAAAAGGGAATAAGGGCGACACGGAAAT |
| | GTTGAATACTCATACTCTTCCTTTTTCAATATTATTGAAGCATTT |
| | ATCAGGGTTATTGTCTCATGAGCGGATACATATTTGAATGTATTT |
| | AGAAAAATAAACAAATAGGGGTTCCGCGCACATTTCCCCGAAAAG |
| | TGCCACCTGAAATTGTAAACGTTAATATTTTGTTAAAATTCGCGT |
| | TAAATTTTTGTTAAATCAGCTCATTTTTTAACCAATAGGCCGAAA |
| | TCGGCAAAATCCCTTATAAATCAAAAGAATAGACCGAGATAGGGT |
| | TGAGTGTTGTTCCAGTTTGGAACAAGAGTCCACTATTAAAGAACG |
| | TGGACTCCAACGTCAAAGGGCGAAAAACCGTCTATCAGGGCGATG |
| | GCCCACTACGTGAACCATCACCCTAATCAAGTTTTTTGGGGTCGA |
| | GGTGCCGTAAAGCACTAAATCGGAACCCTAAAGGGAGCCCCCGAT |
| | TTAGAGCTTGACGGGGAAAGCCGGCGAACGTGGCGAGAAAGGAAG |
| | GGAAGAAAGCGAAAGGAGCGGGCGCTAGGGCGCTGGCAAGTGTAG |
| | CGGTCACGCTGCGCGTAACCACCACACCCGCCGCGCTTAATGCGC |
| | CGCTACAGGGCGCGTC |
| AMI144; | 372-512: Full ITR |
| SEQ ID | 570-873: CMV enhancer |
| NO: 56 | 874-1077: CMV promoter |
| | 1181-1277: SV40 intron |
| | 1282-1290: Kozak sequence |
| | 1291-1371: Aflibercept secretion sequence |
| | 1372-2664: Aflibercept coding sequence after optimization |
| | 2665-2724: 4xGGGGS sequence |
| | 2725-3588: hCOMP-Ang1 coding sequence before optimization |
| | 3612-3660: Poly A sequence |
| | 3688-3828: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGTGAGCTACTGG |
| | GACACCGGCGTGCTGCTGTGCGCCCTGCTGAGCTGCCTGCTGCTG |
| | ACCGGCAGCAGCAGCGGCAGCGACACCGGCAGGCCCTTCGTGGAG |
| | ATGTACTCCGAGATCCCCGAGATCATCCACATGACCGAGGGCAGG |
| | GAGCTGGTGATCCCCTGCAGGGTGACCTCCCCCAACATCACCGTG |
| | ACCCTGAAGAAGTTCCCCCTGGACACCCTGATCCCCGACGGCAAG |
| | AGGATCATCTGGGACTCCAGGAAGGGCTTCATCATCTCCAACGCC |
| | ACCTACAAGGAGATCGGCCTGCTGACCTGCGAGGCCACCGTGAAC |
| | GGCCACCTGTACAAGACCAACTACCTGACCCACAGGCAGACCAAC |
| | ACCATCATCGACGTGGTGCTGTCCCCCTCCCACGGCATCGAGCTG |
| | TCCGTGGGCGAGAAGCTGGTGCTGAACTGCACCGCCAGGACCGAG |
| | CTGAACGTGGGCATCGACTTCAACTGGGAGTACCCCTCCTCCAAG |
| | CACCAGCACAAGAAGCTGGTGAACAGGGACCTGAAGACCCAGTCC |
| | GGCTCCGAGATGAAGAAGTTCCTGTCCACCCTGACCATCGACGGC |
| | GTGACCAGGTCCGACCAGGGCCTGTACACCTGCGCCGCCTCCTCC |
| | GGCCTGATGACCAAGAAGAACTCCACCTTCGTGAGGGTGCACGAG |
| | AAGGACAAGACCCACACCTGCCCCCCCTGCCCCGCCCCCGAGCTG |
| | CTGGGCGGCCCCTCCGTGTTCCTGTTCCCCCCCAAGCCCAAGGAC |
| | ACCCTGATGATCTCCAGGACCCCCGAGGTGACCTGCGTGGTGGTG |
| | GACGTGTCCCACGAGGACCCCGAGGTGAAGTTCAACTGGTACGTG |
| | GACGGCGTGGAGGTGCACAACGCCAAGACCAAGCCCAGGGAGGAG |
| | CAGTACAACTCCACCTACAGGGTGGTGTCCGTGCTGACCGTGCTG |
| | CACCAGGACTGGCTGAACGGCAAGGAGTACAAGTGCAAGGTGTCC |
| | AACAAGGCCCTGCCCGCCCCCATCGAGAAGACCATCTCCAAGGCC |
| | AAGGGCCAGCCCAGGGAGCCCCAGGTGTACACCCTGCCCCCCTCC |
| | AGGGACGAGCTGACCAAGAACCAGGTGTCCCTGACCTGCCTGGTG |
| | AAGGGCTTCTACCCCTCCGACATCGCCGTGGAGTGGGAGTCCAAC |
| | GGCCAGCCCGAGAACAACTACAAGACCACCCCCCCCGTGCTGGAC |
| | TCCGACGGCTCCTTCTTCCTGTACTCCAAGCTGACCGTGGACAAG |
| | TCCAGGTGGCAGCAGGGCAACGTGTTCTCCTGCTCCGTGATGCAC |
| | GAGGCCCTGCACAACCACTACACCCAGAAGTCCCTGTCCCTGTCC |
| | CCCGGCAAGGGCGGAGGCGGAAGCGGCGGAGGCGGATCTGGCGGA |
| | GGCGGCAGCGGCGGCGGCGGCTCTGACCTGGGCCCCCAGATGCTG |
| | AGGGAGCTCCAGGAGACCAATGCTGCTCTTCAGGATGTTAGGGAA |
| | CTGCTGAGGCAGCAGGTGAAGGAGATCACCTTCCTCAGAAACACA |
| | GTGATGGAGTGTGATGCCTGTGGGGACACAGTCCACAACCTGGTC |
| | AACCTGTGCACCAAAGAGGGTGTGCTGCTCAAGGGAGGGAAGAGG |
| | GAGGAGGAGAAGCCCTTCAGGGACTGTGCTGATGTCTACCAGGCT |
| | GGCTTCAACAAGAGTGGGATCTACACCATCTACATCAACAACATG |
| | CCTGAGCCCAAGAAGGTGTTCTGCAACATGGATGTGAATGGGGGG |
| | GGCTGGACTGTGATCCAGCACAGAGAAGATGGCTCCCTGGACTTC |
| | CAGAGGGGCTGGAAGGAATACAAGATGGGGTTTGGGAACCCCTCT |
| | GGGGAGTACTGGCTGGGCAATGAGTTCATCTTTGCCATCACTAGC |
| | CAGAGACAGTACATGCTCAGAATTGAGCTGATGGACTGGGAGGGC |
| | AACAGAGCCTACAGCCAATATGACAGGTTCCACATTGGAAATGAA |
| | AAGCAGAACTACAGGCTGTACCTGAAGGGCCACACTGGGACTGCA |
| | GGCAAGCAGAGCTCACTGATCCTGCATGGAGCTGACTTCTCCACC |
| | AAGGATGCAGACAATGACAACTGCATGTGCAAGTGTGCCCTCATG |
| | CTGACTGGTGGGTGGTGGTTTGATGCTTGTGGGCCCAGCAACCTG |
| | AATGGAATGTTCTACACAGCTGGGCAGAATCATGGCAAGCTCAAT |
| | GGCATCAAGTGGCACTACTTCAAGGGCCCCAGCTACAGCCTGAGG |
| | TCCACCACCATGATGATCAGGCCTCTGGACTTCTGAGCGGGACTC |
| | TGGAATTCGAAAATAAAATATCTTTATTTTCATTACATCTGTGTG |
| | TTGGTTTTTTGTGTGTTCTGCACGTGCGGACCGAGCGGCCGCAGG |
| | AACCCCTAGTGATGGAGTTGGCCACTCCCTCTCTGCGCGCTCGCT |
| | CGCTCACTGAGGCCGGGCGACCAAAGGTCGCCCGACGCCCGGGCT |
| | TTGCCCGGGCGGCCTCAGTGAGCGAGCGAGCGCGCAGCTGCCTGC |
| | AGGCATGCAAGCTGTAGCCAACCACTAGAACTATAGCTAGAGTCC |
| | TGGGCGAACAAACGATGCTCGCCTTCCAGAAAACCGAGGATGCGA |
| | ACCACTTCATCCGGGGTCAGCACCACCGGCAAGCGCCGCGACGGC |
| | CGAGGTCTTCCGATCTCCTGAAGCCAGGGCAGATCCGTGCACAGC |
| | ACCTTGCCGTAGAAGAACAGCAAGGCCGCCAATGCCTGACGATGC |
| | GTGGAGACCGAAACCTTGCGCTCGTTCGCCAGCCAGGACAGAAAT |
| | GCCTCGACTTCGCTGCTGCCCAAGGTTGCCGGGTGACGCACACCG |
| | TGGAAACGGATGAAGGCACGAACCCAGTTGACATAAGCCTGTTCG |
| | GTTCGTAAACTGTAATGCAAGTAGCGTATGCGCTCACGCAACTGG |
| | TCCAGAACCTTGACCGAACGCAGCGGTGGTAACGGCGCAGTGGCG |
| | GTTTTCATGGCTTGTTATGACTGTTTTTTTGTACAGTCTATGCCT |
| | CGGGCATCCAAGCAGCAAGCGCGTTACGCCGTGGGTCGATGTTTG |
| | ATGTTATGGAGCAGCAACGATGTTACGCAGCAGCAACGATGTTAC |
| | GCAGCAGGGCAGTCGCCCTAAAACAAAGTTAGGTGGCTCAAGTAT |
| | GGGCATCATTCGCACATGTAGGCTCGGCCCTGACCAAGTCAAATC |
| | CATGCGGGCTGCTCTTGATCTTTTCGGTCGTGAGTTCGGAGACGT |
| | AGCCACCTACTCCCAACATCAGCCGGACTCCGATTACCTCGGGAA |
| | CTTGCTCCGTAGTAAGACATTCATCGCGCTTGCTGCCTTCGACCA |
| | AGAAGCGGTTGTTGGCGCTCTCGCGGCTTACGTTCTGCCCAGGTT |
| | TGAGCAGCCGCGTAGTGAGATCTATATCTATGATCTCGCAGTCTC |
| | CGGCGAGCACCGGAGGCAGGGCATTGCCACCGCGCTCATCAATCT |
| | CCTCAAGCATGAGGCCAACGCGCTTGGTGCTTATGTGATCTACGT |
| | GCAAGCAGATTACGGTGACGATCCCGCAGTGGCTCTCTATACAAA |
| | GTTGGGCATACGGGAAGAAGTGATGCACTTTGATATCGACCCAAG |
| | TACCGCCACCTAACAATTCGTTCAAGCCGAGATCGGCTTCCCGGC |
| | CGCGGAGTTGTTCGGTAAATTGTCACAACGCCGCGAATATAGTCT |
| | TTACCATGCCCTTGGCCACGCCCCTCTTTAATACGACGGGCAATT |
| | TGCACTTCAGAAAATGAAGAGTTTGCTTTAGCCATAACAAAAGTC |
| | CAGTATGCTTTTTCACAGCATAACTGGACTGATTTCAGTTTACAA |
| | CTATTCTGTCTAGTTTAAGACTTTATTGTCATAGTTTAGATCTAT |
| | TTTGTTCAGTTTAAGACTTTATTGTCCGCCCACACCCGCTTACGC |
| | AGGGCATCCATTTATTACTCAACCGTAACCGATTTTGCCAGGTTA |
| | CGCGGCTGGTCTGCGGTGTGAAATACCGCACAGATGCGTAAGGAG |
| | AAAATACCGCATCAGGCGCTCTTCCGCTTCCTCGCTCACTGACTC |
| | GCTGCGCTCGGTCGTTCGGCTGCGGCGAGCGGTATCAGCTCACTC |
| | AAAGGCGGTAATACGGTTATCCACAGAATCAGGGGATAACGCAGG |
| | AAAGAACATGTGAGCAAAAGGCCAGCAAAAGGCCAGGAACCGTAA |
| | AAAGGCCGCGTTGCTGGCGTTTTTCCATAGGCTCCGCCCCCCTGA |
| | CGAGCATCACAAAAATCGACGCTCAAGTCAGAGGTGGCGAAACCC |
| | GACAGGACTATAAAGATACCAGGCGTTTCCCCCTGGAAGCTCCCT |
| | CGTGCGCTCTCCTGTTCCGACCCTGCCGCTTACCGGATACCTGTC |
| | CGCCTTTCTCCCTTCGGGAAGCGTGGCGCTTTCTCAATGCTCACG |
| | CTGTAGGTATCTCAGTTCGGTGTAGGTCGTTCGCTCCAAGCTGGG |
| | CTGTGTGCACGAACCCCCCGTTCAGCCCGACCGCTGCGCCTTATC |
| | CGGTAACTATCGTCTTGAGTCCAACCCGGTAAGACACGACTTATC |
| | GCCACTGGCAGCAGCCACTGGTAACAGGATTAGCAGAGCGAGGTA |
| | TGTAGGCGGTGCTACAGAGTTCTTGAAGTGGTGGCCTAACTACGG |
| | CTACACTAGAAGGACAGTATTTGGTATCTGCGCTCTGCTGAAGCC |
| | AGTTACCTTCGGAAAAAGAGTTGGTAGCTCTTGATCCGGCAAACA |
| | AACCACCGCTGGTAGCGGTGGTTTTTTTGTTTGCAAGCAGCAGAT |
| | TACGCGCAGAAAAAAAGGATCTCAAGAAGATCCTTTGATCTTTTC |
| | TACGGGGTCTGACGCTCAGTGGAACGAAAACTCACGTTAAGGGAT |
| | TTTGGTCATGAGATTATCAAAAAGGATCTTCACCTAGATCCTTTT |
| | AAATTAAAAATGAAGTTTTAAATCAATCTAAAGTATATATGAGTA |
| | AACTTGGTCTGACAGTTACCAATGCTTAATCAGTGAGGCACCTAT |
| | CTCAGCGATCTGTCTATTTCGTTCATCCATAGTTGCCTGACTCCC |
| | CGTCGTGTAGATAACTACGATACGGGAGGGCTTACCATCTGGCCC |
| | CAGTGCTGCAATGATACCGCGAGACCCACGCTCACCGGCTCCAGA |
| | TTTATCAGCAATAAACCAGCCAGCCGGAAGGGCCGAGCGCAGAAG |
| | TGGTCCTGCAACTTTATCCGCCTCCATCCAGTCTATTAATTGTTG |
| | CCGGGAAGCTAGAGTAAGTAGTTCGCCAGTTAATAGTTTGCGCAA |
| | CGTTGTTGCCATTGCTACAGGCATCGTGGTGTCACGCTCGTCGTT |
| | TGGTATGGCTTCATTCAGCTCCGGTTCCCAACGATCAAGGCGAGT |
| | TACATGATCCCCCATGTTGTGCAAAAAAGCGGTTAGCTCCTTCGG |
| | TCCTCCGATCGTTGTCAGAAGTAAGTTGGCCGCAGTGTTATCACT |
| | CATGGTTATGGCAGCACTGCATAATTCTCTTACTGTCATGCCATC |
| | CGTAAGATGCTTTTCTGTGACTGGTGAGTACTCAACCAAGTCATT |
| | CTGAGAATAGTGTATGCGGCGACCGAGTTGCTCTTGCCCGGCGTC |
| | AATACGGGATAATACCGCGCCACATAGCAGAACTTTAAAAGTGCT |
| | CATCATTGGAAAACGTTCTTCGGGGCGAAAACTCTCAAGGATCTT |
| | ACCGCTGTTGAGATCCAGTTCGATGTAACCCACTCGTGCACCCAA |
| | CTGATCTTCAGCATCTTTTACTTTCACCAGCGTTTCTGGGTGAGC |
| | AAAAACAGGAAGGCAAAATGCCGCAAAAAAGGGAATAAGGGCGAC |
| | ACGGAAATGTTGAATACTCATACTCTTCCTTTTTCAATATTATTG |
| | AAGCATTTATCAGGGTTATTGTCTCATGAGCGGATACATATTTGA |
| | ATGTATTTAGAAAAATAAACAAATAGGGGTTCCGCGCACATTTCC |
| | CCGAAAAGTGCCACCTGAAATTGTAAACGTTAATATTTTGTTAAA |
| | ATTCGCGTTAAATTTTTGTTAAATCAGCTCATTTTTTAACCAATA |
| | GGCCGAAATCGGCAAAATCCCTTATAAATCAAAAGAATAGACCGA |
| | GATAGGGTTGAGTGTTGTTCCAGTTTGGAACAAGAGTCCACTATT |
| | AAAGAACGTGGACTCCAACGTCAAAGGGCGAAAAACCGTCTATCA |
| | GGGCGATGGCCCACTACGTGAACCATCACCCTAATCAAGTTTTTT |
| | GGGGTCGAGGTGCCGTAAAGCACTAAATCGGAACCCTAAAGGGAG |
| | CCCCCGATTTAGAGCTTGACGGGGAAAGCCGGCGAACGTGGCGAG |
| | AAAGGAAGGGAAGAAAGCGAAAGGAGCGGGCGCTAGGGCGCTGGC |
| | AAGTGTAGCGGTCACGCTGCGCGTAACCACCACACCCGCCGCGCT |
| | TAATGCGCCGCTACAGGGCGCGTC |
| AMI145; | 611-755: Full ITR |
| SEQ ID | 801-104: CMV enhancer |
| NO: 57 | 1105-1308: CMV promoter |
| | 1412-1508: SV40 intron |
| | 1513-1521: Kozak sequence |
| | 1522-1602: Aflibercept secretion sequence |
| | 1603-2895: Aflibercept coding sequence after optimization |
| | 2905-2953: Poly A signal |
| | 2954-3194: Human U6 promoter |
| | 3201-3256: shRNA1 against Ang2 |
| | (GGTTCAACGGCATTAAATAtacctgacccataTATTTAATGCCGTTGAACCTTTTT |
| | 3279-3384: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGTGAGC |
| | TACTGGGACACCGGCGTGCTGCTGTGCGCCCTGCTGAGCTGCCTG |
| | CTGCTGACCGGCAGCAGCAGCGGCAGCGACACCGGCAGGCCCTTC |
| | GTGGAGATGTACTCCGAGATCCCCGAGATCATCCACATGACCGAG |
| | GGCAGGGAGCTGGTGATCCCCTGCAGGGTGACCTCCCCCAACATC |
| | ACCGTGACCCTGAAGAAGTTCCCCCTGGACACCCTGATCCCCGAC |
| | GGCAAGAGGATCATCTGGGACTCCAGGAAGGGCTTCATCATCTCC |
| | AACGCCACCTACAAGGAGATCGGCCTGCTGACCTGCGAGGCCACC |
| | GTGAACGGCCACCTGTACAAGACCAACTACCTGACCCACAGGCAG |
| | ACCAACACCATCATCGACGTGGTGCTGTCCCCCTCCCACGGCATC |
| | GAGCTGTCCGTGGGCGAGAAGCTGGTGCTGAACTGCACCGCCAGG |
| | ACCGAGCTGAACGTGGGCATCGACTTCAACTGGGAGTACCCCTCC |
| | TCCAAGCACCAGCACAAGAAGCTGGTGAACAGGGACCTGAAGACC |
| | CAGTCCGGCTCCGAGATGAAGAAGTTCCTGTCCACCCTGACCATC |
| | GACGGCGTGACCAGGTCCGACCAGGGCCTGTACACCTGCGCCGCC |
| | TCCTCCGGCCTGATGACCAAGAAGAACTCCACCTTCGTGAGGGTG |
| | CACGAGAAGGACAAGACCCACACCTGCCCCCCCTGCCCCGCCCCC |
| | GAGCTGCTGGGCGGCCCCTCCGTGTTCCTGTTCCCCCCCAAGCCC |
| | AAGGACACCCTGATGATCTCCAGGACCCCCGAGGTGACCTGCGTG |
| | GTGGTGGACGTGTCCCACGAGGACCCCGAGGTGAAGTTCAACTGG |
| | TACGTGGACGGCGTGGAGGTGCACAACGCCAAGACCAAGCCCAGG |
| | GAGGAGCAGTACAACTCCACCTACAGGGTGGTGTCCGTGCTGACC |
| | GTGCTGCACCAGGACTGGCTGAACGGCAAGGAGTACAAGTGCAAG |
| | GTGTCCAACAAGGCCCTGCCCGCCCCCATCGAGAAGACCATCTCC |
| | AAGGCCAAGGGCCAGCCCAGGGAGCCCCAGGTGTACACCCTGCCC |
| | CCCTCCAGGGACGAGCTGACCAAGAACCAGGTGTCCCTGACCTGC |
| | CTGGTGAAGGGCTTCTACCCCTCCGACATCGCCGTGGAGTGGGAG |
| | TCCAACGGCCAGCCCGAGAACAACTACAAGACCACCCCCCCCGTG |
| | CTGGACTCCGACGGCTCCTTCTTCCTGTACTCCAAGCTGACCGTG |
| | GACAAGTCCAGGTGGCAGCAGGGCAACGTGTTCTCCTGCTCCGTG |
| | ATGCACGAGGCCCTGCACAACCACTACACCCAGAAGTCCCTGTCC |
| | CTGTCCCCCGGCAAGTGATTCGAAAATAAAATATCTTTATTTTCA |
| | TTACATCTGTGTGTTGGTTTTTTGTGTGGAGGGCCTATTTCCCAT |
| | GATTCCTTCATATTTGCATATACGATACAAGGCTGTTAGAGAGAT |
| | AATTGGAATTAATTTGACTGTAAACACAAAGATATTAGTACAAAA |
| | TACGTGACGTAGAAAGTAATAATTTCTTGGGTAGTTTGCAGTTTT |
| | AAAATTATGTTTTAAAATGGACTATCATATGCTTACCGTAACTTG |
| | AAAGTATTTCGATTTCTTGGCTTTATATATCTTGTGGAAAGGACA |
| | AGCTTGGTTCAACGGCATTAAATATACCTGACCCATATATTTAAT |
| | GCCGTTGAACCTTTTTGCATGCTGGGGAGAGATCAACCCCACTCC |
| | CTCTCTGCGCGCTCGCTCGCTCACTGAGGCCGGGCGACCAAAGGT |
| | CGCCCGACGCCCGGGCTTTGCCCGGGCGGCCTCAGTGAGCGAGCG |
| | AGCGCGCAGCAAGCTGTAGCCAACCACTAGAACTATAGCTAGAGT |
| | CCTGGGCGAACAAACGATGCTCGCCTTCCAGAAAACCGAGGATGC |
| | GAACCACTTCATCCGGGGTCAGCACCACCGGCAAGCGCCGCGACG |
| | GCCGAGGTCTTCCGATCTCCTGAAGCCAGGGCAGATCCGTGCACA |
| | GCACCTTGCCGTAGAAGAACAGCAAGGCCGCCAATGCCTGACGAT |
| | GCGTGGAGACCGAAACCTTGCGCTCGTTCGCCAGCCAGGACAGAA |
| | ATGCCTCGACTTCGCTGCTGCCCAAGGTTGCCGGGTGACGCACAC |
| | CGTGGAAACGGATGAAGGCACGAACCCAGTTGACATAAGCCTGTT |
| | CGGTTCGTAAACTGTAATGCAAGTAGCGTATGCGCTCACGCAACT |
| | GGTCCAGAACCTTGACCGAACGCAGCGGTGGTAACGGCGCAGTGG |
| | CGGTTTTCATGGCTTGTTATGACTGTTTTTTTGTACAGTCTATGC |
| | CTCGGGCATCCAAGCAGCAAGCGCGTTACGCCGTGGGTCGATGTT |
| | TGATGTTATGGAGCAGCAACGATGTTACGCAGCAGCAACGATGTT |
| | ACGCAGCAGGGCAGTCGCCCTAAAACAAAGTTAGGTGGCTCAAGT |
| | ATGGGCATCATTCGCACATGTAGGCTCGGCCCTGACCAAGTCAAA |
| | TCCATGCGGGCTGCTCTTGATCTTTTCGGTCGTGAGTTCGGAGAC |
| | GTAGCCACCTACTCCCAACATCAGCCGGACTCCGATTACCTCGGG |
| | AACTTGCTCCGTAGTAAGACATTCATCGCGCTTGCTGCCTTCGAC |
| | CAAGAAGCGGTTGTTGGCGCTCTCGCGGCTTACGTTCTGCCCAGG |
| | TTTGAGCAGCCGCGTAGTGAGATCTATATCTATGATCTCGCAGTC |
| | TCCGGCGAGCACCGGAGGCAGGGCATTGCCACCGCGCTCATCAAT |
| | CTCCTCAAGCATGAGGCCAACGCGCTTGGTGCTTATGTGATCTAC |
| | GTGCAAGCAGATTACGGTGACGATCCCGCAGTGGCTCTCTATACA |
| | AAGTTGGGCATACGGGAAGAAGTGATGCACTTTGATATCGACCCA |
| | AGTACCGCCACCTAACAATTCGTTCAAGCCGAGATCGGCTTCCCG |
| | GCCGCGGAGTTGTTCGGTAAATTGTCACAACGCCGCGAATATAGT |
| | CTTTACCATGCCCTTGGCCACGCCCCTCTTTAATACGACGGGCAA |
| | TTTGCACTTCAGAAAATGAAGAGTTTGCTTTAGCCATAACAAAAG |
| | TCCAGTATGCTTTTTCACAGCATAACTGGACTGATTTCAGTTTAC |
| | AACTATTCTGTCTAGTTTAAGACTTTATTGTCATAGTTTAGATCT |
| | ATTTTGTTCAGTTTAAGACTTTATTGTCCGCCCACACCCGCTTAC |
| | GCAGGGCATCCATTTATTACTCAACCGTAACCGATTTTGCCAGGT |
| | TACGCGGCTGGTCTGCGGTGTGAAATACCGCACAGATGCGTAAGG |
| | AGAAAATACCGCATCAGGCGCTCTTCCGCTTCCTCGCTCACTGAC |
| | TCGCTGCGCTCGGTCGTTCGGCTGCGGCGAGCGGTATCAGCTCAC |
| | TCAAAGGCGGTAATACGGTTATCCACAGAATCAGGGGATAACGCA |
| | GGAAAGAACATGTGAGCAAAAGGCCAGCAAAAGGCCAGGAACCGT |
| | AAAAAGGCCGCGTTGCTGGCGTTTTTCCATAGGCTCCGCCCCCCT |
| | GACGAGCATCACAAAAATCGACGCTCAAGTCAGAGGTGGCGAAAC |
| | CCGACAGGACTATAAAGATACCAGGCGTTTCCCCCTGGAAGCTCC |
| | CTCGTGCGCTCTCCTGTTCCGACCCTGCCGCTTACCGGATACCTG |
| | TCCGCCTTTCTCCCTTCGGGAAGCGTGGCGCTTTCTCAATGCTCA |
| | CGCTGTAGGTATCTCAGTTCGGTGTAGGTCGTTCGCTCCAAGCTG |
| | GGCTGTGTGCACGAACCCCCCGTTCAGCCCGACCGCTGCGCCTTA |
| | TCCGGTAACTATCGTCTTGAGTCCAACCCGGTAAGACACGACTTA |
| | TCGCCACTGGCAGCAGCCACTGGTAACAGGATTAGCAGAGCGAGG |
| | TATGTAGGCGGTGCTACAGAGTTCTTGAAGTGGTGGCCTAACTAC |
| | GGCTACACTAGAAGGACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTT |
| | TTAAATTAAAAATGAAGTTTTAAATCAATCTAAAGTATATATGAG |
| | TAAACTTGGTCTGACAGTTACCAATGCTTAATCAGTGAGGCACCT |
| | ATCTCAGCGATCTGTCTATTTCGTTCATCCATAGTTGCCTGACTC |
| | CCCGTCGTGTAGATAACTACGATACGGGAGGGCTTACCATCTGGC |
| | CCCAGTGCTGCAATGATACCGCGAGACCCACGCTCACCGGCTCCA |
| | GATTTATCAGCAATAAACCAGCCAGCCGGAAGGGCCGAGCGCAGA |
| | AGTGGTCCTGCAACTTTATCCGCCTCCATCCAGTCTATTAATTGT |
| | TGCCGGGAAGCTAGAGTAAGTAGTTCGCCAGTTAATAGTTTGCGC |
| | AACGTTGTTGCCATTGCTACAGGCATCGTGGTGTCACGCTCGTCG |
| | TTTGGTATGGCTTCATTCAGCTCCGGTTCCCAACGATCAAGGCGA |
| | GTTACATGATCCCCCATGTTGTGCAAAAAAGCGGTTAGCTCCTTC |
| | GGTCCTCCGATCGTTGTCAGAAGTAAGTTGGCCGCAGTGTTATCA |
| | CTCATGGTTATGGCAGCACTGCATAATTCTCTTACTGTCATGCCA |
| | TCCGTAAGATGCTTTTCTGTGACTGGTGAGTACTCAACCAAGTCA |
| | TTCTGAGAATAGTGTATGCGGCGACCGAGTTGCTCTTGCCCGGCG |
| | TCAATACGGGATAATACCGCGCCACATAGCAGAACTTTAAAAGTG |
| | CTCATCATTGGAAAACGTTCTTCGGGGCGAAAACTCTCAAGGATC |
| | TTACCGCTGTTGAGATCCAGTTCGATGTAACCCACTCGTGCACCC |
| | AACTGATCTTCAGCATCTTTTACTTTCACCAGCGTTTCTGGGTGA |
| | GCAAAAACAGGAAGGCAAAATGCCGCAAAAAAGGGAATAAGGGCG |
| | ACACGGAAATGTTGAATACTCATACTCTTCCTTTTTCAATATTAT |
| | TGAAGCATTTATCAGGGTTATTGTCTCATGAGCGGATACATATTT |
| | GAATGTATTTAGAAAAATAAACAAATAGGGGTTCCGCGCACATTT |
| | CCCCGAAAAGTGCCACCTGAAATTGTAAACGTTAATATTTTGTTA |
| | AAATTCGCGTTAAATTTTTGTTAAATCAGCTCATTTTTTAACCAA |
| | TAGGCCGAAATCGGCAAAATCCCTTATAAATCAAAAGAATAGACC |
| | GAGATAGGGTTGAGTGTTGTTCCAGTTTGGAACAAGAGTCCACTA |
| | TTAAAGAACGTGGACTCCAACGTCAAAGGGCGAAAAACCGTCTAT |
| | CAGGGCGATGGCCCACTACGTGAACCATCACCCTAATCAAGTTTT |
| | TTGGGGTCGAGGTGCCGTAAAGCACTAAATCGGAACCCTAAAGGG |
| | AGCCCCCGATTTAGAGCTTGACGGGGAAAGCCGGCGAACGTGGCG |
| | AGAAAGGAAGGGAAGAAAGCGAAAGGAGCGGGCGCTAGGGCGCTG |
| | GCAAGTGTAGCGGTCACGCTGCGCGTAACCACCACACCCGCCGCG |
| | CTTAATGCGCCGCTACAGGGCGCGTC |
| AMI146; | 611-755: Full ITR |
| SEQ ID | 801-104: CMV enhancer |
| NO: 58 | 1105-1308: CMV promoter |
| | 1412-1508: SV40 intron |
| | 1513-1521: Kozak sequence |
| | 1522-1578: Human IgG heavy chain secretion sequence |
| | 1579-3012: Ang2 coding sequence (natural) |
| | 3013-3027: 1xGGGGS |
| | 3028-3057: 10xHis tag |
| | 3067-3449: Minimum WPRE sequence |
| | 3462-3510: Poly A sequence |
| | 3533-3638: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTTAAGGGCGTGCAG |
| | TGCTATAACAACTTTCGGAAGAGCATGGACAGCATAGGAAAGAAG |
| | CAATATCAGGTCCAGCATGGGTCCTGCAGCTACACTTTCCTCCTG |
| | CCAGAGATGGACAACTGCCGCTCTTCCTCCAGCCCCTACGTGTCC |
| | AATGCTGTGCAGAGGGACGCGCCGCTCGAATACGATGACTCGGTG |
| | CAGAGGCTGCAAGTGCTGGAGAACATCATGGAAAACAACACTCAG |
| | TGGCTAATGAAGCTTGAGAATTATATCCAGGACAACATGAAGAAA |
| | GAAATGGTAGAGATACAGCAGAATGCAGTACAGAACCAGACGGCT |
| | GTGATGATAGAAATAGGGACAAACCTGTTGAACCAAACAGCGGAG |
| | CAAACGCGGAAGTTAACTGATGTGGAAGCCCAAGTATTAAATCAG |
| | ACCACGAGACTTGAACTTCAGCTCTTGGAACACTCCCTCTCGACA |
| | AACAAATTGGAAAAACAGATTTTGGACCAGACCAGTGAAATAAAC |
| | AAATTGCAAGATAAGAACAGTTTCCTAGAAAAGAAGGTGCTAGCT |
| | ATGGAAGACAAGCACATCATCCAACTACAGTCAATAAAAGAAGAG |
| | AAAGATCAGCTACAGGTGTTAGTATCCAAGCAAAATTCCATCATT |
| | GAAGAACTAGAAAAAAAAATAGTGACTGCCACGGTGAATAATTCA |
| | GTTCTTCAGAAGCAGCAACATGATCTCATGGAGACAGTTAATAAC |
| | TTACTGACTATGATGTCCACATCAAACTCAGCTAAGGACCCCACT |
| | GTTGCTAAAGAAGAACAAATCAGCTTCAGAGACTGTGCTGAAGTA |
| | TTCAAATCAGGACACACCACGAATGGCATCTACACGTTAACATTC |
| | CCTAATTCTACAGAAGAGATCAAGGCCTACTGTGACATGGAAGCT |
| | GGAGGAGGCGGGTGGACAATTATTCAGCGACGTGAGGATGGCAGC |
| | GTTGATTTTCAGAGGACTTGGAAAGAATATAAAGTGGGATTTGGT |
| | AACCCTTCAGGAGAATATTGGCTGGGAAATGAGTTTGTTTCGCAA |
| | CTGACTAATCAGCAACGCTATGTGCTTAAAATACACCTTAAAGAC |
| | TGGGAAGGGAATGAGGCTTACTCATTGTATGAACATTTCTATCTC |
| | TCAAGTGAAGAACTCAATTATAGGATTCACCTTAAAGGACTTACA |
| | GGGACAGCCGGCAAAATAAGCAGCATCAGCCAACCAGGAAATGAT |
| | TTTAGCACAAAGGATGGAGACAACGACAAATGTATTTGCAAATGT |
| | TCACAAATGCTAACAGGAGGCTGGTGGTTTGATGCATGTGGTCCT |
| | TCCAACTTGAACGGAATGTACTATCCACAGAGGCAGAACACAAAT |
| | AAGTTCAACGGCATTAAATGGTACTACTGGAAAGGCTCAGGCTAT |
| | TCGCTCAAGGCCACAACCATGATGATCCGACCAGCAGATTTCGGG |
| | GGTGGAGGCTCTCACCATCACCACCATCATCACCATCACCACTAA |
| | CTCGAGAATCAACCTCTGGATTACAAAATTTGTGAAAGATTGACT |
| | GGTATTCTTAACTATGTTGCTCCTTTTACGCTATGTGGATACGCT |
| | GCTTTAATGCCTTTGTATCATGCTATTGCTTCCCGTATGGCTTTC |
| | ATTTTCTCCTCCTTGTATAAATCCTGGTTGCTGTCTCTTTATGAG |
| | GAGTTGTGGCCCGTTGTCAGGCAACGTGGCGTGGTGTGCACTGTG |
| | TTTGCTGACGCAACCCCCACTGGTTGGGGCATTGCCACCACCTGT |
| | CAGCTCCTTTCCGGGACTTTCGCTTTCCCCCTCCCTATTGCCACG |
| | GCGGAACTCATCGCCGCCTGCCTTGCCCGCTGCTGGACAGGGGCT |
| | CGGCTGTTGGGCACTGACAATTCCGTGGTGGTACCTTCGAAAATA |
| | AAATATCTTTATTTTCATTACATCTGTGTGTTGGTTTTTTGTGTG |
| | GCATGCTGGGGAGAGATCAACCCCACTCCCTCTCTGCGCGCTCGC |
| | TCGCTCACTGAGGCCGGGCGACCAAAGGTCGCCCGACGCCCGGGC |
| | TTTGCCCGGGCGGCCTCAGTGAGCGAGCGAGCGCGCAGCAAGCTG |
| | TAGCCAACCACTAGAACTATAGCTAGAGTCCTGGGCGAACAAACG |
| | ATGCTCGCCTTCCAGAAAACCGAGGATGCGAACCACTTCATCCGG |
| | GGTCAGCACCACCGGCAAGCGCCGCGACGGCCGAGGTCTTCCGAT |
| | CTCCTGAAGCCAGGGCAGATCCGTGCACAGCACCTTGCCGTAGAA |
| | GAACAGCAAGGCCGCCAATGCCTGACGATGCGTGGAGACCGAAAC |
| | CTTGCGCTCGTTCGCCAGCCAGGACAGAAATGCCTCGACTTCGCT |
| | GCTGCCCAAGGTTGCCGGGTGACGCACACCGTGGAAACGGATGAA |
| | GGCACGAACCCAGTTGACATAAGCCTGTTCGGTTCGTAAACTGTA |
| | ATGCAAGTAGCGTATGCGCTCACGCAACTGGTCCAGAACCTTGAC |
| | CGAACGCAGCGGTGGTAACGGCGCAGTGGCGGTTTTCATGGCTTG |
| | TTATGACTGTTTTTTTGTACAGTCTATGCCTCGGGCATCCAAGCA |
| | GCAAGCGCGTTACGCCGTGGGTCGATGTTTGATGTTATGGAGCAG |
| | CAACGATGTTACGCAGCAGCAACGATGTTACGCAGCAGGGCAGTC |
| | GCCCTAAAACAAAGTTAGGTGGCTCAAGTATGGGCATCATTCGCA |
| | CATGTAGGCTCGGCCCTGACCAAGTCAAATCCATGCGGGCTGCTC |
| | TTGATCTTTTCGGTCGTGAGTTCGGAGACGTAGCCACCTACTCCC |
| | AACATCAGCCGGACTCCGATTACCTCGGGAACTTGCTCCGTAGTA |
| | AGACATTCATCGCGCTTGCTGCCTTCGACCAAGAAGCGGTTGTTG |
| | GCGCTCTCGCGGCTTACGTTCTGCCCAGGTTTGAGCAGCCGCGTA |
| | GTGAGATCTATATCTATGATCTCGCAGTCTCCGGCGAGCACCGGA |
| | GGCAGGGCATTGCCACCGCGCTCATCAATCTCCTCAAGCATGAGG |
| | CCAACGCGCTTGGTGCTTATGTGATCTACGTGCAAGCAGATTACG |
| | GTGACGATCCCGCAGTGGCTCTCTATACAAAGTTGGGCATACGGG |
| | AAGAAGTGATGCACTTTGATATCGACCCAAGTACCGCCACCTAAC |
| | AATTCGTTCAAGCCGAGATCGGCTTCCCGGCCGCGGAGTTGTTCG |
| | GTAAATTGTCACAACGCCGCGAATATAGTCTTTACCATGCCCTTG |
| | GCCACGCCCCTCTTTAATACGACGGGCAATTTGCACTTCAGAAAA |
| | TGAAGAGTTTGCTTTAGCCATAACAAAAGTCCAGTATGCTTTTTC |
| | ACAGCATAACTGGACTGATTTCAGTTTACAACTATTCTGTCTAGT |
| | TTAAGACTTTATTGTCATAGTTTAGATCTATTTTGTTCAGTTTAA |
| | GACTTTATTGTCCGCCCACACCCGCTTACGCAGGGCATCCATTTA |
| | TTACTCAACCGTAACCGATTTTGCCAGGTTACGCGGCTGGTCTGC |
| | GGTGTGAAATACCGCACAGATGCGTAAGGAGAAAATACCGCATCA |
| | GGCGCTCTTCCGCTTCCTCGCTCACTGACTCGCTGCGCTCGGTCG |
| | TTCGGCTGCGGCGAGCGGTATCAGCTCACTCAAAGGCGGTAATAC |
| | GGTTATCCACAGAATCAGGGGATAACGCAGGAAAGAACATGTGAG |
| | CAAAAGGCCAGCAAAAGGCCAGGAACCGTAAAAAGGCCGCGTTGC |
| | TGGCGTTTTTCCATAGGCTCCGCCCCCCTGACGAGCATCACAAAA |
| | ATCGACGCTCAAGTCAGAGGTGGCGAAACCCGACAGGACTATAAA |
| | GATACCAGGCGTTTCCCCCTGGAAGCTCCCTCGTGCGCTCTCCTG |
| | TTCCGACCCTGCCGCTTACCGGATACCTGTCCGCCTTTCTCCCTT |
| | CGGGAAGCGTGGCGCTTTCTCAATGCTCACGCTGTAGGTATCTCA |
| | GTTCGGTGTAGGTCGTTCGCTCCAAGCTGGGCTGTGTGCACGAAC |
| | CCCCCGTTCAGCCCGACCGCTGCGCCTTATCCGGTAACTATCGTC |
| | TTGAGTCCAACCCGGTAAGACACGACTTATCGCCACTGGCAGCAG |
| | CCACTGGTAACAGGATTAGCAGAGCGAGGTATGTAGGCGGTGCTA |
| | CAGAGTTCTTGAAGTGGTGGCCTAACTACGGCTACACTAGAAGGA |
| | CAGTATTTGGTATCTGCGCTCTGCTGAAGCCAGTTACCTTCGGAA |
| | AAAGAGTTGGTAGCTCTTGATCCGGCAAACAAACCACCGCTGGTA |
| | GCGGTGGTTTTTTTGTTTGCAAGCAGCAGATTACGCGCAGAAAAA |
| | AAGGATCTCAAGAAGATCCTTTGATCTTTTCTACGGGGTCTGACG |
| | CTCAGTGGAACGAAAACTCACGTTAAGGGATTTTGGTCATGAGAT |
| | TATCAAAAAGGATCTTCACCTAGATCCTTTTAAATTAAAAATGAA |
| | GTTTTAAATCAATCTAAAGTATATATGAGTAAACTTGGTCTGACA |
| | GTTACCAATGCTTAATCAGTGAGGCACCTATCTCAGCGATCTGTC |
| | TATTTCGTTCATCCATAGTTGCCTGACTCCCCGTCGTGTAGATAA |
| | CTACGATACGGGAGGGCTTACCATCTGGCCCCAGTGCTGCAATGA |
| | TACCGCGAGACCCACGCTCACCGGCTCCAGATTTATCAGCAATAA |
| | ACCAGCCAGCCGGAAGGGCCGAGCGCAGAAGTGGTCCTGCAACTT |
| | TATCCGCCTCCATCCAGTCTATTAATTGTTGCCGGGAAGCTAGAG |
| | TAAGTAGTTCGCCAGTTAATAGTTTGCGCAACGTTGTTGCCATTG |
| | CTACAGGCATCGTGGTGTCACGCTCGTCGTTTGGTATGGCTTCAT |
| | TCAGCTCCGGTTCCCAACGATCAAGGCGAGTTACATGATCCCCCA |
| | TGTTGTGCAAAAAAGCGGTTAGCTCCTTCGGTCCTCCGATCGTTG |
| | TCAGAAGTAAGTTGGCCGCAGTGTTATCACTCATGGTTATGGCAG |
| | CACTGCATAATTCTCTTACTGTCATGCCATCCGTAAGATGCTTTT |
| | CTGTGACTGGTGAGTACTCAACCAAGTCATTCTGAGAATAGTGTA |
| | TGCGGCGACCGAGTTGCTCTTGCCCGGCGTCAATACGGGATAATA |
| | CCGCGCCACATAGCAGAACTTTAAAAGTGCTCATCATTGGAAAAC |
| | GTTCTTCGGGGCGAAAACTCTCAAGGATCTTACCGCTGTTGAGAT |
| | CCAGTTCGATGTAACCCACTCGTGCACCCAACTGATCTTCAGCAT |
| | CTTTTACTTTCACCAGCGTTTCTGGGTGAGCAAAAACAGGAAGGC |
| | AAAATGCCGCAAAAAAGGGAATAAGGGCGACACGGAAATGTTGAA |
| | TACTCATACTCTTCCTTTTTCAATATTATTGAAGCATTTATCAGG |
| | GTTATTGTCTCATGAGCGGATACATATTTGAATGTATTTAGAAAA |
| | ATAAACAAATAGGGGTTCCGCGCACATTTCCCCGAAAAGTGCCAC |
| | CTGAAATTGTAAACGTTAATATTTTGTTAAAATTCGCGTTAAATT |
| | TTTGTTAAATCAGCTCATTTTTTAACCAATAGGCCGAAATCGGCA |
| | AAATCCCTTATAAATCAAAAGAATAGACCGAGATAGGGTTGAGTG |
| | TTGTTCCAGTTTGGAACAAGAGTCCACTATTAAAGAACGTGGACT |
| | CCAACGTCAAAGGGCGAAAAACCGTCTATCAGGGCGATGGCCCAC |
| | TACGTGAACCATCACCCTAATCAAGTTTTTTGGGGTCGAGGTGCC |
| | GTAAAGCACTAAATCGGAACCCTAAAGGGAGCCCCCGATTTAGAG |
| | CTTGACGGGGAAAGCCGGCGAACGTGGCGAGAAAGGAAGGGAAGA |
| | AAGCGAAAGGAGCGGGCGCTAGGGCGCTGGCAAGTGTAGCGGTCA |
| | CGCTGCGCGTAACCACCACACCCGCCGCGCTTAATGCGCCGCTAC |
| | AGGGCGCGTC |
| AMI147; | 611-755: Full ITR |
| SEQ ID | 801-104: CMV enhancer |
| NO: 59 | 1105-1308: CMV promoter |
| | 1412-1508: SV40 intron |
| | 1513-1521: Kozak sequence |
| | 1522-1602: Aflibercept secretion sequence |
| | 1603-2895: Aflibercept coding sequence after optimization |
| | 2905-2953: Poly A signal |
| | 2954-3194: Human U6 promoter |
| | 3201-3256: shRNA2 against Ang2 |
| | (GGAAGCTTGAGAATTATAAtacctgacccataTTATAATTCTCAAGCTTCCTTTTT |
| | 3279-3384: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGTGAGC |
| | TACTGGGACACCGGCGTGCTGCTGTGCGCCCTGCTGAGCTGCCTG |
| | CTGCTGACCGGCAGCAGCAGCGGCAGCGACACCGGCAGGCCCTTC |
| | GTGGAGATGTACTCCGAGATCCCCGAGATCATCCACATGACCGAG |
| | GGCAGGGAGCTGGTGATCCCCTGCAGGGTGACCTCCCCCAACATC |
| | ACCGTGACCCTGAAGAAGTTCCCCCTGGACACCCTGATCCCCGAC |
| | GGCAAGAGGATCATCTGGGACTCCAGGAAGGGCTTCATCATCTCC |
| | AACGCCACCTACAAGGAGATCGGCCTGCTGACCTGCGAGGCCACC |
| | GTGAACGGCCACCTGTACAAGACCAACTACCTGACCCACAGGCAG |
| | ACCAACACCATCATCGACGTGGTGCTGTCCCCCTCCCACGGCATC |
| | GAGCTGTCCGTGGGCGAGAAGCTGGTGCTGAACTGCACCGCCAGG |
| | ACCGAGCTGAACGTGGGCATCGACTTCAACTGGGAGTACCCCTCC |
| | TCCAAGCACCAGCACAAGAAGCTGGTGAACAGGGACCTGAAGACC |
| | CAGTCCGGCTCCGAGATGAAGAAGTTCCTGTCCACCCTGACCATC |
| | GACGGCGTGACCAGGTCCGACCAGGGCCTGTACACCTGCGCCGCC |
| | TCCTCCGGCCTGATGACCAAGAAGAACTCCACCTTCGTGAGGGTG |
| | CACGAGAAGGACAAGACCCACACCTGCCCCCCCTGCCCCGCCCCC |
| | GAGCTGCTGGGCGGCCCCTCCGTGTTCCTGTTCCCCCCCAAGCCC |
| | AAGGACACCCTGATGATCTCCAGGACCCCCGAGGTGACCTGCGTG |
| | GTGGTGGACGTGTCCCACGAGGACCCCGAGGTGAAGTTCAACTGG |
| | TACGTGGACGGCGTGGAGGTGCACAACGCCAAGACCAAGCCCAGG |
| | GAGGAGCAGTACAACTCCACCTACAGGGTGGTGTCCGTGCTGACC |
| | GTGCTGCACCAGGACTGGCTGAACGGCAAGGAGTACAAGTGCAAG |
| | GTGTCCAACAAGGCCCTGCCCGCCCCCATCGAGAAGACCATCTCC |
| | AAGGCCAAGGGCCAGCCCAGGGAGCCCCAGGTGTACACCCTGCCC |
| | CCCTCCAGGGACGAGCTGACCAAGAACCAGGTGTCCCTGACCTGC |
| | CTGGTGAAGGGCTTCTACCCCTCCGACATCGCCGTGGAGTGGGAG |
| | TCCAACGGCCAGCCCGAGAACAACTACAAGACCACCCCCCCCGTG |
| | CTGGACTCCGACGGCTCCTTCTTCCTGTACTCCAAGCTGACCGTG |
| | GACAAGTCCAGGTGGCAGCAGGGCAACGTGTTCTCCTGCTCCGTG |
| | ATGCACGAGGCCCTGCACAACCACTACACCCAGAAGTCCCTGTCC |
| | CTGTCCCCCGGCAAGTGATTCGAAAATAAAATATCTTTATTTTCA |
| | TTACATCTGTGTGTTGGTTTTTTGTGTGGAGGGCCTATTTCCCAT |
| | GATTCCTTCATATTTGCATATACGATACAAGGCTGTTAGAGAGAT |
| | AATTGGAATTAATTTGACTGTAAACACAAAGATATTAGTACAAAA |
| | TACGTGACGTAGAAAGTAATAATTTCTTGGGTAGTTTGCAGTTTT |
| | AAAATTATGTTTTAAAATGGACTATCATATGCTTACCGTAACTTG |
| | AAAGTATTTCGATTTCTTGGCTTTATATATCTTGTGGAAAGGACA |
| | AGCTTGGAAGCTTGAGAATTATAATACCTGACCCATATTATAATT |
| | CTCAAGCTTCCTTTTTGCATGCTGGGGAGAGATCAACCCCACTCC |
| | CTCTCTGCGCGCTCGCTCGCTCACTGAGGCCGGGCGACCAAAGGT |
| | CGCCCGACGCCCGGGCTTTGCCCGGGCGGCCTCAGTGAGCGAGCG |
| | AGCGCGCAGCAAGCTGTAGCCAACCACTAGAACTATAGCTAGAGT |
| | CCTGGGCGAACAAACGATGCTCGCCTTCCAGAAAACCGAGGATGC |
| | GAACCACTTCATCCGGGGTCAGCACCACCGGCAAGCGCCGCGACG |
| | GCCGAGGTCTTCCGATCTCCTGAAGCCAGGGCAGATCCGTGCACA |
| | GCACCTTGCCGTAGAAGAACAGCAAGGCCGCCAATGCCTGACGAT |
| | GCGTGGAGACCGAAACCTTGCGCTCGTTCGCCAGCCAGGACAGAA |
| | ATGCCTCGACTTCGCTGCTGCCCAAGGTTGCCGGGTGACGCACAC |
| | CGTGGAAACGGATGAAGGCACGAACCCAGTTGACATAAGCCTGTT |
| | CGGTTCGTAAACTGTAATGCAAGTAGCGTATGCGCTCACGCAACT |
| | GGTCCAGAACCTTGACCGAACGCAGCGGTGGTAACGGCGCAGTGG |
| | CGGTTTTCATGGCTTGTTATGACTGTTTTTTTGTACAGTCTATGC |
| | CTCGGGCATCCAAGCAGCAAGCGCGTTACGCCGTGGGTCGATGTT |
| | TGATGTTATGGAGCAGCAACGATGTTACGCAGCAGCAACGATGTT |
| | ACGCAGCAGGGCAGTCGCCCTAAAACAAAGTTAGGTGGCTCAAGT |
| | ATGGGCATCATTCGCACATGTAGGCTCGGCCCTGACCAAGTCAAA |
| | TCCATGCGGGCTGCTCTTGATCTTTTCGGTCGTGAGTTCGGAGAC |
| | GTAGCCACCTACTCCCAACATCAGCCGGACTCCGATTACCTCGGG |
| | AACTTGCTCCGTAGTAAGACATTCATCGCGCTTGCTGCCTTCGAC |
| | CAAGAAGCGGTTGTTGGCGCTCTCGCGGCTTACGTTCTGCCCAGG |
| | TTTGAGCAGCCGCGTAGTGAGATCTATATCTATGATCTCGCAGTC |
| | TCCGGCGAGCACCGGAGGCAGGGCATTGCCACCGCGCTCATCAAT |
| | CTCCTCAAGCATGAGGCCAACGCGCTTGGTGCTTATGTGATCTAC |
| | GTGCAAGCAGATTACGGTGACGATCCCGCAGTGGCTCTCTATACA |
| | AAGTTGGGCATACGGGAAGAAGTGATGCACTTTGATATCGACCCA |
| | AGTACCGCCACCTAACAATTCGTTCAAGCCGAGATCGGCTTCCCG |
| | GCCGCGGAGTTGTTCGGTAAATTGTCACAACGCCGCGAATATAGT |
| | CTTTACCATGCCCTTGGCCACGCCCCTCTTTAATACGACGGGCAA |
| | TTTGCACTTCAGAAAATGAAGAGTTTGCTTTAGCCATAACAAAAG |
| | TCCAGTATGCTTTTTCACAGCATAACTGGACTGATTTCAGTTTAC |
| | AACTATTCTGTCTAGTTTAAGACTTTATTGTCATAGTTTAGATCT |
| | ATTTTGTTCAGTTTAAGACTTTATTGTCCGCCCACACCCGCTTAC |
| | GCAGGGCATCCATTTATTACTCAACCGTAACCGATTTTGCCAGGT |
| | TACGCGGCTGGTCTGCGGTGTGAAATACCGCACAGATGCGTAAGG |
| | AGAAAATACCGCATCAGGCGCTCTTCCGCTTCCTCGCTCACTGAC |
| | TCGCTGCGCTCGGTCGTTCGGCTGCGGCGAGCGGTATCAGCTCAC |
| | TCAAAGGCGGTAATACGGTTATCCACAGAATCAGGGGATAACGCA |
| | GGAAAGAACATGTGAGCAAAAGGCCAGCAAAAGGCCAGGAACCGT |
| | AAAAAGGCCGCGTTGCTGGCGTTTTTCCATAGGCTCCGCCCCCCT |
| | GACGAGCATCACAAAAATCGACGCTCAAGTCAGAGGTGGCGAAAC |
| | CCGACAGGACTATAAAGATACCAGGCGTTTCCCCCTGGAAGCTCC |
| | CTCGTGCGCTCTCCTGTTCCGACCCTGCCGCTTACCGGATACCTG |
| | TCCGCCTTTCTCCCTTCGGGAAGCGTGGCGCTTTCTCAATGCTCA |
| | CGCTGTAGGTATCTCAGTTCGGTGTAGGTCGTTCGCTCCAAGCTG |
| | GGCTGTGTGCACGAACCCCCCGTTCAGCCCGACCGCTGCGCCTTA |
| | TCCGGTAACTATCGTCTTGAGTCCAACCCGGTAAGACACGACTTA |
| | TCGCCACTGGCAGCAGCCACTGGTAACAGGATTAGCAGAGCGAGG |
| | TATGTAGGCGGTGCTACAGAGTTCTTGAAGTGGTGGCCTAACTAC |
| | GGCTACACTAGAAGGACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTT |
| | TTAAATTAAAAATGAAGTTTTAAATCAATCTAAAGTATATATGAG |
| | TAAACTTGGTCTGACAGTTACCAATGCTTAATCAGTGAGGCACCT |
| | ATCTCAGCGATCTGTCTATTTCGTTCATCCATAGTTGCCTGACTC |
| | CCCGTCGTGTAGATAACTACGATACGGGAGGGCTTACCATCTGGC |
| | CCCAGTGCTGCAATGATACCGCGAGACCCACGCTCACCGGCTCCA |
| | GATTTATCAGCAATAAACCAGCCAGCCGGAAGGGCCGAGCGCAGA |
| | AGTGGTCCTGCAACTTTATCCGCCTCCATCCAGTCTATTAATTGT |
| | TGCCGGGAAGCTAGAGTAAGTAGTTCGCCAGTTAATAGTTTGCGC |
| | AACGTTGTTGCCATTGCTACAGGCATCGTGGTGTCACGCTCGTCG |
| | TTTGGTATGGCTTCATTCAGCTCCGGTTCCCAACGATCAAGGCGA |
| | GTTACATGATCCCCCATGTTGTGCAAAAAAGCGGTTAGCTCCTTC |
| | GGTCCTCCGATCGTTGTCAGAAGTAAGTTGGCCGCAGTGTTATCA |
| | CTCATGGTTATGGCAGCACTGCATAATTCTCTTACTGTCATGCCA |
| | TCCGTAAGATGCTTTTCTGTGACTGGTGAGTACTCAACCAAGTCA |
| | TTCTGAGAATAGTGTATGCGGCGACCGAGTTGCTCTTGCCCGGCG |
| | TCAATACGGGATAATACCGCGCCACATAGCAGAACTTTAAAAGTG |
| | CTCATCATTGGAAAACGTTCTTCGGGGCGAAAACTCTCAAGGATC |
| | TTACCGCTGTTGAGATCCAGTTCGATGTAACCCACTCGTGCACCC |
| | AACTGATCTTCAGCATCTTTTACTTTCACCAGCGTTTCTGGGTGA |
| | GCAAAAACAGGAAGGCAAAATGCCGCAAAAAAGGGAATAAGGGCG |
| | ACACGGAAATGTTGAATACTCATACTCTTCCTTTTTCAATATTAT |
| | TGAAGCATTTATCAGGGTTATTGTCTCATGAGCGGATACATATTT |
| | GAATGTATTTAGAAAAATAAACAAATAGGGGTTCCGCGCACATTT |
| | CCCCGAAAAGTGCCACCTGAAATTGTAAACGTTAATATTTTGTTA |
| | AAATTCGCGTTAAATTTTTGTTAAATCAGCTCATTTTTTAACCAA |
| | TAGGCCGAAATCGGCAAAATCCCTTATAAATCAAAAGAATAGACC |
| | GAGATAGGGTTGAGTGTTGTTCCAGTTTGGAACAAGAGTCCACTA |
| | TTAAAGAACGTGGACTCCAACGTCAAAGGGCGAAAAACCGTCTAT |
| | CAGGGCGATGGCCCACTACGTGAACCATCACCCTAATCAAGTTTT |
| | TTGGGGTCGAGGTGCCGTAAAGCACTAAATCGGAACCCTAAAGGG |
| | AGCCCCCGATTTAGAGCTTGACGGGGAAAGCCGGCGAACGTGGCG |
| | AGAAAGGAAGGGAAGAAAGCGAAAGGAGCGGGCGCTAGGGCGCTG |
| | GCAAGTGTAGCGGTCACGCTGCGCGTAACCACCACACCCGCCGCG |
| | CTTAATGCGCCGCTACAGGGCGCGTC |
| AMI148; | 611-755: Full ITR |
| SEQ ID | 801-104: CMV enhancer |
| NO: 60 | 1105-1308: CMV promoter |
| | 1412-1508: SV40 intron |
| | 1513-1521: Kozak sequence |
| | 1522-1602: Aflibercept secretion sequence |
| | 1603-2895: Aflibercept coding sequence after optimization |
| | 2905-2953: Poly A signal |
| | 2954-3194: Human U6 promoter |
| | 3201-3256: shRNA3 against Ang2 |
| | (GTGAAGAACTCAATTATAAtacctgacccataTTATAATTGAGTTCTTCACTTTTT) |
| | 3279-3384: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGTGAGC |
| | TACTGGGACACCGGCGTGCTGCTGTGCGCCCTGCTGAGCTGCCTG |
| | CTGCTGACCGGCAGCAGCAGCGGCAGCGACACCGGCAGGCCCTTC |
| | GTGGAGATGTACTCCGAGATCCCCGAGATCATCCACATGACCGAG |
| | GGCAGGGAGCTGGTGATCCCCTGCAGGGTGACCTCCCCCAACATC |
| | ACCGTGACCCTGAAGAAGTTCCCCCTGGACACCCTGATCCCCGAC |
| | GGCAAGAGGATCATCTGGGACTCCAGGAAGGGCTTCATCATCTCC |
| | AACGCCACCTACAAGGAGATCGGCCTGCTGACCTGCGAGGCCACC |
| | GTGAACGGCCACCTGTACAAGACCAACTACCTGACCCACAGGCAG |
| | ACCAACACCATCATCGACGTGGTGCTGTCCCCCTCCCACGGCATC |
| | GAGCTGTCCGTGGGCGAGAAGCTGGTGCTGAACTGCACCGCCAGG |
| | ACCGAGCTGAACGTGGGCATCGACTTCAACTGGGAGTACCCCTCC |
| | TCCAAGCACCAGCACAAGAAGCTGGTGAACAGGGACCTGAAGACC |
| | CAGTCCGGCTCCGAGATGAAGAAGTTCCTGTCCACCCTGACCATC |
| | GACGGCGTGACCAGGTCCGACCAGGGCCTGTACACCTGCGCCGCC |
| | TCCTCCGGCCTGATGACCAAGAAGAACTCCACCTTCGTGAGGGTG |
| | CACGAGAAGGACAAGACCCACACCTGCCCCCCCTGCCCCGCCCCC |
| | GAGCTGCTGGGCGGCCCCTCCGTGTTCCTGTTCCCCCCCAAGCCC |
| | AAGGACACCCTGATGATCTCCAGGACCCCCGAGGTGACCTGCGTG |
| | GTGGTGGACGTGTCCCACGAGGACCCCGAGGTGAAGTTCAACTGG |
| | TACGTGGACGGCGTGGAGGTGCACAACGCCAAGACCAAGCCCAGG |
| | GAGGAGCAGTACAACTCCACCTACAGGGTGGTGTCCGTGCTGACC |
| | GTGCTGCACCAGGACTGGCTGAACGGCAAGGAGTACAAGTGCAAG |
| | GTGTCCAACAAGGCCCTGCCCGCCCCCATCGAGAAGACCATCTCC |
| | AAGGCCAAGGGCCAGCCCAGGGAGCCCCAGGTGTACACCCTGCCC |
| | CCCTCCAGGGACGAGCTGACCAAGAACCAGGTGTCCCTGACCTGC |
| | CTGGTGAAGGGCTTCTACCCCTCCGACATCGCCGTGGAGTGGGAG |
| | TCCAACGGCCAGCCCGAGAACAACTACAAGACCACCCCCCCCGTG |
| | CTGGACTCCGACGGCTCCTTCTTCCTGTACTCCAAGCTGACCGTG |
| | GACAAGTCCAGGTGGCAGCAGGGCAACGTGTTCTCCTGCTCCGTG |
| | ATGCACGAGGCCCTGCACAACCACTACACCCAGAAGTCCCTGTCC |
| | CTGTCCCCCGGCAAGTGATTCGAAAATAAAATATCTTTATTTTCA |
| | TTACATCTGTGTGTTGGTTTTTTGTGTGGAGGGCCTATTTCCCAT |
| | GATTCCTTCATATTTGCATATACGATACAAGGCTGTTAGAGAGAT |
| | AATTGGAATTAATTTGACTGTAAACACAAAGATATTAGTACAAAA |
| | TACGTGACGTAGAAAGTAATAATTTCTTGGGTAGTTTGCAGTTTT |
| | AAAATTATGTTTTAAAATGGACTATCATATGCTTACCGTAACTTG |
| | AAAGTATTTCGATTTCTTGGCTTTATATATCTTGTGGAAAGGACA |
| | AGCTTGTGAAGAACTCAATTATAATACCTGACCCATATTATAATT |
| | GAGTTCTTCACTTTTTGCATGCTGGGGAGAGATCAACCCCACTCC |
| | CTCTCTGCGCGCTCGCTCGCTCACTGAGGCCGGGCGACCAAAGGT |
| | CGCCCGACGCCCGGGCTTTGCCCGGGCGGCCTCAGTGAGCGAGCG |
| | AGCGCGCAGCAAGCTGTAGCCAACCACTAGAACTATAGCTAGAGT |
| | CCTGGGCGAACAAACGATGCTCGCCTTCCAGAAAACCGAGGATGC |
| | GAACCACTTCATCCGGGGTCAGCACCACCGGCAAGCGCCGCGACG |
| | GCCGAGGTCTTCCGATCTCCTGAAGCCAGGGCAGATCCGTGCACA |
| | GCACCTTGCCGTAGAAGAACAGCAAGGCCGCCAATGCCTGACGAT |
| | GCGTGGAGACCGAAACCTTGCGCTCGTTCGCCAGCCAGGACAGAA |
| | ATGCCTCGACTTCGCTGCTGCCCAAGGTTGCCGGGTGACGCACAC |
| | CGTGGAAACGGATGAAGGCACGAACCCAGTTGACATAAGCCTGTT |
| | CGGTTCGTAAACTGTAATGCAAGTAGCGTATGCGCTCACGCAACT |
| | GGTCCAGAACCTTGACCGAACGCAGCGGTGGTAACGGCGCAGTGG |
| | CGGTTTTCATGGCTTGTTATGACTGTTTTTTTGTACAGTCTATGC |
| | CTCGGGCATCCAAGCAGCAAGCGCGTTACGCCGTGGGTCGATGTT |
| | TGATGTTATGGAGCAGCAACGATGTTACGCAGCAGCAACGATGTT |
| | ACGCAGCAGGGCAGTCGCCCTAAAACAAAGTTAGGTGGCTCAAGT |
| | ATGGGCATCATTCGCACATGTAGGCTCGGCCCTGACCAAGTCAAA |
| | TCCATGCGGGCTGCTCTTGATCTTTTCGGTCGTGAGTTCGGAGAC |
| | GTAGCCACCTACTCCCAACATCAGCCGGACTCCGATTACCTCGGG |
| | AACTTGCTCCGTAGTAAGACATTCATCGCGCTTGCTGCCTTCGAC |
| | CAAGAAGCGGTTGTTGGCGCTCTCGCGGCTTACGTTCTGCCCAGG |
| | TTTGAGCAGCCGCGTAGTGAGATCTATATCTATGATCTCGCAGTC |
| | TCCGGCGAGCACCGGAGGCAGGGCATTGCCACCGCGCTCATCAAT |
| | CTCCTCAAGCATGAGGCCAACGCGCTTGGTGCTTATGTGATCTAC |
| | GTGCAAGCAGATTACGGTGACGATCCCGCAGTGGCTCTCTATACA |
| | AAGTTGGGCATACGGGAAGAAGTGATGCACTTTGATATCGACCCA |
| | AGTACCGCCACCTAACAATTCGTTCAAGCCGAGATCGGCTTCCCG |
| | GCCGCGGAGTTGTTCGGTAAATTGTCACAACGCCGCGAATATAGT |
| | CTTTACCATGCCCTTGGCCACGCCCCTCTTTAATACGACGGGCAA |
| | TTTGCACTTCAGAAAATGAAGAGTTTGCTTTAGCCATAACAAAAG |
| | TCCAGTATGCTTTTTCACAGCATAACTGGACTGATTTCAGTTTAC |
| | AACTATTCTGTCTAGTTTAAGACTTTATTGTCATAGTTTAGATCT |
| | ATTTTGTTCAGTTTAAGACTTTATTGTCCGCCCACACCCGCTTAC |
| | GCAGGGCATCCATTTATTACTCAACCGTAACCGATTTTGCCAGGT |
| | TACGCGGCTGGTCTGCGGTGTGAAATACCGCACAGATGCGTAAGG |
| | AGAAAATACCGCATCAGGCGCTCTTCCGCTTCCTCGCTCACTGAC |
| | TCGCTGCGCTCGGTCGTTCGGCTGCGGCGAGCGGTATCAGCTCAC |
| | TCAAAGGCGGTAATACGGTTATCCACAGAATCAGGGGATAACGCA |
| | GGAAAGAACATGTGAGCAAAAGGCCAGCAAAAGGCCAGGAACCGT |
| | AAAAAGGCCGCGTTGCTGGCGTTTTTCCATAGGCTCCGCCCCCCT |
| | GACGAGCATCACAAAAATCGACGCTCAAGTCAGAGGTGGCGAAAC |
| | CCGACAGGACTATAAAGATACCAGGCGTTTCCCCCTGGAAGCTCC |
| | CTCGTGCGCTCTCCTGTTCCGACCCTGCCGCTTACCGGATACCTG |
| | TCCGCCTTTCTCCCTTCGGGAAGCGTGGCGCTTTCTCAATGCTCA |
| | CGCTGTAGGTATCTCAGTTCGGTGTAGGTCGTTCGCTCCAAGCTG |
| | GGCTGTGTGCACGAACCCCCCGTTCAGCCCGACCGCTGCGCCTTA |
| | TCCGGTAACTATCGTCTTGAGTCCAACCCGGTAAGACACGACTTA |
| | TCGCCACTGGCAGCAGCCACTGGTAACAGGATTAGCAGAGCGAGG |
| | TATGTAGGCGGTGCTACAGAGTTCTTGAAGTGGTGGCCTAACTAC |
| | GGCTACACTAGAAGGACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTT |
| | TTAAATTAAAAATGAAGTTTTAAATCAATCTAAAGTATATATGAG |
| | TAAACTTGGTCTGACAGTTACCAATGCTTAATCAGTGAGGCACCT |
| | ATCTCAGCGATCTGTCTATTTCGTTCATCCATAGTTGCCTGACTC |
| | CCCGTCGTGTAGATAACTACGATACGGGAGGGCTTACCATCTGGC |
| | CCCAGTGCTGCAATGATACCGCGAGACCCACGCTCACCGGCTCCA |
| | GATTTATCAGCAATAAACCAGCCAGCCGGAAGGGCCGAGCGCAGA |
| | AGTGGTCCTGCAACTTTATCCGCCTCCATCCAGTCTATTAATTGT |
| | TGCCGGGAAGCTAGAGTAAGTAGTTCGCCAGTTAATAGTTTGCGC |
| | AACGTTGTTGCCATTGCTACAGGCATCGTGGTGTCACGCTCGTCG |
| | TTTGGTATGGCTTCATTCAGCTCCGGTTCCCAACGATCAAGGCGA |
| | GTTACATGATCCCCCATGTTGTGCAAAAAAGCGGTTAGCTCCTTC |
| | GGTCCTCCGATCGTTGTCAGAAGTAAGTTGGCCGCAGTGTTATCA |
| | CTCATGGTTATGGCAGCACTGCATAATTCTCTTACTGTCATGCCA |
| | TCCGTAAGATGCTTTTCTGTGACTGGTGAGTACTCAACCAAGTCA |
| | TTCTGAGAATAGTGTATGCGGCGACCGAGTTGCTCTTGCCCGGCG |
| | TCAATACGGGATAATACCGCGCCACATAGCAGAACTTTAAAAGTG |
| | CTCATCATTGGAAAACGTTCTTCGGGGCGAAAACTCTCAAGGATC |
| | TTACCGCTGTTGAGATCCAGTTCGATGTAACCCACTCGTGCACCC |
| | AACTGATCTTCAGCATCTTTTACTTTCACCAGCGTTTCTGGGTGA |
| | GCAAAAACAGGAAGGCAAAATGCCGCAAAAAAGGGAATAAGGGCG |
| | ACACGGAAATGTTGAATACTCATACTCTTCCTTTTTCAATATTAT |
| | TGAAGCATTTATCAGGGTTATTGTCTCATGAGCGGATACATATTT |
| | GAATGTATTTAGAAAAATAAACAAATAGGGGTTCCGCGCACATTT |
| | CCCCGAAAAGTGCCACCTGAAATTGTAAACGTTAATATTTTGTTA |
| | AAATTCGCGTTAAATTTTTGTTAAATCAGCTCATTTTTTAACCAA |
| | TAGGCCGAAATCGGCAAAATCCCTTATAAATCAAAAGAATAGACC |
| | GAGATAGGGTTGAGTGTTGTTCCAGTTTGGAACAAGAGTCCACTA |
| | TTAAAGAACGTGGACTCCAACGTCAAAGGGCGAAAAACCGTCTAT |
| | CAGGGCGATGGCCCACTACGTGAACCATCACCCTAATCAAGTTTT |
| | TTGGGGTCGAGGTGCCGTAAAGCACTAAATCGGAACCCTAAAGGG |
| | AGCCCCCGATTTAGAGCTTGACGGGGAAAGCCGGCGAACGTGGCG |
| | AGAAAGGAAGGGAAGAAAGCGAAAGGAGCGGGCGCTAGGGCGCTG |
| | GCAAGTGTAGCGGTCACGCTGCGCGTAACCACCACACCCGCCGCG |
| | CTTAATGCGCCGCTACAGGGCGCGTC |
| AMI150; | 611-755: Full ITR |
| SEQ ID | 801-104: CMV enhancer |
| NO: 61 | 1105-1308: CMV promoter |
| | 1412-1508: SV40 intron |
| | 1513-1521: Kozak sequence |
| | 1522-1602: Aflibercept secretion sequence |
| | 1603-2895: Aflibercept coding sequence after optimization |
| | 2905-2953: Poly A signal |
| | 2954-3194: Human U6 promoter |
| | 3201-3256: shRNA4 against Ang2 |
| | (GTAACATTCCCTAATTCTAtacctgacccataTAGAATTAGGGAATGTTACTTTTT |
| | 3279-3384: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGTGAGC |
| | TACTGGGACACCGGCGTGCTGCTGTGCGCCCTGCTGAGCTGCCTG |
| | CTGCTGACCGGCAGCAGCAGCGGCAGCGACACCGGCAGGCCCTTC |
| | GTGGAGATGTACTCCGAGATCCCCGAGATCATCCACATGACCGAG |
| | GGCAGGGAGCTGGTGATCCCCTGCAGGGTGACCTCCCCCAACATC |
| | ACCGTGACCCTGAAGAAGTTCCCCCTGGACACCCTGATCCCCGAC |
| | GGCAAGAGGATCATCTGGGACTCCAGGAAGGGCTTCATCATCTCC |
| | AACGCCACCTACAAGGAGATCGGCCTGCTGACCTGCGAGGCCACC |
| | GTGAACGGCCACCTGTACAAGACCAACTACCTGACCCACAGGCAG |
| | ACCAACACCATCATCGACGTGGTGCTGTCCCCCTCCCACGGCATC |
| | GAGCTGTCCGTGGGCGAGAAGCTGGTGCTGAACTGCACCGCCAGG |
| | ACCGAGCTGAACGTGGGCATCGACTTCAACTGGGAGTACCCCTCC |
| | TCCAAGCACCAGCACAAGAAGCTGGTGAACAGGGACCTGAAGACC |
| | CAGTCCGGCTCCGAGATGAAGAAGTTCCTGTCCACCCTGACCATC |
| | GACGGCGTGACCAGGTCCGACCAGGGCCTGTACACCTGCGCCGCC |
| | TCCTCCGGCCTGATGACCAAGAAGAACTCCACCTTCGTGAGGGTG |
| | CACGAGAAGGACAAGACCCACACCTGCCCCCCCTGCCCCGCCCCC |
| | GAGCTGCTGGGCGGCCCCTCCGTGTTCCTGTTCCCCCCCAAGCCC |
| | AAGGACACCCTGATGATCTCCAGGACCCCCGAGGTGACCTGCGTG |
| | GTGGTGGACGTGTCCCACGAGGACCCCGAGGTGAAGTTCAACTGG |
| | TACGTGGACGGCGTGGAGGTGCACAACGCCAAGACCAAGCCCAGG |
| | GAGGAGCAGTACAACTCCACCTACAGGGTGGTGTCCGTGCTGACC |
| | GTGCTGCACCAGGACTGGCTGAACGGCAAGGAGTACAAGTGCAAG |
| | GTGTCCAACAAGGCCCTGCCCGCCCCCATCGAGAAGACCATCTCC |
| | AAGGCCAAGGGCCAGCCCAGGGAGCCCCAGGTGTACACCCTGCCC |
| | CCCTCCAGGGACGAGCTGACCAAGAACCAGGTGTCCCTGACCTGC |
| | CTGGTGAAGGGCTTCTACCCCTCCGACATCGCCGTGGAGTGGGAG |
| | TCCAACGGCCAGCCCGAGAACAACTACAAGACCACCCCCCCCGTG |
| | CTGGACTCCGACGGCTCCTTCTTCCTGTACTCCAAGCTGACCGTG |
| | GACAAGTCCAGGTGGCAGCAGGGCAACGTGTTCTCCTGCTCCGTG |
| | ATGCACGAGGCCCTGCACAACCACTACACCCAGAAGTCCCTGTCC |
| | CTGTCCCCCGGCAAGTGATTCGAAAATAAAATATCTTTATTTTCA |
| | TTACATCTGTGTGTTGGTTTTTTGTGTGGAGGGCCTATTTCCCAT |
| | GATTCCTTCATATTTGCATATACGATACAAGGCTGTTAGAGAGAT |
| | AATTGGAATTAATTTGACTGTAAACACAAAGATATTAGTACAAAA |
| | TACGTGACGTAGAAAGTAATAATTTCTTGGGTAGTTTGCAGTTTT |
| | AAAATTATGTTTTAAAATGGACTATCATATGCTTACCGTAACTTG |
| | AAAGTATTTCGATTTCTTGGCTTTATATATCTTGTGGAAAGGACA |
| | AGCTTGTAACATTCCCTAATTCTATACCTGACCCATATAGAATTA |
| | GGGAATGTTACTTTTTGCATGCTGGGGAGAGATCAACCCCACTCC |
| | CTCTCTGCGCGCTCGCTCGCTCACTGAGGCCGGGCGACCAAAGGT |
| | CGCCCGACGCCCGGGCTTTGCCCGGGCGGCCTCAGTGAGCGAGCG |
| | AGCGCGCAGCAAGCTGTAGCCAACCACTAGAACTATAGCTAGAGT |
| | CCTGGGCGAACAAACGATGCTCGCCTTCCAGAAAACCGAGGATGC |
| | GAACCACTTCATCCGGGGTCAGCACCACCGGCAAGCGCCGCGACG |
| | GCCGAGGTCTTCCGATCTCCTGAAGCCAGGGCAGATCCGTGCACA |
| | GCACCTTGCCGTAGAAGAACAGCAAGGCCGCCAATGCCTGACGAT |
| | GCGTGGAGACCGAAACCTTGCGCTCGTTCGCCAGCCAGGACAGAA |
| | ATGCCTCGACTTCGCTGCTGCCCAAGGTTGCCGGGTGACGCACAC |
| | CGTGGAAACGGATGAAGGCACGAACCCAGTTGACATAAGCCTGTT |
| | CGGTTCGTAAACTGTAATGCAAGTAGCGTATGCGCTCACGCAACT |
| | GGTCCAGAACCTTGACCGAACGCAGCGGTGGTAACGGCGCAGTGG |
| | CGGTTTTCATGGCTTGTTATGACTGTTTTTTTGTACAGTCTATGC |
| | CTCGGGCATCCAAGCAGCAAGCGCGTTACGCCGTGGGTCGATGTT |
| | TGATGTTATGGAGCAGCAACGATGTTACGCAGCAGCAACGATGTT |
| | ACGCAGCAGGGCAGTCGCCCTAAAACAAAGTTAGGTGGCTCAAGT |
| | ATGGGCATCATTCGCACATGTAGGCTCGGCCCTGACCAAGTCAAA |
| | TCCATGCGGGCTGCTCTTGATCTTTTCGGTCGTGAGTTCGGAGAC |
| | GTAGCCACCTACTCCCAACATCAGCCGGACTCCGATTACCTCGGG |
| | AACTTGCTCCGTAGTAAGACATTCATCGCGCTTGCTGCCTTCGAC |
| | CAAGAAGCGGTTGTTGGCGCTCTCGCGGCTTACGTTCTGCCCAGG |
| | TTTGAGCAGCCGCGTAGTGAGATCTATATCTATGATCTCGCAGTC |
| | TCCGGCGAGCACCGGAGGCAGGGCATTGCCACCGCGCTCATCAAT |
| | CTCCTCAAGCATGAGGCCAACGCGCTTGGTGCTTATGTGATCTAC |
| | GTGCAAGCAGATTACGGTGACGATCCCGCAGTGGCTCTCTATACA |
| | AAGTTGGGCATACGGGAAGAAGTGATGCACTTTGATATCGACCCA |
| | AGTACCGCCACCTAACAATTCGTTCAAGCCGAGATCGGCTTCCCG |
| | GCCGCGGAGTTGTTCGGTAAATTGTCACAACGCCGCGAATATAGT |
| | CTTTACCATGCCCTTGGCCACGCCCCTCTTTAATACGACGGGCAA |
| | TTTGCACTTCAGAAAATGAAGAGTTTGCTTTAGCCATAACAAAAG |
| | TCCAGTATGCTTTTTCACAGCATAACTGGACTGATTTCAGTTTAC |
| | AACTATTCTGTCTAGTTTAAGACTTTATTGTCATAGTTTAGATCT |
| | ATTTTGTTCAGTTTAAGACTTTATTGTCCGCCCACACCCGCTTAC |
| | GCAGGGCATCCATTTATTACTCAACCGTAACCGATTTTGCCAGGT |
| | TACGCGGCTGGTCTGCGGTGTGAAATACCGCACAGATGCGTAAGG |
| | AGAAAATACCGCATCAGGCGCTCTTCCGCTTCCTCGCTCACTGAC |
| | TCGCTGCGCTCGGTCGTTCGGCTGCGGCGAGCGGTATCAGCTCAC |
| | TCAAAGGCGGTAATACGGTTATCCACAGAATCAGGGGATAACGCA |
| | GGAAAGAACATGTGAGCAAAAGGCCAGCAAAAGGCCAGGAACCGT |
| | AAAAAGGCCGCGTTGCTGGCGTTTTTCCATAGGCTCCGCCCCCCT |
| | GACGAGCATCACAAAAATCGACGCTCAAGTCAGAGGTGGCGAAAC |
| | CCGACAGGACTATAAAGATACCAGGCGTTTCCCCCTGGAAGCTCC |
| | CTCGTGCGCTCTCCTGTTCCGACCCTGCCGCTTACCGGATACCTG |
| | TCCGCCTTTCTCCCTTCGGGAAGCGTGGCGCTTTCTCAATGCTCA |
| | CGCTGTAGGTATCTCAGTTCGGTGTAGGTCGTTCGCTCCAAGCTG |
| | GGCTGTGTGCACGAACCCCCCGTTCAGCCCGACCGCTGCGCCTTA |
| | TCCGGTAACTATCGTCTTGAGTCCAACCCGGTAAGACACGACTTA |
| | TCGCCACTGGCAGCAGCCACTGGTAACAGGATTAGCAGAGCGAGG |
| | TATGTAGGCGGTGCTACAGAGTTCTTGAAGTGGTGGCCTAACTAC |
| | GGCTACACTAGAAGGACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTT |
| | TTAAATTAAAAATGAAGTTTTAAATCAATCTAAAGTATATATGAG |
| | TAAACTTGGTCTGACAGTTACCAATGCTTAATCAGTGAGGCACCT |
| | ATCTCAGCGATCTGTCTATTTCGTTCATCCATAGTTGCCTGACTC |
| | CCCGTCGTGTAGATAACTACGATACGGGAGGGCTTACCATCTGGC |
| | CCCAGTGCTGCAATGATACCGCGAGACCCACGCTCACCGGCTCCA |
| | GATTTATCAGCAATAAACCAGCCAGCCGGAAGGGCCGAGCGCAGA |
| | AGTGGTCCTGCAACTTTATCCGCCTCCATCCAGTCTATTAATTGT |
| | TGCCGGGAAGCTAGAGTAAGTAGTTCGCCAGTTAATAGTTTGCGC |
| | AACGTTGTTGCCATTGCTACAGGCATCGTGGTGTCACGCTCGTCG |
| | TTTGGTATGGCTTCATTCAGCTCCGGTTCCCAACGATCAAGGCGA |
| | GTTACATGATCCCCCATGTTGTGCAAAAAAGCGGTTAGCTCCTTC |
| | GGTCCTCCGATCGTTGTCAGAAGTAAGTTGGCCGCAGTGTTATCA |
| | CTCATGGTTATGGCAGCACTGCATAATTCTCTTACTGTCATGCCA |
| | TCCGTAAGATGCTTTTCTGTGACTGGTGAGTACTCAACCAAGTCA |
| | TTCTGAGAATAGTGTATGCGGCGACCGAGTTGCTCTTGCCCGGCG |
| | TCAATACGGGATAATACCGCGCCACATAGCAGAACTTTAAAAGTG |
| | CTCATCATTGGAAAACGTTCTTCGGGGCGAAAACTCTCAAGGATC |
| | TTACCGCTGTTGAGATCCAGTTCGATGTAACCCACTCGTGCACCC |
| | AACTGATCTTCAGCATCTTTTACTTTCACCAGCGTTTCTGGGTGA |
| | GCAAAAACAGGAAGGCAAAATGCCGCAAAAAAGGGAATAAGGGCG |
| | ACACGGAAATGTTGAATACTCATACTCTTCCTTTTTCAATATTAT |
| | TGAAGCATTTATCAGGGTTATTGTCTCATGAGCGGATACATATTT |
| | GAATGTATTTAGAAAAATAAACAAATAGGGGTTCCGCGCACATTT |
| | CCCCGAAAAGTGCCACCTGAAATTGTAAACGTTAATATTTTGTTA |
| | AAATTCGCGTTAAATTTTTGTTAAATCAGCTCATTTTTTAACCAA |
| | TAGGCCGAAATCGGCAAAATCCCTTATAAATCAAAAGAATAGACC |
| | GAGATAGGGTTGAGTGTTGTTCCAGTTTGGAACAAGAGTCCACTA |
| | TTAAAGAACGTGGACTCCAACGTCAAAGGGCGAAAAACCGTCTAT |
| | CAGGGCGATGGCCCACTACGTGAACCATCACCCTAATCAAGTTTT |
| | TTGGGGTCGAGGTGCCGTAAAGCACTAAATCGGAACCCTAAAGGG |
| | AGCCCCCGATTTAGAGCTTGACGGGGAAAGCCGGCGAACGTGGCG |
| | AGAAAGGAAGGGAAGAAAGCGAAAGGAGCGGGCGCTAGGGCGCTG |
| | GCAAGTGTAGCGGTCACGCTGCGCGTAACCACCACACCCGCCGCG |
| | CTTAATGCGCCGCTACAGGGCGCGTC |
| | 611-755: Full ITR |
| AMI151; | 801-104: CMV enhancer |
| SEQ ID | 1105-1308: CMV promoter |
| NO: 62 | 1412-1508: SV40 intron |
| | 1513-1521: Kozak sequence |
| | 1522-1602: Aflibercept secretion sequence |
| | 1603-2895: Aflibercept coding sequence after optimization |
| | 2905-2953: Poly A signal |
| | 2954-3194: Human U6 promoter |
| | 3201-3256: shRNA5 against Ang2 |
| | (GACTTGGAAAGAATATAAAtacctgacccataTTTATATTCTTTCCAAGTCTTTTT) |
| | 3279-3384: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGTGAGC |
| | TACTGGGACACCGGCGTGCTGCTGTGCGCCCTGCTGAGCTGCCTG |
| | CTGCTGACCGGCAGCAGCAGCGGCAGCGACACCGGCAGGCCCTTC |
| | GTGGAGATGTACTCCGAGATCCCCGAGATCATCCACATGACCGAG |
| | GGCAGGGAGCTGGTGATCCCCTGCAGGGTGACCTCCCCCAACATC |
| | ACCGTGACCCTGAAGAAGTTCCCCCTGGACACCCTGATCCCCGAC |
| | GGCAAGAGGATCATCTGGGACTCCAGGAAGGGCTTCATCATCTCC |
| | AACGCCACCTACAAGGAGATCGGCCTGCTGACCTGCGAGGCCACC |
| | GTGAACGGCCACCTGTACAAGACCAACTACCTGACCCACAGGCAG |
| | ACCAACACCATCATCGACGTGGTGCTGTCCCCCTCCCACGGCATC |
| | GAGCTGTCCGTGGGCGAGAAGCTGGTGCTGAACTGCACCGCCAGG |
| | ACCGAGCTGAACGTGGGCATCGACTTCAACTGGGAGTACCCCTCC |
| | TCCAAGCACCAGCACAAGAAGCTGGTGAACAGGGACCTGAAGACC |
| | CAGTCCGGCTCCGAGATGAAGAAGTTCCTGTCCACCCTGACCATC |
| | GACGGCGTGACCAGGTCCGACCAGGGCCTGTACACCTGCGCCGCC |
| | TCCTCCGGCCTGATGACCAAGAAGAACTCCACCTTCGTGAGGGTG |
| | CACGAGAAGGACAAGACCCACACCTGCCCCCCCTGCCCCGCCCCC |
| | GAGCTGCTGGGCGGCCCCTCCGTGTTCCTGTTCCCCCCCAAGCCC |
| | AAGGACACCCTGATGATCTCCAGGACCCCCGAGGTGACCTGCGTG |
| | GTGGTGGACGTGTCCCACGAGGACCCCGAGGTGAAGTTCAACTGG |
| | TACGTGGACGGCGTGGAGGTGCACAACGCCAAGACCAAGCCCAGG |
| | GAGGAGCAGTACAACTCCACCTACAGGGTGGTGTCCGTGCTGACC |
| | GTGCTGCACCAGGACTGGCTGAACGGCAAGGAGTACAAGTGCAAG |
| | GTGTCCAACAAGGCCCTGCCCGCCCCCATCGAGAAGACCATCTCC |
| | AAGGCCAAGGGCCAGCCCAGGGAGCCCCAGGTGTACACCCTGCCC |
| | CCCTCCAGGGACGAGCTGACCAAGAACCAGGTGTCCCTGACCTGC |
| | CTGGTGAAGGGCTTCTACCCCTCCGACATCGCCGTGGAGTGGGAG |
| | TCCAACGGCCAGCCCGAGAACAACTACAAGACCACCCCCCCCGTG |
| | CTGGACTCCGACGGCTCCTTCTTCCTGTACTCCAAGCTGACCGTG |
| | GACAAGTCCAGGTGGCAGCAGGGCAACGTGTTCTCCTGCTCCGTG |
| | ATGCACGAGGCCCTGCACAACCACTACACCCAGAAGTCCCTGTCC |
| | CTGTCCCCCGGCAAGTGATTCGAAAATAAAATATCTTTATTTTCA |
| | TTACATCTGTGTGTTGGTTTTTTGTGTGGAGGGCCTATTTCCCAT |
| | GATTCCTTCATATTTGCATATACGATACAAGGCTGTTAGAGAGAT |
| | AATTGGAATTAATTTGACTGTAAACACAAAGATATTAGTACAAAA |
| | TACGTGACGTAGAAAGTAATAATTTCTTGGGTAGTTTGCAGTTTT |
| | AAAATTATGTTTTAAAATGGACTATCATATGCTTACCGTAACTTG |
| | AAAGTATTTCGATTTCTTGGCTTTATATATCTTGTGGAAAGGACA |
| | AGCTTGACTTGGAAAGAATATAAATACCTGACCCATATTTATATT |
| | CTTTCCAAGTCTTTTTGCATGCTGGGGAGAGATCAACCCCACTCC |
| | CTCTCTGCGCGCTCGCTCGCTCACTGAGGCCGGGCGACCAAAGGT |
| | CGCCCGACGCCCGGGCTTTGCCCGGGCGGCCTCAGTGAGCGAGCG |
| | AGCGCGCAGCAAGCTGTAGCCAACCACTAGAACTATAGCTAGAGT |
| | CCTGGGCGAACAAACGATGCTCGCCTTCCAGAAAACCGAGGATGC |
| | GAACCACTTCATCCGGGGTCAGCACCACCGGCAAGCGCCGCGACG |
| | GCCGAGGTCTTCCGATCTCCTGAAGCCAGGGCAGATCCGTGCACA |
| | GCACCTTGCCGTAGAAGAACAGCAAGGCCGCCAATGCCTGACGAT |
| | GCGTGGAGACCGAAACCTTGCGCTCGTTCGCCAGCCAGGACAGAA |
| | ATGCCTCGACTTCGCTGCTGCCCAAGGTTGCCGGGTGACGCACAC |
| | CGTGGAAACGGATGAAGGCACGAACCCAGTTGACATAAGCCTGTT |
| | CGGTTCGTAAACTGTAATGCAAGTAGCGTATGCGCTCACGCAACT |
| | GGTCCAGAACCTTGACCGAACGCAGCGGTGGTAACGGCGCAGTGG |
| | CGGTTTTCATGGCTTGTTATGACTGTTTTTTTGTACAGTCTATGC |
| | CTCGGGCATCCAAGCAGCAAGCGCGTTACGCCGTGGGTCGATGTT |
| | TGATGTTATGGAGCAGCAACGATGTTACGCAGCAGCAACGATGTT |
| | ACGCAGCAGGGCAGTCGCCCTAAAACAAAGTTAGGTGGCTCAAGT |
| | ATGGGCATCATTCGCACATGTAGGCTCGGCCCTGACCAAGTCAAA |
| | TCCATGCGGGCTGCTCTTGATCTTTTCGGTCGTGAGTTCGGAGAC |
| | GTAGCCACCTACTCCCAACATCAGCCGGACTCCGATTACCTCGGG |
| | AACTTGCTCCGTAGTAAGACATTCATCGCGCTTGCTGCCTTCGAC |
| | CAAGAAGCGGTTGTTGGCGCTCTCGCGGCTTACGTTCTGCCCAGG |
| | TTTGAGCAGCCGCGTAGTGAGATCTATATCTATGATCTCGCAGTC |
| | TCCGGCGAGCACCGGAGGCAGGGCATTGCCACCGCGCTCATCAAT |
| | CTCCTCAAGCATGAGGCCAACGCGCTTGGTGCTTATGTGATCTAC |
| | GTGCAAGCAGATTACGGTGACGATCCCGCAGTGGCTCTCTATACA |
| | AAGTTGGGCATACGGGAAGAAGTGATGCACTTTGATATCGACCCA |
| | AGTACCGCCACCTAACAATTCGTTCAAGCCGAGATCGGCTTCCCG |
| | GCCGCGGAGTTGTTCGGTAAATTGTCACAACGCCGCGAATATAGT |
| | CTTTACCATGCCCTTGGCCACGCCCCTCTTTAATACGACGGGCAA |
| | TTTGCACTTCAGAAAATGAAGAGTTTGCTTTAGCCATAACAAAAG |
| | TCCAGTATGCTTTTTCACAGCATAACTGGACTGATTTCAGTTTAC |
| | AACTATTCTGTCTAGTTTAAGACTTTATTGTCATAGTTTAGATCT |
| | ATTTTGTTCAGTTTAAGACTTTATTGTCCGCCCACACCCGCTTAC |
| | GCAGGGCATCCATTTATTACTCAACCGTAACCGATTTTGCCAGGT |
| | TACGCGGCTGGTCTGCGGTGTGAAATACCGCACAGATGCGTAAGG |
| | AGAAAATACCGCATCAGGCGCTCTTCCGCTTCCTCGCTCACTGAC |
| | TCGCTGCGCTCGGTCGTTCGGCTGCGGCGAGCGGTATCAGCTCAC |
| | TCAAAGGCGGTAATACGGTTATCCACAGAATCAGGGGATAACGCA |
| | GGAAAGAACATGTGAGCAAAAGGCCAGCAAAAGGCCAGGAACCGT |
| | AAAAAGGCCGCGTTGCTGGCGTTTTTCCATAGGCTCCGCCCCCCT |
| | GACGAGCATCACAAAAATCGACGCTCAAGTCAGAGGTGGCGAAAC |
| | CCGACAGGACTATAAAGATACCAGGCGTTTCCCCCTGGAAGCTCC |
| | CTCGTGCGCTCTCCTGTTCCGACCCTGCCGCTTACCGGATACCTG |
| | TCCGCCTTTCTCCCTTCGGGAAGCGTGGCGCTTTCTCAATGCTCA |
| | CGCTGTAGGTATCTCAGTTCGGTGTAGGTCGTTCGCTCCAAGCTG |
| | GGCTGTGTGCACGAACCCCCCGTTCAGCCCGACCGCTGCGCCTTA |
| | TCCGGTAACTATCGTCTTGAGTCCAACCCGGTAAGACACGACTTA |
| | TCGCCACTGGCAGCAGCCACTGGTAACAGGATTAGCAGAGCGAGG |
| | TATGTAGGCGGTGCTACAGAGTTCTTGAAGTGGTGGCCTAACTAC |
| | GGCTACACTAGAAGGACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTT |
| | TTAAATTAAAAATGAAGTTTTAAATCAATCTAAAGTATATATGAG |
| | TAAACTTGGTCTGACAGTTACCAATGCTTAATCAGTGAGGCACCT |
| | ATCTCAGCGATCTGTCTATTTCGTTCATCCATAGTTGCCTGACTC |
| | CCCGTCGTGTAGATAACTACGATACGGGAGGGCTTACCATCTGGC |
| | CCCAGTGCTGCAATGATACCGCGAGACCCACGCTCACCGGCTCCA |
| | GATTTATCAGCAATAAACCAGCCAGCCGGAAGGGCCGAGCGCAGA |
| | AGTGGTCCTGCAACTTTATCCGCCTCCATCCAGTCTATTAATTGT |
| | TGCCGGGAAGCTAGAGTAAGTAGTTCGCCAGTTAATAGTTTGCGC |
| | AACGTTGTTGCCATTGCTACAGGCATCGTGGTGTCACGCTCGTCG |
| | TTTGGTATGGCTTCATTCAGCTCCGGTTCCCAACGATCAAGGCGA |
| | GTTACATGATCCCCCATGTTGTGCAAAAAAGCGGTTAGCTCCTTC |
| | GGTCCTCCGATCGTTGTCAGAAGTAAGTTGGCCGCAGTGTTATCA |
| | CTCATGGTTATGGCAGCACTGCATAATTCTCTTACTGTCATGCCA |
| | TCCGTAAGATGCTTTTCTGTGACTGGTGAGTACTCAACCAAGTCA |
| | TTCTGAGAATAGTGTATGCGGCGACCGAGTTGCTCTTGCCCGGCG |
| | TCAATACGGGATAATACCGCGCCACATAGCAGAACTTTAAAAGTG |
| | CTCATCATTGGAAAACGTTCTTCGGGGCGAAAACTCTCAAGGATC |
| | TTACCGCTGTTGAGATCCAGTTCGATGTAACCCACTCGTGCACCC |
| | AACTGATCTTCAGCATCTTTTACTTTCACCAGCGTTTCTGGGTGA |
| | GCAAAAACAGGAAGGCAAAATGCCGCAAAAAAGGGAATAAGGGCG |
| | ACACGGAAATGTTGAATACTCATACTCTTCCTTTTTCAATATTAT |
| | TGAAGCATTTATCAGGGTTATTGTCTCATGAGCGGATACATATTT |
| | GAATGTATTTAGAAAAATAAACAAATAGGGGTTCCGCGCACATTT |
| | CCCCGAAAAGTGCCACCTGAAATTGTAAACGTTAATATTTTGTTA |
| | AAATTCGCGTTAAATTTTTGTTAAATCAGCTCATTTTTTAACCAA |
| | TAGGCCGAAATCGGCAAAATCCCTTATAAATCAAAAGAATAGACC |
| | GAGATAGGGTTGAGTGTTGTTCCAGTTTGGAACAAGAGTCCACTA |
| | TTAAAGAACGTGGACTCCAACGTCAAAGGGCGAAAAACCGTCTAT |
| | CAGGGCGATGGCCCACTACGTGAACCATCACCCTAATCAAGTTTT |
| | TTGGGGTCGAGGTGCCGTAAAGCACTAAATCGGAACCCTAAAGGG |
| | AGCCCCCGATTTAGAGCTTGACGGGGAAAGCCGGCGAACGTGGCG |
| | AGAAAGGAAGGGAAGAAAGCGAAAGGAGCGGGCGCTAGGGCGCTG |
| | GCAAGTGTAGCGGTCACGCTGCGCGTAACCACCACACCCGCCGCG |
| | CTTAATGCGCCGCTACAGGGCGCGTC |
| AMI152; | 611-755: Full ITR |
| SEQ ID | 801-104: CMV enhancer |
| NO: 63 | 1105-1308: CMV promoter |
| | 1412-1508: SV40 intron |
| | 1513-1521: Kozak sequence |
| | 1522-1602: Aflibercept secretion sequence |
| | 1603-2895: Aflibercept coding sequence after optimization |
| | 2905-2953: Poly A signal |
| | 2954-3194: Human U6 promoter |
| | 3201-3256: shRNA6 against Ang2 |
| | (GGTGAAGAACTCAATTATAtacctgacccataTATAATTGAGTTCTTCACCTTTTT |
| | 3279-3384: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGTGAGC |
| | TACTGGGACACCGGCGTGCTGCTGTGCGCCCTGCTGAGCTGCCTG |
| | CTGCTGACCGGCAGCAGCAGCGGCAGCGACACCGGCAGGCCCTTC |
| | GTGGAGATGTACTCCGAGATCCCCGAGATCATCCACATGACCGAG |
| | GGCAGGGAGCTGGTGATCCCCTGCAGGGTGACCTCCCCCAACATC |
| | ACCGTGACCCTGAAGAAGTTCCCCCTGGACACCCTGATCCCCGAC |
| | GGCAAGAGGATCATCTGGGACTCCAGGAAGGGCTTCATCATCTCC |
| | AACGCCACCTACAAGGAGATCGGCCTGCTGACCTGCGAGGCCACC |
| | GTGAACGGCCACCTGTACAAGACCAACTACCTGACCCACAGGCAG |
| | ACCAACACCATCATCGACGTGGTGCTGTCCCCCTCCCACGGCATC |
| | GAGCTGTCCGTGGGCGAGAAGCTGGTGCTGAACTGCACCGCCAGG |
| | ACCGAGCTGAACGTGGGCATCGACTTCAACTGGGAGTACCCCTCC |
| | TCCAAGCACCAGCACAAGAAGCTGGTGAACAGGGACCTGAAGACC |
| | CAGTCCGGCTCCGAGATGAAGAAGTTCCTGTCCACCCTGACCATC |
| | GACGGCGTGACCAGGTCCGACCAGGGCCTGTACACCTGCGCCGCC |
| | TCCTCCGGCCTGATGACCAAGAAGAACTCCACCTTCGTGAGGGTG |
| | CACGAGAAGGACAAGACCCACACCTGCCCCCCCTGCCCCGCCCCC |
| | GAGCTGCTGGGCGGCCCCTCCGTGTTCCTGTTCCCCCCCAAGCCC |
| | AAGGACACCCTGATGATCTCCAGGACCCCCGAGGTGACCTGCGTG |
| | GTGGTGGACGTGTCCCACGAGGACCCCGAGGTGAAGTTCAACTGG |
| | TACGTGGACGGCGTGGAGGTGCACAACGCCAAGACCAAGCCCAGG |
| | GAGGAGCAGTACAACTCCACCTACAGGGTGGTGTCCGTGCTGACC |
| | GTGCTGCACCAGGACTGGCTGAACGGCAAGGAGTACAAGTGCAAG |
| | GTGTCCAACAAGGCCCTGCCCGCCCCCATCGAGAAGACCATCTCC |
| | AAGGCCAAGGGCCAGCCCAGGGAGCCCCAGGTGTACACCCTGCCC |
| | CCCTCCAGGGACGAGCTGACCAAGAACCAGGTGTCCCTGACCTGC |
| | CTGGTGAAGGGCTTCTACCCCTCCGACATCGCCGTGGAGTGGGAG |
| | TCCAACGGCCAGCCCGAGAACAACTACAAGACCACCCCCCCCGTG |
| | CTGGACTCCGACGGCTCCTTCTTCCTGTACTCCAAGCTGACCGTG |
| | GACAAGTCCAGGTGGCAGCAGGGCAACGTGTTCTCCTGCTCCGTG |
| | ATGCACGAGGCCCTGCACAACCACTACACCCAGAAGTCCCTGTCC |
| | CTGTCCCCCGGCAAGTGATTCGAAAATAAAATATCTTTATTTTCA |
| | TTACATCTGTGTGTTGGTTTTTTGTGTGGAGGGCCTATTTCCCAT |
| | GATTCCTTCATATTTGCATATACGATACAAGGCTGTTAGAGAGAT |
| | AATTGGAATTAATTTGACTGTAAACACAAAGATATTAGTACAAAA |
| | TACGTGACGTAGAAAGTAATAATTTCTTGGGTAGTTTGCAGTTTT |
| | AAAATTATGTTTTAAAATGGACTATCATATGCTTACCGTAACTTG |
| | AAAGTATTTCGATTTCTTGGCTTTATATATCTTGTGGAAAGGACA |
| | AGCTTGGTGAAGAACTCAATTATATACCTGACCCATATATAATTG |
| | AGTTCTTCACCTTTTTGCATGCTGGGGAGAGATCAACCCCACTCC |
| | CTCTCTGCGCGCTCGCTCGCTCACTGAGGCCGGGCGACCAAAGGT |
| | CGCCCGACGCCCGGGCTTTGCCCGGGCGGCCTCAGTGAGCGAGCG |
| | AGCGCGCAGCAAGCTGTAGCCAACCACTAGAACTATAGCTAGAGT |
| | CCTGGGCGAACAAACGATGCTCGCCTTCCAGAAAACCGAGGATGC |
| | GAACCACTTCATCCGGGGTCAGCACCACCGGCAAGCGCCGCGACG |
| | GCCGAGGTCTTCCGATCTCCTGAAGCCAGGGCAGATCCGTGCACA |
| | GCACCTTGCCGTAGAAGAACAGCAAGGCCGCCAATGCCTGACGAT |
| | GCGTGGAGACCGAAACCTTGCGCTCGTTCGCCAGCCAGGACAGAA |
| | ATGCCTCGACTTCGCTGCTGCCCAAGGTTGCCGGGTGACGCACAC |
| | CGTGGAAACGGATGAAGGCACGAACCCAGTTGACATAAGCCTGTT |
| | CGGTTCGTAAACTGTAATGCAAGTAGCGTATGCGCTCACGCAACT |
| | GGTCCAGAACCTTGACCGAACGCAGCGGTGGTAACGGCGCAGTGG |
| | CGGTTTTCATGGCTTGTTATGACTGTTTTTTTGTACAGTCTATGC |
| | CTCGGGCATCCAAGCAGCAAGCGCGTTACGCCGTGGGTCGATGTT |
| | TGATGTTATGGAGCAGCAACGATGTTACGCAGCAGCAACGATGTT |
| | ACGCAGCAGGGCAGTCGCCCTAAAACAAAGTTAGGTGGCTCAAGT |
| | ATGGGCATCATTCGCACATGTAGGCTCGGCCCTGACCAAGTCAAA |
| | TCCATGCGGGCTGCTCTTGATCTTTTCGGTCGTGAGTTCGGAGAC |
| | GTAGCCACCTACTCCCAACATCAGCCGGACTCCGATTACCTCGGG |
| | AACTTGCTCCGTAGTAAGACATTCATCGCGCTTGCTGCCTTCGAC |
| | CAAGAAGCGGTTGTTGGCGCTCTCGCGGCTTACGTTCTGCCCAGG |
| | TTTGAGCAGCCGCGTAGTGAGATCTATATCTATGATCTCGCAGTC |
| | TCCGGCGAGCACCGGAGGCAGGGCATTGCCACCGCGCTCATCAAT |
| | CTCCTCAAGCATGAGGCCAACGCGCTTGGTGCTTATGTGATCTAC |
| | GTGCAAGCAGATTACGGTGACGATCCCGCAGTGGCTCTCTATACA |
| | AAGTTGGGCATACGGGAAGAAGTGATGCACTTTGATATCGACCCA |
| | AGTACCGCCACCTAACAATTCGTTCAAGCCGAGATCGGCTTCCCG |
| | GCCGCGGAGTTGTTCGGTAAATTGTCACAACGCCGCGAATATAGT |
| | CTTTACCATGCCCTTGGCCACGCCCCTCTTTAATACGACGGGCAA |
| | TTTGCACTTCAGAAAATGAAGAGTTTGCTTTAGCCATAACAAAAG |
| | TCCAGTATGCTTTTTCACAGCATAACTGGACTGATTTCAGTTTAC |
| | AACTATTCTGTCTAGTTTAAGACTTTATTGTCATAGTTTAGATCT |
| | ATTTTGTTCAGTTTAAGACTTTATTGTCCGCCCACACCCGCTTAC |
| | GCAGGGCATCCATTTATTACTCAACCGTAACCGATTTTGCCAGGT |
| | TACGCGGCTGGTCTGCGGTGTGAAATACCGCACAGATGCGTAAGG |
| | AGAAAATACCGCATCAGGCGCTCTTCCGCTTCCTCGCTCACTGAC |
| | TCGCTGCGCTCGGTCGTTCGGCTGCGGCGAGCGGTATCAGCTCAC |
| | TCAAAGGCGGTAATACGGTTATCCACAGAATCAGGGGATAACGCA |
| | GGAAAGAACATGTGAGCAAAAGGCCAGCAAAAGGCCAGGAACCGT |
| | AAAAAGGCCGCGTTGCTGGCGTTTTTCCATAGGCTCCGCCCCCCT |
| | GACGAGCATCACAAAAATCGACGCTCAAGTCAGAGGTGGCGAAAC |
| | CCGACAGGACTATAAAGATACCAGGCGTTTCCCCCTGGAAGCTCC |
| | CTCGTGCGCTCTCCTGTTCCGACCCTGCCGCTTACCGGATACCTG |
| | TCCGCCTTTCTCCCTTCGGGAAGCGTGGCGCTTTCTCAATGCTCA |
| | CGCTGTAGGTATCTCAGTTCGGTGTAGGTCGTTCGCTCCAAGCTG |
| | GGCTGTGTGCACGAACCCCCCGTTCAGCCCGACCGCTGCGCCTTA |
| | TCCGGTAACTATCGTCTTGAGTCCAACCCGGTAAGACACGACTTA |
| | TCGCCACTGGCAGCAGCCACTGGTAACAGGATTAGCAGAGCGAGG |
| | TATGTAGGCGGTGCTACAGAGTTCTTGAAGTGGTGGCCTAACTAC |
| | GGCTACACTAGAAGGACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTT |
| | TCTACGGGGTCTGACGCTCAGTGGAACGAAAACTCACGTTAAGGG |
| | ATTTTGGTCATGAGATTATCAAAAAGGATCTTCACCTAGATCCTT |
| | TTAAATTAAAAATGAAGTTTTAAATCAATCTAAAGTATATATGAG |
| | TAAACTTGGTCTGACAGTTACCAATGCTTAATCAGTGAGGCACCT |
| | ATCTCAGCGATCTGTCTATTTCGTTCATCCATAGTTGCCTGACTC |
| | CCCGTCGTGTAGATAACTACGATACGGGAGGGCTTACCATCTGGC |
| | CCCAGTGCTGCAATGATACCGCGAGACCCACGCTCACCGGCTCCA |
| | GATTTATCAGCAATAAACCAGCCAGCCGGAAGGGCCGAGCGCAGA |
| | AGTGGTCCTGCAACTTTATCCGCCTCCATCCAGTCTATTAATTGT |
| | TGCCGGGAAGCTAGAGTAAGTAGTTCGCCAGTTAATAGTTTGCGC |
| | AACGTTGTTGCCATTGCTACAGGCATCGTGGTGTCACGCTCGTCG |
| | TTTGGTATGGCTTCATTCAGCTCCGGTTCCCAACGATCAAGGCGA |
| | GTTACATGATCCCCCATGTTGTGCAAAAAAGCGGTTAGCTCCTTC |
| | GGTCCTCCGATCGTTGTCAGAAGTAAGTTGGCCGCAGTGTTATCA |
| | CTCATGGTTATGGCAGCACTGCATAATTCTCTTACTGTCATGCCA |
| | TCCGTAAGATGCTTTTCTGTGACTGGTGAGTACTCAACCAAGTCA |
| | TTCTGAGAATAGTGTATGCGGCGACCGAGTTGCTCTTGCCCGGCG |
| | TCAATACGGGATAATACCGCGCCACATAGCAGAACTTTAAAAGTG |
| | CTCATCATTGGAAAACGTTCTTCGGGGCGAAAACTCTCAAGGATC |
| | TTACCGCTGTTGAGATCCAGTTCGATGTAACCCACTCGTGCACCC |
| | AACTGATCTTCAGCATCTTTTACTTTCACCAGCGTTTCTGGGTGA |
| | GCAAAAACAGGAAGGCAAAATGCCGCAAAAAAGGGAATAAGGGCG |
| | ACACGGAAATGTTGAATACTCATACTCTTCCTTTTTCAATATTAT |
| | TGAAGCATTTATCAGGGTTATTGTCTCATGAGCGGATACATATTT |
| | GAATGTATTTAGAAAAATAAACAAATAGGGGTTCCGCGCACATTT |
| | CCCCGAAAAGTGCCACCTGAAATTGTAAACGTTAATATTTTGTTA |
| | AAATTCGCGTTAAATTTTTGTTAAATCAGCTCATTTTTTAACCAA |
| | TAGGCCGAAATCGGCAAAATCCCTTATAAATCAAAAGAATAGACC |
| | GAGATAGGGTTGAGTGTTGTTCCAGTTTGGAACAAGAGTCCACTA |
| | TTAAAGAACGTGGACTCCAACGTCAAAGGGCGAAAAACCGTCTAT |
| | CAGGGCGATGGCCCACTACGTGAACCATCACCCTAATCAAGTTTT |
| | TTGGGGTCGAGGTGCCGTAAAGCACTAAATCGGAACCCTAAAGGG |
| | AGCCCCCGATTTAGAGCTTGACGGGGAAAGCCGGCGAACGTGGCG |
| | AGAAAGGAAGGGAAGAAAGCGAAAGGAGCGGGCGCTAGGGCGCTG |
| | GCAAGTGTAGCGGTCACGCTGCGCGTAACCACCACACCCGCCGCG |
| | CTTAATGCGCCGCTACAGGGCGCGTC |
| AMI153; | 372-512: Full ITR |
| SEQ ID | 570-873: CMV enhancer |
| NO: 64 | 874-1077: CMV promoter |
| | 1181-1277: SV40 intron |
| | 1282-1290: Kozak sequence |
| | 1291-1371: Aflibercept secretion sequence |
| | 1372-2664: Aflibercept coding sequence after optimization |
| | 2674-2722: Poly A sequence |
| | 2757-3060: CMV enhancer |
| | 3061-3264: CMV promoter |
| | 3368-3464: SV40 intron |
| | 3469-3477: Kozak sequence |
| | 3478-3534: Human IgG heavy chain secretion sequence |
| | 3535-4398: hCOMP-Ang1 coding sequence after optimization |
| | 4422-4470: Poly A sequence |
| | 4498-4638: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGTGAGCTACTGG |
| | GACACCGGCGTGCTGCTGTGCGCCCTGCTGAGCTGCCTGCTGCTG |
| | ACCGGCAGCAGCAGCGGCAGCGACACCGGCAGGCCCTTCGTGGAG |
| | ATGTACTCCGAGATCCCCGAGATCATCCACATGACCGAGGGCAGG |
| | GAGCTGGTGATCCCCTGCAGGGTGACCTCCCCCAACATCACCGTG |
| | ACCCTGAAGAAGTTCCCCCTGGACACCCTGATCCCCGACGGCAAG |
| | AGGATCATCTGGGACTCCAGGAAGGGCTTCATCATCTCCAACGCC |
| | ACCTACAAGGAGATCGGCCTGCTGACCTGCGAGGCCACCGTGAAC |
| | GGCCACCTGTACAAGACCAACTACCTGACCCACAGGCAGACCAAC |
| | ACCATCATCGACGTGGTGCTGTCCCCCTCCCACGGCATCGAGCTG |
| | TCCGTGGGCGAGAAGCTGGTGCTGAACTGCACCGCCAGGACCGAG |
| | CTGAACGTGGGCATCGACTTCAACTGGGAGTACCCCTCCTCCAAG |
| | CACCAGCACAAGAAGCTGGTGAACAGGGACCTGAAGACCCAGTCC |
| | GGCTCCGAGATGAAGAAGTTCCTGTCCACCCTGACCATCGACGGC |
| | GTGACCAGGTCCGACCAGGGCCTGTACACCTGCGCCGCCTCCTCC |
| | GGCCTGATGACCAAGAAGAACTCCACCTTCGTGAGGGTGCACGAG |
| | AAGGACAAGACCCACACCTGCCCCCCCTGCCCCGCCCCCGAGCTG |
| | CTGGGCGGCCCCTCCGTGTTCCTGTTCCCCCCCAAGCCCAAGGAC |
| | ACCCTGATGATCTCCAGGACCCCCGAGGTGACCTGCGTGGTGGTG |
| | GACGTGTCCCACGAGGACCCCGAGGTGAAGTTCAACTGGTACGTG |
| | GACGGCGTGGAGGTGCACAACGCCAAGACCAAGCCCAGGGAGGAG |
| | CAGTACAACTCCACCTACAGGGTGGTGTCCGTGCTGACCGTGCTG |
| | CACCAGGACTGGCTGAACGGCAAGGAGTACAAGTGCAAGGTGTCC |
| | AACAAGGCCCTGCCCGCCCCCATCGAGAAGACCATCTCCAAGGCC |
| | AAGGGCCAGCCCAGGGAGCCCCAGGTGTACACCCTGCCCCCCTCC |
| | AGGGACGAGCTGACCAAGAACCAGGTGTCCCTGACCTGCCTGGTG |
| | AAGGGCTTCTACCCCTCCGACATCGCCGTGGAGTGGGAGTCCAAC |
| | GGCCAGCCCGAGAACAACTACAAGACCACCCCCCCCGTGCTGGAC |
| | TCCGACGGCTCCTTCTTCCTGTACTCCAAGCTGACCGTGGACAAG |
| | TCCAGGTGGCAGCAGGGCAACGTGTTCTCCTGCTCCGTGATGCAC |
| | GAGGCCCTGCACAACCACTACACCCAGAAGTCCCTGTCCCTGTCC |
| | CCCGGCAAGTGATTCGAAAATAAAATATCTTTATTTTCATTACAT |
| | CTGTGTGTTGGTTTTTTGTGTGGCATGCTGGGGAGAGATCAACCG |
| | AATTCGGTACCCGTTACATAACTTACGGTAAATGGCCCGCCTGGC |
| | TGACCGCCCAACGACCCCCGCCCATTGACGTCAATAATGACGTAT |
| | GTTCCCATAGTAACGCCAATAGGGACTTTCCATTGACGTCAATGG |
| | GTGGAGTATTTACGGTAAACTGCCCACTTGGCAGTACATCAAGTG |
| | TATCATATGCCAAGTACGCCCCCTATTGACGTCAATGACGGTAAA |
| | TGGCCCGCCTGGCATTATGCCCAGTACATGACCTTATGGGACTTT |
| | CCTACTTGGCAGTACATCTACGTATTAGTCATCGCTATTACCATG |
| | GTGATGCGGTTTTGGCAGTACATCAATGGGCGTGGATAGCGGTTT |
| | GACTCACGGGGATTTCCAAGTCTCCACCCCATTGACGTCAATGGG |
| | AGTTTGTTTTGGCACCAAAATCAACGGGACTTTCCAAAATGTCGT |
| | AACAACTCCGCCCCATTGACGCAAATGGGCGGTAGGCGTGTACGG |
| | TGGGAGGTCTATATAAGCAGAGCTCGTTTAGTGAACCGTCAGATC |
| | GCCTGGAGACGCCATCCACGCTGTTTTGACCTCCATAGAAGACAC |
| | CGGGACCGATCCAGCCTCCGGACTCTAGAGTTAACTGGTAAGTTT |
| | AGTCTTTTTGTCTTTTATTTCAGGTCCCGGATCCGGTGGTGGTGC |
| | AAATCAAAGAACTGCTCCTCAGTGGATGTTGCCTTTACTTCTAGG |
| | CCTGCCGCCACCATGGAGTTCGGCCTGAGCTGGCTGTTCCTGGTG |
| | GCCATCCTTAAGGGCGTGCAGTGCGACCTGGGCCCCCAGATGCTG |
| | AGAGAGCTGCAGGAGACCAACGCCGCCCTGCAGGACGTGAGAGAG |
| | CTGCTGAGACAGCAGGTGAAGGAGATCACCTTCCTGAGAAACACC |
| | GTGATGGAGTGCGACGCCTGCGGCGACACCGTGCACAACCTGGTG |
| | AACCTGTGCACCAAGGAGGGCGTGCTGCTGAAGGGCGGCAAGAGA |
| | GAGGAGGAGAAGCCCTTCAGAGACTGCGCCGACGTGTACCAGGCC |
| | GGCTTCAACAAGAGCGGCATCTACACCATCTACATCAACAACATG |
| | CCCGAGCCCAAGAAGGTGTTCTGCAACATGGACGTGAACGGCGGC |
| | GGCTGGACCGTGATCCAGCACAGAGAGGACGGCAGCCTGGACTTC |
| | CAGAGAGGCTGGAAGGAGTACAAGATGGGCTTCGGCAACCCCAGC |
| | GGCGAGTACTGGCTGGGCAACGAGTTCATCTTCGCCATCACCAGC |
| | CAGAGACAGTACATGCTGAGAATCGAGCTGATGGACTGGGAGGGC |
| | AACAGAGCCTACAGCCAGTACGACAGATTCCACATCGGCAACGAG |
| | AAGCAGAACTACAGACTGTACCTGAAGGGCCACACCGGCACCGCC |
| | GGCAAGCAGAGCAGCCTGATCCTGCACGGCGCCGACTTCAGCACC |
| | AAGGACGCCGACAACGACAACTGCATGTGCAAGTGCGCCCTGATG |
| | CTGACCGGCGGCTGGTGGTTCGACGCCTGCGGCCCCAGCAACCTG |
| | AACGGCATGTTCTACACCGCCGGCCAGAACCACGGCAAGCTGAAC |
| | GGCATCAAGTGGCACTACTTCAAGGGCCCCAGCTACAGCCTGAGA |
| | AGCACCACCATGATGATCAGACCCCTGGACTTCTGAGCGGGACTC |
| | TGGAATTCGAAAATAAAATATCTTTATTTTCATTACATCTGTGTG |
| | TTGGTTTTTTGTGTGTTCTGCACGTGCGGACCGAGCGGCCGCAGG |
| | AACCCCTAGTGATGGAGTTGGCCACTCCCTCTCTGCGCGCTCGCT |
| | CGCTCACTGAGGCCGGGCGACCAAAGGTCGCCCGACGCCCGGGCT |
| | TTGCCCGGGCGGCCTCAGTGAGCGAGCGAGCGCGCAGCTGCCTGC |
| | AGGCATGCAAGCTGTAGCCAACCACTAGAACTATAGCTAGAGTCC |
| | TGGGCGAACAAACGATGCTCGCCTTCCAGAAAACCGAGGATGCGA |
| | ACCACTTCATCCGGGGTCAGCACCACCGGCAAGCGCCGCGACGGC |
| | CGAGGTCTTCCGATCTCCTGAAGCCAGGGCAGATCCGTGCACAGC |
| | ACCTTGCCGTAGAAGAACAGCAAGGCCGCCAATGCCTGACGATGC |
| | GTGGAGACCGAAACCTTGCGCTCGTTCGCCAGCCAGGACAGAAAT |
| | GCCTCGACTTCGCTGCTGCCCAAGGTTGCCGGGTGACGCACACCG |
| | TGGAAACGGATGAAGGCACGAACCCAGTTGACATAAGCCTGTTCG |
| | GTTCGTAAACTGTAATGCAAGTAGCGTATGCGCTCACGCAACTGG |
| | TCCAGAACCTTGACCGAACGCAGCGGTGGTAACGGCGCAGTGGCG |
| | GTTTTCATGGCTTGTTATGACTGTTTTTTTGTACAGTCTATGCCT |
| | CGGGCATCCAAGCAGCAAGCGCGTTACGCCGTGGGTCGATGTTTG |
| | ATGTTATGGAGCAGCAACGATGTTACGCAGCAGCAACGATGTTAC |
| | GCAGCAGGGCAGTCGCCCTAAAACAAAGTTAGGTGGCTCAAGTAT |
| | GGGCATCATTCGCACATGTAGGCTCGGCCCTGACCAAGTCAAATC |
| | CATGCGGGCTGCTCTTGATCTTTTCGGTCGTGAGTTCGGAGACGT |
| | AGCCACCTACTCCCAACATCAGCCGGACTCCGATTACCTCGGGAA |
| | CTTGCTCCGTAGTAAGACATTCATCGCGCTTGCTGCCTTCGACCA |
| | AGAAGCGGTTGTTGGCGCTCTCGCGGCTTACGTTCTGCCCAGGTT |
| | TGAGCAGCCGCGTAGTGAGATCTATATCTATGATCTCGCAGTCTC |
| | CGGCGAGCACCGGAGGCAGGGCATTGCCACCGCGCTCATCAATCT |
| | CCTCAAGCATGAGGCCAACGCGCTTGGTGCTTATGTGATCTACGT |
| | GCAAGCAGATTACGGTGACGATCCCGCAGTGGCTCTCTATACAAA |
| | GTTGGGCATACGGGAAGAAGTGATGCACTTTGATATCGACCCAAG |
| | TACCGCCACCTAACAATTCGTTCAAGCCGAGATCGGCTTCCCGGC |
| | CGCGGAGTTGTTCGGTAAATTGTCACAACGCCGCGAATATAGTCT |
| | TTACCATGCCCTTGGCCACGCCCCTCTTTAATACGACGGGCAATT |
| | TGCACTTCAGAAAATGAAGAGTTTGCTTTAGCCATAACAAAAGTC |
| | CAGTATGCTTTTTCACAGCATAACTGGACTGATTTCAGTTTACAA |
| | CTATTCTGTCTAGTTTAAGACTTTATTGTCATAGTTTAGATCTAT |
| | TTTGTTCAGTTTAAGACTTTATTGTCCGCCCACACCCGCTTACGC |
| | AGGGCATCCATTTATTACTCAACCGTAACCGATTTTGCCAGGTTA |
| | CGCGGCTGGTCTGCGGTGTGAAATACCGCACAGATGCGTAAGGAG |
| | AAAATACCGCATCAGGCGCTCTTCCGCTTCCTCGCTCACTGACTC |
| | GCTGCGCTCGGTCGTTCGGCTGCGGCGAGCGGTATCAGCTCACTC |
| | AAAGGCGGTAATACGGTTATCCACAGAATCAGGGGATAACGCAGG |
| | AAAGAACATGTGAGCAAAAGGCCAGCAAAAGGCCAGGAACCGTAA |
| | AAAGGCCGCGTTGCTGGCGTTTTTCCATAGGCTCCGCCCCCCTGA |
| | CGAGCATCACAAAAATCGACGCTCAAGTCAGAGGTGGCGAAACCC |
| | GACAGGACTATAAAGATACCAGGCGTTTCCCCCTGGAAGCTCCCT |
| | CGTGCGCTCTCCTGTTCCGACCCTGCCGCTTACCGGATACCTGTC |
| | CGCCTTTCTCCCTTCGGGAAGCGTGGCGCTTTCTCAATGCTCACG |
| | CTGTAGGTATCTCAGTTCGGTGTAGGTCGTTCGCTCCAAGCTGGG |
| | CTGTGTGCACGAACCCCCCGTTCAGCCCGACCGCTGCGCCTTATC |
| | CGGTAACTATCGTCTTGAGTCCAACCCGGTAAGACACGACTTATC |
| | GCCACTGGCAGCAGCCACTGGTAACAGGATTAGCAGAGCGAGGTA |
| | TGTAGGCGGTGCTACAGAGTTCTTGAAGTGGTGGCCTAACTACGG |
| | CTACACTAGAAGGACAGTATTTGGTATCTGCGCTCTGCTGAAGCC |
| | AGTTACCTTCGGAAAAAGAGTTGGTAGCTCTTGATCCGGCAAACA |
| | AACCACCGCTGGTAGCGGTGGTTTTTTTGTTTGCAAGCAGCAGAT |
| | TACGCGCAGAAAAAAAGGATCTCAAGAAGATCCTTTGATCTTTTC |
| | TACGGGGTCTGACGCTCAGTGGAACGAAAACTCACGTTAAGGGAT |
| | TTTGGTCATGAGATTATCAAAAAGGATCTTCACCTAGATCCTTTT |
| | AAATTAAAAATGAAGTTTTAAATCAATCTAAAGTATATATGAGTA |
| | AACTTGGTCTGACAGTTACCAATGCTTAATCAGTGAGGCACCTAT |
| | CTCAGCGATCTGTCTATTTCGTTCATCCATAGTTGCCTGACTCCC |
| | CGTCGTGTAGATAACTACGATACGGGAGGGCTTACCATCTGGCCC |
| | CAGTGCTGCAATGATACCGCGAGACCCACGCTCACCGGCTCCAGA |
| | TTTATCAGCAATAAACCAGCCAGCCGGAAGGGCCGAGCGCAGAAG |
| | TGGTCCTGCAACTTTATCCGCCTCCATCCAGTCTATTAATTGTTG |
| | CCGGGAAGCTAGAGTAAGTAGTTCGCCAGTTAATAGTTTGCGCAA |
| | CGTTGTTGCCATTGCTACAGGCATCGTGGTGTCACGCTCGTCGTT |
| | TGGTATGGCTTCATTCAGCTCCGGTTCCCAACGATCAAGGCGAGT |
| | TACATGATCCCCCATGTTGTGCAAAAAAGCGGTTAGCTCCTTCGG |
| | TCCTCCGATCGTTGTCAGAAGTAAGTTGGCCGCAGTGTTATCACT |
| | CATGGTTATGGCAGCACTGCATAATTCTCTTACTGTCATGCCATC |
| | CGTAAGATGCTTTTCTGTGACTGGTGAGTACTCAACCAAGTCATT |
| | CTGAGAATAGTGTATGCGGCGACCGAGTTGCTCTTGCCCGGCGTC |
| | AATACGGGATAATACCGCGCCACATAGCAGAACTTTAAAAGTGCT |
| | CATCATTGGAAAACGTTCTTCGGGGCGAAAACTCTCAAGGATCTT |
| | ACCGCTGTTGAGATCCAGTTCGATGTAACCCACTCGTGCACCCAA |
| | CTGATCTTCAGCATCTTTTACTTTCACCAGCGTTTCTGGGTGAGC |
| | AAAAACAGGAAGGCAAAATGCCGCAAAAAAGGGAATAAGGGCGAC |
| | ACGGAAATGTTGAATACTCATACTCTTCCTTTTTCAATATTATTG |
| | AAGCATTTATCAGGGTTATTGTCTCATGAGCGGATACATATTTGA |
| | ATGTATTTAGAAAAATAAACAAATAGGGGTTCCGCGCACATTTCC |
| | CCGAAAAGTGCCACCTGAAATTGTAAACGTTAATATTTTGTTAAA |
| | ATTCGCGTTAAATTTTTGTTAAATCAGCTCATTTTTTAACCAATA |
| | GGCCGAAATCGGCAAAATCCCTTATAAATCAAAAGAATAGACCGA |
| | GATAGGGTTGAGTGTTGTTCCAGTTTGGAACAAGAGTCCACTATT |
| | AAAGAACGTGGACTCCAACGTCAAAGGGCGAAAAACCGTCTATCA |
| | GGGCGATGGCCCACTACGTGAACCATCACCCTAATCAAGTTTTTT |
| | GGGGTCGAGGTGCCGTAAAGCACTAAATCGGAACCCTAAAGGGAG |
| | CCCCCGATTTAGAGCTTGACGGGGAAAGCCGGCGAACGTGGCGAG |
| | AAAGGAAGGGAAGAAAGCGAAAGGAGCGGGCGCTAGGGCGCTGGC |
| | AAGTGTAGCGGTCACGCTGCGCGTAACCACCACACCCGCCGCGCT |
| | TAATGCGCCGCTACAGGGCGCGTC |
| AMI154; | 372-512: Full ITR |
| SEQ ID | 570-873: CMV enhancer |
| NO: 65 | 874-1077: CMV promoter |
| | 1181-1277: SV40 intron |
| | 1282-1290: Kozak sequence |
| | 1291-1371: Aflibercept secretion sequence |
| | 1372-2664: Aflibercept coding sequence after optimization |
| | 2665-2682: Furin sequence |
| | 2683-2748: F2A sequence |
| | 2749-2805: Human IgG heavy chain secretion sequence |
| | 2806-3669: hCOMP-Ang1 coding sequence after optimization |
| | 3693-3741: Poly A sequence |
| | 3769-3909: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGTGAGCTACTGG |
| | GACACCGGCGTGCTGCTGTGCGCCCTGCTGAGCTGCCTGCTGCTG |
| | ACCGGCAGCAGCAGCGGCAGCGACACCGGCAGGCCCTTCGTGGAG |
| | ATGTACTCCGAGATCCCCGAGATCATCCACATGACCGAGGGCAGG |
| | GAGCTGGTGATCCCCTGCAGGGTGACCTCCCCCAACATCACCGTG |
| | ACCCTGAAGAAGTTCCCCCTGGACACCCTGATCCCCGACGGCAAG |
| | AGGATCATCTGGGACTCCAGGAAGGGCTTCATCATCTCCAACGCC |
| | ACCTACAAGGAGATCGGCCTGCTGACCTGCGAGGCCACCGTGAAC |
| | GGCCACCTGTACAAGACCAACTACCTGACCCACAGGCAGACCAAC |
| | ACCATCATCGACGTGGTGCTGTCCCCCTCCCACGGCATCGAGCTG |
| | TCCGTGGGCGAGAAGCTGGTGCTGAACTGCACCGCCAGGACCGAG |
| | CTGAACGTGGGCATCGACTTCAACTGGGAGTACCCCTCCTCCAAG |
| | CACCAGCACAAGAAGCTGGTGAACAGGGACCTGAAGACCCAGTCC |
| | GGCTCCGAGATGAAGAAGTTCCTGTCCACCCTGACCATCGACGGC |
| | GTGACCAGGTCCGACCAGGGCCTGTACACCTGCGCCGCCTCCTCC |
| | GGCCTGATGACCAAGAAGAACTCCACCTTCGTGAGGGTGCACGAG |
| | AAGGACAAGACCCACACCTGCCCCCCCTGCCCCGCCCCCGAGCTG |
| | CTGGGCGGCCCCTCCGTGTTCCTGTTCCCCCCCAAGCCCAAGGAC |
| | ACCCTGATGATCTCCAGGACCCCCGAGGTGACCTGCGTGGTGGTG |
| | GACGTGTCCCACGAGGACCCCGAGGTGAAGTTCAACTGGTACGTG |
| | GACGGCGTGGAGGTGCACAACGCCAAGACCAAGCCCAGGGAGGAG |
| | CAGTACAACTCCACCTACAGGGTGGTGTCCGTGCTGACCGTGCTG |
| | CACCAGGACTGGCTGAACGGCAAGGAGTACAAGTGCAAGGTGTCC |
| | AACAAGGCCCTGCCCGCCCCCATCGAGAAGACCATCTCCAAGGCC |
| | AAGGGCCAGCCCAGGGAGCCCCAGGTGTACACCCTGCCCCCCTCC |
| | AGGGACGAGCTGACCAAGAACCAGGTGTCCCTGACCTGCCTGGTG |
| | AAGGGCTTCTACCCCTCCGACATCGCCGTGGAGTGGGAGTCCAAC |
| | GGCCAGCCCGAGAACAACTACAAGACCACCCCCCCCGTGCTGGAC |
| | TCCGACGGCTCCTTCTTCCTGTACTCCAAGCTGACCGTGGACAAG |
| | TCCAGGTGGCAGCAGGGCAACGTGTTCTCCTGCTCCGTGATGCAC |
| | GAGGCCCTGCACAACCACTACACCCAGAAGTCCCTGTCCCTGTCC |
| | CCCGGCAAGAGAAGAAAGAGAGCCCCCGTGAAGCAGACCCTGAAC |
| | TTCGACCTGCTGAAGCTGGCCGGCGACGTGGAGAGCAACCCCGGC |
| | CCCATGGAGTTCGGCCTGAGCTGGCTGTTCCTGGTGGCCATCCTT |
| | AAGGGCGTGCAGTGCGACCTGGGCCCCCAGATGCTGAGAGAGCTG |
| | CAGGAGACCAACGCCGCCCTGCAGGACGTGAGAGAGCTGCTGAGA |
| | CAGCAGGTGAAGGAGATCACCTTCCTGAGAAACACCGTGATGGAG |
| | TGCGACGCCTGCGGCGACACCGTGCACAACCTGGTGAACCTGTGC |
| | ACCAAGGAGGGCGTGCTGCTGAAGGGCGGCAAGAGAGAGGAGGAG |
| | AAGCCCTTCAGAGACTGCGCCGACGTGTACCAGGCCGGCTTCAAC |
| | AAGAGCGGCATCTACACCATCTACATCAACAACATGCCCGAGCCC |
| | AAGAAGGTGTTCTGCAACATGGACGTGAACGGCGGCGGCTGGACC |
| | GTGATCCAGCACAGAGAGGACGGCAGCCTGGACTTCCAGAGAGGC |
| | TGGAAGGAGTACAAGATGGGCTTCGGCAACCCCAGCGGCGAGTAC |
| | TGGCTGGGCAACGAGTTCATCTTCGCCATCACCAGCCAGAGACAG |
| | TACATGCTGAGAATCGAGCTGATGGACTGGGAGGGCAACAGAGCC |
| | TACAGCCAGTACGACAGATTCCACATCGGCAACGAGAAGCAGAAC |
| | TACAGACTGTACCTGAAGGGCCACACCGGCACCGCCGGCAAGCAG |
| | AGCAGCCTGATCCTGCACGGCGCCGACTTCAGCACCAAGGACGCC |
| | GACAACGACAACTGCATGTGCAAGTGCGCCCTGATGCTGACCGGC |
| | GGCTGGTGGTTCGACGCCTGCGGCCCCAGCAACCTGAACGGCATG |
| | TTCTACACCGCCGGCCAGAACCACGGCAAGCTGAACGGCATCAAG |
| | TGGCACTACTTCAAGGGCCCCAGCTACAGCCTGAGAAGCACCACC |
| | ATGATGATCAGACCCCTGGACTTCTGAGCGGGACTCTGGAATTCG |
| | AAAATAAAATATCTTTATTTTCATTACATCTGTGTGTTGGTTTTT |
| | TGTGTGTTCTGCACGTGCGGACCGAGCGGCCGCAGGAACCCCTAG |
| | TGATGGAGTTGGCCACTCCCTCTCTGCGCGCTCGCTCGCTCACTG |
| | AGGCCGGGCGACCAAAGGTCGCCCGACGCCCGGGCTTTGCCCGGG |
| | CGGCCTCAGTGAGCGAGCGAGCGCGCAGCTGCCTGCAGGCATGCA |
| | AGCTGTAGCCAACCACTAGAACTATAGCTAGAGTCCTGGGCGAAC |
| | AAACGATGCTCGCCTTCCAGAAAACCGAGGATGCGAACCACTTCA |
| | TCCGGGGTCAGCACCACCGGCAAGCGCCGCGACGGCCGAGGTCTT |
| | CCGATCTCCTGAAGCCAGGGCAGATCCGTGCACAGCACCTTGCCG |
| | TAGAAGAACAGCAAGGCCGCCAATGCCTGACGATGCGTGGAGACC |
| | GAAACCTTGCGCTCGTTCGCCAGCCAGGACAGAAATGCCTCGACT |
| | TCGCTGCTGCCCAAGGTTGCCGGGTGACGCACACCGTGGAAACGG |
| | ATGAAGGCACGAACCCAGTTGACATAAGCCTGTTCGGTTCGTAAA |
| | CTGTAATGCAAGTAGCGTATGCGCTCACGCAACTGGTCCAGAACC |
| | TTGACCGAACGCAGCGGTGGTAACGGCGCAGTGGCGGTTTTCATG |
| | GCTTGTTATGACTGTTTTTTTGTACAGTCTATGCCTCGGGCATCC |
| | AAGCAGCAAGCGCGTTACGCCGTGGGTCGATGTTTGATGTTATGG |
| | AGCAGCAACGATGTTACGCAGCAGCAACGATGTTACGCAGCAGGG |
| | CAGTCGCCCTAAAACAAAGTTAGGTGGCTCAAGTATGGGCATCAT |
| | TCGCACATGTAGGCTCGGCCCTGACCAAGTCAAATCCATGCGGGC |
| | TGCTCTTGATCTTTTCGGTCGTGAGTTCGGAGACGTAGCCACCTA |
| | CTCCCAACATCAGCCGGACTCCGATTACCTCGGGAACTTGCTCCG |
| | TAGTAAGACATTCATCGCGCTTGCTGCCTTCGACCAAGAAGCGGT |
| | TGTTGGCGCTCTCGCGGCTTACGTTCTGCCCAGGTTTGAGCAGCC |
| | GCGTAGTGAGATCTATATCTATGATCTCGCAGTCTCCGGCGAGCA |
| | CCGGAGGCAGGGCATTGCCACCGCGCTCATCAATCTCCTCAAGCA |
| | TGAGGCCAACGCGCTTGGTGCTTATGTGATCTACGTGCAAGCAGA |
| | TTACGGTGACGATCCCGCAGTGGCTCTCTATACAAAGTTGGGCAT |
| | ACGGGAAGAAGTGATGCACTTTGATATCGACCCAAGTACCGCCAC |
| | CTAACAATTCGTTCAAGCCGAGATCGGCTTCCCGGCCGCGGAGTT |
| | GTTCGGTAAATTGTCACAACGCCGCGAATATAGTCTTTACCATGC |
| | CCTTGGCCACGCCCCTCTTTAATACGACGGGCAATTTGCACTTCA |
| | GAAAATGAAGAGTTTGCTTTAGCCATAACAAAAGTCCAGTATGCT |
| | TTTTCACAGCATAACTGGACTGATTTCAGTTTACAACTATTCTGT |
| | CTAGTTTAAGACTTTATTGTCATAGTTTAGATCTATTTTGTTCAG |
| | TTTAAGACTTTATTGTCCGCCCACACCCGCTTACGCAGGGCATCC |
| | ATTTATTACTCAACCGTAACCGATTTTGCCAGGTTACGCGGCTGG |
| | TCTGCGGTGTGAAATACCGCACAGATGCGTAAGGAGAAAATACCG |
| | CATCAGGCGCTCTTCCGCTTCCTCGCTCACTGACTCGCTGCGCTC |
| | GGTCGTTCGGCTGCGGCGAGCGGTATCAGCTCACTCAAAGGCGGT |
| | AATACGGTTATCCACAGAATCAGGGGATAACGCAGGAAAGAACAT |
| | GTGAGCAAAAGGCCAGCAAAAGGCCAGGAACCGTAAAAAGGCCGC |
| | GTTGCTGGCGTTTTTCCATAGGCTCCGCCCCCCTGACGAGCATCA |
| | CAAAAATCGACGCTCAAGTCAGAGGTGGCGAAACCCGACAGGACT |
| | ATAAAGATACCAGGCGTTTCCCCCTGGAAGCTCCCTCGTGCGCTC |
| | TCCTGTTCCGACCCTGCCGCTTACCGGATACCTGTCCGCCTTTCT |
| | CCCTTCGGGAAGCGTGGCGCTTTCTCAATGCTCACGCTGTAGGTA |
| | TCTCAGTTCGGTGTAGGTCGTTCGCTCCAAGCTGGGCTGTGTGCA |
| | CGAACCCCCCGTTCAGCCCGACCGCTGCGCCTTATCCGGTAACTA |
| | TCGTCTTGAGTCCAACCCGGTAAGACACGACTTATCGCCACTGGC |
| | AGCAGCCACTGGTAACAGGATTAGCAGAGCGAGGTATGTAGGCGG |
| | TGCTACAGAGTTCTTGAAGTGGTGGCCTAACTACGGCTACACTAG |
| | AAGGACAGTATTTGGTATCTGCGCTCTGCTGAAGCCAGTTACCTT |
| | CGGAAAAAGAGTTGGTAGCTCTTGATCCGGCAAACAAACCACCGC |
| | TGGTAGCGGTGGTTTTTTTGTTTGCAAGCAGCAGATTACGCGCAG |
| | AAAAAAAGGATCTCAAGAAGATCCTTTGATCTTTTCTACGGGGTC |
| | TGACGCTCAGTGGAACGAAAACTCACGTTAAGGGATTTTGGTCAT |
| | GAGATTATCAAAAAGGATCTTCACCTAGATCCTTTTAAATTAAAA |
| | ATGAAGTTTTAAATCAATCTAAAGTATATATGAGTAAACTTGGTC |
| | TGACAGTTACCAATGCTTAATCAGTGAGGCACCTATCTCAGCGAT |
| | CTGTCTATTTCGTTCATCCATAGTTGCCTGACTCCCCGTCGTGTA |
| | GATAACTACGATACGGGAGGGCTTACCATCTGGCCCCAGTGCTGC |
| | AATGATACCGCGAGACCCACGCTCACCGGCTCCAGATTTATCAGC |
| | AATAAACCAGCCAGCCGGAAGGGCCGAGCGCAGAAGTGGTCCTGC |
| | AACTTTATCCGCCTCCATCCAGTCTATTAATTGTTGCCGGGAAGC |
| | TAGAGTAAGTAGTTCGCCAGTTAATAGTTTGCGCAACGTTGTTGC |
| | CATTGCTACAGGCATCGTGGTGTCACGCTCGTCGTTTGGTATGGC |
| | TTCATTCAGCTCCGGTTCCCAACGATCAAGGCGAGTTACATGATC |
| | CCCCATGTTGTGCAAAAAAGCGGTTAGCTCCTTCGGTCCTCCGAT |
| | CGTTGTCAGAAGTAAGTTGGCCGCAGTGTTATCACTCATGGTTAT |
| | GGCAGCACTGCATAATTCTCTTACTGTCATGCCATCCGTAAGATG |
| | CTTTTCTGTGACTGGTGAGTACTCAACCAAGTCATTCTGAGAATA |
| | GTGTATGCGGCGACCGAGTTGCTCTTGCCCGGCGTCAATACGGGA |
| | TAATACCGCGCCACATAGCAGAACTTTAAAAGTGCTCATCATTGG |
| | AAAACGTTCTTCGGGGCGAAAACTCTCAAGGATCTTACCGCTGTT |
| | GAGATCCAGTTCGATGTAACCCACTCGTGCACCCAACTGATCTTC |
| | AGCATCTTTTACTTTCACCAGCGTTTCTGGGTGAGCAAAAACAGG |
| | AAGGCAAAATGCCGCAAAAAAGGGAATAAGGGCGACACGGAAATG |
| | TTGAATACTCATACTCTTCCTTTTTCAATATTATTGAAGCATTTA |
| | TCAGGGTTATTGTCTCATGAGCGGATACATATTTGAATGTATTTA |
| | GAAAAATAAACAAATAGGGGTTCCGCGCACATTTCCCCGAAAAGT |
| | GCCACCTGAAATTGTAAACGTTAATATTTTGTTAAAATTCGCGTT |
| | AAATTTTTGTTAAATCAGCTCATTTTTTAACCAATAGGCCGAAAT |
| | CGGCAAAATCCCTTATAAATCAAAAGAATAGACCGAGATAGGGTT |
| | GAGTGTTGTTCCAGTTTGGAACAAGAGTCCACTATTAAAGAACGT |
| | GGACTCCAACGTCAAAGGGCGAAAAACCGTCTATCAGGGCGATGG |
| | CCCACTACGTGAACCATCACCCTAATCAAGTTTTTTGGGGTCGAG |
| | GTGCCGTAAAGCACTAAATCGGAACCCTAAAGGGAGCCCCCGATT |
| | TAGAGCTTGACGGGGAAAGCCGGCGAACGTGGCGAGAAAGGAAGG |
| | GAAGAAAGCGAAAGGAGCGGGCGCTAGGGCGCTGGCAAGTGTAGC |
| | GGTCACGCTGCGCGTAACCACCACACCCGCCGCGCTTAATGCGCC |
| | GCTACAGGGCGCGTC |
| AMI155; | 372-512: Full ITR |
| SEQ ID | 570-873: CMV enhancer |
| NO: 66 | 874-1077: CMV promoter |
| | 1181-1277: SV40 intron |
| | 1282-1290: Kozak sequence |
| | 1291-1347: Human IgG heavy chain secretion sequence |
| | 1348-1710: Lucentis Vh coding sequence after optimization |
| | 1711-1770: 4xGGGGS sequence |
| | 1771-2106: Lucentis VI coding sequence after optimization |
| | 2107-2124: Furin sequence |
| | 2125-2190:F2A sequence |
| | 2191-2247: Human IgG heavy chain secretion sequence |
| | 2248-3111: hCOMP-Ang1 coding sequence after optimization |
| | 3135-3183: Poly A sequence |
| | 3211-3351: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGAGTTCGGCCTG |
| | AGCTGGCTGTTCCTGGTGGCCATCCTGAAGGGCGTGCAGTGCGAG |
| | GTGCAGCTGGTGGAGAGCGGCGGCGGCCTGGTGCAGCCCGGCGGC |
| | AGCCTGAGACTGAGCTGCGCCGCCAGCGGCTACGACTTCACCCAC |
| | TACGGCATGAACTGGGTGAGACAGGCCCCCGGCAAGGGCCTGGAG |
| | TGGGTGGGCTGGATCAACACCTACACCGGCGAGCCCACCTACGCC |
| | GCCGACTTCAAGAGAAGATTCACCTTCAGCCTGGACACCAGCAAG |
| | AGCACCGCCTACCTGCAGATGAACAGCCTGAGAGCCGAGGACACC |
| | GCCGTGTACTACTGCGCCAAGTACCCCTACTACTACGGCACCAGC |
| | CACTGGTACTTCGACGTGTGGGGCCAGGGCACCCTGGTGACCGTG |
| | GGCGGAGGCGGAAGCGGCGGAGGCGGATCTGGCGGAGGCGGCAGC |
| | GGCGGCGGCGGCTCTGACATCCAGCTGACCCAGAGCCCCAGCAGC |
| | CTGAGCGCCAGCGTGGGCGACAGAGTGACCATCACCTGCAGCGCC |
| | AGCCAGGACATCAGCAACTACCTGAACTGGTACCAGCAGAAGCCC |
| | GGCAAGGCCCCCAAGGTGCTGATCTACTTCACCAGCAGCCTGCAC |
| | AGCGGCGTGCCCAGCAGATTCAGCGGCAGCGGCAGCGGCACCGAC |
| | TTCACCCTGACCATCAGCAGCCTGCAGCCCGAGGACTTCGCCACC |
| | TACTACTGCCAGCAGTACAGCACCGTGCCCTGGACCTTCGGCCAG |
| | GGCACCAAGGTGGAGATCAAGAGAACCGTGGCCGCCAGAAGAAAG |
| | AGAGCCCCCGTGAAGCAGACCCTGAACTTCGACCTGCTGAAGCTG |
| | GCCGGCGACGTGGAGAGCAACCCCGGCCCCATGGAGTTCGGCCTG |
| | AGCTGGCTGTTCCTGGTGGCCATCCTTAAGGGCGTGCAGTGCGAC |
| | CTGGGCCCCCAGATGCTGAGAGAGCTGCAGGAGACCAACGCCGCC |
| | CTGCAGGACGTGAGAGAGCTGCTGAGACAGCAGGTGAAGGAGATC |
| | ACCTTCCTGAGAAACACCGTGATGGAGTGCGACGCCTGCGGCGAC |
| | ACCGTGCACAACCTGGTGAACCTGTGCACCAAGGAGGGCGTGCTG |
| | CTGAAGGGCGGCAAGAGAGAGGAGGAGAAGCCCTTCAGAGACTGC |
| | GCCGACGTGTACCAGGCCGGCTTCAACAAGAGCGGCATCTACACC |
| | ATCTACATCAACAACATGCCCGAGCCCAAGAAGGTGTTCTGCAAC |
| | ATGGACGTGAACGGCGGCGGCTGGACCGTGATCCAGCACAGAGAG |
| | GACGGCAGCCTGGACTTCCAGAGAGGCTGGAAGGAGTACAAGATG |
| | GGCTTCGGCAACCCCAGCGGCGAGTACTGGCTGGGCAACGAGTTC |
| | ATCTTCGCCATCACCAGCCAGAGACAGTACATGCTGAGAATCGAG |
| | CTGATGGACTGGGAGGGCAACAGAGCCTACAGCCAGTACGACAGA |
| | TTCCACATCGGCAACGAGAAGCAGAACTACAGACTGTACCTGAAG |
| | GGCCACACCGGCACCGCCGGCAAGCAGAGCAGCCTGATCCTGCAC |
| | GGCGCCGACTTCAGCACCAAGGACGCCGACAACGACAACTGCATG |
| | TGCAAGTGCGCCCTGATGCTGACCGGCGGCTGGTGGTTCGACGCC |
| | TGCGGCCCCAGCAACCTGAACGGCATGTTCTACACCGCCGGCCAG |
| | AACCACGGCAAGCTGAACGGCATCAAGTGGCACTACTTCAAGGGC |
| | CCCAGCTACAGCCTGAGAAGCACCACCATGATGATCAGACCCCTG |
| | GACTTCTGAGCGGGACTCTGGAATTCGAAAATAAAATATCTTTAT |
| | TTTCATTACATCTGTGTGTTGGTTTTTTGTGTGTTCTGCACGTGC |
| | GGACCGAGCGGCCGCAGGAACCCCTAGTGATGGAGTTGGCCACTC |
| | CCTCTCTGCGCGCTCGCTCGCTCACTGAGGCCGGGCGACCAAAGG |
| | TCGCCCGACGCCCGGGCTTTGCCCGGGCGGCCTCAGTGAGCGAGC |
| | GAGCGCGCAGCTGCCTGCAGGCATGCAAGCTGTAGCCAACCACTA |
| | GAACTATAGCTAGAGTCCTGGGCGAACAAACGATGCTCGCCTTCC |
| | AGAAAACCGAGGATGCGAACCACTTCATCCGGGGTCAGCACCACC |
| | GGCAAGCGCCGCGACGGCCGAGGTCTTCCGATCTCCTGAAGCCAG |
| | GGCAGATCCGTGCACAGCACCTTGCCGTAGAAGAACAGCAAGGCC |
| | GCCAATGCCTGACGATGCGTGGAGACCGAAACCTTGCGCTCGTTC |
| | GCCAGCCAGGACAGAAATGCCTCGACTTCGCTGCTGCCCAAGGTT |
| | GCCGGGTGACGCACACCGTGGAAACGGATGAAGGCACGAACCCAG |
| | TTGACATAAGCCTGTTCGGTTCGTAAACTGTAATGCAAGTAGCGT |
| | ATGCGCTCACGCAACTGGTCCAGAACCTTGACCGAACGCAGCGGT |
| | GGTAACGGCGCAGTGGCGGTTTTCATGGCTTGTTATGACTGTTTT |
| | TTTGTACAGTCTATGCCTCGGGCATCCAAGCAGCAAGCGCGTTAC |
| | GCCGTGGGTCGATGTTTGATGTTATGGAGCAGCAACGATGTTACG |
| | CAGCAGCAACGATGTTACGCAGCAGGGCAGTCGCCCTAAAACAAA |
| | GTTAGGTGGCTCAAGTATGGGCATCATTCGCACATGTAGGCTCGG |
| | CCCTGACCAAGTCAAATCCATGCGGGCTGCTCTTGATCTTTTCGG |
| | TCGTGAGTTCGGAGACGTAGCCACCTACTCCCAACATCAGCCGGA |
| | CTCCGATTACCTCGGGAACTTGCTCCGTAGTAAGACATTCATCGC |
| | GCTTGCTGCCTTCGACCAAGAAGCGGTTGTTGGCGCTCTCGCGGC |
| | TTACGTTCTGCCCAGGTTTGAGCAGCCGCGTAGTGAGATCTATAT |
| | CTATGATCTCGCAGTCTCCGGCGAGCACCGGAGGCAGGGCATTGC |
| | CACCGCGCTCATCAATCTCCTCAAGCATGAGGCCAACGCGCTTGG |
| | TGCTTATGTGATCTACGTGCAAGCAGATTACGGTGACGATCCCGC |
| | AGTGGCTCTCTATACAAAGTTGGGCATACGGGAAGAAGTGATGCA |
| | CTTTGATATCGACCCAAGTACCGCCACCTAACAATTCGTTCAAGC |
| | CGAGATCGGCTTCCCGGCCGCGGAGTTGTTCGGTAAATTGTCACA |
| | ACGCCGCGAATATAGTCTTTACCATGCCCTTGGCCACGCCCCTCT |
| | TTAATACGACGGGCAATTTGCACTTCAGAAAATGAAGAGTTTGCT |
| | TTAGCCATAACAAAAGTCCAGTATGCTTTTTCACAGCATAACTGG |
| | ACTGATTTCAGTTTACAACTATTCTGTCTAGTTTAAGACTTTATT |
| | GTCATAGTTTAGATCTATTTTGTTCAGTTTAAGACTTTATTGTCC |
| | GCCCACACCCGCTTACGCAGGGCATCCATTTATTACTCAACCGTA |
| | ACCGATTTTGCCAGGTTACGCGGCTGGTCTGCGGTGTGAAATACC |
| | GCACAGATGCGTAAGGAGAAAATACCGCATCAGGCGCTCTTCCGC |
| | TTCCTCGCTCACTGACTCGCTGCGCTCGGTCGTTCGGCTGCGGCG |
| | AGCGGTATCAGCTCACTCAAAGGCGGTAATACGGTTATCCACAGA |
| | ATCAGGGGATAACGCAGGAAAGAACATGTGAGCAAAAGGCCAGCA |
| | AAAGGCCAGGAACCGTAAAAAGGCCGCGTTGCTGGCGTTTTTCCA |
| | TAGGCTCCGCCCCCCTGACGAGCATCACAAAAATCGACGCTCAAG |
| | TCAGAGGTGGCGAAACCCGACAGGACTATAAAGATACCAGGCGTT |
| | TCCCCCTGGAAGCTCCCTCGTGCGCTCTCCTGTTCCGACCCTGCC |
| | GCTTACCGGATACCTGTCCGCCTTTCTCCCTTCGGGAAGCGTGGC |
| | GCTTTCTCAATGCTCACGCTGTAGGTATCTCAGTTCGGTGTAGGT |
| | CGTTCGCTCCAAGCTGGGCTGTGTGCACGAACCCCCCGTTCAGCC |
| | CGACCGCTGCGCCTTATCCGGTAACTATCGTCTTGAGTCCAACCC |
| | GGTAAGACACGACTTATCGCCACTGGCAGCAGCCACTGGTAACAG |
| | GATTAGCAGAGCGAGGTATGTAGGCGGTGCTACAGAGTTCTTGAA |
| | GTGGTGGCCTAACTACGGCTACACTAGAAGGACAGTATTTGGTAT |
| | CTGCGCTCTGCTGAAGCCAGTTACCTTCGGAAAAAGAGTTGGTAG |
| | CTCTTGATCCGGCAAACAAACCACCGCTGGTAGCGGTGGTTTTTT |
| | TGTTTGCAAGCAGCAGATTACGCGCAGAAAAAAAGGATCTCAAGA |
| | AGATCCTTTGATCTTTTCTACGGGGTCTGACGCTCAGTGGAACGA |
| | AAACTCACGTTAAGGGATTTTGGTCATGAGATTATCAAAAAGGAT |
| | CTTCACCTAGATCCTTTTAAATTAAAAATGAAGTTTTAAATCAAT |
| | CTAAAGTATATATGAGTAAACTTGGTCTGACAGTTACCAATGCTT |
| | AATCAGTGAGGCACCTATCTCAGCGATCTGTCTATTTCGTTCATC |
| | CATAGTTGCCTGACTCCCCGTCGTGTAGATAACTACGATACGGGA |
| | GGGCTTACCATCTGGCCCCAGTGCTGCAATGATACCGCGAGACCC |
| | ACGCTCACCGGCTCCAGATTTATCAGCAATAAACCAGCCAGCCGG |
| | AAGGGCCGAGCGCAGAAGTGGTCCTGCAACTTTATCCGCCTCCAT |
| | CCAGTCTATTAATTGTTGCCGGGAAGCTAGAGTAAGTAGTTCGCC |
| | AGTTAATAGTTTGCGCAACGTTGTTGCCATTGCTACAGGCATCGT |
| | GGTGTCACGCTCGTCGTTTGGTATGGCTTCATTCAGCTCCGGTTC |
| | CCAACGATCAAGGCGAGTTACATGATCCCCCATGTTGTGCAAAAA |
| | AGCGGTTAGCTCCTTCGGTCCTCCGATCGTTGTCAGAAGTAAGTT |
| | GGCCGCAGTGTTATCACTCATGGTTATGGCAGCACTGCATAATTC |
| | TCTTACTGTCATGCCATCCGTAAGATGCTTTTCTGTGACTGGTGA |
| | GTACTCAACCAAGTCATTCTGAGAATAGTGTATGCGGCGACCGAG |
| | TTGCTCTTGCCCGGCGTCAATACGGGATAATACCGCGCCACATAG |
| | CAGAACTTTAAAAGTGCTCATCATTGGAAAACGTTCTTCGGGGCG |
| | AAAACTCTCAAGGATCTTACCGCTGTTGAGATCCAGTTCGATGTA |
| | ACCCACTCGTGCACCCAACTGATCTTCAGCATCTTTTACTTTCAC |
| | CAGCGTTTCTGGGTGAGCAAAAACAGGAAGGCAAAATGCCGCAAA |
| | AAAGGGAATAAGGGCGACACGGAAATGTTGAATACTCATACTCTT |
| | CCTTTTTCAATATTATTGAAGCATTTATCAGGGTTATTGTCTCAT |
| | GAGCGGATACATATTTGAATGTATTTAGAAAAATAAACAAATAGG |
| | GGTTCCGCGCACATTTCCCCGAAAAGTGCCACCTGAAATTGTAAA |
| | CGTTAATATTTTGTTAAAATTCGCGTTAAATTTTTGTTAAATCAG |
| | CTCATTTTTTAACCAATAGGCCGAAATCGGCAAAATCCCTTATAA |
| | ATCAAAAGAATAGACCGAGATAGGGTTGAGTGTTGTTCCAGTTTG |
| | GAACAAGAGTCCACTATTAAAGAACGTGGACTCCAACGTCAAAGG |
| | GCGAAAAACCGTCTATCAGGGCGATGGCCCACTACGTGAACCATC |
| | ACCCTAATCAAGTTTTTTGGGGTCGAGGTGCCGTAAAGCACTAAA |
| | TCGGAACCCTAAAGGGAGCCCCCGATTTAGAGCTTGACGGGGAAA |
| | GCCGGCGAACGTGGCGAGAAAGGAAGGGAAGAAAGCGAAAGGAGC |
| | GGGCGCTAGGGCGCTGGCAAGTGTAGCGGTCACGCTGCGCGTAAC |
| | CACCACACCCGCCGCGCTTAATGCGCCGCTACAGGGCGCGTC |
| AMI156; | 372-512: Full ITR |
| SEQ ID | 570-873: CMV enhancer |
| NO: 67 | 874-1077: CMV promoter |
| | 1181-1277: SV40 intron |
| | 1282-1290: Kozak sequence |
| | 1291-1347: Human IgG heavy chain secretion sequence |
| | 1348-1710: Lucentis Vh coding sequence after optimization |
| | 1711-1770: 4xGGGGS sequence |
| | 1771-2106: Lucentis VI coding sequence after optimization |
| | 2117-2165: Poly A sequence |
| | 2172-2475: CMV enhancer |
| | 2476-2679: CMV promoter |
| | 2783-2879: SV40 intron |
| | 2884-2892: kozak sequence |
| | 2893-2949: Human IgG heavy chain secretion sequence |
| | 2950-3813: hCOMP-Ang1 coding sequence after optimization |
| | 3837-3885: Poly A sequence |
| | 3913-4053: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGAGTTCGGCCTG |
| | AGCTGGCTGTTCCTGGTGGCCATCCTGAAGGGCGTGCAGTGCGAG |
| | GTGCAGCTGGTGGAGAGCGGCGGCGGCCTGGTGCAGCCCGGCGGC |
| | AGCCTGAGACTGAGCTGCGCCGCCAGCGGCTACGACTTCACCCAC |
| | TACGGCATGAACTGGGTGAGACAGGCCCCCGGCAAGGGCCTGGAG |
| | TGGGTGGGCTGGATCAACACCTACACCGGCGAGCCCACCTACGCC |
| | GCCGACTTCAAGAGAAGATTCACCTTCAGCCTGGACACCAGCAAG |
| | AGCACCGCCTACCTGCAGATGAACAGCCTGAGAGCCGAGGACACC |
| | GCCGTGTACTACTGCGCCAAGTACCCCTACTACTACGGCACCAGC |
| | CACTGGTACTTCGACGTGTGGGGCCAGGGCACCCTGGTGACCGTG |
| | GGCGGAGGCGGAAGCGGCGGAGGCGGATCTGGCGGAGGCGGCAGC |
| | GGCGGCGGCGGCTCTGACATCCAGCTGACCCAGAGCCCCAGCAGC |
| | CTGAGCGCCAGCGTGGGCGACAGAGTGACCATCACCTGCAGCGCC |
| | AGCCAGGACATCAGCAACTACCTGAACTGGTACCAGCAGAAGCCC |
| | GGCAAGGCCCCCAAGGTGCTGATCTACTTCACCAGCAGCCTGCAC |
| | AGCGGCGTGCCCAGCAGATTCAGCGGCAGCGGCAGCGGCACCGAC |
| | TTCACCCTGACCATCAGCAGCCTGCAGCCCGAGGACTTCGCCACC |
| | TACTACTGCCAGCAGTACAGCACCGTGCCCTGGACCTTCGGCCAG |
| | GGCACCAAGGTGGAGATCAAGAGAACCGTGGCCGCCTGATTCGAA |
| | AAATAAAATATCTTTATTTTCATTACATCTGTGTGTTGGTTTTTT |
| | GTGTGGGTACCCGTTACATAACTTACGGTAAATGGCCCGCCTGGC |
| | TGACCGCCCAACGACCCCCGCCCATTGACGTCAATAATGACGTAT |
| | GTTCCCATAGTAACGCCAATAGGGACTTTCCATTGACGTCAATGG |
| | GTGGAGTATTTACGGTAAACTGCCCACTTGGCAGTACATCAAGTG |
| | TATCATATGCCAAGTACGCCCCCTATTGACGTCAATGACGGTAAA |
| | TGGCCCGCCTGGCATTATGCCCAGTACATGACCTTATGGGACTTT |
| | CCTACTTGGCAGTACATCTACGTATTAGTCATCGCTATTACCATG |
| | GTGATGCGGTTTTGGCAGTACATCAATGGGCGTGGATAGCGGTTT |
| | GACTCACGGGGATTTCCAAGTCTCCACCCCATTGACGTCAATGGG |
| | AGTTTGTTTTGGCACCAAAATCAACGGGACTTTCCAAAATGTCGT |
| | AACAACTCCGCCCCATTGACGCAAATGGGCGGTAGGCGTGTACGG |
| | TGGGAGGTCTATATAAGCAGAGCTCGTTTAGTGAACCGTCAGATC |
| | GCCTGGAGACGCCATCCACGCTGTTTTGACCTCCATAGAAGACAC |
| | CGGGACCGATCCAGCCTCCGGACTCTAGAGTTAACTGGTAAGTTT |
| | AGTCTTTTTGTCTTTTATTTCAGGTCCCGGATCCGGTGGTGGTGC |
| | AAATCAAAGAACTGCTCCTCAGTGGATGTTGCCTTTACTTCTAGG |
| | CCTGCCGCCACCATGGAGTTCGGCCTGAGCTGGCTGTTCCTGGTG |
| | GCCATCCTTAAGGGCGTGCAGTGCGACCTGGGCCCCCAGATGCTG |
| | AGAGAGCTGCAGGAGACCAACGCCGCCCTGCAGGACGTGAGAGAG |
| | CTGCTGAGACAGCAGGTGAAGGAGATCACCTTCCTGAGAAACACC |
| | GTGATGGAGTGCGACGCCTGCGGCGACACCGTGCACAACCTGGTG |
| | AACCTGTGCACCAAGGAGGGCGTGCTGCTGAAGGGCGGCAAGAGA |
| | GAGGAGGAGAAGCCCTTCAGAGACTGCGCCGACGTGTACCAGGCC |
| | GGCTTCAACAAGAGCGGCATCTACACCATCTACATCAACAACATG |
| | CCCGAGCCCAAGAAGGTGTTCTGCAACATGGACGTGAACGGCGGC |
| | GGCTGGACCGTGATCCAGCACAGAGAGGACGGCAGCCTGGACTTC |
| | CAGAGAGGCTGGAAGGAGTACAAGATGGGCTTCGGCAACCCCAGC |
| | GGCGAGTACTGGCTGGGCAACGAGTTCATCTTCGCCATCACCAGC |
| | CAGAGACAGTACATGCTGAGAATCGAGCTGATGGACTGGGAGGGC |
| | AACAGAGCCTACAGCCAGTACGACAGATTCCACATCGGCAACGAG |
| | AAGCAGAACTACAGACTGTACCTGAAGGGCCACACCGGCACCGCC |
| | GGCAAGCAGAGCAGCCTGATCCTGCACGGCGCCGACTTCAGCACC |
| | AAGGACGCCGACAACGACAACTGCATGTGCAAGTGCGCCCTGATG |
| | CTGACCGGCGGCTGGTGGTTCGACGCCTGCGGCCCCAGCAACCTG |
| | AACGGCATGTTCTACACCGCCGGCCAGAACCACGGCAAGCTGAAC |
| | GGCATCAAGTGGCACTACTTCAAGGGCCCCAGCTACAGCCTGAGA |
| | AGCACCACCATGATGATCAGACCCCTGGACTTCTGAGCGGGACTC |
| | TGGAATTCGAAAATAAAATATCTTTATTTTCATTACATCTGTGTG |
| | TTGGTTTTTTGTGTGTTCTGCACGTGCGGACCGAGCGGCCGCAGG |
| | AACCCCTAGTGATGGAGTTGGCCACTCCCTCTCTGCGCGCTCGCT |
| | CGCTCACTGAGGCCGGGCGACCAAAGGTCGCCCGACGCCCGGGCT |
| | TTGCCCGGGCGGCCTCAGTGAGCGAGCGAGCGCGCAGCTGCCTGC |
| | AGGCATGCAAGCTGTAGCCAACCACTAGAACTATAGCTAGAGTCC |
| | TGGGCGAACAAACGATGCTCGCCTTCCAGAAAACCGAGGATGCGA |
| | ACCACTTCATCCGGGGTCAGCACCACCGGCAAGCGCCGCGACGGC |
| | CGAGGTCTTCCGATCTCCTGAAGCCAGGGCAGATCCGTGCACAGC |
| | ACCTTGCCGTAGAAGAACAGCAAGGCCGCCAATGCCTGACGATGC |
| | GTGGAGACCGAAACCTTGCGCTCGTTCGCCAGCCAGGACAGAAAT |
| | GCCTCGACTTCGCTGCTGCCCAAGGTTGCCGGGTGACGCACACCG |
| | TGGAAACGGATGAAGGCACGAACCCAGTTGACATAAGCCTGTTCG |
| | GTTCGTAAACTGTAATGCAAGTAGCGTATGCGCTCACGCAACTGG |
| | TCCAGAACCTTGACCGAACGCAGCGGTGGTAACGGCGCAGTGGCG |
| | GTTTTCATGGCTTGTTATGACTGTTTTTTTGTACAGTCTATGCCT |
| | CGGGCATCCAAGCAGCAAGCGCGTTACGCCGTGGGTCGATGTTTG |
| | ATGTTATGGAGCAGCAACGATGTTACGCAGCAGCAACGATGTTAC |
| | GCAGCAGGGCAGTCGCCCTAAAACAAAGTTAGGTGGCTCAAGTAT |
| | GGGCATCATTCGCACATGTAGGCTCGGCCCTGACCAAGTCAAATC |
| | CATGCGGGCTGCTCTTGATCTTTTCGGTCGTGAGTTCGGAGACGT |
| | AGCCACCTACTCCCAACATCAGCCGGACTCCGATTACCTCGGGAA |
| | CTTGCTCCGTAGTAAGACATTCATCGCGCTTGCTGCCTTCGACCA |
| | AGAAGCGGTTGTTGGCGCTCTCGCGGCTTACGTTCTGCCCAGGTT |
| | TGAGCAGCCGCGTAGTGAGATCTATATCTATGATCTCGCAGTCTC |
| | CGGCGAGCACCGGAGGCAGGGCATTGCCACCGCGCTCATCAATCT |
| | CCTCAAGCATGAGGCCAACGCGCTTGGTGCTTATGTGATCTACGT |
| | GCAAGCAGATTACGGTGACGATCCCGCAGTGGCTCTCTATACAAA |
| | GTTGGGCATACGGGAAGAAGTGATGCACTTTGATATCGACCCAAG |
| | TACCGCCACCTAACAATTCGTTCAAGCCGAGATCGGCTTCCCGGC |
| | CGCGGAGTTGTTCGGTAAATTGTCACAACGCCGCGAATATAGTCT |
| | TTACCATGCCCTTGGCCACGCCCCTCTTTAATACGACGGGCAATT |
| | TGCACTTCAGAAAATGAAGAGTTTGCTTTAGCCATAACAAAAGTC |
| | CAGTATGCTTTTTCACAGCATAACTGGACTGATTTCAGTTTACAA |
| | CTATTCTGTCTAGTTTAAGACTTTATTGTCATAGTTTAGATCTAT |
| | TTTGTTCAGTTTAAGACTTTATTGTCCGCCCACACCCGCTTACGC |
| | AGGGCATCCATTTATTACTCAACCGTAACCGATTTTGCCAGGTTA |
| | CGCGGCTGGTCTGCGGTGTGAAATACCGCACAGATGCGTAAGGAG |
| | AAAATACCGCATCAGGCGCTCTTCCGCTTCCTCGCTCACTGACTC |
| | GCTGCGCTCGGTCGTTCGGCTGCGGCGAGCGGTATCAGCTCACTC |
| | AAAGGCGGTAATACGGTTATCCACAGAATCAGGGGATAACGCAGG |
| | AAAGAACATGTGAGCAAAAGGCCAGCAAAAGGCCAGGAACCGTAA |
| | AAAGGCCGCGTTGCTGGCGTTTTTCCATAGGCTCCGCCCCCCTGA |
| | CGAGCATCACAAAAATCGACGCTCAAGTCAGAGGTGGCGAAACCC |
| | GACAGGACTATAAAGATACCAGGCGTTTCCCCCTGGAAGCTCCCT |
| | CGTGCGCTCTCCTGTTCCGACCCTGCCGCTTACCGGATACCTGTC |
| | CGCCTTTCTCCCTTCGGGAAGCGTGGCGCTTTCTCAATGCTCACG |
| | CTGTAGGTATCTCAGTTCGGTGTAGGTCGTTCGCTCCAAGCTGGG |
| | CTGTGTGCACGAACCCCCCGTTCAGCCCGACCGCTGCGCCTTATC |
| | CGGTAACTATCGTCTTGAGTCCAACCCGGTAAGACACGACTTATC |
| | GCCACTGGCAGCAGCCACTGGTAACAGGATTAGCAGAGCGAGGTA |
| | TGTAGGCGGTGCTACAGAGTTCTTGAAGTGGTGGCCTAACTACGG |
| | CTACACTAGAAGGACAGTATTTGGTATCTGCGCTCTGCTGAAGCC |
| | AGTTACCTTCGGAAAAAGAGTTGGTAGCTCTTGATCCGGCAAACA |
| | AACCACCGCTGGTAGCGGTGGTTTTTTTGTTTGCAAGCAGCAGAT |
| | TACGCGCAGAAAAAAAGGATCTCAAGAAGATCCTTTGATCTTTTC |
| | TACGGGGTCTGACGCTCAGTGGAACGAAAACTCACGTTAAGGGAT |
| | TTTGGTCATGAGATTATCAAAAAGGATCTTCACCTAGATCCTTTT |
| | AAATTAAAAATGAAGTTTTAAATCAATCTAAAGTATATATGAGTA |
| | AACTTGGTCTGACAGTTACCAATGCTTAATCAGTGAGGCACCTAT |
| | CTCAGCGATCTGTCTATTTCGTTCATCCATAGTTGCCTGACTCCC |
| | CGTCGTGTAGATAACTACGATACGGGAGGGCTTACCATCTGGCCC |
| | CAGTGCTGCAATGATACCGCGAGACCCACGCTCACCGGCTCCAGA |
| | TTTATCAGCAATAAACCAGCCAGCCGGAAGGGCCGAGCGCAGAAG |
| | TGGTCCTGCAACTTTATCCGCCTCCATCCAGTCTATTAATTGTTG |
| | CCGGGAAGCTAGAGTAAGTAGTTCGCCAGTTAATAGTTTGCGCAA |
| | CGTTGTTGCCATTGCTACAGGCATCGTGGTGTCACGCTCGTCGTT |
| | TGGTATGGCTTCATTCAGCTCCGGTTCCCAACGATCAAGGCGAGT |
| | TACATGATCCCCCATGTTGTGCAAAAAAGCGGTTAGCTCCTTCGG |
| | TCCTCCGATCGTTGTCAGAAGTAAGTTGGCCGCAGTGTTATCACT |
| | CATGGTTATGGCAGCACTGCATAATTCTCTTACTGTCATGCCATC |
| | CGTAAGATGCTTTTCTGTGACTGGTGAGTACTCAACCAAGTCATT |
| | CTGAGAATAGTGTATGCGGCGACCGAGTTGCTCTTGCCCGGCGTC |
| | AATACGGGATAATACCGCGCCACATAGCAGAACTTTAAAAGTGCT |
| | CATCATTGGAAAACGTTCTTCGGGGCGAAAACTCTCAAGGATCTT |
| | ACCGCTGTTGAGATCCAGTTCGATGTAACCCACTCGTGCACCCAA |
| | CTGATCTTCAGCATCTTTTACTTTCACCAGCGTTTCTGGGTGAGC |
| | AAAAACAGGAAGGCAAAATGCCGCAAAAAAGGGAATAAGGGCGAC |
| | ACGGAAATGTTGAATACTCATACTCTTCCTTTTTCAATATTATTG |
| | AAGCATTTATCAGGGTTATTGTCTCATGAGCGGATACATATTTGA |
| | ATGTATTTAGAAAAATAAACAAATAGGGGTTCCGCGCACATTTCC |
| | CCGAAAAGTGCCACCTGAAATTGTAAACGTTAATATTTTGTTAAA |
| | ATTCGCGTTAAATTTTTGTTAAATCAGCTCATTTTTTAACCAATA |
| | GGCCGAAATCGGCAAAATCCCTTATAAATCAAAAGAATAGACCGA |
| | GATAGGGTTGAGTGTTGTTCCAGTTTGGAACAAGAGTCCACTATT |
| | AAAGAACGTGGACTCCAACGTCAAAGGGCGAAAAACCGTCTATCA |
| | GGGCGATGGCCCACTACGTGAACCATCACCCTAATCAAGTTTTTT |
| | GGGGTCGAGGTGCCGTAAAGCACTAAATCGGAACCCTAAAGGGAG |
| | CCCCCGATTTAGAGCTTGACGGGGAAAGCCGGCGAACGTGGCGAG |
| | AAAGGAAGGGAAGAAAGCGAAAGGAGCGGGCGCTAGGGCGCTGGC |
| | AAGTGTAGCGGTCACGCTGCGCGTAACCACCACACCCGCCGCGCT |
| | TAATGCGCCGCTACAGGGCGCGTC |
| AMI157; | 611-755: Full ITR |
| SEQ ID | 801-1104: CMV enhancer |
| NO: 68 | 1105-1308: CMV promoter |
| | 1412-1508: SV40 intron |
| | 1513-1521: Kozak sequence |
| | 1522-1578: Human IgG heavy chain secretion sequence |
| | 1579-1941: Lucentis Vh coding sequence after optimization |
| | 1942-2001: 4xGGGGS linker |
| | 2002-2337: Lucentis VI coding sequence after optimization |
| | 2348-2396: Poly A signal |
| | 2397-2637: Human U6 promoter |
| | 2644-2699: shRNA1 against Ang2 |
| | (GGTTCAACGGCATTAAATAtacctgacccataTATTTAATGCCGTTGAACCTTTTT |
| | 2722-2827: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTGAAGGGCGTGCAG |
| | TGCGAGGTGCAGCTGGTGGAGAGCGGCGGCGGCCTGGTGCAGCCC |
| | GGCGGCAGCCTGAGACTGAGCTGCGCCGCCAGCGGCTACGACTTC |
| | ACCCACTACGGCATGAACTGGGTGAGACAGGCCCCCGGCAAGGGC |
| | CTGGAGTGGGTGGGCTGGATCAACACCTACACCGGCGAGCCCACC |
| | TACGCCGCCGACTTCAAGAGAAGATTCACCTTCAGCCTGGACACC |
| | AGCAAGAGCACCGCCTACCTGCAGATGAACAGCCTGAGAGCCGAG |
| | GACACCGCCGTGTACTACTGCGCCAAGTACCCCTACTACTACGGC |
| | ACCAGCCACTGGTACTTCGACGTGTGGGGCCAGGGCACCCTGGTG |
| | ACCGTGGGCGGAGGCGGAAGCGGCGGAGGCGGATCTGGCGGAGGC |
| | GGCAGCGGCGGCGGCGGCTCTGACATCCAGCTGACCCAGAGCCCC |
| | AGCAGCCTGAGCGCCAGCGTGGGCGACAGAGTGACCATCACCTGC |
| | AGCGCCAGCCAGGACATCAGCAACTACCTGAACTGGTACCAGCAG |
| | AAGCCCGGCAAGGCCCCCAAGGTGCTGATCTACTTCACCAGCAGC |
| | CTGCACAGCGGCGTGCCCAGCAGATTCAGCGGCAGCGGCAGCGGC |
| | ACCGACTTCACCCTGACCATCAGCAGCCTGCAGCCCGAGGACTTC |
| | GCCACCTACTACTGCCAGCAGTACAGCACCGTGCCCTGGACCTTC |
| | GGCCAGGGCACCAAGGTGGAGATCAAGAGAACCGTGGCCGCCTGA |
| | TTCGAAAAATAAAATATCTTTATTTTCATTACATCTGTGTGTTGG |
| | TTTTTTGTGTGGAGGGCCTATTTCCCATGATTCCTTCATATTTGC |
| | ATATACGATACAAGGCTGTTAGAGAGATAATTGGAATTAATTTGA |
| | CTGTAAACACAAAGATATTAGTACAAAATACGTGACGTAGAAAGT |
| | AATAATTTCTTGGGTAGTTTGCAGTTTTAAAATTATGTTTTAAAA |
| | TGGACTATCATATGCTTACCGTAACTTGAAAGTATTTCGATTTCT |
| | TGGCTTTATATATCTTGTGGAAAGGACAAGCTTGGTTCAACGGCA |
| | TTAAATATACCTGACCCATATATTTAATGCCGTTGAACCTTTTTG |
| | CATGCTGGGGAGAGATCAACCCCACTCCCTCTCTGCGCGCTCGCT |
| | CGCTCACTGAGGCCGGGCGACCAAAGGTCGCCCGACGCCCGGGCT |
| | TTGCCCGGGCGGCCTCAGTGAGCGAGCGAGCGCGCAGCAAGCTGT |
| | AGCCAACCACTAGAACTATAGCTAGAGTCCTGGGCGAACAAACGA |
| | TGCTCGCCTTCCAGAAAACCGAGGATGCGAACCACTTCATCCGGG |
| | GTCAGCACCACCGGCAAGCGCCGCGACGGCCGAGGTCTTCCGATC |
| | TCCTGAAGCCAGGGCAGATCCGTGCACAGCACCTTGCCGTAGAAG |
| | AACAGCAAGGCCGCCAATGCCTGACGATGCGTGGAGACCGAAACC |
| | TTGCGCTCGTTCGCCAGCCAGGACAGAAATGCCTCGACTTCGCTG |
| | CTGCCCAAGGTTGCCGGGTGACGCACACCGTGGAAACGGATGAAG |
| | GCACGAACCCAGTTGACATAAGCCTGTTCGGTTCGTAAACTGTAA |
| | TGCAAGTAGCGTATGCGCTCACGCAACTGGTCCAGAACCTTGACC |
| | GAACGCAGCGGTGGTAACGGCGCAGTGGCGGTTTTCATGGCTTGT |
| | TATGACTGTTTTTTTGTACAGTCTATGCCTCGGGCATCCAAGCAG |
| | CAAGCGCGTTACGCCGTGGGTCGATGTTTGATGTTATGGAGCAGC |
| | AACGATGTTACGCAGCAGCAACGATGTTACGCAGCAGGGCAGTCG |
| | CCCTAAAACAAAGTTAGGTGGCTCAAGTATGGGCATCATTCGCAC |
| | ATGTAGGCTCGGCCCTGACCAAGTCAAATCCATGCGGGCTGCTCT |
| | TGATCTTTTCGGTCGTGAGTTCGGAGACGTAGCCACCTACTCCCA |
| | ACATCAGCCGGACTCCGATTACCTCGGGAACTTGCTCCGTAGTAA |
| | GACATTCATCGCGCTTGCTGCCTTCGACCAAGAAGCGGTTGTTGG |
| | CGCTCTCGCGGCTTACGTTCTGCCCAGGTTTGAGCAGCCGCGTAG |
| | TGAGATCTATATCTATGATCTCGCAGTCTCCGGCGAGCACCGGAG |
| | GCAGGGCATTGCCACCGCGCTCATCAATCTCCTCAAGCATGAGGC |
| | CAACGCGCTTGGTGCTTATGTGATCTACGTGCAAGCAGATTACGG |
| | TGACGATCCCGCAGTGGCTCTCTATACAAAGTTGGGCATACGGGA |
| | AGAAGTGATGCACTTTGATATCGACCCAAGTACCGCCACCTAACA |
| | ATTCGTTCAAGCCGAGATCGGCTTCCCGGCCGCGGAGTTGTTCGG |
| | TAAATTGTCACAACGCCGCGAATATAGTCTTTACCATGCCCTTGG |
| | CCACGCCCCTCTTTAATACGACGGGCAATTTGCACTTCAGAAAAT |
| | GAAGAGTTTGCTTTAGCCATAACAAAAGTCCAGTATGCTTTTTCA |
| | CAGCATAACTGGACTGATTTCAGTTTACAACTATTCTGTCTAGTT |
| | TAAGACTTTATTGTCATAGTTTAGATCTATTTTGTTCAGTTTAAG |
| | ACTTTATTGTCCGCCCACACCCGCTTACGCAGGGCATCCATTTAT |
| | TACTCAACCGTAACCGATTTTGCCAGGTTACGCGGCTGGTCTGCG |
| | GTGTGAAATACCGCACAGATGCGTAAGGAGAAAATACCGCATCAG |
| | GCGCTCTTCCGCTTCCTCGCTCACTGACTCGCTGCGCTCGGTCGT |
| | TCGGCTGCGGCGAGCGGTATCAGCTCACTCAAAGGCGGTAATACG |
| | GTTATCCACAGAATCAGGGGATAACGCAGGAAAGAACATGTGAGC |
| | AAAAGGCCAGCAAAAGGCCAGGAACCGTAAAAAGGCCGCGTTGCT |
| | GGCGTTTTTCCATAGGCTCCGCCCCCCTGACGAGCATCACAAAAA |
| | TCGACGCTCAAGTCAGAGGTGGCGAAACCCGACAGGACTATAAAG |
| | ATACCAGGCGTTTCCCCCTGGAAGCTCCCTCGTGCGCTCTCCTGT |
| | TCCGACCCTGCCGCTTACCGGATACCTGTCCGCCTTTCTCCCTTC |
| | GGGAAGCGTGGCGCTTTCTCAATGCTCACGCTGTAGGTATCTCAG |
| | TTCGGTGTAGGTCGTTCGCTCCAAGCTGGGCTGTGTGCACGAACC |
| | CCCCGTTCAGCCCGACCGCTGCGCCTTATCCGGTAACTATCGTCT |
| | TGAGTCCAACCCGGTAAGACACGACTTATCGCCACTGGCAGCAGC |
| | CACTGGTAACAGGATTAGCAGAGCGAGGTATGTAGGCGGTGCTAC |
| | AGAGTTCTTGAAGTGGTGGCCTAACTACGGCTACACTAGAAGGAC |
| | AGTATTTGGTATCTGCGCTCTGCTGAAGCCAGTTACCTTCGGAAA |
| | AAGAGTTGGTAGCTCTTGATCCGGCAAACAAACCACCGCTGGTAG |
| | CGGTGGTTTTTTTGTTTGCAAGCAGCAGATTACGCGCAGAAAAAA |
| | AGGATCTCAAGAAGATCCTTTGATCTTTTCTACGGGGTCTGACGC |
| | TCAGTGGAACGAAAACTCACGTTAAGGGATTTTGGTCATGAGATT |
| | ATCAAAAAGGATCTTCACCTAGATCCTTTTAAATTAAAAATGAAG |
| | TTTTAAATCAATCTAAAGTATATATGAGTAAACTTGGTCTGACAG |
| | TTACCAATGCTTAATCAGTGAGGCACCTATCTCAGCGATCTGTCT |
| | ATTTCGTTCATCCATAGTTGCCTGACTCCCCGTCGTGTAGATAAC |
| | TACGATACGGGAGGGCTTACCATCTGGCCCCAGTGCTGCAATGAT |
| | ACCGCGAGACCCACGCTCACCGGCTCCAGATTTATCAGCAATAAA |
| | CCAGCCAGCCGGAAGGGCCGAGCGCAGAAGTGGTCCTGCAACTTT |
| | ATCCGCCTCCATCCAGTCTATTAATTGTTGCCGGGAAGCTAGAGT |
| | AAGTAGTTCGCCAGTTAATAGTTTGCGCAACGTTGTTGCCATTGC |
| | TACAGGCATCGTGGTGTCACGCTCGTCGTTTGGTATGGCTTCATT |
| | CAGCTCCGGTTCCCAACGATCAAGGCGAGTTACATGATCCCCCAT |
| | GTTGTGCAAAAAAGCGGTTAGCTCCTTCGGTCCTCCGATCGTTGT |
| | CAGAAGTAAGTTGGCCGCAGTGTTATCACTCATGGTTATGGCAGC |
| | ACTGCATAATTCTCTTACTGTCATGCCATCCGTAAGATGCTTTTC |
| | TGTGACTGGTGAGTACTCAACCAAGTCATTCTGAGAATAGTGTAT |
| | GCGGCGACCGAGTTGCTCTTGCCCGGCGTCAATACGGGATAATAC |
| | CGCGCCACATAGCAGAACTTTAAAAGTGCTCATCATTGGAAAACG |
| | TTCTTCGGGGCGAAAACTCTCAAGGATCTTACCGCTGTTGAGATC |
| | CAGTTCGATGTAACCCACTCGTGCACCCAACTGATCTTCAGCATC |
| | TTTTACTTTCACCAGCGTTTCTGGGTGAGCAAAAACAGGAAGGCA |
| | AAATGCCGCAAAAAAGGGAATAAGGGCGACACGGAAATGTTGAAT |
| | ACTCATACTCTTCCTTTTTCAATATTATTGAAGCATTTATCAGGG |
| | TTATTGTCTCATGAGCGGATACATATTTGAATGTATTTAGAAAAA |
| | TAAACAAATAGGGGTTCCGCGCACATTTCCCCGAAAAGTGCCACC |
| | TGAAATTGTAAACGTTAATATTTTGTTAAAATTCGCGTTAAATTT |
| | TTGTTAAATCAGCTCATTTTTTAACCAATAGGCCGAAATCGGCAA |
| | AATCCCTTATAAATCAAAAGAATAGACCGAGATAGGGTTGAGTGT |
| | TGTTCCAGTTTGGAACAAGAGTCCACTATTAAAGAACGTGGACTC |
| | CAACGTCAAAGGGCGAAAAACCGTCTATCAGGGCGATGGCCCACT |
| | ACGTGAACCATCACCCTAATCAAGTTTTTTGGGGTCGAGGTGCCG |
| | TAAAGCACTAAATCGGAACCCTAAAGGGAGCCCCCGATTTAGAGC |
| | TTGACGGGGAAAGCCGGCGAACGTGGCGAGAAAGGAAGGGAAGAA |
| | AGCGAAAGGAGCGGGCGCTAGGGCGCTGGCAAGTGTAGCGGTCAC |
| | GCTGCGCGTAACCACCACACCCGCCGCGCTTAATGCGCCGCTACA |
| | GGGCGCGTC |
| AMI1581 | 611-755: Full ITR |
| SEQ ID | 801-1104: CMV enhancer |
| NO: 69 | 1105-1308: CMV promoter |
| | 1412-1508: SV40 intron |
| | 1513-1521: Kozak sequence |
| | 1522-1578: Human IgG heavy chain secretion sequence |
| | 1579-1941: Lucentis Vh coding sequence after optimization |
| | 1942-2001: 4xGGGGS linker |
| | 2002-2337: Lucentis VI coding sequence after optimization |
| | 2348-2396: Poly A signal |
| | 2397-2637: Human U6 promoter |
| | 2644-2699: shRNA2 against Ang2 |
| | (GGAAGCTTGAGAATTATAAtacctgacccataTTATAATTCTCAAGCTTCCTTTTT) |
| | 2722-2827: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTGAAGGGCGTGCAG |
| | TGCGAGGTGCAGCTGGTGGAGAGCGGCGGCGGCCTGGTGCAGCCC |
| | GGCGGCAGCCTGAGACTGAGCTGCGCCGCCAGCGGCTACGACTTC |
| | ACCCACTACGGCATGAACTGGGTGAGACAGGCCCCCGGCAAGGGC |
| | CTGGAGTGGGTGGGCTGGATCAACACCTACACCGGCGAGCCCACC |
| | TACGCCGCCGACTTCAAGAGAAGATTCACCTTCAGCCTGGACACC |
| | AGCAAGAGCACCGCCTACCTGCAGATGAACAGCCTGAGAGCCGAG |
| | GACACCGCCGTGTACTACTGCGCCAAGTACCCCTACTACTACGGC |
| | ACCAGCCACTGGTACTTCGACGTGTGGGGCCAGGGCACCCTGGTG |
| | ACCGTGGGCGGAGGCGGAAGCGGCGGAGGCGGATCTGGCGGAGGC |
| | GGCAGCGGCGGCGGCGGCTCTGACATCCAGCTGACCCAGAGCCCC |
| | AGCAGCCTGAGCGCCAGCGTGGGCGACAGAGTGACCATCACCTGC |
| | AGCGCCAGCCAGGACATCAGCAACTACCTGAACTGGTACCAGCAG |
| | AAGCCCGGCAAGGCCCCCAAGGTGCTGATCTACTTCACCAGCAGC |
| | CTGCACAGCGGCGTGCCCAGCAGATTCAGCGGCAGCGGCAGCGGC |
| | ACCGACTTCACCCTGACCATCAGCAGCCTGCAGCCCGAGGACTTC |
| | GCCACCTACTACTGCCAGCAGTACAGCACCGTGCCCTGGACCTTC |
| | GGCCAGGGCACCAAGGTGGAGATCAAGAGAACCGTGGCCGCCTGA |
| | TTCGAAAAATAAAATATCTTTATTTTCATTACATCTGTGTGTTGG |
| | TTTTTTGTGTGGAGGGCCTATTTCCCATGATTCCTTCATATTTGC |
| | ATATACGATACAAGGCTGTTAGAGAGATAATTGGAATTAATTTGA |
| | CTGTAAACACAAAGATATTAGTACAAAATACGTGACGTAGAAAGT |
| | AATAATTTCTTGGGTAGTTTGCAGTTTTAAAATTATGTTTTAAAA |
| | TGGACTATCATATGCTTACCGTAACTTGAAAGTATTTCGATTTCT |
| | TGGCTTTATATATCTTGTGGAAAGGACAAGCTTGGAAGCTTGAGA |
| | ATTATAATACCTGACCCATATTATAATTCTCAAGCTTCCTTTTTG |
| | CATGCTGGGGAGAGATCAACCCCACTCCCTCTCTGCGCGCTCGCT |
| | CGCTCACTGAGGCCGGGCGACCAAAGGTCGCCCGACGCCCGGGCT |
| | TTGCCCGGGCGGCCTCAGTGAGCGAGCGAGCGCGCAGCAAGCTGT |
| | AGCCAACCACTAGAACTATAGCTAGAGTCCTGGGCGAACAAACGA |
| | TGCTCGCCTTCCAGAAAACCGAGGATGCGAACCACTTCATCCGGG |
| | GTCAGCACCACCGGCAAGCGCCGCGACGGCCGAGGTCTTCCGATC |
| | TCCTGAAGCCAGGGCAGATCCGTGCACAGCACCTTGCCGTAGAAG |
| | AACAGCAAGGCCGCCAATGCCTGACGATGCGTGGAGACCGAAACC |
| | TTGCGCTCGTTCGCCAGCCAGGACAGAAATGCCTCGACTTCGCTG |
| | CTGCCCAAGGTTGCCGGGTGACGCACACCGTGGAAACGGATGAAG |
| | GCACGAACCCAGTTGACATAAGCCTGTTCGGTTCGTAAACTGTAA |
| | TGCAAGTAGCGTATGCGCTCACGCAACTGGTCCAGAACCTTGACC |
| | GAACGCAGCGGTGGTAACGGCGCAGTGGCGGTTTTCATGGCTTGT |
| | TATGACTGTTTTTTTGTACAGTCTATGCCTCGGGCATCCAAGCAG |
| | CAAGCGCGTTACGCCGTGGGTCGATGTTTGATGTTATGGAGCAGC |
| | AACGATGTTACGCAGCAGCAACGATGTTACGCAGCAGGGCAGTCG |
| | CCCTAAAACAAAGTTAGGTGGCTCAAGTATGGGCATCATTCGCAC |
| | ATGTAGGCTCGGCCCTGACCAAGTCAAATCCATGCGGGCTGCTCT |
| | TGATCTTTTCGGTCGTGAGTTCGGAGACGTAGCCACCTACTCCCA |
| | ACATCAGCCGGACTCCGATTACCTCGGGAACTTGCTCCGTAGTAA |
| | GACATTCATCGCGCTTGCTGCCTTCGACCAAGAAGCGGTTGTTGG |
| | CGCTCTCGCGGCTTACGTTCTGCCCAGGTTTGAGCAGCCGCGTAG |
| | TGAGATCTATATCTATGATCTCGCAGTCTCCGGCGAGCACCGGAG |
| | GCAGGGCATTGCCACCGCGCTCATCAATCTCCTCAAGCATGAGGC |
| | CAACGCGCTTGGTGCTTATGTGATCTACGTGCAAGCAGATTACGG |
| | TGACGATCCCGCAGTGGCTCTCTATACAAAGTTGGGCATACGGGA |
| | AGAAGTGATGCACTTTGATATCGACCCAAGTACCGCCACCTAACA |
| | ATTCGTTCAAGCCGAGATCGGCTTCCCGGCCGCGGAGTTGTTCGG |
| | TAAATTGTCACAACGCCGCGAATATAGTCTTTACCATGCCCTTGG |
| | CCACGCCCCTCTTTAATACGACGGGCAATTTGCACTTCAGAAAAT |
| | GAAGAGTTTGCTTTAGCCATAACAAAAGTCCAGTATGCTTTTTCA |
| | CAGCATAACTGGACTGATTTCAGTTTACAACTATTCTGTCTAGTT |
| | TAAGACTTTATTGTCATAGTTTAGATCTATTTTGTTCAGTTTAAG |
| | ACTTTATTGTCCGCCCACACCCGCTTACGCAGGGCATCCATTTAT |
| | TACTCAACCGTAACCGATTTTGCCAGGTTACGCGGCTGGTCTGCG |
| | GTGTGAAATACCGCACAGATGCGTAAGGAGAAAATACCGCATCAG |
| | GCGCTCTTCCGCTTCCTCGCTCACTGACTCGCTGCGCTCGGTCGT |
| | TCGGCTGCGGCGAGCGGTATCAGCTCACTCAAAGGCGGTAATACG |
| | GTTATCCACAGAATCAGGGGATAACGCAGGAAAGAACATGTGAGC |
| | AAAAGGCCAGCAAAAGGCCAGGAACCGTAAAAAGGCCGCGTTGCT |
| | GGCGTTTTTCCATAGGCTCCGCCCCCCTGACGAGCATCACAAAAA |
| | TCGACGCTCAAGTCAGAGGTGGCGAAACCCGACAGGACTATAAAG |
| | ATACCAGGCGTTTCCCCCTGGAAGCTCCCTCGTGCGCTCTCCTGT |
| | TCCGACCCTGCCGCTTACCGGATACCTGTCCGCCTTTCTCCCTTC |
| | GGGAAGCGTGGCGCTTTCTCAATGCTCACGCTGTAGGTATCTCAG |
| | TTCGGTGTAGGTCGTTCGCTCCAAGCTGGGCTGTGTGCACGAACC |
| | CCCCGTTCAGCCCGACCGCTGCGCCTTATCCGGTAACTATCGTCT |
| | TGAGTCCAACCCGGTAAGACACGACTTATCGCCACTGGCAGCAGC |
| | CACTGGTAACAGGATTAGCAGAGCGAGGTATGTAGGCGGTGCTAC |
| | AGAGTTCTTGAAGTGGTGGCCTAACTACGGCTACACTAGAAGGAC |
| | AGTATTTGGTATCTGCGCTCTGCTGAAGCCAGTTACCTTCGGAAA |
| | AAGAGTTGGTAGCTCTTGATCCGGCAAACAAACCACCGCTGGTAG |
| | CGGTGGTTTTTTTGTTTGCAAGCAGCAGATTACGCGCAGAAAAAA |
| | AGGATCTCAAGAAGATCCTTTGATCTTTTCTACGGGGTCTGACGC |
| | TCAGTGGAACGAAAACTCACGTTAAGGGATTTTGGTCATGAGATT |
| | ATCAAAAAGGATCTTCACCTAGATCCTTTTAAATTAAAAATGAAG |
| | TTTTAAATCAATCTAAAGTATATATGAGTAAACTTGGTCTGACAG |
| | TTACCAATGCTTAATCAGTGAGGCACCTATCTCAGCGATCTGTCT |
| | ATTTCGTTCATCCATAGTTGCCTGACTCCCCGTCGTGTAGATAAC |
| | TACGATACGGGAGGGCTTACCATCTGGCCCCAGTGCTGCAATGAT |
| | ACCGCGAGACCCACGCTCACCGGCTCCAGATTTATCAGCAATAAA |
| | CCAGCCAGCCGGAAGGGCCGAGCGCAGAAGTGGTCCTGCAACTTT |
| | ATCCGCCTCCATCCAGTCTATTAATTGTTGCCGGGAAGCTAGAGT |
| | AAGTAGTTCGCCAGTTAATAGTTTGCGCAACGTTGTTGCCATTGC |
| | TACAGGCATCGTGGTGTCACGCTCGTCGTTTGGTATGGCTTCATT |
| | CAGCTCCGGTTCCCAACGATCAAGGCGAGTTACATGATCCCCCAT |
| | GTTGTGCAAAAAAGCGGTTAGCTCCTTCGGTCCTCCGATCGTTGT |
| | CAGAAGTAAGTTGGCCGCAGTGTTATCACTCATGGTTATGGCAGC |
| | ACTGCATAATTCTCTTACTGTCATGCCATCCGTAAGATGCTTTTC |
| | TGTGACTGGTGAGTACTCAACCAAGTCATTCTGAGAATAGTGTAT |
| | GCGGCGACCGAGTTGCTCTTGCCCGGCGTCAATACGGGATAATAC |
| | CGCGCCACATAGCAGAACTTTAAAAGTGCTCATCATTGGAAAACG |
| | TTCTTCGGGGCGAAAACTCTCAAGGATCTTACCGCTGTTGAGATC |
| | CAGTTCGATGTAACCCACTCGTGCACCCAACTGATCTTCAGCATC |
| | TTTTACTTTCACCAGCGTTTCTGGGTGAGCAAAAACAGGAAGGCA |
| | AAATGCCGCAAAAAAGGGAATAAGGGCGACACGGAAATGTTGAAT |
| | ACTCATACTCTTCCTTTTTCAATATTATTGAAGCATTTATCAGGG |
| | TTATTGTCTCATGAGCGGATACATATTTGAATGTATTTAGAAAAA |
| | TAAACAAATAGGGGTTCCGCGCACATTTCCCCGAAAAGTGCCACC |
| | TGAAATTGTAAACGTTAATATTTTGTTAAAATTCGCGTTAAATTT |
| | TTGTTAAATCAGCTCATTTTTTAACCAATAGGCCGAAATCGGCAA |
| | AATCCCTTATAAATCAAAAGAATAGACCGAGATAGGGTTGAGTGT |
| | TGTTCCAGTTTGGAACAAGAGTCCACTATTAAAGAACGTGGACTC |
| | CAACGTCAAAGGGCGAAAAACCGTCTATCAGGGCGATGGCCCACT |
| | ACGTGAACCATCACCCTAATCAAGTTTTTTGGGGTCGAGGTGCCG |
| | TAAAGCACTAAATCGGAACCCTAAAGGGAGCCCCCGATTTAGAGC |
| | TTGACGGGGAAAGCCGGCGAACGTGGCGAGAAAGGAAGGGAAGAA |
| | AGCGAAAGGAGCGGGCGCTAGGGCGCTGGCAAGTGTAGCGGTCAC |
| | GCTGCGCGTAACCACCACACCCGCCGCGCTTAATGCGCCGCTACA |
| | GGGCGCGTC |
| AMI159; | 611-755: Full ITR |
| SEQ ID | 801-1104: CMV enhancer |
| NO: 70 | 1105-1308: CMV promoter |
| | 1412-1508: SV40 intron |
| | 1513-1521: Kozak sequence |
| | 1522-1578: Human IgG heavy chain secretion sequence |
| | 1579-1941: Lucentis Vh coding sequence after optimization |
| | 1942-2001: 4xGGGGS linker |
| | 2002-2337: Lucentis VI coding sequence after optimization |
| | 2348-2396: Poly A signal |
| | 2397-2637: Human U6 promoter |
| | 2644-2699: shRNA3 against Ang2 |
| | (GTGAAGAACTCAATTATAAtacctgacccataTTATAATTGAGTTCTTCACTTTTT) |
| | 2722-2827: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTGAAGGGCGTGCAG |
| | TGCGAGGTGCAGCTGGTGGAGAGCGGCGGCGGCCTGGTGCAGCCC |
| | GGCGGCAGCCTGAGACTGAGCTGCGCCGCCAGCGGCTACGACTTC |
| | ACCCACTACGGCATGAACTGGGTGAGACAGGCCCCCGGCAAGGGC |
| | CTGGAGTGGGTGGGCTGGATCAACACCTACACCGGCGAGCCCACC |
| | TACGCCGCCGACTTCAAGAGAAGATTCACCTTCAGCCTGGACACC |
| | AGCAAGAGCACCGCCTACCTGCAGATGAACAGCCTGAGAGCCGAG |
| | GACACCGCCGTGTACTACTGCGCCAAGTACCCCTACTACTACGGC |
| | ACCAGCCACTGGTACTTCGACGTGTGGGGCCAGGGCACCCTGGTG |
| | ACCGTGGGCGGAGGCGGAAGCGGCGGAGGCGGATCTGGCGGAGGC |
| | GGCAGCGGCGGCGGCGGCTCTGACATCCAGCTGACCCAGAGCCCC |
| | AGCAGCCTGAGCGCCAGCGTGGGCGACAGAGTGACCATCACCTGC |
| | AGCGCCAGCCAGGACATCAGCAACTACCTGAACTGGTACCAGCAG |
| | AAGCCCGGCAAGGCCCCCAAGGTGCTGATCTACTTCACCAGCAGC |
| | CTGCACAGCGGCGTGCCCAGCAGATTCAGCGGCAGCGGCAGCGGC |
| | ACCGACTTCACCCTGACCATCAGCAGCCTGCAGCCCGAGGACTTC |
| | GCCACCTACTACTGCCAGCAGTACAGCACCGTGCCCTGGACCTTC |
| | GGCCAGGGCACCAAGGTGGAGATCAAGAGAACCGTGGCCGCCTGA |
| | TTCGAAAATAAAATATCTTTATTTTCATTACATCTGTGTGTTGGT |
| | TTTTTGTGTGGAGGGCCTATTTCCCATGATTCCTTCATATTTGCA |
| | TATACGATACAAGGCTGTTAGAGAGATAATTGGAATTAATTTGAC |
| | TGTAAACACAAAGATATTAGTACAAAATACGTGACGTAGAAAGTA |
| | ATAATTTCTTGGGTAGTTTGCAGTTTTAAAATTATGTTTTAAAAT |
| | GGACTATCATATGCTTACCGTAACTTGAAAGTATTTCGATTTCTT |
| | GGCTTTATATATCTTGTGGAAAGGACAAGCTTGTGAAGAACTCAA |
| | TTATAATACCTGACCCATATTATAATTGAGTTCTTCACTTTTTGC |
| | ATGCTGGGGAGAGATCAACCCCACTCCCTCTCTGCGCGCTCGCTC |
| | GCTCACTGAGGCCGGGCGACCAAAGGTCGCCCGACGCCCGGGCTT |
| | TGCCCGGGCGGCCTCAGTGAGCGAGCGAGCGCGCAGCAAGCTGTA |
| | GCCAACCACTAGAACTATAGCTAGAGTCCTGGGCGAACAAACGAT |
| | GCTCGCCTTCCAGAAAACCGAGGATGCGAACCACTTCATCCGGGG |
| | TCAGCACCACCGGCAAGCGCCGCGACGGCCGAGGTCTTCCGATCT |
| | CCTGAAGCCAGGGCAGATCCGTGCACAGCACCTTGCCGTAGAAGA |
| | ACAGCAAGGCCGCCAATGCCTGACGATGCGTGGAGACCGAAACCT |
| | TGCGCTCGTTCGCCAGCCAGGACAGAAATGCCTCGACTTCGCTGC |
| | TGCCCAAGGTTGCCGGGTGACGCACACCGTGGAAACGGATGAAGG |
| | CACGAACCCAGTTGACATAAGCCTGTTCGGTTCGTAAACTGTAAT |
| | GCAAGTAGCGTATGCGCTCACGCAACTGGTCCAGAACCTTGACCG |
| | AACGCAGCGGTGGTAACGGCGCAGTGGCGGTTTTCATGGCTTGTT |
| | ATGACTGTTTTTTTGTACAGTCTATGCCTCGGGCATCCAAGCAGC |
| | AAGCGCGTTACGCCGTGGGTCGATGTTTGATGTTATGGAGCAGCA |
| | ACGATGTTACGCAGCAGCAACGATGTTACGCAGCAGGGCAGTCGC |
| | CCTAAAACAAAGTTAGGTGGCTCAAGTATGGGCATCATTCGCACA |
| | TGTAGGCTCGGCCCTGACCAAGTCAAATCCATGCGGGCTGCTCTT |
| | GATCTTTTCGGTCGTGAGTTCGGAGACGTAGCCACCTACTCCCAA |
| | CATCAGCCGGACTCCGATTACCTCGGGAACTTGCTCCGTAGTAAG |
| | ACATTCATCGCGCTTGCTGCCTTCGACCAAGAAGCGGTTGTTGGC |
| | GCTCTCGCGGCTTACGTTCTGCCCAGGTTTGAGCAGCCGCGTAGT |
| | GAGATCTATATCTATGATCTCGCAGTCTCCGGCGAGCACCGGAGG |
| | CAGGGCATTGCCACCGCGCTCATCAATCTCCTCAAGCATGAGGCC |
| | AACGCGCTTGGTGCTTATGTGATCTACGTGCAAGCAGATTACGGT |
| | GACGATCCCGCAGTGGCTCTCTATACAAAGTTGGGCATACGGGAA |
| | GAAGTGATGCACTTTGATATCGACCCAAGTACCGCCACCTAACAA |
| | TTCGTTCAAGCCGAGATCGGCTTCCCGGCCGCGGAGTTGTTCGGT |
| | AAATTGTCACAACGCCGCGAATATAGTCTTTACCATGCCCTTGGC |
| | CACGCCCCTCTTTAATACGACGGGCAATTTGCACTTCAGAAAATG |
| | AAGAGTTTGCTTTAGCCATAACAAAAGTCCAGTATGCTTTTTCAC |
| | AGCATAACTGGACTGATTTCAGTTTACAACTATTCTGTCTAGTTT |
| | AAGACTTTATTGTCATAGTTTAGATCTATTTTGTTCAGTTTAAGA |
| | CTTTATTGTCCGCCCACACCCGCTTACGCAGGGCATCCATTTATT |
| | ACTCAACCGTAACCGATTTTGCCAGGTTACGCGGCTGGTCTGCGG |
| | TGTGAAATACCGCACAGATGCGTAAGGAGAAAATACCGCATCAGG |
| | CGCTCTTCCGCTTCCTCGCTCACTGACTCGCTGCGCTCGGTCGTT |
| | CGGCTGCGGCGAGCGGTATCAGCTCACTCAAAGGCGGTAATACGG |
| | TTATCCACAGAATCAGGGGATAACGCAGGAAAGAACATGTGAGCA |
| | AAAGGCCAGCAAAAGGCCAGGAACCGTAAAAAGGCCGCGTTGCTG |
| | GCGTTTTTCCATAGGCTCCGCCCCCCTGACGAGCATCACAAAAAT |
| | CGACGCTCAAGTCAGAGGTGGCGAAACCCGACAGGACTATAAAGA |
| | TACCAGGCGTTTCCCCCTGGAAGCTCCCTCGTGCGCTCTCCTGTT |
| | CCGACCCTGCCGCTTACCGGATACCTGTCCGCCTTTCTCCCTTCG |
| | GGAAGCGTGGCGCTTTCTCAATGCTCACGCTGTAGGTATCTCAGT |
| | TCGGTGTAGGTCGTTCGCTCCAAGCTGGGCTGTGTGCACGAACCC |
| | CCCGTTCAGCCCGACCGCTGCGCCTTATCCGGTAACTATCGTCTT |
| | GAGTCCAACCCGGTAAGACACGACTTATCGCCACTGGCAGCAGCC |
| | ACTGGTAACAGGATTAGCAGAGCGAGGTATGTAGGCGGTGCTACA |
| | GAGTTCTTGAAGTGGTGGCCTAACTACGGCTACACTAGAAGGACA |
| | GTATTTGGTATCTGCGCTCTGCTGAAGCCAGTTACCTTCGGAAAA |
| | AGAGTTGGTAGCTCTTGATCCGGCAAACAAACCACCGCTGGTAGC |
| | GGTGGTTTTTTTGTTTGCAAGCAGCAGATTACGCGCAGAAAAAAA |
| | GGATCTCAAGAAGATCCTTTGATCTTTTCTACGGGGTCTGACGCT |
| | CAGTGGAACGAAAACTCACGTTAAGGGATTTTGGTCATGAGATTA |
| | TCAAAAAGGATCTTCACCTAGATCCTTTTAAATTAAAAATGAAGT |
| | TTTAAATCAATCTAAAGTATATATGAGTAAACTTGGTCTGACAGT |
| | TACCAATGCTTAATCAGTGAGGCACCTATCTCAGCGATCTGTCTA |
| | TTTCGTTCATCCATAGTTGCCTGACTCCCCGTCGTGTAGATAACT |
| | ACGATACGGGAGGGCTTACCATCTGGCCCCAGTGCTGCAATGATA |
| | CCGCGAGACCCACGCTCACCGGCTCCAGATTTATCAGCAATAAAC |
| | CAGCCAGCCGGAAGGGCCGAGCGCAGAAGTGGTCCTGCAACTTTA |
| | TCCGCCTCCATCCAGTCTATTAATTGTTGCCGGGAAGCTAGAGTA |
| | AGTAGTTCGCCAGTTAATAGTTTGCGCAACGTTGTTGCCATTGCT |
| | ACAGGCATCGTGGTGTCACGCTCGTCGTTTGGTATGGCTTCATTC |
| | AGCTCCGGTTCCCAACGATCAAGGCGAGTTACATGATCCCCCATG |
| | TTGTGCAAAAAAGCGGTTAGCTCCTTCGGTCCTCCGATCGTTGTC |
| | AGAAGTAAGTTGGCCGCAGTGTTATCACTCATGGTTATGGCAGCA |
| | CTGCATAATTCTCTTACTGTCATGCCATCCGTAAGATGCTTTTCT |
| | GTGACTGGTGAGTACTCAACCAAGTCATTCTGAGAATAGTGTATG |
| | CGGCGACCGAGTTGCTCTTGCCCGGCGTCAATACGGGATAATACC |
| | GCGCCACATAGCAGAACTTTAAAAGTGCTCATCATTGGAAAACGT |
| | TCTTCGGGGCGAAAACTCTCAAGGATCTTACCGCTGTTGAGATCC |
| | AGTTCGATGTAACCCACTCGTGCACCCAACTGATCTTCAGCATCT |
| | TTTACTTTCACCAGCGTTTCTGGGTGAGCAAAAACAGGAAGGCAA |
| | AATGCCGCAAAAAAGGGAATAAGGGCGACACGGAAATGTTGAATA |
| | CTCATACTCTTCCTTTTTCAATATTATTGAAGCATTTATCAGGGT |
| | TATTGTCTCATGAGCGGATACATATTTGAATGTATTTAGAAAAAT |
| | AAACAAATAGGGGTTCCGCGCACATTTCCCCGAAAAGTGCCACCT |
| | GAAATTGTAAACGTTAATATTTTGTTAAAATTCGCGTTAAATTTT |
| | TGTTAAATCAGCTCATTTTTTAACCAATAGGCCGAAATCGGCAAA |
| | ATCCCTTATAAATCAAAAGAATAGACCGAGATAGGGTTGAGTGTT |
| | GTTCCAGTTTGGAACAAGAGTCCACTATTAAAGAACGTGGACTCC |
| | AACGTCAAAGGGCGAAAAACCGTCTATCAGGGCGATGGCCCACTA |
| | CGTGAACCATCACCCTAATCAAGTTTTTTGGGGTCGAGGTGCCGT |
| | AAAGCACTAAATCGGAACCCTAAAGGGAGCCCCCGATTTAGAGCT |
| | TGACGGGGAAAGCCGGCGAACGTGGCGAGAAAGGAAGGGAAGAAA |
| | GCGAAAGGAGCGGGCGCTAGGGCGCTGGCAAGTGTAGCGGTCACG |
| | CTGCGCGTAACCACCACACCCGCCGCGCTTAATGCGCCGCTACAG |
| | GGCGCGTC |
| AMI160; | 611-755: Full ITR |
| SEQ ID | 801-1104: CMV enhancer |
| NO: 71 | 1105-1308: CMV promoter |
| | 1412-1508: SV40 intron |
| | 1513-1521: Kozak sequence |
| | 1522-1578: Human IgG heavy chain secretion sequence |
| | 1579-1941: Lucentis Vh coding sequence after optimization |
| | 1942-2001: 4xGGGGS linker |
| | 2002-2337: Lucentis VI coding sequence after optimization |
| | 2347-2395: Poly A signal |
| | 2396-2636: Human U6 promoter |
| | 2643-2698: shRNA4 against Ang2 |
| | (GTAACATTCCCTAATTCTAtacctgacccataTAGAATTAGGGAATGTTACTTTTT |
| | 2721-2826: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTGAAGGGCGTGCAG |
| | TGCGAGGTGCAGCTGGTGGAGAGCGGCGGCGGCCTGGTGCAGCCC |
| | GGCGGCAGCCTGAGACTGAGCTGCGCCGCCAGCGGCTACGACTTC |
| | ACCCACTACGGCATGAACTGGGTGAGACAGGCCCCCGGCAAGGGC |
| | CTGGAGTGGGTGGGCTGGATCAACACCTACACCGGCGAGCCCACC |
| | TACGCCGCCGACTTCAAGAGAAGATTCACCTTCAGCCTGGACACC |
| | AGCAAGAGCACCGCCTACCTGCAGATGAACAGCCTGAGAGCCGAG |
| | GACACCGCCGTGTACTACTGCGCCAAGTACCCCTACTACTACGGC |
| | ACCAGCCACTGGTACTTCGACGTGTGGGGCCAGGGCACCCTGGTG |
| | ACCGTGGGCGGAGGCGGAAGCGGCGGAGGCGGATCTGGCGGAGGC |
| | GGCAGCGGCGGCGGCGGCTCTGACATCCAGCTGACCCAGAGCCCC |
| | AGCAGCCTGAGCGCCAGCGTGGGCGACAGAGTGACCATCACCTGC |
| | AGCGCCAGCCAGGACATCAGCAACTACCTGAACTGGTACCAGCAG |
| | AAGCCCGGCAAGGCCCCCAAGGTGCTGATCTACTTCACCAGCAGC |
| | CTGCACAGCGGCGTGCCCAGCAGATTCAGCGGCAGCGGCAGCGGC |
| | ACCGACTTCACCCTGACCATCAGCAGCCTGCAGCCCGAGGACTTC |
| | GCCACCTACTACTGCCAGCAGTACAGCACCGTGCCCTGGACCTTC |
| | GGCCAGGGCACCAAGGTGGAGATCAAGAGAACCGTGGCCGCCTGA |
| | TTCGAAAATAAAATATCTTTATTTTCATTACATCTGTGTGTTGGT |
| | TTTTTGTGTGGAGGGCCTATTTCCCATGATTCCTTCATATTTGCA |
| | TATACGATACAAGGCTGTTAGAGAGATAATTGGAATTAATTTGAC |
| | TGTAAACACAAAGATATTAGTACAAAATACGTGACGTAGAAAGTA |
| | ATAATTTCTTGGGTAGTTTGCAGTTTTAAAATTATGTTTTAAAAT |
| | GGACTATCATATGCTTACCGTAACTTGAAAGTATTTCGATTTCTT |
| | GGCTTTATATATCTTGTGGAAAGGACAAGCTTGTAACATTCCCTA |
| | ATTCTATACCTGACCCATATAGAATTAGGGAATGTTACTTTTTGC |
| | ATGCTGGGGAGAGATCAACCCCACTCCCTCTCTGCGCGCTCGCTC |
| | GCTCACTGAGGCCGGGCGACCAAAGGTCGCCCGACGCCCGGGCTT |
| | TGCCCGGGCGGCCTCAGTGAGCGAGCGAGCGCGCAGCAAGCTGTA |
| | GCCAACCACTAGAACTATAGCTAGAGTCCTGGGCGAACAAACGAT |
| | GCTCGCCTTCCAGAAAACCGAGGATGCGAACCACTTCATCCGGGG |
| | TCAGCACCACCGGCAAGCGCCGCGACGGCCGAGGTCTTCCGATCT |
| | CCTGAAGCCAGGGCAGATCCGTGCACAGCACCTTGCCGTAGAAGA |
| | ACAGCAAGGCCGCCAATGCCTGACGATGCGTGGAGACCGAAACCT |
| | TGCGCTCGTTCGCCAGCCAGGACAGAAATGCCTCGACTTCGCTGC |
| | TGCCCAAGGTTGCCGGGTGACGCACACCGTGGAAACGGATGAAGG |
| | CACGAACCCAGTTGACATAAGCCTGTTCGGTTCGTAAACTGTAAT |
| | GCAAGTAGCGTATGCGCTCACGCAACTGGTCCAGAACCTTGACCG |
| | AACGCAGCGGTGGTAACGGCGCAGTGGCGGTTTTCATGGCTTGTT |
| | ATGACTGTTTTTTTGTACAGTCTATGCCTCGGGCATCCAAGCAGC |
| | AAGCGCGTTACGCCGTGGGTCGATGTTTGATGTTATGGAGCAGCA |
| | ACGATGTTACGCAGCAGCAACGATGTTACGCAGCAGGGCAGTCGC |
| | CCTAAAACAAAGTTAGGTGGCTCAAGTATGGGCATCATTCGCACA |
| | TGTAGGCTCGGCCCTGACCAAGTCAAATCCATGCGGGCTGCTCTT |
| | GATCTTTTCGGTCGTGAGTTCGGAGACGTAGCCACCTACTCCCAA |
| | CATCAGCCGGACTCCGATTACCTCGGGAACTTGCTCCGTAGTAAG |
| | ACATTCATCGCGCTTGCTGCCTTCGACCAAGAAGCGGTTGTTGGC |
| | GCTCTCGCGGCTTACGTTCTGCCCAGGTTTGAGCAGCCGCGTAGT |
| | GAGATCTATATCTATGATCTCGCAGTCTCCGGCGAGCACCGGAGG |
| | CAGGGCATTGCCACCGCGCTCATCAATCTCCTCAAGCATGAGGCC |
| | AACGCGCTTGGTGCTTATGTGATCTACGTGCAAGCAGATTACGGT |
| | GACGATCCCGCAGTGGCTCTCTATACAAAGTTGGGCATACGGGAA |
| | GAAGTGATGCACTTTGATATCGACCCAAGTACCGCCACCTAACAA |
| | TTCGTTCAAGCCGAGATCGGCTTCCCGGCCGCGGAGTTGTTCGGT |
| | AAATTGTCACAACGCCGCGAATATAGTCTTTACCATGCCCTTGGC |
| | CACGCCCCTCTTTAATACGACGGGCAATTTGCACTTCAGAAAATG |
| | AAGAGTTTGCTTTAGCCATAACAAAAGTCCAGTATGCTTTTTCAC |
| | AGCATAACTGGACTGATTTCAGTTTACAACTATTCTGTCTAGTTT |
| | AAGACTTTATTGTCATAGTTTAGATCTATTTTGTTCAGTTTAAGA |
| | CTTTATTGTCCGCCCACACCCGCTTACGCAGGGCATCCATTTATT |
| | ACTCAACCGTAACCGATTTTGCCAGGTTACGCGGCTGGTCTGCGG |
| | TGTGAAATACCGCACAGATGCGTAAGGAGAAAATACCGCATCAGG |
| | CGCTCTTCCGCTTCCTCGCTCACTGACTCGCTGCGCTCGGTCGTT |
| | CGGCTGCGGCGAGCGGTATCAGCTCACTCAAAGGCGGTAATACGG |
| | TTATCCACAGAATCAGGGGATAACGCAGGAAAGAACATGTGAGCA |
| | AAAGGCCAGCAAAAGGCCAGGAACCGTAAAAAGGCCGCGTTGCTG |
| | GCGTTTTTCCATAGGCTCCGCCCCCCTGACGAGCATCACAAAAAT |
| | CGACGCTCAAGTCAGAGGTGGCGAAACCCGACAGGACTATAAAGA |
| | TACCAGGCGTTTCCCCCTGGAAGCTCCCTCGTGCGCTCTCCTGTT |
| | CCGACCCTGCCGCTTACCGGATACCTGTCCGCCTTTCTCCCTTCG |
| | GGAAGCGTGGCGCTTTCTCAATGCTCACGCTGTAGGTATCTCAGT |
| | TCGGTGTAGGTCGTTCGCTCCAAGCTGGGCTGTGTGCACGAACCC |
| | CCCGTTCAGCCCGACCGCTGCGCCTTATCCGGTAACTATCGTCTT |
| | GAGTCCAACCCGGTAAGACACGACTTATCGCCACTGGCAGCAGCC |
| | ACTGGTAACAGGATTAGCAGAGCGAGGTATGTAGGCGGTGCTACA |
| | GAGTTCTTGAAGTGGTGGCCTAACTACGGCTACACTAGAAGGACA |
| | GTATTTGGTATCTGCGCTCTGCTGAAGCCAGTTACCTTCGGAAAA |
| | AGAGTTGGTAGCTCTTGATCCGGCAAACAAACCACCGCTGGTAGC |
| | GGTGGTTTTTTTGTTTGCAAGCAGCAGATTACGCGCAGAAAAAAA |
| | GGATCTCAAGAAGATCCTTTGATCTTTTCTACGGGGTCTGACGCT |
| | CAGTGGAACGAAAACTCACGTTAAGGGATTTTGGTCATGAGATTA |
| | TCAAAAAGGATCTTCACCTAGATCCTTTTAAATTAAAAATGAAGT |
| | TTTAAATCAATCTAAAGTATATATGAGTAAACTTGGTCTGACAGT |
| | TACCAATGCTTAATCAGTGAGGCACCTATCTCAGCGATCTGTCTA |
| | TTTCGTTCATCCATAGTTGCCTGACTCCCCGTCGTGTAGATAACT |
| | ACGATACGGGAGGGCTTACCATCTGGCCCCAGTGCTGCAATGATA |
| | CCGCGAGACCCACGCTCACCGGCTCCAGATTTATCAGCAATAAAC |
| | CAGCCAGCCGGAAGGGCCGAGCGCAGAAGTGGTCCTGCAACTTTA |
| | TCCGCCTCCATCCAGTCTATTAATTGTTGCCGGGAAGCTAGAGTA |
| | AGTAGTTCGCCAGTTAATAGTTTGCGCAACGTTGTTGCCATTGCT |
| | ACAGGCATCGTGGTGTCACGCTCGTCGTTTGGTATGGCTTCATTC |
| | AGCTCCGGTTCCCAACGATCAAGGCGAGTTACATGATCCCCCATG |
| | TTGTGCAAAAAAGCGGTTAGCTCCTTCGGTCCTCCGATCGTTGTC |
| | AGAAGTAAGTTGGCCGCAGTGTTATCACTCATGGTTATGGCAGCA |
| | CTGCATAATTCTCTTACTGTCATGCCATCCGTAAGATGCTTTTCT |
| | GTGACTGGTGAGTACTCAACCAAGTCATTCTGAGAATAGTGTATG |
| | CGGCGACCGAGTTGCTCTTGCCCGGCGTCAATACGGGATAATACC |
| | GCGCCACATAGCAGAACTTTAAAAGTGCTCATCATTGGAAAACGT |
| | TCTTCGGGGCGAAAACTCTCAAGGATCTTACCGCTGTTGAGATCC |
| | AGTTCGATGTAACCCACTCGTGCACCCAACTGATCTTCAGCATCT |
| | TTTACTTTCACCAGCGTTTCTGGGTGAGCAAAAACAGGAAGGCAA |
| | AATGCCGCAAAAAAGGGAATAAGGGCGACACGGAAATGTTGAATA |
| | CTCATACTCTTCCTTTTTCAATATTATTGAAGCATTTATCAGGGT |
| | TATTGTCTCATGAGCGGATACATATTTGAATGTATTTAGAAAAAT |
| | AAACAAATAGGGGTTCCGCGCACATTTCCCCGAAAAGTGCCACCT |
| | GAAATTGTAAACGTTAATATTTTGTTAAAATTCGCGTTAAATTTT |
| | TGTTAAATCAGCTCATTTTTTAACCAATAGGCCGAAATCGGCAAA |
| | ATCCCTTATAAATCAAAAGAATAGACCGAGATAGGGTTGAGTGTT |
| | GTTCCAGTTTGGAACAAGAGTCCACTATTAAAGAACGTGGACTCC |
| | AACGTCAAAGGGCGAAAAACCGTCTATCAGGGCGATGGCCCACTA |
| | CGTGAACCATCACCCTAATCAAGTTTTTTGGGGTCGAGGTGCCGT |
| | AAAGCACTAAATCGGAACCCTAAAGGGAGCCCCCGATTTAGAGCT |
| | TGACGGGGAAAGCCGGCGAACGTGGCGAGAAAGGAAGGGAAGAAA |
| | GCGAAAGGAGCGGGCGCTAGGGCGCTGGCAAGTGTAGCGGTCACG |
| | CTGCGCGTAACCACCACACCCGCCGCGCTTAATGCGCCGCTACAG |
| | GGCGCGTC |
| AMI161; | 611-755: Full ITR |
| SEQ ID | 801-1104: CMV enhancer |
| NO: 72 | 1105-1308: CMV promoter |
| | 1412-1508: SV40 intron |
| | 1513-1521: Kozak sequence |
| | 1522-1578: Human IgG heavy chain secretion sequence |
| | 1579-1941: Lucentis Vh coding sequence after optimization |
| | 1942-2001: 4xGGGGS linker |
| | 2002-2337: Lucentis VI coding sequence after optimization |
| | 2342-2390: Poly A signal |
| | 2391-2631: Human U6 promoter |
| | 2638-2693: shRNA5 against Ang2 |
| | (GACTTGGAAAGAATATAAAtacctgacccataTTTATATTCTTTCCAAGTCTTTTT) |
| | 2716-2821: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTGAAGGGCGTGCAG |
| | TGCGAGGTGCAGCTGGTGGAGAGCGGCGGCGGCCTGGTGCAGCCC |
| | GGCGGCAGCCTGAGACTGAGCTGCGCCGCCAGCGGCTACGACTTC |
| | ACCCACTACGGCATGAACTGGGTGAGACAGGCCCCCGGCAAGGGC |
| | CTGGAGTGGGTGGGCTGGATCAACACCTACACCGGCGAGCCCACC |
| | TACGCCGCCGACTTCAAGAGAAGATTCACCTTCAGCCTGGACACC |
| | AGCAAGAGCACCGCCTACCTGCAGATGAACAGCCTGAGAGCCGAG |
| | GACACCGCCGTGTACTACTGCGCCAAGTACCCCTACTACTACGGC |
| | ACCAGCCACTGGTACTTCGACGTGTGGGGCCAGGGCACCCTGGTG |
| | ACCGTGGGCGGAGGCGGAAGCGGCGGAGGCGGATCTGGCGGAGGC |
| | GGCAGCGGCGGCGGCGGCTCTGACATCCAGCTGACCCAGAGCCCC |
| | AGCAGCCTGAGCGCCAGCGTGGGCGACAGAGTGACCATCACCTGC |
| | AGCGCCAGCCAGGACATCAGCAACTACCTGAACTGGTACCAGCAG |
| | AAGCCCGGCAAGGCCCCCAAGGTGCTGATCTACTTCACCAGCAGC |
| | CTGCACAGCGGCGTGCCCAGCAGATTCAGCGGCAGCGGCAGCGGC |
| | ACCGACTTCACCCTGACCATCAGCAGCCTGCAGCCCGAGGACTTC |
| | GCCACCTACTACTGCCAGCAGTACAGCACCGTGCCCTGGACCTTC |
| | GGCCAGGGCACCAAGGTGGAGATCAAGAGAACCGTGGCCGCCTGA |
| | TAATAAAATATCTTTATTTTCATTACATCTGTGTGTTGGTTTTTT |
| | GTGTGGAGGGCCTATTTCCCATGATTCCTTCATATTTGCATATAC |
| | GATACAAGGCTGTTAGAGAGATAATTGGAATTAATTTGACTGTAA |
| | ACACAAAGATATTAGTACAAAATACGTGACGTAGAAAGTAATAAT |
| | TTCTTGGGTAGTTTGCAGTTTTAAAATTATGTTTTAAAATGGACT |
| | ATCATATGCTTACCGTAACTTGAAAGTATTTCGATTTCTTGGCTT |
| | TATATATCTTGTGGAAAGGACAAGCTTGACTTGGAAAGAATATAA |
| | ATACCTGACCCATATTTATATTCTTTCCAAGTCTTTTTGCATGCT |
| | GGGGAGAGATCAACCCCACTCCCTCTCTGCGCGCTCGCTCGCTCA |
| | CTGAGGCCGGGCGACCAAAGGTCGCCCGACGCCCGGGCTTTGCCC |
| | GGGCGGCCTCAGTGAGCGAGCGAGCGCGCAGCAAGCTGTAGCCAA |
| | CCACTAGAACTATAGCTAGAGTCCTGGGCGAACAAACGATGCTCG |
| | CCTTCCAGAAAACCGAGGATGCGAACCACTTCATCCGGGGTCAGC |
| | ACCACCGGCAAGCGCCGCGACGGCCGAGGTCTTCCGATCTCCTGA |
| | AGCCAGGGCAGATCCGTGCACAGCACCTTGCCGTAGAAGAACAGC |
| | AAGGCCGCCAATGCCTGACGATGCGTGGAGACCGAAACCTTGCGC |
| | TCGTTCGCCAGCCAGGACAGAAATGCCTCGACTTCGCTGCTGCCC |
| | AAGGTTGCCGGGTGACGCACACCGTGGAAACGGATGAAGGCACGA |
| | ACCCAGTTGACATAAGCCTGTTCGGTTCGTAAACTGTAATGCAAG |
| | TAGCGTATGCGCTCACGCAACTGGTCCAGAACCTTGACCGAACGC |
| | AGCGGTGGTAACGGCGCAGTGGCGGTTTTCATGGCTTGTTATGAC |
| | TGTTTTTTTGTACAGTCTATGCCTCGGGCATCCAAGCAGCAAGCG |
| | CGTTACGCCGTGGGTCGATGTTTGATGTTATGGAGCAGCAACGAT |
| | GTTACGCAGCAGCAACGATGTTACGCAGCAGGGCAGTCGCCCTAA |
| | AACAAAGTTAGGTGGCTCAAGTATGGGCATCATTCGCACATGTAG |
| | GCTCGGCCCTGACCAAGTCAAATCCATGCGGGCTGCTCTTGATCT |
| | TTTCGGTCGTGAGTTCGGAGACGTAGCCACCTACTCCCAACATCA |
| | GCCGGACTCCGATTACCTCGGGAACTTGCTCCGTAGTAAGACATT |
| | CATCGCGCTTGCTGCCTTCGACCAAGAAGCGGTTGTTGGCGCTCT |
| | CGCGGCTTACGTTCTGCCCAGGTTTGAGCAGCCGCGTAGTGAGAT |
| | CTATATCTATGATCTCGCAGTCTCCGGCGAGCACCGGAGGCAGGG |
| | CATTGCCACCGCGCTCATCAATCTCCTCAAGCATGAGGCCAACGC |
| | GCTTGGTGCTTATGTGATCTACGTGCAAGCAGATTACGGTGACGA |
| | TCCCGCAGTGGCTCTCTATACAAAGTTGGGCATACGGGAAGAAGT |
| | GATGCACTTTGATATCGACCCAAGTACCGCCACCTAACAATTCGT |
| | TCAAGCCGAGATCGGCTTCCCGGCCGCGGAGTTGTTCGGTAAATT |
| | GTCACAACGCCGCGAATATAGTCTTTACCATGCCCTTGGCCACGC |
| | CCCTCTTTAATACGACGGGCAATTTGCACTTCAGAAAATGAAGAG |
| | TTTGCTTTAGCCATAACAAAAGTCCAGTATGCTTTTTCACAGCAT |
| | AACTGGACTGATTTCAGTTTACAACTATTCTGTCTAGTTTAAGAC |
| | TTTATTGTCATAGTTTAGATCTATTTTGTTCAGTTTAAGACTTTA |
| | TTGTCCGCCCACACCCGCTTACGCAGGGCATCCATTTATTACTCA |
| | ACCGTAACCGATTTTGCCAGGTTACGCGGCTGGTCTGCGGTGTGA |
| | AATACCGCACAGATGCGTAAGGAGAAAATACCGCATCAGGCGCTC |
| | TTCCGCTTCCTCGCTCACTGACTCGCTGCGCTCGGTCGTTCGGCT |
| | GCGGCGAGCGGTATCAGCTCACTCAAAGGCGGTAATACGGTTATC |
| | CACAGAATCAGGGGATAACGCAGGAAAGAACATGTGAGCAAAAGG |
| | CCAGCAAAAGGCCAGGAACCGTAAAAAGGCCGCGTTGCTGGCGTT |
| | TTTCCATAGGCTCCGCCCCCCTGACGAGCATCACAAAAATCGACG |
| | CTCAAGTCAGAGGTGGCGAAACCCGACAGGACTATAAAGATACCA |
| | GGCGTTTCCCCCTGGAAGCTCCCTCGTGCGCTCTCCTGTTCCGAC |
| | CCTGCCGCTTACCGGATACCTGTCCGCCTTTCTCCCTTCGGGAAG |
| | CGTGGCGCTTTCTCAATGCTCACGCTGTAGGTATCTCAGTTCGGT |
| | GTAGGTCGTTCGCTCCAAGCTGGGCTGTGTGCACGAACCCCCCGT |
| | TCAGCCCGACCGCTGCGCCTTATCCGGTAACTATCGTCTTGAGTC |
| | CAACCCGGTAAGACACGACTTATCGCCACTGGCAGCAGCCACTGG |
| | TAACAGGATTAGCAGAGCGAGGTATGTAGGCGGTGCTACAGAGTT |
| | CTTGAAGTGGTGGCCTAACTACGGCTACACTAGAAGGACAGTATT |
| | TGGTATCTGCGCTCTGCTGAAGCCAGTTACCTTCGGAAAAAGAGT |
| | TGGTAGCTCTTGATCCGGCAAACAAACCACCGCTGGTAGCGGTGG |
| | TTTTTTTGTTTGCAAGCAGCAGATTACGCGCAGAAAAAAAGGATC |
| | TCAAGAAGATCCTTTGATCTTTTCTACGGGGTCTGACGCTCAGTG |
| | GAACGAAAACTCACGTTAAGGGATTTTGGTCATGAGATTATCAAA |
| | AAGGATCTTCACCTAGATCCTTTTAAATTAAAAATGAAGTTTTAA |
| | ATCAATCTAAAGTATATATGAGTAAACTTGGTCTGACAGTTACCA |
| | ATGCTTAATCAGTGAGGCACCTATCTCAGCGATCTGTCTATTTCG |
| | TTCATCCATAGTTGCCTGACTCCCCGTCGTGTAGATAACTACGAT |
| | ACGGGAGGGCTTACCATCTGGCCCCAGTGCTGCAATGATACCGCG |
| | AGACCCACGCTCACCGGCTCCAGATTTATCAGCAATAAACCAGCC |
| | AGCCGGAAGGGCCGAGCGCAGAAGTGGTCCTGCAACTTTATCCGC |
| | CTCCATCCAGTCTATTAATTGTTGCCGGGAAGCTAGAGTAAGTAG |
| | TTCGCCAGTTAATAGTTTGCGCAACGTTGTTGCCATTGCTACAGG |
| | CATCGTGGTGTCACGCTCGTCGTTTGGTATGGCTTCATTCAGCTC |
| | CGGTTCCCAACGATCAAGGCGAGTTACATGATCCCCCATGTTGTG |
| | CAAAAAAGCGGTTAGCTCCTTCGGTCCTCCGATCGTTGTCAGAAG |
| | TAAGTTGGCCGCAGTGTTATCACTCATGGTTATGGCAGCACTGCA |
| | TAATTCTCTTACTGTCATGCCATCCGTAAGATGCTTTTCTGTGAC |
| | TGGTGAGTACTCAACCAAGTCATTCTGAGAATAGTGTATGCGGCG |
| | ACCGAGTTGCTCTTGCCCGGCGTCAATACGGGATAATACCGCGCC |
| | ACATAGCAGAACTTTAAAAGTGCTCATCATTGGAAAACGTTCTTC |
| | GGGGCGAAAACTCTCAAGGATCTTACCGCTGTTGAGATCCAGTTC |
| | GATGTAACCCACTCGTGCACCCAACTGATCTTCAGCATCTTTTAC |
| | TTTCACCAGCGTTTCTGGGTGAGCAAAAACAGGAAGGCAAAATGC |
| | CGCAAAAAAGGGAATAAGGGCGACACGGAAATGTTGAATACTCAT |
| | ACTCTTCCTTTTTCAATATTATTGAAGCATTTATCAGGGTTATTG |
| | TCTCATGAGCGGATACATATTTGAATGTATTTAGAAAAATAAACA |
| | AATAGGGGTTCCGCGCACATTTCCCCGAAAAGTGCCACCTGAAAT |
| | TGTAAACGTTAATATTTTGTTAAAATTCGCGTTAAATTTTTGTTA |
| | AATCAGCTCATTTTTTAACCAATAGGCCGAAATCGGCAAAATCCC |
| | TTATAAATCAAAAGAATAGACCGAGATAGGGTTGAGTGTTGTTCC |
| | AGTTTGGAACAAGAGTCCACTATTAAAGAACGTGGACTCCAACGT |
| | CAAAGGGCGAAAAACCGTCTATCAGGGCGATGGCCCACTACGTGA |
| | ACCATCACCCTAATCAAGTTTTTTGGGGTCGAGGTGCCGTAAAGC |
| | ACTAAATCGGAACCCTAAAGGGAGCCCCCGATTTAGAGCTTGACG |
| | GGGAAAGCCGGCGAACGTGGCGAGAAAGGAAGGGAAGAAAGCGAA |
| | AGGAGCGGGCGCTAGGGCGCTGGCAAGTGTAGCGGTCACGCTGCG |
| | CGTAACCACCACACCCGCCGCGCTTAATGCGCCGCTACAGGGCGC |
| | GTC |
| AMI162; | 611-755: Full ITR |
| SEQ ID | 801-1104: CMV enhancer |
| NO: 73 | 1105-1308: CMV promoter |
| | 1412-1508: SV40 intron |
| | 1513-1521: Kozak sequence |
| | 1522-1578: Human IgG heavy chain secretion sequence |
| | 1579-1941: Lucentis Vh coding sequence after optimization |
| | 1942-2001: 4xGGGGS linker |
| | 2002-2337: Lucentis VI coding sequence after optimization |
| | 2342-2390: Poly A signal |
| | 2391-2631: Human U6 promoter |
| | 2638-2693: shRNA6 against Ang2 |
| | (GGTGAAGAACTCAATTATAtacctgacccataTATAATTGAGTTCTTCACCTTTTT) |
| | 2716-2821: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTGAAGGGCGTGCAG |
| | TGCGAGGTGCAGCTGGTGGAGAGCGGCGGCGGCCTGGTGCAGCCC |
| | GGCGGCAGCCTGAGACTGAGCTGCGCCGCCAGCGGCTACGACTTC |
| | ACCCACTACGGCATGAACTGGGTGAGACAGGCCCCCGGCAAGGGC |
| | CTGGAGTGGGTGGGCTGGATCAACACCTACACCGGCGAGCCCACC |
| | TACGCCGCCGACTTCAAGAGAAGATTCACCTTCAGCCTGGACACC |
| | AGCAAGAGCACCGCCTACCTGCAGATGAACAGCCTGAGAGCCGAG |
| | GACACCGCCGTGTACTACTGCGCCAAGTACCCCTACTACTACGGC |
| | ACCAGCCACTGGTACTTCGACGTGTGGGGCCAGGGCACCCTGGTG |
| | ACCGTGGGCGGAGGCGGAAGCGGCGGAGGCGGATCTGGCGGAGGC |
| | GGCAGCGGCGGCGGCGGCTCTGACATCCAGCTGACCCAGAGCCCC |
| | AGCAGCCTGAGCGCCAGCGTGGGCGACAGAGTGACCATCACCTGC |
| | AGCGCCAGCCAGGACATCAGCAACTACCTGAACTGGTACCAGCAG |
| | AAGCCCGGCAAGGCCCCCAAGGTGCTGATCTACTTCACCAGCAGC |
| | CTGCACAGCGGCGTGCCCAGCAGATTCAGCGGCAGCGGCAGCGGC |
| | ACCGACTTCACCCTGACCATCAGCAGCCTGCAGCCCGAGGACTTC |
| | GCCACCTACTACTGCCAGCAGTACAGCACCGTGCCCTGGACCTTC |
| | GGCCAGGGCACCAAGGTGGAGATCAAGAGAACCGTGGCCGCCTGA |
| | TAATAAAATATCTTTATTTTCATTACATCTGTGTGTTGGTTTTTT |
| | GTGTGGAGGGCCTATTTCCCATGATTCCTTCATATTTGCATATAC |
| | GATACAAGGCTGTTAGAGAGATAATTGGAATTAATTTGACTGTAA |
| | ACACAAAGATATTAGTACAAAATACGTGACGTAGAAAGTAATAAT |
| | TTCTTGGGTAGTTTGCAGTTTTAAAATTATGTTTTAAAATGGACT |
| | ATCATATGCTTACCGTAACTTGAAAGTATTTCGATTTCTTGGCTT |
| | TATATATCTTGTGGAAAGGACAAGCTTGGTGAAGAACTCAATTAT |
| | ATACCTGACCCATATATAATTGAGTTCTTCACCTTTTTGCATGCT |
| | GGGGAGAGATCAACCCCACTCCCTCTCTGCGCGCTCGCTCGCTCA |
| | CTGAGGCCGGGCGACCAAAGGTCGCCCGACGCCCGGGCTTTGCCC |
| | GGGCGGCCTCAGTGAGCGAGCGAGCGCGCAGCAAGCTGTAGCCAA |
| | CCACTAGAACTATAGCTAGAGTCCTGGGCGAACAAACGATGCTCG |
| | CCTTCCAGAAAACCGAGGATGCGAACCACTTCATCCGGGGTCAGC |
| | ACCACCGGCAAGCGCCGCGACGGCCGAGGTCTTCCGATCTCCTGA |
| | AGCCAGGGCAGATCCGTGCACAGCACCTTGCCGTAGAAGAACAGC |
| | AAGGCCGCCAATGCCTGACGATGCGTGGAGACCGAAACCTTGCGC |
| | TCGTTCGCCAGCCAGGACAGAAATGCCTCGACTTCGCTGCTGCCC |
| | AAGGTTGCCGGGTGACGCACACCGTGGAAACGGATGAAGGCACGA |
| | ACCCAGTTGACATAAGCCTGTTCGGTTCGTAAACTGTAATGCAAG |
| | TAGCGTATGCGCTCACGCAACTGGTCCAGAACCTTGACCGAACGC |
| | AGCGGTGGTAACGGCGCAGTGGCGGTTTTCATGGCTTGTTATGAC |
| | TGTTTTTTTGTACAGTCTATGCCTCGGGCATCCAAGCAGCAAGCG |
| | CGTTACGCCGTGGGTCGATGTTTGATGTTATGGAGCAGCAACGAT |
| | GTTACGCAGCAGCAACGATGTTACGCAGCAGGGCAGTCGCCCTAA |
| | AACAAAGTTAGGTGGCTCAAGTATGGGCATCATTCGCACATGTAG |
| | GCTCGGCCCTGACCAAGTCAAATCCATGCGGGCTGCTCTTGATCT |
| | TTTCGGTCGTGAGTTCGGAGACGTAGCCACCTACTCCCAACATCA |
| | GCCGGACTCCGATTACCTCGGGAACTTGCTCCGTAGTAAGACATT |
| | CATCGCGCTTGCTGCCTTCGACCAAGAAGCGGTTGTTGGCGCTCT |
| | CGCGGCTTACGTTCTGCCCAGGTTTGAGCAGCCGCGTAGTGAGAT |
| | CTATATCTATGATCTCGCAGTCTCCGGCGAGCACCGGAGGCAGGG |
| | CATTGCCACCGCGCTCATCAATCTCCTCAAGCATGAGGCCAACGC |
| | GCTTGGTGCTTATGTGATCTACGTGCAAGCAGATTACGGTGACGA |
| | TCCCGCAGTGGCTCTCTATACAAAGTTGGGCATACGGGAAGAAGT |
| | GATGCACTTTGATATCGACCCAAGTACCGCCACCTAACAATTCGT |
| | TCAAGCCGAGATCGGCTTCCCGGCCGCGGAGTTGTTCGGTAAATT |
| | GTCACAACGCCGCGAATATAGTCTTTACCATGCCCTTGGCCACGC |
| | CCCTCTTTAATACGACGGGCAATTTGCACTTCAGAAAATGAAGAG |
| | TTTGCTTTAGCCATAACAAAAGTCCAGTATGCTTTTTCACAGCAT |
| | AACTGGACTGATTTCAGTTTACAACTATTCTGTCTAGTTTAAGAC |
| | TTTATTGTCATAGTTTAGATCTATTTTGTTCAGTTTAAGACTTTA |
| | TTGTCCGCCCACACCCGCTTACGCAGGGCATCCATTTATTACTCA |
| | ACCGTAACCGATTTTGCCAGGTTACGCGGCTGGTCTGCGGTGTGA |
| | AATACCGCACAGATGCGTAAGGAGAAAATACCGCATCAGGCGCTC |
| | TTCCGCTTCCTCGCTCACTGACTCGCTGCGCTCGGTCGTTCGGCT |
| | GCGGCGAGCGGTATCAGCTCACTCAAAGGCGGTAATACGGTTATC |
| | CACAGAATCAGGGGATAACGCAGGAAAGAACATGTGAGCAAAAGG |
| | CCAGCAAAAGGCCAGGAACCGTAAAAAGGCCGCGTTGCTGGCGTT |
| | TTTCCATAGGCTCCGCCCCCCTGACGAGCATCACAAAAATCGACG |
| | CTCAAGTCAGAGGTGGCGAAACCCGACAGGACTATAAAGATACCA |
| | GGCGTTTCCCCCTGGAAGCTCCCTCGTGCGCTCTCCTGTTCCGAC |
| | CCTGCCGCTTACCGGATACCTGTCCGCCTTTCTCCCTTCGGGAAG |
| | CGTGGCGCTTTCTCAATGCTCACGCTGTAGGTATCTCAGTTCGGT |
| | GTAGGTCGTTCGCTCCAAGCTGGGCTGTGTGCACGAACCCCCCGT |
| | TCAGCCCGACCGCTGCGCCTTATCCGGTAACTATCGTCTTGAGTC |
| | CAACCCGGTAAGACACGACTTATCGCCACTGGCAGCAGCCACTGG |
| | TAACAGGATTAGCAGAGCGAGGTATGTAGGCGGTGCTACAGAGTT |
| | CTTGAAGTGGTGGCCTAACTACGGCTACACTAGAAGGACAGTATT |
| | TGGTATCTGCGCTCTGCTGAAGCCAGTTACCTTCGGAAAAAGAGT |
| | TGGTAGCTCTTGATCCGGCAAACAAACCACCGCTGGTAGCGGTGG |
| | TTTTTTTGTTTGCAAGCAGCAGATTACGCGCAGAAAAAAAGGATC |
| | TCAAGAAGATCCTTTGATCTTTTCTACGGGGTCTGACGCTCAGTG |
| | GAACGAAAACTCACGTTAAGGGATTTTGGTCATGAGATTATCAAA |
| | AAGGATCTTCACCTAGATCCTTTTAAATTAAAAATGAAGTTTTAA |
| | ATCAATCTAAAGTATATATGAGTAAACTTGGTCTGACAGTTACCA |
| | ATGCTTAATCAGTGAGGCACCTATCTCAGCGATCTGTCTATTTCG |
| | TTCATCCATAGTTGCCTGACTCCCCGTCGTGTAGATAACTACGAT |
| | ACGGGAGGGCTTACCATCTGGCCCCAGTGCTGCAATGATACCGCG |
| | AGACCCACGCTCACCGGCTCCAGATTTATCAGCAATAAACCAGCC |
| | AGCCGGAAGGGCCGAGCGCAGAAGTGGTCCTGCAACTTTATCCGC |
| | CTCCATCCAGTCTATTAATTGTTGCCGGGAAGCTAGAGTAAGTAG |
| | TTCGCCAGTTAATAGTTTGCGCAACGTTGTTGCCATTGCTACAGG |
| | CATCGTGGTGTCACGCTCGTCGTTTGGTATGGCTTCATTCAGCTC |
| | CGGTTCCCAACGATCAAGGCGAGTTACATGATCCCCCATGTTGTG |
| | CAAAAAAGCGGTTAGCTCCTTCGGTCCTCCGATCGTTGTCAGAAG |
| | TAAGTTGGCCGCAGTGTTATCACTCATGGTTATGGCAGCACTGCA |
| | TAATTCTCTTACTGTCATGCCATCCGTAAGATGCTTTTCTGTGAC |
| | TGGTGAGTACTCAACCAAGTCATTCTGAGAATAGTGTATGCGGCG |
| | ACCGAGTTGCTCTTGCCCGGCGTCAATACGGGATAATACCGCGCC |
| | ACATAGCAGAACTTTAAAAGTGCTCATCATTGGAAAACGTTCTTC |
| | GGGGCGAAAACTCTCAAGGATCTTACCGCTGTTGAGATCCAGTTC |
| | GATGTAACCCACTCGTGCACCCAACTGATCTTCAGCATCTTTTAC |
| | TTTCACCAGCGTTTCTGGGTGAGCAAAAACAGGAAGGCAAAATGC |
| | CGCAAAAAAGGGAATAAGGGCGACACGGAAATGTTGAATACTCAT |
| | ACTCTTCCTTTTTCAATATTATTGAAGCATTTATCAGGGTTATTG |
| | TCTCATGAGCGGATACATATTTGAATGTATTTAGAAAAATAAACA |
| | AATAGGGGTTCCGCGCACATTTCCCCGAAAAGTGCCACCTGAAAT |
| | TGTAAACGTTAATATTTTGTTAAAATTCGCGTTAAATTTTTGTTA |
| | AATCAGCTCATTTTTTAACCAATAGGCCGAAATCGGCAAAATCCC |
| | TTATAAATCAAAAGAATAGACCGAGATAGGGTTGAGTGTTGTTCC |
| | AGTTTGGAACAAGAGTCCACTATTAAAGAACGTGGACTCCAACGT |
| | CAAAGGGCGAAAAACCGTCTATCAGGGCGATGGCCCACTACGTGA |
| | ACCATCACCCTAATCAAGTTTTTTGGGGTCGAGGTGCCGTAAAGC |
| | ACTAAATCGGAACCCTAAAGGGAGCCCCCGATTTAGAGCTTGACG |
| | GGGAAAGCCGGCGAACGTGGCGAGAAAGGAAGGGAAGAAAGCGAA |
| | AGGAGCGGGCGCTAGGGCGCTGGCAAGTGTAGCGGTCACGCTGCG |
| | CGTAACCACCACACCCGCCGCGCTTAATGCGCCGCTACAGGGCGC |
| | GTC |
| AMI163; | 611-755: Full ITR |
| SEQ ID | 801-1104: CMV enhancer |
| NO: 74 | 1105-1308: CMV promoter |
| | 1412-1508: SV40 intron |
| | 1513-1521: Kozak sequence |
| | 1522-1578: Human IgG heavy chain secretion sequence |
| | 1579-1941: Lucentis Vh coding sequence after optimization |
| | 1942-2001: 4xGGGGS linker |
| | 2002-2337: Lucentis VI coding sequence after optimization |
| | 2347-2395: Poly A signal |
| | 2396-2636: Human U6 promoter |
| | 2643-2698: shRNA scramble |
| | (GTGCATATGAACGTAACTAtacctgacccataTAGTTACGTTCATATGCACTTTTT) |
| | 2722-2827: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTGAAGGGCGTGCAG |
| | TGCGAGGTGCAGCTGGTGGAGAGCGGCGGCGGCCTGGTGCAGCCC |
| | GGCGGCAGCCTGAGACTGAGCTGCGCCGCCAGCGGCTACGACTTC |
| | ACCCACTACGGCATGAACTGGGTGAGACAGGCCCCCGGCAAGGGC |
| | CTGGAGTGGGTGGGCTGGATCAACACCTACACCGGCGAGCCCACC |
| | TACGCCGCCGACTTCAAGAGAAGATTCACCTTCAGCCTGGACACC |
| | AGCAAGAGCACCGCCTACCTGCAGATGAACAGCCTGAGAGCCGAG |
| | GACACCGCCGTGTACTACTGCGCCAAGTACCCCTACTACTACGGC |
| | ACCAGCCACTGGTACTTCGACGTGTGGGGCCAGGGCACCCTGGTG |
| | ACCGTGGGCGGAGGCGGAAGCGGCGGAGGCGGATCTGGCGGAGGC |
| | GGCAGCGGCGGCGGCGGCTCTGACATCCAGCTGACCCAGAGCCCC |
| | AGCAGCCTGAGCGCCAGCGTGGGCGACAGAGTGACCATCACCTGC |
| | AGCGCCAGCCAGGACATCAGCAACTACCTGAACTGGTACCAGCAG |
| | AAGCCCGGCAAGGCCCCCAAGGTGCTGATCTACTTCACCAGCAGC |
| | CTGCACAGCGGCGTGCCCAGCAGATTCAGCGGCAGCGGCAGCGGC |
| | ACCGACTTCACCCTGACCATCAGCAGCCTGCAGCCCGAGGACTTC |
| | GCCACCTACTACTGCCAGCAGTACAGCACCGTGCCCTGGACCTTC |
| | GGCCAGGGCACCAAGGTGGAGATCAAGAGAACCGTGGCCGCCTGA |
| | TTCGAAAATAAAATATCTTTATTTTCATTACATCTGTGTGTTGGT |
| | TTTTTGTGTGGAGGGCCTATTTCCCATGATTCCTTCATATTTGCA |
| | TATACGATACAAGGCTGTTAGAGAGATAATTGGAATTAATTTGAC |
| | TGTAAACACAAAGATATTAGTACAAAATACGTGACGTAGAAAGTA |
| | ATAATTTCTTGGGTAGTTTGCAGTTTTAAAATTATGTTTTAAAAT |
| | GGACTATCATATGCTTACCGTAACTTGAAAGTATTTCGATTTCTT |
| | GGCTTTATATATCTTGTGGAAAGGACAAGCTTGTGCATATGAACG |
| | TAACTATACCTGACCCATATAGTTACGTTCATATGCACTTTTTGC |
| | ATGCTGGGGAGAGATCAACCCCACTCCCTCTCTGCGCGCTCGCTC |
| | GCTCACTGAGGCCGGGCGACCAAAGGTCGCCCGACGCCCGGGCTT |
| | TGCCCGGGCGGCCTCAGTGAGCGAGCGAGCGCGCAGCAAGCTGTA |
| | GCCAACCACTAGAACTATAGCTAGAGTCCTGGGCGAACAAACGAT |
| | GCTCGCCTTCCAGAAAACCGAGGATGCGAACCACTTCATCCGGGG |
| | TCAGCACCACCGGCAAGCGCCGCGACGGCCGAGGTCTTCCGATCT |
| | CCTGAAGCCAGGGCAGATCCGTGCACAGCACCTTGCCGTAGAAGA |
| | ACAGCAAGGCCGCCAATGCCTGACGATGCGTGGAGACCGAAACCT |
| | TGCGCTCGTTCGCCAGCCAGGACAGAAATGCCTCGACTTCGCTGC |
| | TGCCCAAGGTTGCCGGGTGACGCACACCGTGGAAACGGATGAAGG |
| | CACGAACCCAGTTGACATAAGCCTGTTCGGTTCGTAAACTGTAAT |
| | GCAAGTAGCGTATGCGCTCACGCAACTGGTCCAGAACCTTGACCG |
| | AACGCAGCGGTGGTAACGGCGCAGTGGCGGTTTTCATGGCTTGTT |
| | ATGACTGTTTTTTTGTACAGTCTATGCCTCGGGCATCCAAGCAGC |
| | AAGCGCGTTACGCCGTGGGTCGATGTTTGATGTTATGGAGCAGCA |
| | ACGATGTTACGCAGCAGCAACGATGTTACGCAGCAGGGCAGTCGC |
| | CCTAAAACAAAGTTAGGTGGCTCAAGTATGGGCATCATTCGCACA |
| | TGTAGGCTCGGCCCTGACCAAGTCAAATCCATGCGGGCTGCTCTT |
| | GATCTTTTCGGTCGTGAGTTCGGAGACGTAGCCACCTACTCCCAA |
| | CATCAGCCGGACTCCGATTACCTCGGGAACTTGCTCCGTAGTAAG |
| | ACATTCATCGCGCTTGCTGCCTTCGACCAAGAAGCGGTTGTTGGC |
| | GCTCTCGCGGCTTACGTTCTGCCCAGGTTTGAGCAGCCGCGTAGT |
| | GAGATCTATATCTATGATCTCGCAGTCTCCGGCGAGCACCGGAGG |
| | CAGGGCATTGCCACCGCGCTCATCAATCTCCTCAAGCATGAGGCC |
| | AACGCGCTTGGTGCTTATGTGATCTACGTGCAAGCAGATTACGGT |
| | GACGATCCCGCAGTGGCTCTCTATACAAAGTTGGGCATACGGGAA |
| | GAAGTGATGCACTTTGATATCGACCCAAGTACCGCCACCTAACAA |
| | TTCGTTCAAGCCGAGATCGGCTTCCCGGCCGCGGAGTTGTTCGGT |
| | AAATTGTCACAACGCCGCGAATATAGTCTTTACCATGCCCTTGGC |
| | CACGCCCCTCTTTAATACGACGGGCAATTTGCACTTCAGAAAATG |
| | AAGAGTTTGCTTTAGCCATAACAAAAGTCCAGTATGCTTTTTCAC |
| | AGCATAACTGGACTGATTTCAGTTTACAACTATTCTGTCTAGTTT |
| | AAGACTTTATTGTCATAGTTTAGATCTATTTTGTTCAGTTTAAGA |
| | CTTTATTGTCCGCCCACACCCGCTTACGCAGGGCATCCATTTATT |
| | ACTCAACCGTAACCGATTTTGCCAGGTTACGCGGCTGGTCTGCGG |
| | TGTGAAATACCGCACAGATGCGTAAGGAGAAAATACCGCATCAGG |
| | CGCTCTTCCGCTTCCTCGCTCACTGACTCGCTGCGCTCGGTCGTT |
| | CGGCTGCGGCGAGCGGTATCAGCTCACTCAAAGGCGGTAATACGG |
| | TTATCCACAGAATCAGGGGATAACGCAGGAAAGAACATGTGAGCA |
| | AAAGGCCAGCAAAAGGCCAGGAACCGTAAAAAGGCCGCGTTGCTG |
| | GCGTTTTTCCATAGGCTCCGCCCCCCTGACGAGCATCACAAAAAT |
| | CGACGCTCAAGTCAGAGGTGGCGAAACCCGACAGGACTATAAAGA |
| | TACCAGGCGTTTCCCCCTGGAAGCTCCCTCGTGCGCTCTCCTGTT |
| | CCGACCCTGCCGCTTACCGGATACCTGTCCGCCTTTCTCCCTTCG |
| | GGAAGCGTGGCGCTTTCTCAATGCTCACGCTGTAGGTATCTCAGT |
| | TCGGTGTAGGTCGTTCGCTCCAAGCTGGGCTGTGTGCACGAACCC |
| | CCCGTTCAGCCCGACCGCTGCGCCTTATCCGGTAACTATCGTCTT |
| | GAGTCCAACCCGGTAAGACACGACTTATCGCCACTGGCAGCAGCC |
| | ACTGGTAACAGGATTAGCAGAGCGAGGTATGTAGGCGGTGCTACA |
| | GAGTTCTTGAAGTGGTGGCCTAACTACGGCTACACTAGAAGGACA |
| | GTATTTGGTATCTGCGCTCTGCTGAAGCCAGTTACCTTCGGAAAA |
| | AGAGTTGGTAGCTCTTGATCCGGCAAACAAACCACCGCTGGTAGC |
| | GGTGGTTTTTTTGTTTGCAAGCAGCAGATTACGCGCAGAAAAAAA |
| | GGATCTCAAGAAGATCCTTTGATCTTTTCTACGGGGTCTGACGCT |
| | CAGTGGAACGAAAACTCACGTTAAGGGATTTTGGTCATGAGATTA |
| | TCAAAAAGGATCTTCACCTAGATCCTTTTAAATTAAAAATGAAGT |
| | TTTAAATCAATCTAAAGTATATATGAGTAAACTTGGTCTGACAGT |
| | TACCAATGCTTAATCAGTGAGGCACCTATCTCAGCGATCTGTCTA |
| | TTTCGTTCATCCATAGTTGCCTGACTCCCCGTCGTGTAGATAACT |
| | ACGATACGGGAGGGCTTACCATCTGGCCCCAGTGCTGCAATGATA |
| | CCGCGAGACCCACGCTCACCGGCTCCAGATTTATCAGCAATAAAC |
| | CAGCCAGCCGGAAGGGCCGAGCGCAGAAGTGGTCCTGCAACTTTA |
| | TCCGCCTCCATCCAGTCTATTAATTGTTGCCGGGAAGCTAGAGTA |
| | AGTAGTTCGCCAGTTAATAGTTTGCGCAACGTTGTTGCCATTGCT |
| | ACAGGCATCGTGGTGTCACGCTCGTCGTTTGGTATGGCTTCATTC |
| | AGCTCCGGTTCCCAACGATCAAGGCGAGTTACATGATCCCCCATG |
| | TTGTGCAAAAAAGCGGTTAGCTCCTTCGGTCCTCCGATCGTTGTC |
| | AGAAGTAAGTTGGCCGCAGTGTTATCACTCATGGTTATGGCAGCA |
| | CTGCATAATTCTCTTACTGTCATGCCATCCGTAAGATGCTTTTCT |
| | GTGACTGGTGAGTACTCAACCAAGTCATTCTGAGAATAGTGTATG |
| | CGGCGACCGAGTTGCTCTTGCCCGGCGTCAATACGGGATAATACC |
| | GCGCCACATAGCAGAACTTTAAAAGTGCTCATCATTGGAAAACGT |
| | TCTTCGGGGCGAAAACTCTCAAGGATCTTACCGCTGTTGAGATCC |
| | AGTTCGATGTAACCCACTCGTGCACCCAACTGATCTTCAGCATCT |
| | TTTACTTTCACCAGCGTTTCTGGGTGAGCAAAAACAGGAAGGCAA |
| | AATGCCGCAAAAAAGGGAATAAGGGCGACACGGAAATGTTGAATA |
| | CTCATACTCTTCCTTTTTCAATATTATTGAAGCATTTATCAGGGT |
| | TATTGTCTCATGAGCGGATACATATTTGAATGTATTTAGAAAAAT |
| | AAACAAATAGGGGTTCCGCGCACATTTCCCCGAAAAGTGCCACCT |
| | GAAATTGTAAACGTTAATATTTTGTTAAAATTCGCGTTAAATTTT |
| | TGTTAAATCAGCTCATTTTTTAACCAATAGGCCGAAATCGGCAAA |
| | ATCCCTTATAAATCAAAAGAATAGACCGAGATAGGGTTGAGTGTT |
| | GTTCCAGTTTGGAACAAGAGTCCACTATTAAAGAACGTGGACTCC |
| | AACGTCAAAGGGCGAAAAACCGTCTATCAGGGCGATGGCCCACTA |
| | CGTGAACCATCACCCTAATCAAGTTTTTTGGGGTCGAGGTGCCGT |
| | AAAGCACTAAATCGGAACCCTAAAGGGAGCCCCCGATTTAGAGCT |
| | TGACGGGGAAAGCCGGCGAACGTGGCGAGAAAGGAAGGGAAGAAA |
| | GCGAAAGGAGCGGGCGCTAGGGCGCTGGCAAGTGTAGCGGTCACG |
| | CTGCGCGTAACCACCACACCCGCCGCGCTTAATGCGCCGCTACAG |
| | GGCGCGTC |
| AMI166; | 372-512: Full ITR |
| SEQ ID | 570-873: CMV enhancer |
| NO: 75 | 874-1077: CMV promoter |
| | 1181-1277: SV40 intron |
| | 1282-1290: Kozak sequence |
| | 1291-1371: VEGF-Trap secretion sequence |
| | 1372-2667: VEGF-Trap optimized coding sequence |
| | 2674-2772: Poly A signal |
| | 2757-3060: CMV enhancer |
| | 3061-3264: CMV promoter |
| | 3368-3464: SV40 intron |
| | 3469-3477: kozak sequence |
| | 3478-3534: Human IgG heavy chain secretion sequence |
| | 3535-4281: TNFa-ScFv |
| | 4291-4339: Poly A signal |
| | 4384-4524: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGTGAGCTACTGG |
| | GACACCGGCGTGCTGCTGTGCGCCCTGCTGAGCTGCCTGCTGCTG |
| | ACCGGCAGCAGCAGCGGCAGCGACACCGGCAGGCCCTTCGTGGAG |
| | ATGTACTCCGAGATCCCCGAGATCATCCACATGACCGAGGGCAGG |
| | GAGCTGGTGATCCCCTGCAGGGTGACCTCCCCCAACATCACCGTG |
| | ACCCTGAAGAAGTTCCCCCTGGACACCCTGATCCCCGACGGCAAG |
| | AGGATCATCTGGGACTCCAGGAAGGGCTTCATCATCTCCAACGCC |
| | ACCTACAAGGAGATCGGCCTGCTGACCTGCGAGGCCACCGTGAAC |
| | GGCCACCTGTACAAGACCAACTACCTGACCCACAGGCAGACCAAC |
| | ACCATCATCGACGTGGTGCTGTCCCCCTCCCACGGCATCGAGCTG |
| | TCCGTGGGCGAGAAGCTGGTGCTGAACTGCACCGCCAGGACCGAG |
| | CTGAACGTGGGCATCGACTTCAACTGGGAGTACCCCTCCTCCAAG |
| | CACCAGCACAAGAAGCTGGTGAACAGGGACCTGAAGACCCAGTCC |
| | GGCTCCGAGATGAAGAAGTTCCTGTCCACCCTGACCATCGACGGC |
| | GTGACCAGGTCCGACCAGGGCCTGTACACCTGCGCCGCCTCCTCC |
| | GGCCTGATGACCAAGAAGAACTCCACCTTCGTGAGGGTGCACGAG |
| | AAGGACAAGACCCACACCTGCCCCCCCTGCCCCGCCCCCGAGCTG |
| | CTGGGCGGCCCCTCCGTGTTCCTGTTCCCCCCCAAGCCCAAGGAC |
| | ACCCTGATGATCTCCAGGACCCCCGAGGTGACCTGCGTGGTGGTG |
| | GACGTGTCCCACGAGGACCCCGAGGTGAAGTTCAACTGGTACGTG |
| | GACGGCGTGGAGGTGCACAACGCCAAGACCAAGCCCAGGGAGGAG |
| | CAGTACAACTCCACCTACAGGGTGGTGTCCGTGCTGACCGTGCTG |
| | CACCAGGACTGGCTGAACGGCAAGGAGTACAAGTGCAAGGTGTCC |
| | AACAAGGCCCTGCCCGCCCCCATCGAGAAGACCATCTCCAAGGCC |
| | AAGGGCCAGCCCAGGGAGCCCCAGGTGTACACCCTGCCCCCCTCC |
| | AGGGACGAGCTGACCAAGAACCAGGTGTCCCTGACCTGCCTGGTG |
| | AAGGGCTTCTACCCCTCCGACATCGCCGTGGAGTGGGAGTCCAAC |
| | GGCCAGCCCGAGAACAACTACAAGACCACCCCCCCCGTGCTGGAC |
| | TCCGACGGCTCCTTCTTCCTGTACTCCAAGCTGACCGTGGACAAG |
| | TCCAGGTGGCAGCAGGGCAACGTGTTCTCCTGCTCCGTGATGCAC |
| | GAGGCCCTGCACAACCACTACACCCAGAAGTCCCTGTCCCTGTCC |
| | CCCGGCAAGTGATTCGAAAATAAAATATCTTTATTTTCATTACAT |
| | CTGTGTGTTGGTTTTTTGTGTGGCATGCTGGGGAGAGATCAACCG |
| | AATTCGGTACCCGTTACATAACTTACGGTAAATGGCCCGCCTGGC |
| | TGACCGCCCAACGACCCCCGCCCATTGACGTCAATAATGACGTAT |
| | GTTCCCATAGTAACGCCAATAGGGACTTTCCATTGACGTCAATGG |
| | GTGGAGTATTTACGGTAAACTGCCCACTTGGCAGTACATCAAGTG |
| | TATCATATGCCAAGTACGCCCCCTATTGACGTCAATGACGGTAAA |
| | TGGCCCGCCTGGCATTATGCCCAGTACATGACCTTATGGGACTTT |
| | CCTACTTGGCAGTACATCTACGTATTAGTCATCGCTATTACCATG |
| | GTGATGCGGTTTTGGCAGTACATCAATGGGCGTGGATAGCGGTTT |
| | GACTCACGGGGATTTCCAAGTCTCCACCCCATTGACGTCAATGGG |
| | AGTTTGTTTTGGCACCAAAATCAACGGGACTTTCCAAAATGTCGT |
| | AACAACTCCGCCCCATTGACGCAAATGGGCGGTAGGCGTGTACGG |
| | TGGGAGGTCTATATAAGCAGAGCTCGTTTAGTGAACCGTCAGATC |
| | GCCTGGAGACGCCATCCACGCTGTTTTGACCTCCATAGAAGACAC |
| | CGGGACCGATCCAGCCTCCGGACTCTAGAGTTAACTGGTAAGTTT |
| | AGTCTTTTTGTCTTTTATTTCAGGTCCCGGATCCGGTGGTGGTGC |
| | AAATCAAAGAACTGCTCCTCAGTGGATGTTGCCTTTACTTCTAGG |
| | CCTGCCGCCACCATGGAGTTCGGCCTGAGCTGGCTGTTCCTGGTG |
| | GCCATCCTTAAGGGCGTGCAGTGCGAGGTGCAGCTGGTGGAGAGC |
| | GGAGGCGGTCTGGTGCAGCCAGGCAGGAGCCTGAGGCTGAGCTGC |
| | GCCGCCAGCGGCTTCACCTTCGACGACTACGCCATGCACTGGGTG |
| | AGGCAGGCCCCAGGCAAGGGCCTGGAGTGGGTGAGCGCCATCACC |
| | TGGAACAGCGGTCACATCGACTACGCCGACAGCGTGGAGGGTAGG |
| | TTCACCATCAGCAGGGACAACGCCAAGAACAGCCTGTACCTGCAG |
| | ATGAACAGCCTGAGGGCCGAGGACACCGCCGTGTACTACTGCGCC |
| | AAGGTGAGCTACCTGAGCACCGCCAGCAGCCTGGACTACTGGGGT |
| | CAGGGCACCCTGGTGACCGTGAGCAGCGGTGGAGGAGGTAGCGGT |
| | GGCGGTGGTAGCGGTGGCGGAGGCAGCGGTGGAGGTGGCAGCGAC |
| | ATCCAGATGACCCAGAGCCCTAGCAGCCTGAGCGCTAGCGTGGGT |
| | GACAGGGTGACCATCACCTGCAGGGCCAGCCAGGGCATCAGGAAC |
| | TACCTGGCCTGGTACCAGCAGAAGCCCGGCAAGGCCCCCAAGCTG |
| | CTGATCTACGCCGCCAGCACCCTGCAGAGCGGCGTGCCCAGCAGG |
| | TTCAGCGGCAGCGGCAGCGGCACCGACTTCACCCTGACCATCAGC |
| | AGCCTGCAGCCCGAGGACGTGGCCACCTACTACTGCCAGAGGTAC |
| | AACAGGGCCCCCTACACCTTCGGCCAGGGCACCAAGGTGGAGATC |
| | AAGAGGTGATTCGAAAATAAAATATCTTTATTTTCATTACATCTG |
| | TGTGTTGGTTTTTTGTGTGGCATGCTGGGGAGAGATCAACCCACG |
| | TGCGGACCGAGCGGCCGCAGGAACCCCTAGTGATGGAGTTGGCCA |
| | CTCCCTCTCTGCGCGCTCGCTCGCTCACTGAGGCCGGGCGACCAA |
| | AGGTCGCCCGACGCCCGGGCTTTGCCCGGGCGGCCTCAGTGAGCG |
| | AGCGAGCGCGCAGCTGCCTGCAGGCATGCAAGCTGTAGCCAACCA |
| | CTAGAACTATAGCTAGAGTCCTGGGCGAACAAACGATGCTCGCCT |
| | TCCAGAAAACCGAGGATGCGAACCACTTCATCCGGGGTCAGCACC |
| | ACCGGCAAGCGCCGCGACGGCCGAGGTCTTCCGATCTCCTGAAGC |
| | CAGGGCAGATCCGTGCACAGCACCTTGCCGTAGAAGAACAGCAAG |
| | GCCGCCAATGCCTGACGATGCGTGGAGACCGAAACCTTGCGCTCG |
| | TTCGCCAGCCAGGACAGAAATGCCTCGACTTCGCTGCTGCCCAAG |
| | GTTGCCGGGTGACGCACACCGTGGAAACGGATGAAGGCACGAACC |
| | CAGTTGACATAAGCCTGTTCGGTTCGTAAACTGTAATGCAAGTAG |
| | CGTATGCGCTCACGCAACTGGTCCAGAACCTTGACCGAACGCAGC |
| | GGTGGTAACGGCGCAGTGGCGGTTTTCATGGCTTGTTATGACTGT |
| | TTTTTTGTACAGTCTATGCCTCGGGCATCCAAGCAGCAAGCGCGT |
| | TACGCCGTGGGTCGATGTTTGATGTTATGGAGCAGCAACGATGTT |
| | ACGCAGCAGCAACGATGTTACGCAGCAGGGCAGTCGCCCTAAAAC |
| | AAAGTTAGGTGGCTCAAGTATGGGCATCATTCGCACATGTAGGCT |
| | CGGCCCTGACCAAGTCAAATCCATGCGGGCTGCTCTTGATCTTTT |
| | CGGTCGTGAGTTCGGAGACGTAGCCACCTACTCCCAACATCAGCC |
| | GGACTCCGATTACCTCGGGAACTTGCTCCGTAGTAAGACATTCAT |
| | CGCGCTTGCTGCCTTCGACCAAGAAGCGGTTGTTGGCGCTCTCGC |
| | GGCTTACGTTCTGCCCAGGTTTGAGCAGCCGCGTAGTGAGATCTA |
| | TATCTATGATCTCGCAGTCTCCGGCGAGCACCGGAGGCAGGGCAT |
| | TGCCACCGCGCTCATCAATCTCCTCAAGCATGAGGCCAACGCGCT |
| | TGGTGCTTATGTGATCTACGTGCAAGCAGATTACGGTGACGATCC |
| | CGCAGTGGCTCTCTATACAAAGTTGGGCATACGGGAAGAAGTGAT |
| | GCACTTTGATATCGACCCAAGTACCGCCACCTAACAATTCGTTCA |
| | AGCCGAGATCGGCTTCCCGGCCGCGGAGTTGTTCGGTAAATTGTC |
| | ACAACGCCGCGAATATAGTCTTTACCATGCCCTTGGCCACGCCCC |
| | TCTTTAATACGACGGGCAATTTGCACTTCAGAAAATGAAGAGTTT |
| | GCTTTAGCCATAACAAAAGTCCAGTATGCTTTTTCACAGCATAAC |
| | TGGACTGATTTCAGTTTACAACTATTCTGTCTAGTTTAAGACTTT |
| | ATTGTCATAGTTTAGATCTATTTTGTTCAGTTTAAGACTTTATTG |
| | TCCGCCCACACCCGCTTACGCAGGGCATCCATTTATTACTCAACC |
| | GTAACCGATTTTGCCAGGTTACGCGGCTGGTCTGCGGTGTGAAAT |
| | ACCGCACAGATGCGTAAGGAGAAAATACCGCATCAGGCGCTCTTC |
| | CGCTTCCTCGCTCACTGACTCGCTGCGCTCGGTCGTTCGGCTGCG |
| | GCGAGCGGTATCAGCTCACTCAAAGGCGGTAATACGGTTATCCAC |
| | AGAATCAGGGGATAACGCAGGAAAGAACATGTGAGCAAAAGGCCA |
| | GCAAAAGGCCAGGAACCGTAAAAAGGCCGCGTTGCTGGCGTTTTT |
| | CCATAGGCTCCGCCCCCCTGACGAGCATCACAAAAATCGACGCTC |
| | AAGTCAGAGGTGGCGAAACCCGACAGGACTATAAAGATACCAGGC |
| | GTTTCCCCCTGGAAGCTCCCTCGTGCGCTCTCCTGTTCCGACCCT |
| | GCCGCTTACCGGATACCTGTCCGCCTTTCTCCCTTCGGGAAGCGT |
| | GGCGCTTTCTCAATGCTCACGCTGTAGGTATCTCAGTTCGGTGTA |
| | GGTCGTTCGCTCCAAGCTGGGCTGTGTGCACGAACCCCCCGTTCA |
| | GCCCGACCGCTGCGCCTTATCCGGTAACTATCGTCTTGAGTCCAA |
| | CCCGGTAAGACACGACTTATCGCCACTGGCAGCAGCCACTGGTAA |
| | CAGGATTAGCAGAGCGAGGTATGTAGGCGGTGCTACAGAGTTCTT |
| | GAAGTGGTGGCCTAACTACGGCTACACTAGAAGGACAGTATTTGG |
| | TATCTGCGCTCTGCTGAAGCCAGTTACCTTCGGAAAAAGAGTTGG |
| | TAGCTCTTGATCCGGCAAACAAACCACCGCTGGTAGCGGTGGTTT |
| | TTTTGTTTGCAAGCAGCAGATTACGCGCAGAAAAAAAGGATCTCA |
| | AGAAGATCCTTTGATCTTTTCTACGGGGTCTGACGCTCAGTGGAA |
| | CGAAAACTCACGTTAAGGGATTTTGGTCATGAGATTATCAAAAAG |
| | GATCTTCACCTAGATCCTTTTAAATTAAAAATGAAGTTTTAAATC |
| | AATCTAAAGTATATATGAGTAAACTTGGTCTGACAGTTACCAATG |
| | CTTAATCAGTGAGGCACCTATCTCAGCGATCTGTCTATTTCGTTC |
| | ATCCATAGTTGCCTGACTCCCCGTCGTGTAGATAACTACGATACG |
| | GGAGGGCTTACCATCTGGCCCCAGTGCTGCAATGATACCGCGAGA |
| | CCCACGCTCACCGGCTCCAGATTTATCAGCAATAAACCAGCCAGC |
| | CGGAAGGGCCGAGCGCAGAAGTGGTCCTGCAACTTTATCCGCCTC |
| | CATCCAGTCTATTAATTGTTGCCGGGAAGCTAGAGTAAGTAGTTC |
| | GCCAGTTAATAGTTTGCGCAACGTTGTTGCCATTGCTACAGGCAT |
| | CGTGGTGTCACGCTCGTCGTTTGGTATGGCTTCATTCAGCTCCGG |
| | TTCCCAACGATCAAGGCGAGTTACATGATCCCCCATGTTGTGCAA |
| | AAAAGCGGTTAGCTCCTTCGGTCCTCCGATCGTTGTCAGAAGTAA |
| | GTTGGCCGCAGTGTTATCACTCATGGTTATGGCAGCACTGCATAA |
| | TTCTCTTACTGTCATGCCATCCGTAAGATGCTTTTCTGTGACTGG |
| | TGAGTACTCAACCAAGTCATTCTGAGAATAGTGTATGCGGCGACC |
| | GAGTTGCTCTTGCCCGGCGTCAATACGGGATAATACCGCGCCACA |
| | TAGCAGAACTTTAAAAGTGCTCATCATTGGAAAACGTTCTTCGGG |
| | GCGAAAACTCTCAAGGATCTTACCGCTGTTGAGATCCAGTTCGAT |
| | GTAACCCACTCGTGCACCCAACTGATCTTCAGCATCTTTTACTTT |
| | CACCAGCGTTTCTGGGTGAGCAAAAACAGGAAGGCAAAATGCCGC |
| | AAAAAAGGGAATAAGGGCGACACGGAAATGTTGAATACTCATACT |
| | CTTCCTTTTTCAATATTATTGAAGCATTTATCAGGGTTATTGTCT |
| | CATGAGCGGATACATATTTGAATGTATTTAGAAAAATAAACAAAT |
| | AGGGGTTCCGCGCACATTTCCCCGAAAAGTGCCACCTGAAATTGT |
| | AAACGTTAATATTTTGTTAAAATTCGCGTTAAATTTTTGTTAAAT |
| | CAGCTCATTTTTTAACCAATAGGCCGAAATCGGCAAAATCCCTTA |
| | TAAATCAAAAGAATAGACCGAGATAGGGTTGAGTGTTGTTCCAGT |
| | TTGGAACAAGAGTCCACTATTAAAGAACGTGGACTCCAACGTCAA |
| | AGGGCGAAAAACCGTCTATCAGGGCGATGGCCCACTACGTGAACC |
| | ATCACCCTAATCAAGTTTTTTGGGGTCGAGGTGCCGTAAAGCACT |
| | AAATCGGAACCCTAAAGGGAGCCCCCGATTTAGAGCTTGACGGGG |
| | AAAGCCGGCGAACGTGGCGAGAAAGGAAGGGAAGAAAGCGAAAGG |
| | AGCGGGCGCTAGGGCGCTGGCAAGTGTAGCGGTCACGCTGCGCGT |
| | AACCACCACACCCGCCGCGCTTAATGCGCCGCTACAGGGCGCGTC |
| AMI167; | 372-512: Full ITR |
| SEQ ID | 570-873: CMV enhancer |
| NO: 76 | 874-1077: CMV promoter |
| | 1181-1277: SV40 intron |
| | 1282-1290: Kozak sequence |
| | 1291-1347: Human IgG heavy chain secretion sequence |
| | 1348-2106: Lucentis-ScFv optimized coding sequence |
| | 2117-2165: Poly A signal |
| | 2172-2475: CMV enhancer |
| | 2476-2679: CMV promoter |
| | 2783-2879: SV40 intron |
| | 2884-2892: kozak sequence |
| | 2893-2949: Human IgG heavy chain secretion sequence |
| | 2950-3696: TNFa-ScFv |
| | 3706-3754: Poly A signal |
| | 3799-3939: Full ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGA |
| | GCTTACAGCTTCCTGCAGGCAGCTGCGCGCTCGCTCGCTCACTGA |
| | GGCCGCCCGGGCAAAGCCCGGGCGTCGGGCGACCTTTGGTCGCCC |
| | GGCCTCAGTGAGCGAGCGAGCGCGCAGAGAGGGAGTGGCCAACTC |
| | CATCACTAGGGGTTCCTGCGGCCGCACGCGTTGACATTGATTATT |
| | GACTAGGAAGCTGATCTGAATTCGGTACCCGTTACATAACTTACG |
| | GTAAATGGCCCGCCTGGCTGACCGCCCAACGACCCCCGCCCATTG |
| | ACGTCAATAATGACGTATGTTCCCATAGTAACGCCAATAGGGACT |
| | TTCCATTGACGTCAATGGGTGGAGTATTTACGGTAAACTGCCCAC |
| | TTGGCAGTACATCAAGTGTATCATATGCCAAGTACGCCCCCTATT |
| | GACGTCAATGACGGTAAATGGCCCGCCTGGCATTATGCCCAGTAC |
| | ATGACCTTATGGGACTTTCCTACTTGGCAGTACATCTACGTATTA |
| | GTCATCGCTATTACCATGGTGATGCGGTTTTGGCAGTACATCAAT |
| | GGGCGTGGATAGCGGTTTGACTCACGGGGATTTCCAAGTCTCCAC |
| | CCCATTGACGTCAATGGGAGTTTGTTTTGGCACCAAAATCAACGG |
| | GACTTTCCAAAATGTCGTAACAACTCCGCCCCATTGACGCAAATG |
| | GGCGGTAGGCGTGTACGGTGGGAGGTCTATATAAGCAGAGCTCGT |
| | TTAGTGAACCGTCAGATCGCCTGGAGACGCCATCCACGCTGTTTT |
| | GACCTCCATAGAAGACACCGGGACCGATCCAGCCTCCGGACTCTA |
| | GAGTTAACTGGTAAGTTTAGTCTTTTTGTCTTTTATTTCAGGTCC |
| | CGGATCCGGTGGTGGTGCAAATCAAAGAACTGCTCCTCAGTGGAT |
| | GTTGCCTTTACTTCTAGGCCTGCCGCCACCATGGAGTTCGGCCTG |
| | AGCTGGCTGTTCCTGGTGGCCATCCTGAAGGGCGTGCAGTGCGAG |
| | GTGCAGCTGGTGGAGAGCGGCGGCGGCCTGGTGCAGCCCGGCGGC |
| | AGCCTGAGACTGAGCTGCGCCGCCAGCGGCTACGACTTCACCCAC |
| | TACGGCATGAACTGGGTGAGACAGGCCCCCGGCAAGGGCCTGGAG |
| | TGGGTGGGCTGGATCAACACCTACACCGGCGAGCCCACCTACGCC |
| | GCCGACTTCAAGAGAAGATTCACCTTCAGCCTGGACACCAGCAAG |
| | AGCACCGCCTACCTGCAGATGAACAGCCTGAGAGCCGAGGACACC |
| | GCCGTGTACTACTGCGCCAAGTACCCCTACTACTACGGCACCAGC |
| | CACTGGTACTTCGACGTGTGGGGCCAGGGCACCCTGGTGACCGTG |
| | GGCGGAGGCGGAAGCGGCGGAGGCGGATCTGGCGGAGGCGGCAGC |
| | GGCGGCGGCGGCTCTGACATCCAGCTGACCCAGAGCCCCAGCAGC |
| | CTGAGCGCCAGCGTGGGCGACAGAGTGACCATCACCTGCAGCGCC |
| | AGCCAGGACATCAGCAACTACCTGAACTGGTACCAGCAGAAGCCC |
| | GGCAAGGCCCCCAAGGTGCTGATCTACTTCACCAGCAGCCTGCAC |
| | AGCGGCGTGCCCAGCAGATTCAGCGGCAGCGGCAGCGGCACCGAC |
| | TTCACCCTGACCATCAGCAGCCTGCAGCCCGAGGACTTCGCCACC |
| | TACTACTGCCAGCAGTACAGCACCGTGCCCTGGACCTTCGGCCAG |
| | GGCACCAAGGTGGAGATCAAGAGAACCGTGGCCGCCTGATTCGAA |
| | AAATAAAATATCTTTATTTTCATTACATCTGTGTGTTGGTTTTTT |
| | GTGTGGGTACCCGTTACATAACTTACGGTAAATGGCCCGCCTGGC |
| | TGACCGCCCAACGACCCCCGCCCATTGACGTCAATAATGACGTAT |
| | GTTCCCATAGTAACGCCAATAGGGACTTTCCATTGACGTCAATGG |
| | GTGGAGTATTTACGGTAAACTGCCCACTTGGCAGTACATCAAGTG |
| | TATCATATGCCAAGTACGCCCCCTATTGACGTCAATGACGGTAAA |
| | TGGCCCGCCTGGCATTATGCCCAGTACATGACCTTATGGGACTTT |
| | CCTACTTGGCAGTACATCTACGTATTAGTCATCGCTATTACCATG |
| | GTGATGCGGTTTTGGCAGTACATCAATGGGCGTGGATAGCGGTTT |
| | GACTCACGGGGATTTCCAAGTCTCCACCCCATTGACGTCAATGGG |
| | AGTTTGTTTTGGCACCAAAATCAACGGGACTTTCCAAAATGTCGT |
| | AACAACTCCGCCCCATTGACGCAAATGGGCGGTAGGCGTGTACGG |
| | TGGGAGGTCTATATAAGCAGAGCTCGTTTAGTGAACCGTCAGATC |
| | GCCTGGAGACGCCATCCACGCTGTTTTGACCTCCATAGAAGACAC |
| | CGGGACCGATCCAGCCTCCGGACTCTAGAGTTAACTGGTAAGTTT |
| | AGTCTTTTTGTCTTTTATTTCAGGTCCCGGATCCGGTGGTGGTGC |
| | AAATCAAAGAACTGCTCCTCAGTGGATGTTGCCTTTACTTCTAGG |
| | CCTGCCGCCACCATGGAGTTCGGCCTGAGCTGGCTGTTCCTGGTG |
| | GCCATCCTTAAGGGCGTGCAGTGCGAGGTGCAGCTGGTGGAGAGC |
| | GGAGGCGGTCTGGTGCAGCCAGGCAGGAGCCTGAGGCTGAGCTGC |
| | GCCGCCAGCGGCTTCACCTTCGACGACTACGCCATGCACTGGGTG |
| | AGGCAGGCCCCAGGCAAGGGCCTGGAGTGGGTGAGCGCCATCACC |
| | TGGAACAGCGGTCACATCGACTACGCCGACAGCGTGGAGGGTAGG |
| | TTCACCATCAGCAGGGACAACGCCAAGAACAGCCTGTACCTGCAG |
| | ATGAACAGCCTGAGGGCCGAGGACACCGCCGTGTACTACTGCGCC |
| | AAGGTGAGCTACCTGAGCACCGCCAGCAGCCTGGACTACTGGGGT |
| | CAGGGCACCCTGGTGACCGTGAGCAGCGGTGGAGGAGGTAGCGGT |
| | GGCGGTGGTAGCGGTGGCGGAGGCAGCGGTGGAGGTGGCAGCGAC |
| | ATCCAGATGACCCAGAGCCCTAGCAGCCTGAGCGCTAGCGTGGGT |
| | GACAGGGTGACCATCACCTGCAGGGCCAGCCAGGGCATCAGGAAC |
| | TACCTGGCCTGGTACCAGCAGAAGCCCGGCAAGGCCCCCAAGCTG |
| | CTGATCTACGCCGCCAGCACCCTGCAGAGCGGCGTGCCCAGCAGG |
| | TTCAGCGGCAGCGGCAGCGGCACCGACTTCACCCTGACCATCAGC |
| | AGCCTGCAGCCCGAGGACGTGGCCACCTACTACTGCCAGAGGTAC |
| | AACAGGGCCCCCTACACCTTCGGCCAGGGCACCAAGGTGGAGATC |
| | AAGAGGTGATTCGAAAATAAAATATCTTTATTTTCATTACATCTG |
| | TGTGTTGGTTTTTTGTGTGGCATGCTGGGGAGAGATCAACCCACG |
| | TGCGGACCGAGCGGCCGCAGGAACCCCTAGTGATGGAGTTGGCCA |
| | CTCCCTCTCTGCGCGCTCGCTCGCTCACTGAGGCCGGGCGACCAA |
| | AGGTCGCCCGACGCCCGGGCTTTGCCCGGGCGGCCTCAGTGAGCG |
| | AGCGAGCGCGCAGCTGCCTGCAGGCATGCAAGCTGTAGCCAACCA |
| | CTAGAACTATAGCTAGAGTCCTGGGCGAACAAACGATGCTCGCCT |
| | TCCAGAAAACCGAGGATGCGAACCACTTCATCCGGGGTCAGCACC |
| | ACCGGCAAGCGCCGCGACGGCCGAGGTCTTCCGATCTCCTGAAGC |
| | CAGGGCAGATCCGTGCACAGCACCTTGCCGTAGAAGAACAGCAAG |
| | GCCGCCAATGCCTGACGATGCGTGGAGACCGAAACCTTGCGCTCG |
| | TTCGCCAGCCAGGACAGAAATGCCTCGACTTCGCTGCTGCCCAAG |
| | GTTGCCGGGTGACGCACACCGTGGAAACGGATGAAGGCACGAACC |
| | CAGTTGACATAAGCCTGTTCGGTTCGTAAACTGTAATGCAAGTAG |
| | CGTATGCGCTCACGCAACTGGTCCAGAACCTTGACCGAACGCAGC |
| | GGTGGTAACGGCGCAGTGGCGGTTTTCATGGCTTGTTATGACTGT |
| | TTTTTTGTACAGTCTATGCCTCGGGCATCCAAGCAGCAAGCGCGT |
| | TACGCCGTGGGTCGATGTTTGATGTTATGGAGCAGCAACGATGTT |
| | ACGCAGCAGCAACGATGTTACGCAGCAGGGCAGTCGCCCTAAAAC |
| | AAAGTTAGGTGGCTCAAGTATGGGCATCATTCGCACATGTAGGCT |
| | CGGCCCTGACCAAGTCAAATCCATGCGGGCTGCTCTTGATCTTTT |
| | CGGTCGTGAGTTCGGAGACGTAGCCACCTACTCCCAACATCAGCC |
| | GGACTCCGATTACCTCGGGAACTTGCTCCGTAGTAAGACATTCAT |
| | CGCGCTTGCTGCCTTCGACCAAGAAGCGGTTGTTGGCGCTCTCGC |
| | GGCTTACGTTCTGCCCAGGTTTGAGCAGCCGCGTAGTGAGATCTA |
| | TATCTATGATCTCGCAGTCTCCGGCGAGCACCGGAGGCAGGGCAT |
| | TGCCACCGCGCTCATCAATCTCCTCAAGCATGAGGCCAACGCGCT |
| | TGGTGCTTATGTGATCTACGTGCAAGCAGATTACGGTGACGATCC |
| | CGCAGTGGCTCTCTATACAAAGTTGGGCATACGGGAAGAAGTGAT |
| | GCACTTTGATATCGACCCAAGTACCGCCACCTAACAATTCGTTCA |
| | AGCCGAGATCGGCTTCCCGGCCGCGGAGTTGTTCGGTAAATTGTC |
| | ACAACGCCGCGAATATAGTCTTTACCATGCCCTTGGCCACGCCCC |
| | TCTTTAATACGACGGGCAATTTGCACTTCAGAAAATGAAGAGTTT |
| | GCTTTAGCCATAACAAAAGTCCAGTATGCTTTTTCACAGCATAAC |
| | TGGACTGATTTCAGTTTACAACTATTCTGTCTAGTTTAAGACTTT |
| | ATTGTCATAGTTTAGATCTATTTTGTTCAGTTTAAGACTTTATTG |
| | TCCGCCCACACCCGCTTACGCAGGGCATCCATTTATTACTCAACC |
| | GTAACCGATTTTGCCAGGTTACGCGGCTGGTCTGCGGTGTGAAAT |
| | ACCGCACAGATGCGTAAGGAGAAAATACCGCATCAGGCGCTCTTC |
| | CGCTTCCTCGCTCACTGACTCGCTGCGCTCGGTCGTTCGGCTGCG |
| | GCGAGCGGTATCAGCTCACTCAAAGGCGGTAATACGGTTATCCAC |
| | AGAATCAGGGGATAACGCAGGAAAGAACATGTGAGCAAAAGGCCA |
| | GCAAAAGGCCAGGAACCGTAAAAAGGCCGCGTTGCTGGCGTTTTT |
| | CCATAGGCTCCGCCCCCCTGACGAGCATCACAAAAATCGACGCTC |
| | AAGTCAGAGGTGGCGAAACCCGACAGGACTATAAAGATACCAGGC |
| | GTTTCCCCCTGGAAGCTCCCTCGTGCGCTCTCCTGTTCCGACCCT |
| | GCCGCTTACCGGATACCTGTCCGCCTTTCTCCCTTCGGGAAGCGT |
| | GGCGCTTTCTCAATGCTCACGCTGTAGGTATCTCAGTTCGGTGTA |
| | GGTCGTTCGCTCCAAGCTGGGCTGTGTGCACGAACCCCCCGTTCA |
| | GCCCGACCGCTGCGCCTTATCCGGTAACTATCGTCTTGAGTCCAA |
| | CCCGGTAAGACACGACTTATCGCCACTGGCAGCAGCCACTGGTAA |
| | CAGGATTAGCAGAGCGAGGTATGTAGGCGGTGCTACAGAGTTCTT |
| | GAAGTGGTGGCCTAACTACGGCTACACTAGAAGGACAGTATTTGG |
| | TATCTGCGCTCTGCTGAAGCCAGTTACCTTCGGAAAAAGAGTTGG |
| | TAGCTCTTGATCCGGCAAACAAACCACCGCTGGTAGCGGTGGTTT |
| | TTTTGTTTGCAAGCAGCAGATTACGCGCAGAAAAAAAGGATCTCA |
| | AGAAGATCCTTTGATCTTTTCTACGGGGTCTGACGCTCAGTGGAA |
| | CGAAAACTCACGTTAAGGGATTTTGGTCATGAGATTATCAAAAAG |
| | GATCTTCACCTAGATCCTTTTAAATTAAAAATGAAGTTTTAAATC |
| | AATCTAAAGTATATATGAGTAAACTTGGTCTGACAGTTACCAATG |
| | CTTAATCAGTGAGGCACCTATCTCAGCGATCTGTCTATTTCGTTC |
| | ATCCATAGTTGCCTGACTCCCCGTCGTGTAGATAACTACGATACG |
| | GGAGGGCTTACCATCTGGCCCCAGTGCTGCAATGATACCGCGAGA |
| | CCCACGCTCACCGGCTCCAGATTTATCAGCAATAAACCAGCCAGC |
| | CGGAAGGGCCGAGCGCAGAAGTGGTCCTGCAACTTTATCCGCCTC |
| | CATCCAGTCTATTAATTGTTGCCGGGAAGCTAGAGTAAGTAGTTC |
| | GCCAGTTAATAGTTTGCGCAACGTTGTTGCCATTGCTACAGGCAT |
| | CGTGGTGTCACGCTCGTCGTTTGGTATGGCTTCATTCAGCTCCGG |
| | TTCCCAACGATCAAGGCGAGTTACATGATCCCCCATGTTGTGCAA |
| | AAAAGCGGTTAGCTCCTTCGGTCCTCCGATCGTTGTCAGAAGTAA |
| | GTTGGCCGCAGTGTTATCACTCATGGTTATGGCAGCACTGCATAA |
| | TTCTCTTACTGTCATGCCATCCGTAAGATGCTTTTCTGTGACTGG |
| | TGAGTACTCAACCAAGTCATTCTGAGAATAGTGTATGCGGCGACC |
| | GAGTTGCTCTTGCCCGGCGTCAATACGGGATAATACCGCGCCACA |
| | TAGCAGAACTTTAAAAGTGCTCATCATTGGAAAACGTTCTTCGGG |
| | GCGAAAACTCTCAAGGATCTTACCGCTGTTGAGATCCAGTTCGAT |
| | GTAACCCACTCGTGCACCCAACTGATCTTCAGCATCTTTTACTTT |
| | CACCAGCGTTTCTGGGTGAGCAAAAACAGGAAGGCAAAATGCCGC |
| | AAAAAAGGGAATAAGGGCGACACGGAAATGTTGAATACTCATACT |
| | CTTCCTTTTTCAATATTATTGAAGCATTTATCAGGGTTATTGTCT |
| | CATGAGCGGATACATATTTGAATGTATTTAGAAAAATAAACAAAT |
| | AGGGGTTCCGCGCACATTTCCCCGAAAAGTGCCACCTGAAATTGT |
| | AAACGTTAATATTTTGTTAAAATTCGCGTTAAATTTTTGTTAAAT |
| | CAGCTCATTTTTTAACCAATAGGCCGAAATCGGCAAAATCCCTTA |
| | TAAATCAAAAGAATAGACCGAGATAGGGTTGAGTGTTGTTCCAGT |
| | TTGGAACAAGAGTCCACTATTAAAGAACGTGGACTCCAACGTCAA |
| | AGGGCGAAAAACCGTCTATCAGGGCGATGGCCCACTACGTGAACC |
| | ATCACCCTAATCAAGTTTTTTGGGGTCGAGGTGCCGTAAAGCACT |
| | AAATCGGAACCCTAAAGGGAGCCCCCGATTTAGAGCTTGACGGGG |
| | AAAGCCGGCGAACGTGGCGAGAAAGGAAGGGAAGAAAGCGAAAGG |
| | AGCGGGCGCTAGGGCGCTGGCAAGTGTAGCGGTCACGCTGCGCGT |
| | AACCACCACACCCGCCGCGCTTAATGCGCCGCTACAGGGCGCGTC |
| AMI169; | 611-755: Full ITR |
| SEQ ID | 801-1104: CMV enhancer |
| NO: 77 | 1105-1308: CMV promoter |
| | 1412-1508: SV40 intron |
| | 1513-1521: Kozak sequence |
| | 1522-1578: Human IgG heavy chain secretion sequence |
| | 1579-1686: CNP36 optimized coding sequence |
| | 1687-1770: Furin-F2A sequence |
| | 1771-1827: Human IgG heavy chain secretion sequence |
| | 1828-2586: Lucentis-ScFv optimized coding sequence |
| | 2596-2644: Poly A signal |
| | 2667-2772: Truncated ITR |
| | CATTCGCCATTCAGGCTGCAAATAAGCGTTGATATTCAGTCAATT |
| | ACAAACATTAATAACGAAGAGATGACAGAAAAATTTTCATTCTGT |
| | GACAGAGAAAAAGTAGCCGAAGATGACGGTTTGTCACATGGAGTT |
| | GGCAGGATGTTTGATTAAAAACATAACAGGAAGAAAAATGCCCCG |
| | CTGTGGGCGGACAAAATAGTTGGGAACTGGGAGGGGTGGAAATGG |
| | AGTTTTTAAGGATTATTTAGGGAAGAGTGACAAAATAGATGGGAA |
| | CTGGGTGTAGCGTCGTAAGCTAATACGAAAATTAAAAATGACAAA |
| | ATAGTTTGGAACTAGATTTCACTTATCTGGTTCGGATCTCCTAGG |
| | CTCAAGCAGTGATCAGATCCAGACATGATAAGATACATTGATGAG |
| | TTTGGACAAACCACAACTAGAATGCAGTGAAAAAAATGCTTTATT |
| | TGTGAAATTTGTGATGCTATTGCTTTATTTGTAACCATTATAAGC |
| | TGCAATAAACAAGTTAACAACAACAATTGCATTCATTTTATGTTT |
| | CAGGTTCAGGGGGAGGTGTGGGAGGTTTTTTAAAGCAAGTAAAAC |
| | CTCTACAAATGTGGTATGGCTGATTATGATCCTCTAGTACTTCTC |
| | GACAAGCTCGGATCCTGGCGCGCTCGCTCGCTCACTGAGGCCGCC |
| | CGGGCAAAGCCCGGGCGTCGGGCGACCTTTGGTCGCCCGGCCTCA |
| | GTGAGCGAGCGAGCGCGCAGAGAGGGAGTGGCCAACTCCATCACT |
| | AGGGGTTCCTAGGAAGCTGATCTGAATTCGGTACCCGTTACATAA |
| | CTTACGGTAAATGGCCCGCCTGGCTGACCGCCCAACGACCCCCGC |
| | CCATTGACGTCAATAATGACGTATGTTCCCATAGTAACGCCAATA |
| | GGGACTTTCCATTGACGTCAATGGGTGGAGTATTTACGGTAAACT |
| | GCCCACTTGGCAGTACATCAAGTGTATCATATGCCAAGTACGCCC |
| | CCTATTGACGTCAATGACGGTAAATGGCCCGCCTGGCATTATGCC |
| | CAGTACATGACCTTATGGGACTTTCCTACTTGGCAGTACATCTAC |
| | GTATTAGTCATCGCTATTACCATGGTGATGCGGTTTTGGCAGTAC |
| | ATCAATGGGCGTGGATAGCGGTTTGACTCACGGGGATTTCCAAGT |
| | CTCCACCCCATTGACGTCAATGGGAGTTTGTTTTGGCACCAAAAT |
| | CAACGGGACTTTCCAAAATGTCGTAACAACTCCGCCCCATTGACG |
| | CAAATGGGCGGTAGGCGTGTACGGTGGGAGGTCTATATAAGCAGA |
| | GCTCGTTTAGTGAACCGTCAGATCGCCTGGAGACGCCATCCACGC |
| | TGTTTTGACCTCCATAGAAGACACCGGGACCGATCCAGCCTCCGG |
| | ACTCTAGAGTTAACTGGTAAGTTTAGTCTTTTTGTCTTTTATTTC |
| | AGGTCCCGGATCCGGTGGTGGTGCAAATCAAAGAACTGCTCCTCA |
| | GTGGATGTTGCCTTTACTTCTAGGCCTGCCGCCACCATGGAGTTC |
| | GGCCTGAGCTGGCTGTTCCTGGTGGCCATCCTTAAGGGCGTGCAG |
| | TGCGAGCACCCCAACGCGCGCAAATACAAAGGAGCCAACAAGAAG |
| | GGCTTGTCCAAGGGCTGCTTCGGCCTCAAGCTGGACCGAATCGGC |
| | TCCATGAGCGGCCTGGGATGTAGAAGAAAGAGAGCCCCCGTGAAG |
| | CAGACCCTGAACTTCGACCTGCTGAAGCTGGCCGGCGACGTGGAG |
| | AGCAACCCCGGCCCCATGGAGTTCGGCCTGAGCTGGCTGTTCCTG |
| | GTGGCCATCCTTAAGGGCGTGCAGTGCGAGGTGCAGCTGGTGGAG |
| | AGCGGCGGCGGCCTGGTGCAGCCCGGCGGCAGCCTGAGACTGAGC |
| | TGCGCCGCCAGCGGCTACGACTTCACCCACTACGGCATGAACTGG |
| | GTGAGACAGGCCCCCGGCAAGGGCCTGGAGTGGGTGGGCTGGATC |
| | AACACCTACACCGGCGAGCCCACCTACGCCGCCGACTTCAAGAGA |
| | AGATTCACCTTCAGCCTGGACACCAGCAAGAGCACCGCCTACCTG |
| | CAGATGAACAGCCTGAGAGCCGAGGACACCGCCGTGTACTACTGC |
| | GCCAAGTACCCCTACTACTACGGCACCAGCCACTGGTACTTCGAC |
| | GTGTGGGGCCAGGGCACCCTGGTGACCGTGGGCGGAGGCGGAAGC |
| | GGCGGAGGCGGATCTGGCGGAGGCGGCAGCGGCGGCGGCGGCTCT |
| | GACATCCAGCTGACCCAGAGCCCCAGCAGCCTGAGCGCCAGCGTG |
| | GGCGACAGAGTGACCATCACCTGCAGCGCCAGCCAGGACATCAGC |
| | AACTACCTGAACTGGTACCAGCAGAAGCCCGGCAAGGCCCCCAAG |
| | GTGCTGATCTACTTCACCAGCAGCCTGCACAGCGGCGTGCCCAGC |
| | AGATTCAGCGGCAGCGGCAGCGGCACCGACTTCACCCTGACCATC |
| | AGCAGCCTGCAGCCCGAGGACTTCGCCACCTACTACTGCCAGCAG |
| | TACAGCACCGTGCCCTGGACCTTCGGCCAGGGCACCAAGGTGGAG |
| | ATCAAGAGAACCGTGGCCGCCTGATTCGAAAATAAAATATCTTTA |
| | TTTTCATTACATCTGTGTGTTGGTTTTTTGTGTGGCATGCTGGGG |
| | AGAGATCAACCCCACTCCCTCTCTGCGCGCTCGCTCGCTCACTGA |
| | GGCCGGGCGACCAAAGGTCGCCCGACGCCCGGGCTTTGCCCGGGC |
| | GGCCTCAGTGAGCGAGCGAGCGCGCAGCAAGCTGTAGCCAACCAC |
| | TAGAACTATAGCTAGAGTCCTGGGCGAACAAACGATGCTCGCCTT |
| | CCAGAAAACCGAGGATGCGAACCACTTCATCCGGGGTCAGCACCA |
| | CCGGCAAGCGCCGCGACGGCCGAGGTCTTCCGATCTCCTGAAGCC |
| | AGGGCAGATCCGTGCACAGCACCTTGCCGTAGAAGAACAGCAAGG |
| | CCGCCAATGCCTGACGATGCGTGGAGACCGAAACCTTGCGCTCGT |
| | TCGCCAGCCAGGACAGAAATGCCTCGACTTCGCTGCTGCCCAAGG |
| | TTGCCGGGTGACGCACACCGTGGAAACGGATGAAGGCACGAACCC |
| | AGTTGACATAAGCCTGTTCGGTTCGTAAACTGTAATGCAAGTAGC |
| | GTATGCGCTCACGCAACTGGTCCAGAACCTTGACCGAACGCAGCG |
| | GTGGTAACGGCGCAGTGGCGGTTTTCATGGCTTGTTATGACTGTT |
| | TTTTTGTACAGTCTATGCCTCGGGCATCCAAGCAGCAAGCGCGTT |
| | ACGCCGTGGGTCGATGTTTGATGTTATGGAGCAGCAACGATGTTA |
| | CGCAGCAGCAACGATGTTACGCAGCAGGGCAGTCGCCCTAAAACA |
| | AAGTTAGGTGGCTCAAGTATGGGCATCATTCGCACATGTAGGCTC |
| | GGCCCTGACCAAGTCAAATCCATGCGGGCTGCTCTTGATCTTTTC |
| | GGTCGTGAGTTCGGAGACGTAGCCACCTACTCCCAACATCAGCCG |
| | GACTCCGATTACCTCGGGAACTTGCTCCGTAGTAAGACATTCATC |
| | GCGCTTGCTGCCTTCGACCAAGAAGCGGTTGTTGGCGCTCTCGCG |
| | GCTTACGTTCTGCCCAGGTTTGAGCAGCCGCGTAGTGAGATCTAT |
| | ATCTATGATCTCGCAGTCTCCGGCGAGCACCGGAGGCAGGGCATT |
| | GCCACCGCGCTCATCAATCTCCTCAAGCATGAGGCCAACGCGCTT |
| | GGTGCTTATGTGATCTACGTGCAAGCAGATTACGGTGACGATCCC |
| | GCAGTGGCTCTCTATACAAAGTTGGGCATACGGGAAGAAGTGATG |
| | CACTTTGATATCGACCCAAGTACCGCCACCTAACAATTCGTTCAA |
| | GCCGAGATCGGCTTCCCGGCCGCGGAGTTGTTCGGTAAATTGTCA |
| | CAACGCCGCGAATATAGTCTTTACCATGCCCTTGGCCACGCCCCT |
| | CTTTAATACGACGGGCAATTTGCACTTCAGAAAATGAAGAGTTTG |
| | CTTTAGCCATAACAAAAGTCCAGTATGCTTTTTCACAGCATAACT |
| | GGACTGATTTCAGTTTACAACTATTCTGTCTAGTTTAAGACTTTA |
| | TTGTCATAGTTTAGATCTATTTTGTTCAGTTTAAGACTTTATTGT |
| | CCGCCCACACCCGCTTACGCAGGGCATCCATTTATTACTCAACCG |
| | TAACCGATTTTGCCAGGTTACGCGGCTGGTCTGCGGTGTGAAATA |
| | CCGCACAGATGCGTAAGGAGAAAATACCGCATCAGGCGCTCTTCC |
| | GCTTCCTCGCTCACTGACTCGCTGCGCTCGGTCGTTCGGCTGCGG |
| | CGAGCGGTATCAGCTCACTCAAAGGCGGTAATACGGTTATCCACA |
| | GAATCAGGGGATAACGCAGGAAAGAACATGTGAGCAAAAGGCCAG |
| | CAAAAGGCCAGGAACCGTAAAAAGGCCGCGTTGCTGGCGTTTTTC |
| | CATAGGCTCCGCCCCCCTGACGAGCATCACAAAAATCGACGCTCA |
| | AGTCAGAGGTGGCGAAACCCGACAGGACTATAAAGATACCAGGCG |
| | TTTCCCCCTGGAAGCTCCCTCGTGCGCTCTCCTGTTCCGACCCTG |
| | CCGCTTACCGGATACCTGTCCGCCTTTCTCCCTTCGGGAAGCGTG |
| | GCGCTTTCTCAATGCTCACGCTGTAGGTATCTCAGTTCGGTGTAG |
| | GTCGTTCGCTCCAAGCTGGGCTGTGTGCACGAACCCCCCGTTCAG |
| | CCCGACCGCTGCGCCTTATCCGGTAACTATCGTCTTGAGTCCAAC |
| | CCGGTAAGACACGACTTATCGCCACTGGCAGCAGCCACTGGTAAC |
| | AGGATTAGCAGAGCGAGGTATGTAGGCGGTGCTACAGAGTTCTTG |
| | AAGTGGTGGCCTAACTACGGCTACACTAGAAGGACAGTATTTGGT |
| | ATCTGCGCTCTGCTGAAGCCAGTTACCTTCGGAAAAAGAGTTGGT |
| | AGCTCTTGATCCGGCAAACAAACCACCGCTGGTAGCGGTGGTTTT |
| | TTTGTTTGCAAGCAGCAGATTACGCGCAGAAAAAAAGGATCTCAA |
| | GAAGATCCTTTGATCTTTTCTACGGGGTCTGACGCTCAGTGGAAC |
| | GAAAACTCACGTTAAGGGATTTTGGTCATGAGATTATCAAAAAGG |
| | ATCTTCACCTAGATCCTTTTAAATTAAAAATGAAGTTTTAAATCA |
| | ATCTAAAGTATATATGAGTAAACTTGGTCTGACAGTTACCAATGC |
| | TTAATCAGTGAGGCACCTATCTCAGCGATCTGTCTATTTCGTTCA |
| | TCCATAGTTGCCTGACTCCCCGTCGTGTAGATAACTACGATACGG |
| | GAGGGCTTACCATCTGGCCCCAGTGCTGCAATGATACCGCGAGAC |
| | CCACGCTCACCGGCTCCAGATTTATCAGCAATAAACCAGCCAGCC |
| | GGAAGGGCCGAGCGCAGAAGTGGTCCTGCAACTTTATCCGCCTCC |
| | ATCCAGTCTATTAATTGTTGCCGGGAAGCTAGAGTAAGTAGTTCG |
| | CCAGTTAATAGTTTGCGCAACGTTGTTGCCATTGCTACAGGCATC |
| | GTGGTGTCACGCTCGTCGTTTGGTATGGCTTCATTCAGCTCCGGT |
| | TCCCAACGATCAAGGCGAGTTACATGATCCCCCATGTTGTGCAAA |
| | AAAGCGGTTAGCTCCTTCGGTCCTCCGATCGTTGTCAGAAGTAAG |
| | TTGGCCGCAGTGTTATCACTCATGGTTATGGCAGCACTGCATAAT |
| | TCTCTTACTGTCATGCCATCCGTAAGATGCTTTTCTGTGACTGGT |
| | GAGTACTCAACCAAGTCATTCTGAGAATAGTGTATGCGGCGACCG |
| | AGTTGCTCTTGCCCGGCGTCAATACGGGATAATACCGCGCCACAT |
| | AGCAGAACTTTAAAAGTGCTCATCATTGGAAAACGTTCTTCGGGG |
| | CGAAAACTCTCAAGGATCTTACCGCTGTTGAGATCCAGTTCGATG |
| | TAACCCACTCGTGCACCCAACTGATCTTCAGCATCTTTTACTTTC |
| | ACCAGCGTTTCTGGGTGAGCAAAAACAGGAAGGCAAAATGCCGCA |
| | AAAAAGGGAATAAGGGCGACACGGAAATGTTGAATACTCATACTC |
| | TTCCTTTTTCAATATTATTGAAGCATTTATCAGGGTTATTGTCTC |
| | ATGAGCGGATACATATTTGAATGTATTTAGAAAAATAAACAAATA |
| | GGGGTTCCGCGCACATTTCCCCGAAAAGTGCCACCTGAAATTGTA |
| | AACGTTAATATTTTGTTAAAATTCGCGTTAAATTTTTGTTAAATC |
| | AGCTCATTTTTTAACCAATAGGCCGAAATCGGCAAAATCCCTTAT |
| | AAATCAAAAGAATAGACCGAGATAGGGTTGAGTGTTGTTCCAGTT |
| | TGGAACAAGAGTCCACTATTAAAGAACGTGGACTCCAACGTCAAA |
| | GGGCGAAAAACCGTCTATCAGGGCGATGGCCCACTACGTGAACCA |
| | TCACCCTAATCAAGTTTTTTGGGGTCGAGGTGCCGTAAAGCACTA |
| | AATCGGAACCCTAAAGGGAGCCCCCGATTTAGAGCTTGACGGGGA |
| | AAGCCGGCGAACGTGGCGAGAAAGGAAGGGAAGAAAGCGAAAGGA |
| | GCGGGCGCTAGGGCGCTGGCAAGTGTAGCGGTCACGCTGCGCGTA |
| | ACCACCACACCCGCCGCGCTTAATGCGCCGCTACAGGGCGCGTC |

### Maintenance of HEK293 LTV Cells

HEK293 LTV cell line was cultured in DMEM media containing 100 units/mL of penicillin and 100 μg/mL of streptomycin (P/S) (Corning) and 10% FBS (ATCC). It usually doubled in 24 hours. For regular maintenance, the cells were split 1:10 once a week.

Transient Transfection of HEK293 LTV Cells with Plasmid DNA

The cells were seeded in 6-well plates at 1×10\hat{ }6 cells/well in 2 mL DMEM media containing 100 units/mL of penicillin and 100 μg/mL of streptomycin (P/S) and 10% FBS and cultured overnight. At the day of transfection, the old media were removed and replaced with Opti-MEM media. Transient transfection was performed by diluting 1 μg shRNA plasmid and 1 μg Ang2 plasmid in 100 μL of Opti-MEM and 4 μL of PEI (1 μg/μL) in 100 μL of Opti-MEM, mixing both diluted solutions and incubating for 10 min, and then adding to the cells dropwise. Four days after transfection, 500 μL media were collected for assays and replenished with 500 μL fresh Opti-MEM media. After additional 3 days of incubation, all media were collected for assays.

### Enzyme-Linked Immunosorbent Assay (ELISA)

ELISA assays were performed as follows: a 50 μL/well of capture antibody diluted in coating buffer (3.7 g sodium bicarbonate, 0.64 g sodium carbonate in 1 L of Milli Q water, pH 9.6) at 5 μg/mL was coated on 96-well plates overnight at 4° C. with sealing cover. The next day the coating solution was discarded, and plate was tapped on paper towel to remove excessive solution. A 300 μL/well of blocking solution (Commercial casein blocking buffer in PBS+0.1% Tween 20) was added and the plate was sealed and incubated for 2 hours at 37° C. After incubation the blocking buffer was discarded, and excessive buffer was removed by tapping the plate on a paper towel. The samples to be tested were diluted in coating buffer and 50 μL/well of diluted samples were added and incubated at 37° C. for 2 hours. After incubation the solution was discarded, and plate tapped on a paper towel to remove excessive solution. After washing with 300 μL/well of washing buffer (1×PBS with 0.1% Tween-20, expires in 30 days after preparation) for 6 times, the plate was tapped on a paper towel to remove excessive solution and detection antibody diluted 1:100 in coating buffer was added 50 μL/well and plate was incubated at 37° C. for 2 hours. After incubation, the solution was discarded, and plate tapped on a paper towel to remove excessive solution. Streptavidin-TRP diluted in blocking buffer at 1:5000 was added 50 μL/well and plate incubated at 37° C. for 1 hour. After incubation, the solution was discarded, and plate tapped on a paper towel to remove excessive solution. The plate was washed with washing buffer 300 μL/well for 6 times and excessive solution was removed by tapping the plate on a paper towel. Color reaction solution TMB was added 50 μL/well and the reaction was carried out for 15-20 min (or shorter time period if the color was saturated) at room temperature under dark. The color reaction was stopped by adding 50 μL/well stop solution and OD at 450 nm was read with OD at 600 nm as reference with 15 min after adding the stop solution.

### Generation of Recombinant Baculoviruses for AAV Production

Recombinant baculoviruses (rBVs) were generated using the Bac-to-Bac Baculovirus Expression System according to the manufacturer's instruction (Invitrogen, Carlsbad, CA). Briefly, the pFB shuttle plasmids containing the target genes were each diluted into 1 ng/μL in TE buffer, and 2 ng of each DNA was mixed with 20 μL of Δcath-DH10Bac competent bacteria containing a bacmid DNA molecule with the cathepsin gene deleted (Virovek, Hayward, CA) and incubated on ice for 30 min followed by heat-shock at 42° C. for 30 seconds. After incubating on ice for 2 min, the bacteria were cultured at 37° C. for 4 hours to recover and then plated on agar plates containing 50 μg/mL of kanamycin, 7 μg/mL of gentamycin, 10 μg/mL of tetracycline, 40 μg/mL of IPTG, and 100 μg/mL of X-gal. After 48 hours of incubation at 37° C., 2 white colonies containing the recombinant bacmid DNAs were picked and miniprep bacmid DNAs purified under sterile condition. About 5 μg of each bacmid DNA and 10 μL of GeneJet Reagent (SignaGen Laboratories, Fredrick, MD) were respectively diluted in 100 μL ESFAF media (Expression Systems, Davis, CA) and then mixed together for about 30 min to form the transfection mixture. Sf9 cells were plated in a 6-well plate at 1.5e+6 cells/well in 2 mL ESFAF media at 28° C. for about 30 min. After removing the old media from the Sf9 cells, each transfection mixture was diluted in 800 μL ESFAF media and then added to the Sf9 cells. After incubation at 28° C. overnight, each well was added with additional 1 mL ESFAF media. After a total incubation time of 4 days, media containing the rBVs were collected and amplified at 1:200 ratio to generate sufficient quantity of rBVs ready for use in the AAV production process.

### AAV Production and Purification

The rBVs carrying the AAV2 Rep and mutant capsid genes and the target expression cassettes respectively were used to co-infect Sf9-V432AG cells for AAV production. Briefly, 10 moi of rBV-Cap-Rep and 5 moi of rBV-target cassettes were used to co-infect the Sf9 cell line at density of ˜5e+6 cells/mL with 50% fresh ESFAF media for 3 days at 28° C. with shaking speed of 180 rpm in a shaker incubator. At the end of infection, cell pellets were collected by centrifugation at 3,000 rpm for 10 min. The cells were lysed in Sf9 lysis buffer containing 50 mM Tris-HCl, pH8.0, 2 mM MgCl2, 1% sarkosyl, 1% Triton X-100, and 125 units/mL Benzonase with vigorous vortex followed by shaking at 350 rpm, 37° C. for 1 hour. At the end of shaking, salt concentration was increased to 500 mM by vortexing and the lysates were cleared by centrifugation at 8,000 rpm for 20 min at 4° C. The cleared lysates were transferred to ultraclear centrifuge tubes for SW28 swing bucket rotor which contain 5 mL of 1.50 g/cc and 10 mL of 1.30 g/cc cesium chloride solutions. After centrifugation at 28,000 rpm, 15° C. for ˜18 hours, the AAV bands were collected with syringes and transferred to ultraclear centrifuge tubes for the 70 ti centrifuge rotor. The centrifuge tubes were filled with 1.38 g/cc cesium chloride solution and heat-sealed. The AAV samples were subjected to a second round of ultracentrifugation at 65,000 rpm, 15° C. for ˜18 hours and AAV bands were collected with syringes. The purified AAV samples were buffer-exchanged into PBS buffer containing 0.001% Pluronic F-68 and filter-sterilized with 0.22 um syringe filters. The sterilized AAV samples were stored at 4° C. within a month and then transferred to −80° C. for long term storage. AAV titer was determined with real-time PCR method using the QuantStudio 7 Flex Real-Time PCR System (Invitrogen).

### HEK293 Transductions by AAV and ELISA Quantification

HEK293 cells were seeded at 1.0E+6 cells/well in 2 mL EMEM containing 10% FBS onto 6-well plate and incubated at 37° C. and 5% CO₂. After 24 hrs, AAV was added to each well at 100,000 vg/cell (MOI) and placed into incubator. Twenty-four hrs after the transduction, old medium was replaced with fresh complete medium. Four days after transductions, culture supernatants were collected, and an in-house ELISA developed to quantify VEGF-Trap and COMP-Ang1 protein was performed. All transductions were performed in duplicates. The cultures for production of proteins for these transient expression or AAV transduction, HEK293 cell cultures were conducted with ultralow IgG serum or serum free media.

Protein Purification with Column Chromatography

Cell culture fluid of HEK293 transfected with plasmid DNA coding POIs or transduced with corresponding AAV were filtered with 0.2 μm filter to remove particulates and loaded onto the column (1-mL size) of the MabSelect prismA of the protein A column chromatography at flow rate of 1.5˜2.0 mL/min. The column was washed with wash buffer (20 mM Tris-HCl, pH 7.3, 150 mM NaCl, 5 mM EDTA) and eluted with elution buffer (0.1 M sodium acetate, pH 3.0-3.6), and neutralized with 1/10 of the neutralization buffer (1.0 M Tris-HCl, pH 10). The neutralized protein solution was buffer exchanged to 1×PBS containing 0.01% (w/v) Pluronic F68 or Tween 20 and filtrated sterilely s through a 0.2 μm syringe filter pre-wet with PBS. The final preparations were stored at −80° C.

Results—Plasmids Constructed for this Study

A total of 16 plasmids were constructed for this project to study the functions of aflibercept, angiopoietin 1 and 2, and their synergetic effect on neovascularization. The constructed plasmids include AMI071, AMI077, AMI136, AMI142, AMI143, AMI144, AMI145, AMI146, AMI147, AMI148, AMI149, AMI150, AMI151, AMI152, AMI153, and AMI154, AMI155, AMI156, AMI157, AMI158, AMI159, AMI160, AMI161, AMI162, AMI163, AMI166, AMI167, and AMI169. Detailed AAV construct sequences and respective regulator elements are listed in Table 4.

### Optimized Ang1 Coding Sequence Improved Protein Expression

Non-optimized and optimized hCOMP-Ang1 sequences were cloned into the identical plasmid backbone respectively to create AMI071 and AMI077. Recombinant baculoviruses were generated and used to infect Sf9 cells to produce AAVs. Purified AAV2.N54 vectors were used to transduce HEK293 cells and Ang1 protein levels were determined using ELISA assays. The results are shown in Table 5, which shows that the optimized Ang1 coding sequence improved protein expression level by more than 5 folds.

TABLE 5
Optimized Ang1 coding sequence for increasing Ang1 expression

| | | Angl level | |
|---|---|---|---|
| Clone no. | AAV | (ng/mL) | Fold |
| AMI071 | AAV2.N54-CMV-hCOMP-Ang1 | 5808.9 | 1 |
| AMI077 | AAV2.N54-CMV-hCOMP-Ang1-GC | 30343.1 | 5.22 |

Custom-Designed shRNA Against Ang2 Inhibited Ang2 Expression

A series of shRNA against human Ang2 gene was cloned under control of the human U6 promoter. The plasmids containing these shRNAs were transfected into HEK293 cells together with a plasmid expressing the human Ang2 protein in 6-well plates. Four days after transfection, 500 μL of media from each well were harvested and the same volume of fresh media were added to the wells and the cells were cultured for another 3 days. Then all media were harvested and the expression of VEGF-Trap and human Ang2 were determined with ELISA assays. The results of Ang2 shRNA are shown in Table 6. The results demonstrate that all of the shRNAs have inhibitory effect on the Ang2 expression among with the shRNA3 and shRNA4 as the most efficient. When all of the plasmids were packaged into AAV2.N54 vectors, and the resulted vectors were transduced into HEK293 cells for 4 days. All of the shRNAs show inhibitory effect on Ang2 expression. The shRNA3 and shRNA4 show the most efficient inhibitory effect on Ang2 expression (Table 7).

TABLE 6
shRNA inhibition of angiopoietin 2 expression in plasmid transfected HEK293 cells

| | | Decrease of Ang2 | Decrease of Ang2 |
|---|---|---|---|
| Clone | | expression 4 days post | expression 7 days post |
| no. | Plasmid | transfection (%) | transfection (%) |
| AMI145 | pFB-scCMV-SV40in- | −97.1 | −99.6 |
| | Aflibercept-GCRS(TCC)- | | |
| | hU6-shRNA1-Ang2 | | |
| AMI147 | pFB-scCMV-SV40in- | −96.7 | −99.1 |
| | Aflibercept-GCRS(TCC)- | | |
| | hU6-shRNA2-Ang2 | | |
| AMI148 | pFB-CMV-SV40in- | −100.0 | −100.0 |
| | Aflibercept-GCRS(TCC)- | | |
| | hU6-shRNA3-Ang2 | | |
| AMI149 | pFB-scCMV-SV40in- | 0 | 0 |
| | Aflibercept-GCRS(TCC)- | | |
| | hU6-shRNA-scramble-Ang2 | | |
| AMI150 | pFB-scCMV-SV40in- | −99.8 | −100.0 |
| | Aflibercept-GCRS(TCC)- | | |
| | hU6-shRNA4-Ang2 | | |
| AMI151 | pFB-scCMV-SV40in- | −88.8 | −88.9 |
| | Aflibercept-GCRS(TCC)- | | |
| | hU6-shRNA5-Ang2 | | |
| AMI152 | pFB-scCMV-SV40in- | −98.6 | −99.2 |
| | Aflibercept-GCRS(TCC)- | | |
| | hU6-shRNA6-Ang2 | | |

TABLE 7
shRNA inhibition of angiopoietin 2 expression
in AAV2-N54 transduced HEK293 cells

| Clone | | Decrease of Ang2 expression 4 days |
|---|---|---|
| no. | Self-complementary AAV2.N54 vector | post transduction (%) |
| AMI145 | AAV2.N54-CMV-SV40in-Aflibercept- | −45.2 |
| | GCRS(TCC)-hU6-shRNA1-Ang2 | |
| AMI147 | AAV2.N54-CMV-SV40in-Aflibercept- | −50.6 |
| | GCRS(TCC)-hU6-shRNA2-Ang2 | |
| AMI148 | AAV2.N54-CMV-SV40in-Aflibercept- | −100.0 |
| | GCRS(TCC)-hU6-shRNA3-Ang2 | |
| AMI149 | AAV2.N54-CMV-SV40in-Aflibercept- | 0 |
| | GCRS(TCC)-hU6-shRNA-scrambled | |
| AMI150 | AAV2.N54-CMV-SV40in-Aflibercept- | −98.5 |
| | GCRS(TCC)-hU6-shRNA4-Ang2 | |
| AMI151 | AAV2.N54-CMV-SV40in-Aflibercept- | −62.8 |
| | GCRS(TCC)-hU6-shRNA5-Ang2 | |
| AMI152 | AAV2.N54-CMV-SV40in-Aflibercept- | −74.5 |
| | GCRS(TCC)-hU6-shRNA6-Ang2 | |

Dual Cassettes Worked Better than Fusion Protein Constructs for Target Gene Expressions

In order to target multiple pathways, both VEGF-Trap and angiopoietin 1 (Ang1) genes were cloned into one plasmid flanked by both AAV ITRs in dual cassettes or fusion protein configurations. These plasmids were used to produce AAV2.N54 vectors. In dual cassette configurations, each of the VEGF-Trap and Ang1 was driven respectively by the CMV enhancer/promoter followed by the SV40 intron and terminated by the synthetic poly A sequence. In fusion protein configurations, the VEGF-Trap protein was fused with either Ang1 by Furin and F2A sequences or 4 units of GGGGS linkers. The former configuration yielded two separate proteins after translation by cleavage at the F2A site (VKQTLNFDLLKLAGDVESNPGP, SEQ ID NO: 15). The latter configuration yielded a single fusion protein after translation. The results indicate that dual cassettes yielded higher protein expression for VEGF-Trap (AMI136 and AMI153) than fusion protein constructs either with Furin-F2A (AMI1142 and AMI1154) or 4×GGGGS linker (AMI144). The Furin-F2A cleavage polypeptide sequence comprises a polypeptide sequence of RRKRKQTLNFDLLKLAGDVESNPGP (SEQ ID NO: 16). The dual construct with optimized Ang1 coding sequence (AMI153) yielded higher Ang1 expression than any other constructs but VEGF-Trap was decreased (Table 8) and FIG. 5. Table 9 and Table 10 summarize additional AAVs and their VEGF inhibitory effects combined with either: increased Ang1 expression (Table 9); or decreased Ang2 via Ang2 shRNA (Table 10, Ang2 shRNA 1-6).

TABLE 8
Expression levels of target proteins in
dual or fusion cassette configurations

| | | | hCOMP- |
|---|---|---|---|
| Clone | | Aflibercept | Ang1-FLD |
| no. | AAV | (μg/mL) | (μg/mL) |
| AMI136 | AAV2.N54-CMV-VEGF- | 6.1 | 0.7 |
| | Trap-CMV-Ang1 | | |
| AMI142 | AAV2.N54-CMV-VEGF- | 1.8 | 0.3 |
| | Trap-Furin-F2A-Ang1 | | |
| AMI144 | AAV2-N54-CMV-VEGF- | 1.2 | 0.8 |
| | Trap-4xGGGGS-Ang1 | | |
| AMI153 | AAV2.N54-CMV-VEGF- | 2.7 | 2.6 |
| | Trap-CMV-Ang1-GC | | |
| AMI154 | AAV2.N54-CMV-VEGF- | 1.9 | 0.4 |
| | Trap-Furin-F2A-Ang1-GC | | |

TABLE 9
Expression levels of VEGF and Ang1 in dual or fusion cassette configurations

| | | ANG1 | | | Protein | VEGF- | | |
|---|---|---|---|---|---|---|---|---|
| | | GC | Dual | | (s) | Trap | ANG1 | Selected |
| Construct | Details | optimized | cassette | Linker | expressed | (ng/mL) | (ng/mL) | candidate |
| N54- | AMI054- | NA | NA | NA | VEGF- | 18084.1 | −44.2 | YES |
| AMI120 | AMI120- | | | | Trap | | | (AVMX- |
| | scCMV- | | | | | | | 110) |
| | Aflibercept- | | | | | | | |
| | GCRS(TCC) | | | | | | | |
| N54- | AMI054- | No | NA | NA | ANG1 | −10.1 | 24920 | No |
| AMI071 | AMI071- | | | | | | | |
| | scCMV- | | | | | | | |
| | Ang1- | | | | | | | |
| | FLAG | | | | | | | |
| N54- | AMI054- | Yes | NA | NA | ANG1 | −15.4 | 71625 | No |
| AMI077 | AMI077- | | | | | | | |
| | scCMV- | | | | | | | |
| | Ang1- | | | | | | | |
| | FLAG-GC | | | | | | | |
| N54- | AMI054- | No | Yes | No | VEGF- | 6097.5 | 703.5 | No |
| AMI136 | AMI136- | | | | Trap & | | | |
| | Aflibercept- | | | | ANG1 | | | |
| | GCRS(TCC)- | | | | | | | |
| | Ang1 | | | | | | | |
| N54- | AMI054- | Yes | Yes | No | VEGF- | 2739.9 | 2583.6 | YES |
| AMI153 | AMI153- | | | | Trap & | | | |
| | Aflibercept- | | | | ANG1 | | | |
| | Ang1-GC | | | | | | | |
| N54- | AMI054- | No | No | QBI | VEGF- | 1712.9 | −48 | No |
| AMI143 | AMI143- | | | SP163 | Trap & | | | |
| | Aflibercept- | | | | ANG1 | | | |
| | QBI SP163- | | | | fusion | | | |
| | Ang1 | | | | | | | |
| N54- | AMI054- | No | No | 4GS | VEGF- | 1181.8 | 782.1 | No |
| AMI144 | AMI144- | | | | Trap & | | | |
| | Aflibercept- | | | | ANG1 | | | |
| | 4xGGGGS- | | | | fusion | | | |
| | Ang1 | | | | | | | |
| N54- | AMI054- | Yes | No | Furin | VEGF- | 1930.6 | 399.5 | No |
| AMI154 | AMI153- | | | | Trap & | | | |
| | Aflibercept- | | | | ANG1 | | | |
| | Furin-F2A- | | | | | | | |
| | Ang1-GC | | | | | | | |
| N54- | AMI054- | No | No | Furin | VEGF- | 1805.2 | 321.1 | No |
| AMI142 | AMI142- | | | | Trap & | | | |
| | Aflibercept- | | | | ANG1 | | | |
| | Furin-F2A- | | | | | | | |
| | Ang1 | | | | | | | |

TABLE 10
Expression levels of VEGF and Ang2 in
dual or fusion cassette configurations

| | | VEGF- | | |
|---|---|---|---|---|
| | Protein | Trap | ANG2 | % |
| Construct | Expressed | (ng/mL) | (ng/mL) | Inhibition |
| AMI54- | shRNA 1 + | 1147.7 | 341.8 | −5.2 |
| AMI 145 | VEGF- | | | |
| | trap + | | | |
| | ANG2 | | | |
| AMI54- | ANG2 | −23.9 | 360.5 | 0 |
| AMI 146 | | | | |
| AMI54- | shRNA 2 + | 952.6 | 295.7 | −18 |
| AMI 147 | VEGF- | | | |
| | trap + | | | |
| | ANG2 | | | |
| AMIS4- | shRNA 3 + | 732.2 | 6.7 | −98.1 |
| AMI 148 | VEGF- | | | |
| | Trap + | | | |
| | ANG2 | | | |
| AMI54- | scrambled + | 870.8 | 402.1 | 11.5 |
| AMI 149 | VEGF- | | | |
| | trap + | | | |
| | ANG2 | | | |
| AMI54- | shRNA 4 + | 8458.3 | 152.2 | −57.8 |
| AMI 150 | VEGF- | | | |
| | trap + | | | |
| | ANG2 | | | |
| AMI54- | shRNA 5 + | 1454.5 | 316.8 | −12.1 |
| AMI 151 | VEGF- | | | |
| | trap + | | | |
| | ANG2 | | | |
| AMI54- | shRNA 6 + | 919.7 | 208.8 | −42.1 |
| AMI 152 | VEGF- | | | |
| | trap + | | | |
| | ANG2 | | | |
| HEK293 | NA | −0.3 | 0.83 | |

### Example 2. Therapeutic Efficacy of AAV Vector Comprising the Non-Naturally Occurring Polynucleotide in Laser-Induced Choroidal Neovascularization (CNV) Model in Mice

Example 2 illustrates evaluation of inhibition of neovascularization in a laser-induced model of choroidal neovascularization (CNV) in the mouse (Mus musculus; C57BL/6J; male; 8-12 weeks old) model.

### Treatments

Control Article: AAV2.N54. Δ120 carrying a null mutated AVMX-110 (“sham” vector), will be used at the medium dose, 4e+8 vg/eye.

Test Articles: AAV vectors carrying different transgenes will be evaluated at a concentration of 4e+8 vg/eye. Each vector will be diluted into formulation buffer at 4e+8 vg/μl.

Dosing: The mice will be dosed with the AAVs, bilaterally, 28 days prior to laser. Vehicle will be dosed 3 days prior to laser. The AAV preparations will be withdrawn from the vial with a 5 μm filter (B Braun Filter Needle (FN5120) 5-micron filter in female Luer lock connector with 20 Ga.×1½ in. thin wall needle for withdrawal or injection of medication from rubber-stoppered vials (Product code: 415025) or equivalent filter needle is acceptable. Table 17 illustrates the CNV study experimental design. Table 18 summarizes the test system, including animals, housing, and environmental Conditions. Table 19 illustrates the diet and water provided for the mice used in the CNV study.

TABLE 17
CNV study experimental design

| | | OU | | | | |
|---|---|---|---|---|---|---|
| | No. of | Treatment/ | Volume/ | Treatment | CNV | Experimental |
| Group | Animals | Dose | Route | Day | Induction | Endpoints |
| 1 | 8 | Vehicle | 1.0 μL | Day −28 | OU: CNV | DAY 7: |
| | | Control | IVT | | Laser Day | Fluorescein |
| 2 | 8 | AAV2.N54- | | | 0 | angiography: n = 8 |
| | | Δ120 | | | | mice/group |
| | | 4e+8 vg/eye | | | | Following Day 7 |
| | | “sham” | | | | imaging, serum |
| | | vector | | | | and eyes will be |
| 3 | 8 | AVMX-110 | | | | collected from |
| | | 4e+8 vg/eye | | | | n = 8 mice/group |
| | | Positive | | | | for Sponsor |
| | | control | | | | ELISAs |
| 4 | 8 | AAV1.N54- | | | | |
| | | 120-140 | | | | |
| | | 4e+8 vg/eye | | | | |
| 5 | 8 | AAV6.N54- | | | | |
| | | 120-141 | | | | |
| | | 4e+8 vg/eye | | | | |
| 6 | 8 | AAV2.N54- | | | | |
| | | 120-136 | | | | |
| | | 4e+8 vg/eye | | | | |
| 7 | 8 | AAV2.N54- | | | | |
| | | 120-153 | | | | |
| | | 4e+8 vg/eye | | | | |
| 8 | 8 | AAV2.N54- | | | | |
| | | 120-150 | | | | |
| | | 4e+8 vg/eye | | | | |
| 9 | 8 | AAV2.N54- | | | | |
| | | 120-148 | | | | |
| | | 4e+8 vg/eye | | | | |

CNV: Choroidal Neovascularization,
IVT: Intravitreal,
N/A: not applicable

TABLE 18
Test System: animals, housing, and environmental conditions

| | |
|---|---|
| Species/Strain | Mouse (Mus Musculus)/C57BL/6 |
| Source | Charles River or Taconic Farms |
| Age Range at First Dosing | Approximately 8-12 weeks |
| Weight Range at First Dosing | 20 ± 5 grams |
| Identification | Tail marking, ear punch, and cage card |
| Physical Examination Time | During acclimation |
| Caging | Innovive disposable mouse caging |
| Number per cage | 1-5 |
| Environmental Conditions | Photoperiod: 12 hrs light/12 hrs darkness |
| | Temperature: 68-79° F. |

TABLE 19
Animal Diet and Water

| | | |
|---|---|---|
| Feed | Type | Lab Diet |
| | Name | 5P76 Prolab isopro irradiated |
| | Availability | ad libitum |
| | Analysis for | Not routinely performed, |
| | Contaminants | No contaminants expected |
| Water | Source | Durham City Water |
| | Availability | ad libitum via water bottles |
| | | with sipper tubes. |
| | Analysis for | Not routinely performed, |
| | Contaminants | No contaminants found |

### Animal Health and Acclimation

Animals will be acclimated to the study environment for a minimum of 3 days. At the completion of the acclimation period, each animal will be physically examined by a laboratory animal technician for determination of suitability for study participation. Examinations will include, but will not be limited to, the skin and external ears, eyes, abdomen, behavior, and general body condition. Animals determined to be in good health will be released to the study.

### Randomization and Study Identification

Animals will be randomly assigned to study groups according to facility Standard Operating Procedures (SOPs). Animals will be uniquely identified by corresponding cage card number, ear punch and number.

### Intravitreal Injection

On Day −28 prior to injection, mice will be given buprenorphine 0.01-0.05 mg/kg SQ. Animals will then be tranquilized for the intravitreal injections and one drop of 0.5% proparacaine HCL will be applied to both eyes. Alternatively, mice may be anesthetized with inhaled isoflurane. The conjunctiva will be gently grasped with Dumont #4 forceps, and the injection will be made using a 33 G needle and a Hamilton Syringe. After dispensing the syringe contents, the syringe needle will be slowly withdrawn. Following the injection procedure, 1 drop of Ofloxacin ophthalmic solution will be applied topically to the ocular surface with eye lube.

### Laser-Induced CNV Procedure

On Day 0, mice will be given buprenorphine 0.01-0.05 mg/kg SQ. A topical mydriatic (1.0% Tropicamide HCL, and 2.5% phenylephrine HCL) will be applied at least 15 minutes prior to the laser procedure. The mice will be tranquilized with an intraperitoneal injection of ketamine/xylazine. The cornea will be kept moistened using topical eyewash, and body temperature will be maintained using hot pads. An 532 nm diode laser delivered through a slit-lamp will be used to create 4 single laser spots surrounding the optic nerve. Both mouse eyes will have laser treatment according to the schedule in the Experimental Design on Day 0. Eye lube will be placed after laser.

### Parameters to be Measured

Examination. Mortality and morbidity will be observed daily along with cage-side observations with particular attention paid to both eyes.

Fluorescein angiography (FA). FA will be done on both eyes on Day 7 after laser. Mydriasis for FA will be done using a topical mydriatic (1.0% Tropicamide HCL, and 2.5% phenylephrine HCL; one drop in each eye 15 minutes prior to examination). The mice will be tranquilized with an intraperitoneal injection of ketamine/xylazine. Retinal photography will be performed approximately 1 minute after intravenous sodium fluorescein injection (12 mg/kg).

Euthanasia. At the timepoints in the experimental design table above, animals will be euthanized via carbon dioxide asphyxiation and death will be confirmed by cervical dislocation. Following euthanasia, both eyes of elected animals will be collected for flat mount analysis or for PK tissue analysis.

Ocular Tissue Collection for Homogenization. The eyes will be enucleated, and the retina and RPE/choroid segments will be dissected from fresh eyes and snap frozen. The tissues will be placed into appropriate pre-weighed labeled analytical vials, immediately reweighed to determine sample weight, and placed on dry ice until being transferred to a freezer. Samples will be weighed on a balance capable of measuring out to 4 decimal places. Serum (2 mL polypropylene screw cap tube) and Retina/RPE/Choroid/sclera (“eye cup”) (2 mL Precellys Homogenization Tubes) will be collected Samples will be homogenized in phosphate-buffered saline (PBS). A Precellys Evolution tabletop homogenizer will be used (3×6500 rpm [each cycle 30 sec], delay 30 seconds), and the samples returned to the −80° C. freezer.

### Example 3. In Vitro and In Vivo Bioanalytical Analysis of Non-GLP AVMX-112 (Ang1)

Example 3 illustrates a study for in vitro expression of AVMX-112 (a dual gene construct for expressing Aflibercept and Ang1). In vitro permeability assay using FITC-Dextran showed significant protection from leakage in presence of Ang1. In vivo mouse laser-induced choroidal neovascularization (CNV) model showed significant wound healing with AVMX-112. Table 20 illustrates constructs used in the study utilizing AVMX-112.

TABLE 20
Constructs used in the study utilizing AVMX-112

| | Codon Optimization | | |
|---|---|---|---|
| Constructs code | Aflibercept | ANG1 | Description |
| AMI120 | Yes | NA | scCMV-Aflibercept- |
| (AVMX-110) | | | GCRS(TCC) |
| AMI071 | NA | No | scCMV-Ang1-FLAG |
| AMI077 | NA | Yes | scCMV-Ang1-FLAG-GC |
| AMI136 | Yes | No | ssCMV-Aflibercept- |
| | | | GCRS(TCC)-Ang1 |
| AMI153 | Yes | Yes | ssCMV-Aflibercept- |
| | | | GCRS(TCC)-Ang1-GC |

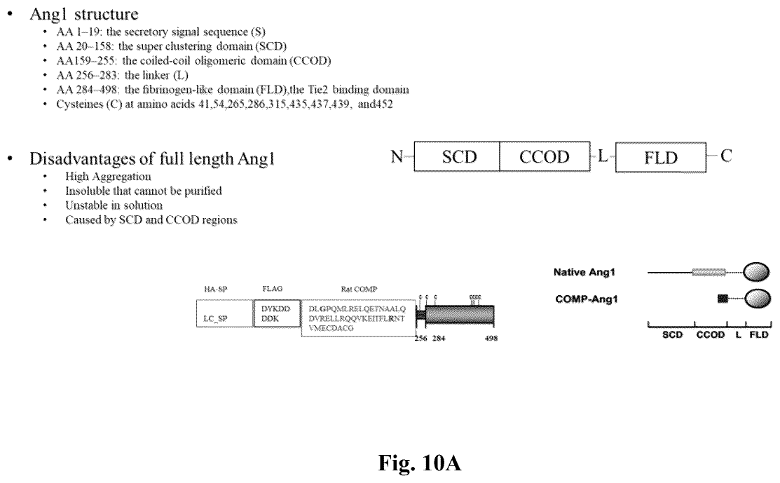

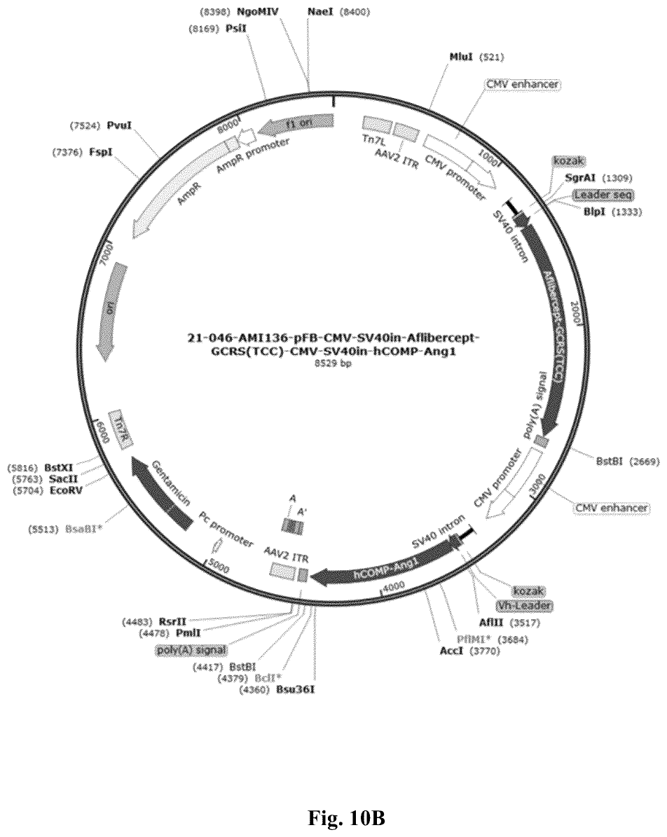

The constructs used in this study expressed dual genes. The common gene of interest (GOI) was Aflibercept (AVMX-110) and human Ang1. Ang1 full length protein tends to form aggregation; hence, shorter sequence had been used for producing Ang1 consisting of aa284-498. The sequence retains a part of coiled-coil domain of rat cartilage oligomeric matrix protein (COMP) on its N-terminus. FIG. 10A illustrates exemplary information about Ang1, COMP-Ang1, and disadvantages of full length Ang1. FIG. 10B illustrates an exemplary dual expression AAV construct. Since each GOI was flanked with two separate CMV promoters, Aflibercept and COMP-Ang1 were expressed as separate proteins. AMI071 and AMI077 were COMP-Ang1 constructs without Aflibercept. They were not dual gene constructs and were used in this study for in vitro expression. AVMX-110 expressed Aflibercept only and had been used for in vitro and in vivo efficacy potency comparison.

### Mechanism of Action

CNV is pathological growth of new blood vessels from the existing choroidal vessels. This leads to loss of vision in late stages. Vascular endothelial growth factor (VEGF) plays a leading role in the pathological progression of CNV. Aflibercept (Eylea) protein is one of the leading protein drugs available to treat CNV. However, Aflibercept and other anti-VEGF drugs have disadvantages such as need for repeated and continuous administration or refractoriness/tachyphylaxis, which is rapid diminishing of response to successive doses. Hence, there is a need for other mechanisms which can support Aflibercept anti-VEGF activity. Angiopoietins especially Ang1 facilitates nonleaky, non-inflammatory, functional, and stable vessels. Ang1 reduces inflammation-induced vascular leakage and inflammatory cell infiltration by tightening cell junctions and reducing adhesion molecules. Table 21 lists materials used in AVMX-112 study. All the AAV constructs for this example were Sf9 produced and underwent two cycles of CsCl ultracentrifugation. Titer of the constructs was checked by using qPCR.

TABLE 21
Materials used in AVMX-112 study

| Material | Vendor | Cat# |
|---|---|---|
| HEK293 | ATCC | CRL-1573 |
| DMEM, high glucose, | Thermofisher Scientific | 10569044 |
| GlutaMAX ™ Supplement, | | |
| pyruvate | | |
| Fetal Bovine Serum | ATCC | 30-2020 |
| VEGF₁₆₅, human HEK293 | GenScript | Z03073 |
| expressed | | |
| Aflibercept | Regeneron | NDC 61755-005-01 |
| Goat pAb Hu IgG (Biotin) | Abcam | ab98618 |
| Streptavidin-HRP | Abcam | ab7403 |
| FITC-CM-Dextran | Millipore Sigma | 53379-IG |
| Nunc maxisorb Flat-bottom 96- | Invitrogen | 44-2404-21 |
| well plate | | |
| 6 Well Cell Culture Plate with | Greiner bio-one/Cellstar | 657 160 |
| lid | | |
| Corning HTS transwell 24 well | Millipore Sigma | CLS3399-12EA |
| permeable supports | | |
| Fisherbrand Surface Treated | Fisher Scientific | FB012937 |
| Sterile Tissue Culture Flasks, | | |
| Vented Cap | | |

### In Vitro Expression and Quantification

HEK293 cells were transduced with AAV constructs mentioned in Table 20. After transduction, supernatants were collected and the Aflibercept and COMP-Ang1 expression level was quantified.

### In Vitro Vascular Permeability Assay

In vitro vascular permeability assay was performed using Human Umbilical Vein Endothelial cells (HUVEC. 5.0E+04 cells were plated onto the apical compartment of 24-well transwell plate in serum free medium. Transwell apical compartment was pre-coated with collagen before plating cells following standard protocol (Application Note 26 “Fabrication of Collagen I Gels,” ibidi USA, Inc., Fitchburg, WI). After incubation for 72 hours in 37° C. incubator, cells were treated with 20 ng/mL VEGF in presence and absence of 253 ng/mL (5 times molar concentration higher than VEGF) Aflibercept and/or 250 ng/mL (5 times molar concentration higher than VEGF) COMP-Ang1 proteins for one hour. After treatment, the medium was placed with fresh medium in the basolateral chamber, and 1 mg/mL FITC-Dextran was added to the apical chamber and incubated for 30 minutes. After 30 minutes, 50 μL was taken from the basolateral compartment and supplemented with 300 μL of phenol red free DMEM. 100 μL of this sample was transferred onto black 96-well plates. Reading was measured in triplicates with fluorescence intensity at 490/520 nm excitation/emission spectrum.

### In Vivo Mouse CNV Model

For in vivo studies, as described before mouse CNV model was used and different constructs were inject intravitreosly (IVT) at a dose of 4E+08 vg/eye. AAV constructs were injected 28 days prior to the FA analysis of the ANG1 constructs, its comparison with AVMX-110 and Aflibercept expression in serum and ocular samples obtained from animals that were injected intravitreously (IVT) with non-GLP AAV constructs with different gene products for efficacy study in mouse CNV model. The titer injected IVT was 4.8E+08 vg/eye for all the groups. Study conducted involved groups shown in Table 22.

TABLE 22
List of AAV constructs/controls injected
IVT and serum/ocular samples received

| | | | No. of | No. of |
|---|---|---|---|---|
| | | Gene | serum | ocular |
| Group | Construct | Product | samples | samples |
| 1 | Vehicle | NA | 7 | 14 |
| 2 | AAV2.N54-Δ120 | None | 8 | 16 |
| 3 | AAV2.N54-120 | Aflibercept | 8 | 16 |
| 4 | AAV2.N54-120-136 | Aflibercept + ANG1 | 6 | 12 |
| 5 | AAV2.N54-120-153 | Aflibercept + ANG1 | 7 | 14 |

### Homogenization of Ocular Samples

Ocular and serum samples were obtained from after euthanasia. The homogenized tissues were further sonicated by keeping the samples on ice and sonication for 20 second with an interval of 20 seconds thrice. Sonicated ocular samples were then centrifuged at 13,000 rpm for 3-4 minutes. The supernatant was then collected and used to determine the Aflibercept levels using standardized VEGF-Trap ELISA mentioned in the introduction section.

### In Vitro Expression in HEK293 Cells

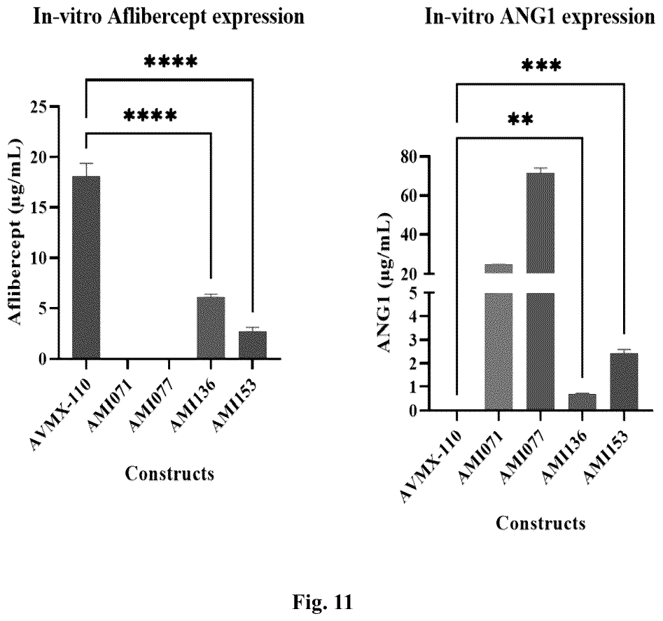

Aflibercept and Ang1 expression in dual gene constructs was compared with single gene constructs (FIG. 11). Table 23 illustrates the expression profile of Aflibercept and Ang1 in all the constructs used in the study. Non-optimized ANG1 construct (AMI136) produced higher concentration of Aflibercept but lower concentration of Ang1, but AMI153 had equal expression of Aflibercept and ANG1. Statistical analysis was performed using GraphPad Prism software.

TABLE 23
Aflibercept and ANG1 expression in vitro

| | Codon Optimization | | Aflibercept | ANG1 |
|---|---|---|---|---|
| Constructs code | Aflibercept | ANG1 | (μg/mL) | (μg/mL) |
| AMI120 (AVMX-110) | Yes | NA | 18.1 ± 1.3 | NA |
| AMI071 | NA | No | NA | 24.9 ± 0.1 |
| AMI077 | NA | Yes | NA | 71.7 ± 2.6 |
| AMI136 | Yes | No | 6.1 ± 0.3 | 0.7 ± 0.0 |
| AMI153 | Yes | Yes | 2.8 ± 0.4 | 2.4 ± 0.2 |

### In Vitro Vascular Permeability Assay

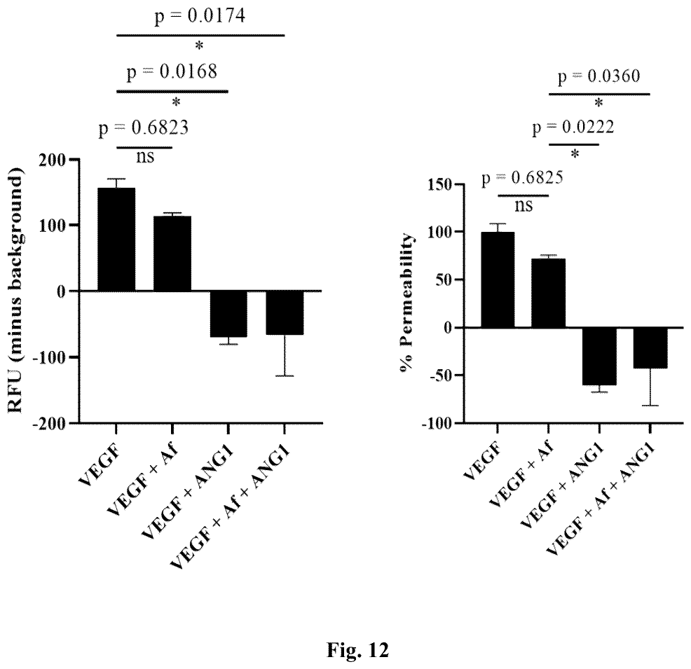

In vitro permeability assay showed the effect of different proteins on the permeability of FITC-dextran to pass through the HUVEC cells monolayer. Purified protein had been used in the assay instead of AAV constructs. FIG. 12 illustrates that VEGF promoted leakage but Aflibercept and Ang1 acted to reduce the leakage of FITC dextran. Ang1 had significantly higher leakage protection when compared to Aflibercept alone. Together, Aflibercept and Aed1 acted to significantly reduce permeability compared to VEGF or VEGF in combination with Aflibercept.

### Fluorescein Angiography (FA) Analysis

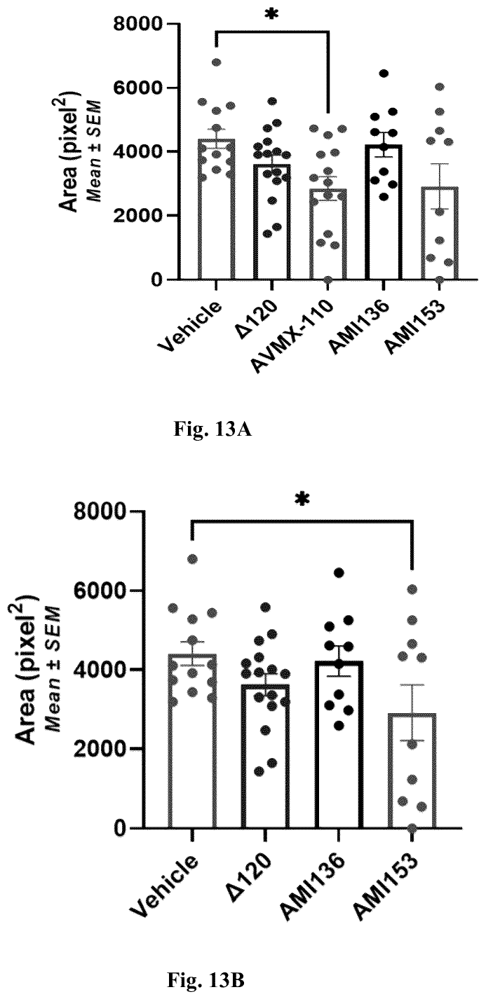

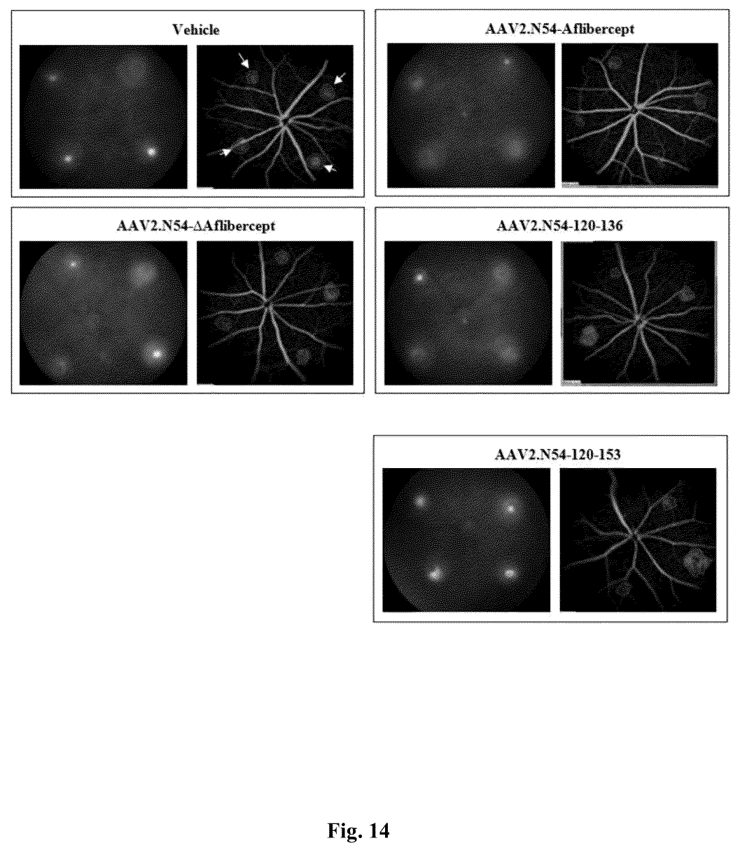

FA data after laser injury was compared between the groups. Statistical analysis was performed by comparing every other group to vehicle control. FIG. 13A and FIG. 13B show the results of Ang1 and VEGF-Trap constructs comparison as bar graph±SEM and statistical analysis using one-way ANOVA and multiple comparison using Dunnett testing. FIG. 14 illustrates representative FA images from different groups. When vehicle was used to compare rest of the groups, there was no significant difference between vehicle and sham control groups as expected. However, AAV2.N54-120 group of animals showed significant laser injury recovery. When AAV2.N54-120 group animals were excluded from the analysis, AAV2.N54-153 animals also showed significant difference from the vehicle group animals. The area of lesion with p values for different groups is summarized in Table 24.

TABLE 24
Comparison of lesion area for different study groups

| | | Lesion Area | p value | p value |
|---|---|---|---|---|
| Group | Treatment | (pixel2) ± S.D. | (+AVMX-110) | (−AVMX-110) |
| 1 | Vehicle | 4410.5 ± 1083 | — | — |
| 2 | AAV2.N54-ΔAflibercept | 3620.4 ± 1114.3 | 0.3894 (ns) | 0.3274 (ns) |
| 3 | AAV2.N54-Aflibercept | 2852.2 ± 1443.5 | 0.0195 (*) | — |
| 4 | AAV2.N54-120-136 | 4221.9 ± 1216.1 | 0.9937 (ns) | 0.9795 (ns) |
| 5 | AAV2.N54-120-153 | 2915.1 ± 2234.3 | 0.0526 (ns) | 0.0429 (*) |

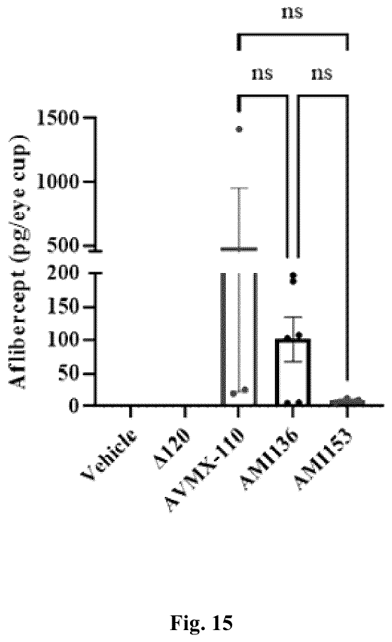

VEGF-Trap concentration was expressed in pg of Aflibercept per eye cup. Eye cup consisted of retina, sclera, choroid and retina (FIG. 15). The expression in AVMX-110, which was AAV2.N54-Aflibercept, showed higher level of Aflibercept expression compared to other groups (Table 25).

TABLE 25
Aflibercept expression in ocular samples

| Construct | Aflibercept (pg/eye cup) |
|---|---|
| Vehicle | <LoQ |
| AAV2.N54-ΔAflibercept | <LoQ |
| AAV2.N54-Aflibercept | 485.1 ± 802.1 |
| AAV2.N54-120-136 | 100.7 ± 83.4 |
| AAV2.N54-120-153 | 9.3 ± 3.3 |

LoQ = Limits of Detection

In conclusion, in vitro expression showed significant increase in expression of Ang1 after codon optimization whether in single or dual gene constructs. In vitro permeability assay also showed significant leakage protection by Ang1. Aflibercept and Ang1 worked synergistic to reduce the leakage caused by VEGF. AVMX-110 efficiently reduced the lesion area in mouse CNV model. Ang1 construct AMI153 also showed efficacy comparable to AVMX-110 even though Aflibercept expression of AMI153 was much lower than AVMX-110. However, AMI136, which had higher Aflibercept expression compared to AMI153, didn't show efficacy. This illustrates the importance of Ang1 in this model.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A non-naturally occurring polynucleotide comprising one or more expression cassettes for expressing:
a) a VEGF inhibitor; and
b) a receptor tyrosine kinase (RTK)/Tie2 or an activator of RTK/Tie2.

2. The non-naturally occurring polynucleotide of claim 1, wherein a) and b) are expressed as separate polypeptides or as a contiguous polypeptide cleavable into separate polypeptides comprising the VEGF inhibitor, and the RTK/Tie2 or the activator of RTK/Tie2.

3. The non-naturally occurring polynucleotide of claim 2, wherein the contiguous polypeptide comprises a protease cleavable sequence, a Furin cleavable sequence, or a self-cleaving polypeptide sequence.

4.-9. (canceled)

10. The non-naturally occurring polynucleotide of claim 1, wherein the VEGF inhibitor comprises an anti-VEGF antibody or VEGF-TRAP.

11.-15. (canceled)

16. The non-naturally occurring polynucleotide of claim 1, wherein the activator of the RTK/Tie2 comprises a angiopoietin-1 (Ang-1), angiopoietin-2 (Ang-2), angiopoietin-3 (Ang-3), angiopoietin-4 (Ang-4), or a functional fragment of any one thereof.

17. (canceled)

18. (canceled)

19. The non-naturally occurring polynucleotide of claim 16, wherein the Ang-1 comprises or consists of a polypeptide sequence that is at least 70%, at least 75%, at least 80%, at least 85%, at least 90%, at least 95%, or at least 99% identical to SEQ ID NO: 3.

20. (canceled)

21. The non-naturally occurring polynucleotide of claim 16, wherein the functional fragment of the Ang-1 comprises or consists of a fibronectin-like domain (FLD).

22. The non-naturally occurring polynucleotide of claim 21, wherein the FLD comprises or consists of a polypeptide sequence that is at least 70%, at least 75%, at least 80%, at least 85%, at least 90%, at least 95%, or at least 99% identical to SEQ ID NO: 5.

23. The non-naturally occurring polynucleotide of claim 21, wherein the FLD is fused to a soluble polypeptide.

24. (canceled)

25. The non-naturally occurring polynucleotide of claim 23, wherein the soluble polypeptide comprises or consists of a polypeptide sequence that is at least 70%, at least 75%, at least 80%, at least 85%, at least 90%, at least 95%, at least 99%, or more identical to SEQ ID NO: 2 or SEQ ID NO: 1.

26. (canceled)

27. The non-naturally occurring polynucleotide of claim 1, wherein the activator of the RTK/Tie2 is at least 70%, at least 75%, at least 80%, at least 85%, at least 90%, at least 95%, or at least 99% identical to SEQ ID NO: 6.

28. The non-naturally occurring polynucleotide of claim 1, wherein the activator of the RTK/Tie2 comprises an antibody or a fragment thereof.

29. (canceled)

30. The non-naturally occurring polynucleotide of claim 1, wherein the activator of the RTK/Tie2 binds to and inhibits Ang-2.

31. (canceled)

32. The non-naturally occurring polynucleotide of claim 1, wherein the antibody or the fragment thereof comprises or consists of a polypeptide sequence that is at least 70%, at least 75%, at least 80%, at least 85%, at least 90%, at least 95%, or at least 99% identical to any one of SEQ ID NOs: 139-141, a fragment thereof, or a combination thereof.

33. The non-naturally occurring polynucleotide of claim 1, wherein the activator of the RTK/Tie2 comprises an inhibitory RNA.

34. The non-naturally occurring polynucleotide of claim 33, wherein the inhibitory RNA comprises a shRNA, siRNA, miRNA, or a combination thereof.

35.-38. (canceled)

39. The non-naturally occurring polynucleotide of claim 1, wherein the one or more expression cassettes comprise one or more promoters, one or more internal ribosome entry sites (IRES), or both.

40.-72. (canceled)

73. A viral vector comprising the non-naturally occurring polynucleotide of claim 1.

74. The viral vector of claim 73, wherein the viral vector is an adeno-associated viral (AAV) vector.

75.-81. (canceled)

82. A method for treating a disease or a condition in a subject in need thereof, the method comprising administering to the subject a therapeutically effective amount of the non-naturally occurring polynucleotide of claim 1, thereby treating the disease or the condition in the subject.

83.-95. (canceled)
